(19) United States
(12) Patent Application Publication
(10) Pub. No.: US 2023/0309387 A1
(43) Pub. Date: Sep. 28, 2023

(54) ORGANIC ELECTROLUMINESCENT MATERIAL, A PLURALITY OF HOST MATERIALS, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Jeong-Eun YANG, Gyeonggi-do (KR); Hee-Ryong KANG, Gyeonggi-do (KR); Jeong-Hwan JEON, Gyeonggi-do (KR); Mi-Ja LEE, Gyeonggi-do (KR); Sang-Hee CHO, Gyeonggi-do (KR); Young-Jae KIM, Gyeonggi-do (KR)

(21) Appl. No.: 18/181,832

(22) Filed: Mar. 10, 2023

(30) Foreign Application Priority Data
Mar. 11, 2022 (KR) 10-2022-0030613

(51) Int. Cl. H10K 85/60; C07D 413/12; C09K 11/06

(52) U.S. Cl. CPC H10K 85/636; C07D 413/12; C09K 11/06; H10K 85/622; H10K 85/654; H10K 85/6574; H10K 50/11

(57) ABSTRACT

The present disclosure relates to an organic electroluminescent material comprising an organic electroluminescent compound represented by formula 1, and an organic electroluminescent device comprising the same; and a plurality of host materials comprising at least one first host compound and at least one second host compound, and an organic electroluminescent device comprising the same. An organic electroluminescent device with improved driving voltage and/or luminous efficiency can be provided by comprising the compound according to the present disclosure as an organic electroluminescent material or a plurality of host materials.

### TECHNICAL FIELD

The present disclosure relates to an organic electroluminescent material, a plurality of host materials, and organic electroluminescent device comprising the same.

### BACKGROUND ART

In 1987, Tang et al. of Eastman Kodak first developed a small molecular green organic electroluminescent device (OLED) by using TPD/Alq₃ bilayer consisting of a light-emitting layer and a charge transport layer. Thereafter, the development of OLEDs was rapidly effected and OLEDs have been commercialized. Currently, OLEDs mainly use phosphorescent materials having excellent luminous efficiency in panel implementation. OLEDs having high luminous efficiency and/or long lifespan are required for long-term use and high resolution of a display.

In order to improve luminous efficiency, driving voltage, and/or lifespan, various materials or concepts for an organic layer of an organic electroluminescent device have been proposed, but they were not satisfactory in practical use. Accordingly, there has been a continuous need to develop an organic electroluminescent device having more improved performances, for example, improved driving voltage, luminous efficiency, power efficiency, and/or lifespan properties, compared to an organic electroluminescent device previously disclosed.

On the other hand, Korean Patent Application Laid-Open Nos. 2018-0055206 and 2019-0002951 disclose a compound having the structure in which one or more substituents linked directly or via a linker group are linked to a moiety having a 5-membered aromatic ring or a 5-membered aromatic heterocyclic ring bonded, as a basic backbone. However, the aforementioned references fail to specifically disclose organic electroluminescent materials comprising specific compounds claimed in the present disclosure or host materials of a combination of specific compounds.

### DISCLOSURE OF INVENTION

### Technical Problem

An object of the present disclosure is to provide an organic electroluminescent material and a plurality of host materials capable of producing an organic electroluminescent device with low driving voltage and/or high luminous efficiency. Another object of the present disclosure is to provide an organic electroluminescent device having low driving voltage and/or high luminous efficiency by comprising the organic electroluminescent material or the plurality of host materials according to the present disclosure.

### Solution to Problem

As a result of intensive research to solve the above technical problems, the present inventors found that the above objective can be achieved by an organic electroluminescent material comprising a compound represented by the following formula 1. In addition, the present inventors found that the above objective can be achieved by a plurality of host materials comprising at least one first host compound and at least one second host compound, wherein the first host compound is represented by the following formula 1, and the second host compound is represented by the following formula 2.

The compound represented by formula 1 of the present disclosure may be applied to an organic electroluminescent device as an organic electroluminescent material or as a plurality of host materials in combination with a compound represented by following formula 2.

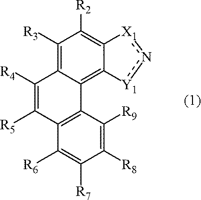

In formula 1,
X₁ and Y₁ each independently represent —O—, —S— or -CR₁=;
provided that one of X₁ and Y₁ represents -CR₁=, and the other of X₁ and Y₁ represents —O— or —S—;
R₁ represents a substituted or unsubstituted (C1-C30)alkyl, a substituted or unsubstituted (C6-C30)aryl, or a substituted or unsubstituted (3- to 30-membered)heteroaryl;
R₂ to R₉ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of (C3-C30) aliphatic ring and (C6-C30) aromatic ring, a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted mono- or di- (C6-C30)arylamino, a substituted or unsubstituted (C1-C30)alkyl(C6-C30)arylamino, a substituted or unsubstituted mono- or di- (3- to 30-membered)heteroarylamino, a substituted or unsubstituted (C1-C30)alkyl(3- to 30-membered)heteroarylamino, a substituted or unsubstituted (C2-C30)alkenyl(C6-C30) arylamino, a substituted or unsubstituted (C2-C30)alkenyl(3- to 30-membered)heteroarylamino, a substituted or unsubstituted (C6-C30)aryl(3- to 30-membered)heteroarylamino, or -L₁-N(Ar₁)(Ar₂) or may be linked to an adjacent substituent(s) to form a ring(s);
L₁ each independently represents a single bond, a substituted or unsubstituted (C6-C30)arylene, or a substituted or unsubstituted (3- to 30-membered)heteroarylene; and
Ar₁ and Ar₂ each independently represent a substituted or unsubstituted (C6-C30) aryl, or a substituted or unsubstituted (3- to 30-membered)heteroaryl;

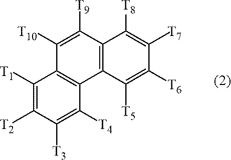

in formula 2,
   at least one of T₅ and T₆, and T₇ and T₈ is linked to each other to form a ring together with the following formula 3; 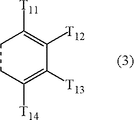
in formulas 2 and 3,
   T₁ to T₄, T₉ to T₁₄, and T₅ to T₈ that do not form a ring(s) together with formula 3 each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted mono- or di- (C6-C30)arylamino, a substituted or unsubstituted (C1-C30)alkyl(C6-C30)arylamino, a substituted or unsubstituted mono- or di- (3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted (C6-C30)aryl(3- to 30-membered)heteroarylamino, or -L₂-Ar₃, provided that at least one of T₁ to T₁₄ represents -L₂-Ar₃;
   L₂ each independently represents a single bond, a substituted or unsubstituted (C6-C30)arylene, or a substituted or unsubstituted (3- to 30-membered)heteroarylene;
   Ar₃ each independently represents a substituted or unsubstituted (C6-C30)aryl or a substituted or unsubstituted (3- to 30-membered)heteroaryl; 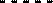
   represents a fusion site with formula 2;
   the heteroaryl and the heteroarylene contain one or more heteroatoms selected from B, N, O, S, Si and P.

### Advantageous Effects of Invention

The organic electroluminescent material and the plurality of host materials according to the present disclosure exhibit performance suitable for use in an organic electroluminescent device. In addition, an organic electroluminescent device having lower driving voltage and/or higher luminous efficiency compared to a conventional organic electroluminescent device may be manufactured by comprising compounds according to the present disclosure as an organic electroluminescent material or as a plurality of host materials, and it is possible to manufacture a display system or a lighting system using the same.

### Mode for the Invention

Hereinafter, the present disclosure will be described in detail. However, the following description is intended to explain the present disclosure, and is not meant to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of organic electroluminescent materials” in the present disclosure means an organic electroluminescent material(s) comprising a combination of two or more compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of two or more compounds that may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. The two or more compounds may be comprised in the same layer or different layers, and may be mixture-evaporated or co-evaporated, or may be individually evaporated.

The term “a plurality of host materials” in the present disclosure means an organic electroluminescent material(s) comprising a combination of two or more host materials. It may mean both a material before being comprised in an organic electroluminescent device (for example, before vapor deposition) and a material after being comprised in an organic electroluminescent device (for example, after vapor deposition). The plurality of host materials of the present disclosure may be comprised in any light-emitting layer constituting an organic electroluminescent device, wherein the two or more compounds comprised in the plurality of host materials may be comprised together in one light-emitting layer or may be respectively comprised in different light-emitting layers. When two or more host materials are comprised in one layer, for example, they may be mixture-evaporated to form a layer or separately and simultaneously co-evaporated to form a layer.

Herein, the term “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. The term “(C2-C30)alkenyl” in the present disclosure is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” in the present disclosure is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl” is meant to be a monocyclic or polycyclic hydrocarbon having 3 to 30 ring backbone carbon atoms, and preferably 3 to 20 ring backbone carbon atoms, more preferably 3 to 7 ring backbone carbon atoms. Examples of the cycloalkyl may include cyclopropyl, cyclobutyl, cyclopentyl, cyclohexyl, cyclopentylmethyl, cyclohexylmethyl, etc. The term “(3- to 7-membered)heterocycloalkyl” in the present disclosure is meant to be a cycloalkyl having 3 to 7 ring backbone atoms, preferably 5 to 7 ring backbone atoms and containing at least one heteroatom(s) selected from the group consisting of B, N, O, S, Si, and P, preferably the group consisting of O, S, and N. The above heterocycloalkyl includes tetrahydrofuran, pyrrolidine, thiolane, tetrahydropyran, etc. The term “(C6-C30)aryl(ene)” in the present disclosure is meant to be a monocyclic or fused ring radical derived from an aromatic hydrocarbon having 6 to 30 ring backbone carbon atoms. The number of ring backbone carbon atoms is preferably 6 to 25, and more preferably 6 to 18. The above aryl may be partially saturated, and may comprise a spiro structure. The above aryl may include phenyl, biphenyl, terphenyl, naphthyl, binaphthyl, phenylnaphthyl, naphthylphenyl, phenylterphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, azulenyl, tetramethyldihydrophenanthrenyl, etc. More specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenylyl, 4″-t-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl(ene)” in the present disclosure is meant to be an aryl having 3 to 30 ring backbone atoms and including at least one, preferably 1 to 4 heteroatom(s) selected from the group consisting of B, N, O, S, Si, and P. It may be a monocyclic ring or a fused ring condensed with at least one benzene ring, and may be partially saturated. In addition, the above heteroaryl(ene) comprises one formed by linking at least one heteroaryl or aryl group to a heteroaryl group via a single bond(s), and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, dibenzoselenophenyl, naphthobenzofuranyl, naphthobenzothiophenyl, benzofuroquinolyl, benzofuroquinazolinyl, benzofuronaphthyridinyl, benzofuropyrimidinyl, naphthofuropyrimidinyl, benzothienoquinolyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphtofuropyrazinyl, benzothienopyrazinyl, naphthothienopyrazinyl, acenaphthopyrimidinyl, pyrazinoindolyl, benzopyrazinoindolyl, benzoimidazolyl, benzothiazolyl, benzoisothiazolyl, benzoisoxazolyl, benzooxazolyl, naphthooxazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, dibenzoquinoxalinyl, naphthyridinyl, carbazolyl, benzocarbazolyl, dibenzocarbazolyl, phenoxazinyl, phenothiazinyl, phenanthridinyl, phenanthroimidazolyl, benzodioxolyl, dihydroacridinyl, benzotriazolephenazine, imidazopyridine, chromenoquinazolinyl, thiochromenoquinazolinyl, dimethylbenzoperimidinyl, indolocarbazolyl, indenocarbazolyl, etc. More specifically, the heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1- pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. The term “a fused ring group of a (C3-C30)aliphatic ring(s) and a (C6-C30) aromatic ring(s)” is meant to be a functional group in which at least one aliphatic ring(s) having 3 to 30 ring backbone carbon atoms, preferably 3 to 25 ring backbone carbon atoms, and more preferably 3 to 18 ring backbone carbon atoms and at least one aromatic ring(s) having 6 to 30 ring backbone carbon atoms, preferably 6 to 25 ring backbone carbon atoms, and more preferably 6 to 18 ring backbone carbon atoms are fused. For example, the fused ring group may include a fused ring group of at least one benzene and at least one cyclohexane, or a fused ring group of at least one naphthalene and at least one cyclopentane, etc. In the present disclosure, the carbon atom of the fused ring group of a (C3-C30) aliphatic ring(s) and a (C6-C30) aromatic ring(s) may be replaced with at least one heteroatom(s) selected from B, N, O, S, Si, and P, preferably at least one heteroatom(s) selected from N, O, and S. In the present disclosure, “halogen” includes F, Cl, Br, and l.

In addition, “ortho (o-),” “meta (m-),” and “para (p-)” are prefixes, which represent the relative positions of substituents, respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

The term “a ring formed by a linkage of adjacent substituents” means that at least two adjacent substituents are linked to or fused with each other to form a substituted or unsubstituted mono- or polycyclic (3- to 30-membered) alicyclic or aromatic ring, or the combination thereof, preferably a substituted or unsubstituted mono- or polycyclic (5- to 25-membered) alicyclic or aromatic ring, or the combination thereof. In addition, the formed ring may contain at least one heteroatom selected from B, N, O, S, Si, and P, preferably at least one heteroatom selected from N, O, and S. According to one embodiment of the present disclosure, the number of ring backbone atoms is 5 to 20, and according to another embodiment of the present disclosure, the number of ring backbone atoms is 5 to 15.

In addition, “substituted” in the expression “substituted or unsubstituted” means that a hydrogen atom in a certain functional group is replaced with another atom or another functional group (i.e., a substituent), and also includes that the hydrogen atom is replaced with a group formed by a linkage of two or more substituents of the above substituents. For example, the “group formed by a linkage of two or more substituents” may be pyridine-triazine. That is, pyridine-triazine may be interpreted as a heteroaryl substituent, or as a substituent in which two heteroaryls are linked. Herein, the substituent(s) of the substituted alkyl, the substituted aryl, the substituted arylene, the substituted heteroaryl, the substituted heteroarylene, the substituted cycloalkyl, the substituted alkoxy, the substituted trialkylsilyl, the substituted dialkylarylsilyl, the substituted alkyldiarylsilyl, the substituted triarylsilyl, the substituted fused ring group of an aliphatic ring(s) and an aromatic ring(s), the substituted mono- or di- alkylamino, the substituted mono- or di- alkenylamino, the substituted alkylalkenylamino, the substituted mono- or di- arylamino, the substituted alkylarylamino, the substituted mono- or di- heteroarylamino, the substituted alkylheteroarylamino, the substituted alkenylarylamino, the substituted alkenylheteroarylamino, and the substituted arylheteroarylamino in the formulas of the present disclosure each independently are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of a (C1-C30)alkyl(s) and a (C6-C30)aryl(s); a (C6-C30)aryl unsubstituted or substituted with at least one of deuterium, a cyano(s), a halogen(s), a (C1-C30)alkyl(s), a (C3-C30)cycloalkyl(s), a tri(C1-C30)alkylsilyl(s), a tri(C6-C30)arylsilyl(s), a (C6-C30)aryl(s), and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di- (C1-C30)alkylamino; a mono- or di- (C2-C30)alkenylamino; a (C1-C30)alkyl(C2-C30)alkenylamino; a mono- or di- (C6-C30)arylamino; a (C1-C30)alkyl(C6-C30)arylamino; a mono- or di- (3- to 30-membered)heteroarylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituent(s), each independently, are at least one selected from the group consisting of a (C1-C20)alkyl; a (3- to 30-membered)heteroaryl unsubstituted or substituted with a (C6-C25)aryl(s); and a (C6-C30)aryl unsubstituted or substituted with at least one of deuterium, a cyano(s), a halogen(s), a (C1-C20)alkyl(s), a (C3-C15)cycloalkyl(s), a tri(C1-C20)alkylsilyl(s), a tri(C6-C25)arylsilyl(s), a (C6-C28)aryl(s) and a (3- to 25-membered)heteroaryl(s). According to another embodiment of the present disclosure, the substituent(s), each independently, are at least one selected from the group consisting of a (C1-C10)alkyl; a (3- to 28-membered)heteroaryl unsubstituted or substituted with a (C6-C18)aryl(s); and a (C6-C30)aryl unsubstituted or substituted with at least one of deuterium, a cyano(s), a halogen(s), a (C1-C10)alkyl(s), a (C3-C10)cycloalkyl(s), a tri(C1-C10)alkylsilyl(s), a tri(C6-C18)arylsilyl(s), a (C6-C18)aryl(s) and a (3- to 18-membered)heteroaryl(s). Specifically, the substituent(s), each independently, may be at least one selected from the group consisting of a phenyl unsubstituted or substituted with at least one of a methyl(s), a t-butyl(s), a cyclohexane(s), a fluoro(s), a naphthyl(s), a carbazolyl(s), a trimethylsilyl(s), a triphenylsilyl(s), and a cyano(s); a biphenyl; a terphenyl; a naphthyl unsubstituted or substituted with a phenyl(s); a phenanthrenyl; a chrysenyl; an anthracenyl; a triphenylenyl; a dimethylfluorenyl; a diphenylfluorenyl; a spirobifluorenyl; a (22-membered)aryl; a dibenzofuranyl unsubstituted or substituted with at least one of a phenyl(s) and a biphenyl(s); a dibenzothiophenyl; a carbazolyl unsubstituted or substituted with a phenyl(s); a phenoxazinyl; a benzothiophenyl; a naphthooxazolyl unsubstituted or substituted with a phenyl(s); and a pyridyl unsubstituted or substituted with a phenyl(s).

An organic electroluminescent material according to one embodiment of the present disclosure comprises the compound represented by formula 1.

Hereinafter, the compound represented by formula 1 will be described in more detail.

In formula 1, X₁ and Y₁ each independently represent —O—, —S— or -CR1=, provided that one of X₁ and Y₁ represents -CR1=, and the other of X₁ and Y₁ represents —O— or —S— .

In formula 1, R₁ represents a substituted or unsubstituted (C1-C30)alkyl, a substituted or unsubstituted (C6-C30)aryl, or a substituted or unsubstituted (3- to 30-membered)heteroaryl. According to one embodiment of the present disclosure, R₁ represents a substituted or unsubstituted (C1-C20)alkyl, a substituted or unsubstituted (C6-C18)aryl, or a substituted or unsubstituted (3- to 25-membered)heteroaryl. According to another embodiment of the present disclosure, R₁ represents an unsubstituted (C1-C10)alkyl, an unsubstituted (C6-C15)aryl, or an unsubstituted (3- to 18-membered)heteroaryl. For example, R₁ may be a t-butyl, a phenyl, a naphthyl, a pyridyl, a dibenzofuranyl, etc.

In formula 1, R₂ to R₉ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di- (C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di- (C6-C30)arylamino, a substituted or unsubstituted (C1-C30)alkyl(C6-C30)arylamino, a substituted or unsubstituted mono- or di- (3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted (C6-C30)aryl(3- to 30-membered)heteroarylamino, or -L₁-N(Ar₁)(Ar₂), or may be linked to an adjacent substituent(s) to form a ring(s). According to one embodiment of the present disclosure, R₂ to R₉ each independently represent hydrogen, deuterium, a substituted or unsubstituted (C6-C28)aryl, a substituted or unsubstituted (3- to 25-membered)heteroaryl, or -L₁-N(Ar₁)(Ar₂). According to another embodiment of the present disclosure, R₂ to R₉ each independently represent hydrogen; deuterium; a (C6-C20)aryl unsubstituted or substituted with a (C6-C18)aryl(s); a (6- to 20-membered)heteroaryl unsubstituted or substituted with at least one of a (C6-C18)aryl(s) and a (6- to 20-membered)heteroaryl(s); or -L₁-N(Ar₁)(Ar₂). For example, R₂ to R₉ may each independently be hydrogen; deuterium; a phenyl; a naphthyl; an anthracenyl unsubstituted or substituted with a phenyl(s); a dibenzofuranyl; a carbazolyl unsubstituted or substituted with a phenyl(s); a pyrimidinyl unsubstituted or substituted with a phenyl(s); a triazinyl unsubstituted or substituted with at least one of a phenyl(s) and a dibenzofuranyl(s); -L₁-N(Ar₁)(Ar₂), etc.

L₁ each independently may be a single bond, a substituted or unsubstituted (C6-C30)arylene, or a substituted or unsubstituted (3- to 30-membered)heteroarylene. For example, L₁ may be a single bond.

Ar₁ and Ar₂ each independently represent a substituted or unsubstituted (C6-C30)aryl, or a substituted or unsubstituted (3- to 30-membered)heteroaryl. According to one embodiment of the present disclosure, Ar₁ and Ar₂ each independently may be a substituted or unsubstituted (C6-C18)aryl, or a substituted or unsubstituted (3- to 20-membered)heteroaryl. According to another embodiment of the present disclosure, Ar₁ and Ar₂ each independently may be an unsubstituted (C6-C15)aryl, or an unsubstituted (3- to 15-membered)heteroaryl. For example, Ar₁ and Ar₂ each independently may be a phenyl, a biphenyl, a dibenzofuranyl, etc.

According to another embodiment of the present disclosure, R₁, Ar₁ and Ar₂ each independently may be a substituted or unsubstituted phenyl, a substituted or unsubstituted naphthyl, a substituted or unsubstituted biphenyl, a substituted or unsubstituted terphenyl, a substituted or unsubstituted phenanthrenyl, a substituted or unsubstituted anthracenyl, a substituted or unsubstituted fluorenyl, a substituted or unsubstituted benzofluorenyl, substituted or unsubstituted triphenylenyl, substituted or unsubstituted spirobifluorenyl, a substituted or unsubstituted pyridyl, a substituted or unsubstituted triazinyl, a substituted or unsubstituted pyrimidinyl, a substituted or unsubstituted quinolyl, a substituted or unsubstituted quinazolinyl, a substituted or unsubstituted quinoxalinyl, a substituted or unsubstituted benzoquinazolinyl, a substituted or unsubstituted benzoquinoxalinyl, a substituted or unsubstituted benzofuropyrimidinyl, a substituted or unsubstituted carbazolyl, a substituted or unsubstituted dibenzothiophenyl, a substituted or unsubstituted benzothiophenyl, a substituted or unsubstituted dibenzofuranyl, a substituted or unsubstituted benzofuranyl, a substituted or unsubstituted naphthyridinyl, a substituted or unsubstituted benzonaphthofuranyl, or a substituted or unsubstituted benzonaphthothiophenyl.

According to one embodiment of the present disclosure, formula 1 may be represented by the following formula 4.

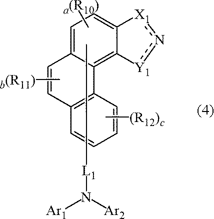

In formula 4, R₁₀ to R₁₂ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di- (C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di- (C6-C30)arylamino, a substituted or unsubstituted (C1-C30)alkyl(C6-C30)arylamino, a substituted or unsubstituted mono- or di- (3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or may be linked to an adjacent substituent(s) to form a ring(s); a and b each independently represent an integer of 1 or 2, and c represents an integer of 1 to 4, where if a to c are an integer of 2 or more, each of R₁₀ to each of R₁₂ may be the same or different; and X₁, Y₁, L₁, Ar₁, and Ar₂ are as defined in formula 1.

The compound represented by formula 1 may be at least one selected from the following compounds, but is not limited thereto.

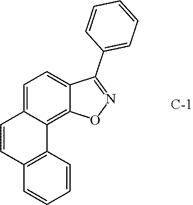 [SMILES: c1ccc(-c2noc3c2ccc2ccc4ccccc4c23)cc1]

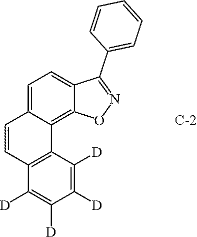 [SMILES: [2H]c1c([2H])c([2H])c2c(ccc3ccc4c(-c5ccccc5)noc4c32)c1[2H]]

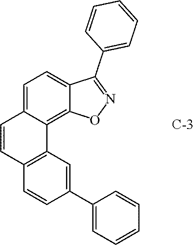 [SMILES: c1ccc(-c2ccc3ccc4ccc5c(-c6ccccc6)noc5c4c3c2)cc1]

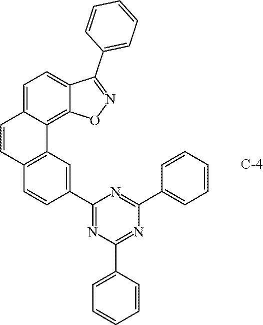 [SMILES: c1ccc(-c2nc(-c3ccccc3)nc(-c3ccc4ccc5ccc6c(-c7ccccc7)noc6c5c4c3)n2)cc1]

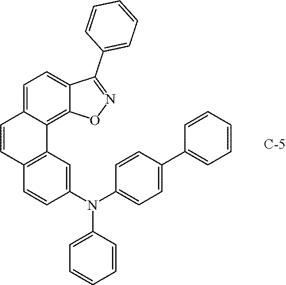 [SMILES: c1ccc(-c2ccc(N(c3ccccc3)c3ccc4ccc5ccc6c(-c7ccccc7)noc6c5c4c3)cc2)cc1]

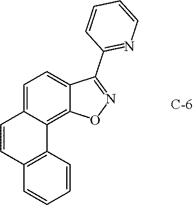 [SMILES: c1ccc(-c2noc3c2ccc2ccc4ccccc4c23)nc1]

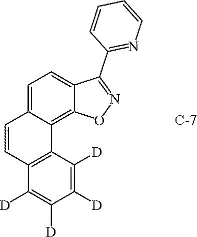 [SMILES: [2H]c1c([2H])c([2H])c2c(ccc3ccc4c(-c5ccccn5)noc4c32)c1[2H]]

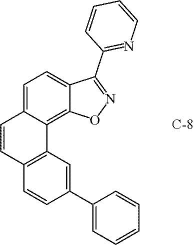 [SMILES: c1ccc(-c2ccc3ccc4ccc5c(-c6ccccn6)noc5c4c3c2)cc1]

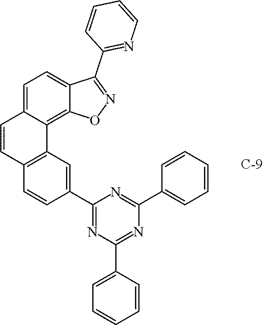 [SMILES: c1ccc(-c2nc(-c3ccccc3)nc(-c3ccc4ccc5ccc6c(-c7ccccn7)noc6c5c4c3)n2)cc1]

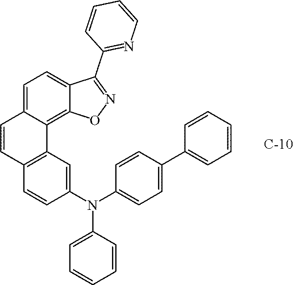 [SMILES: c1ccc(-c2ccc(N(c3ccccc3)c3ccc4ccc5ccc6c(-c7ccccn7)noc6c5c4c3)cc2)cc1]

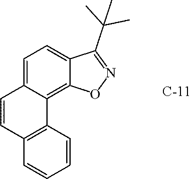 [SMILES: CC(C)(C)c1noc2c1ccc1ccc3ccccc3c12]

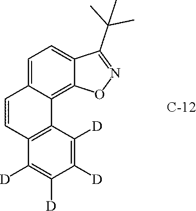 [SMILES: [2H]c1c([2H])c([2H])c2c(ccc3ccc4c(C(C)(C)C)noc4c32)c1[2H]]

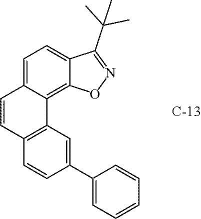 [SMILES: CC(C)(C)c1noc2c1ccc1ccc3ccc(-c4ccccc4)cc3c12]

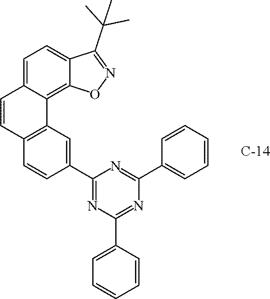 [SMILES: CC(C)(C)c1noc2c1ccc1ccc3ccc(-c4nc(-c5ccccc5)nc(-c5ccccc5)n4)cc3c12]

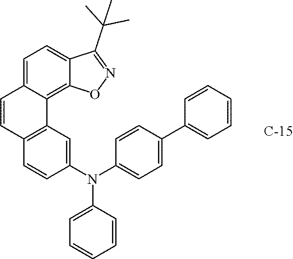 [SMILES: CC(C)(C)c1noc2c1ccc1ccc3ccc(N(c4ccccc4)c4ccc(-c5ccccc5)cc4)cc3c12]

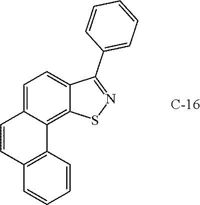 [SMILES: c1ccc(-c2nsc3c2ccc2ccc4ccccc4c23)cc1]

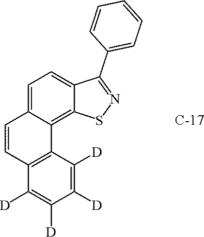 [SMILES: [2H]c1c([2H])c([2H])c2c(ccc3ccc4c(-c5ccccc5)nsc4c32)c1[2H]]

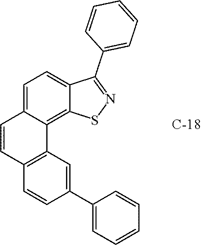 [SMILES: c1ccc(-c2ccc3ccc4ccc5c(-c6ccccc6)nsc5c4c3c2)cc1]

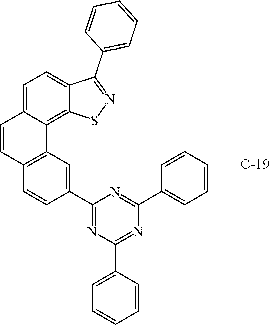 [SMILES: c1ccc(-c2nc(-c3ccccc3)nc(-c3ccc4ccc5ccc6c(-c7ccccc7)nsc6c5c4c3)n2)cc1]

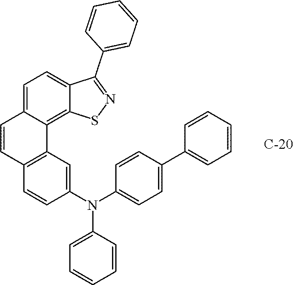 [SMILES: c1ccc(-c2ccc(N(c3ccccc3)c3ccc4ccc5ccc6c(-c7ccccc7)nsc6c5c4c3)cc2)cc1]

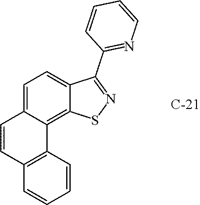 [SMILES: c1ccc(-c2nsc3c2ccc2ccc4ccccc4c23)nc1]

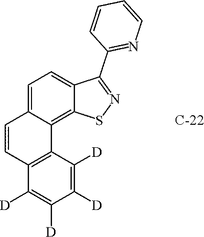 [SMILES: [2H]c1c([2H])c([2H])c2c(ccc3ccc4c(-c5ccccn5)nsc4c32)c1[2H]]

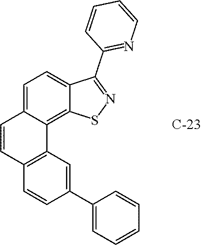 [SMILES: c1ccc(-c2ccc3ccc4ccc5c(-c6ccccn6)nsc5c4c3c2)cc1]

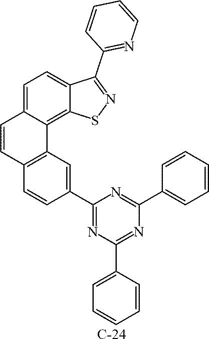

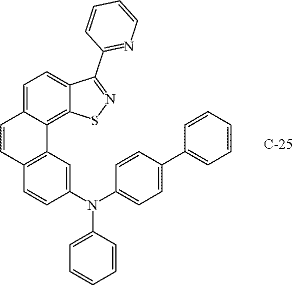 [SMILES: c1ccc(-c2ccc(N(c3ccccc3)c3ccc4ccc5ccc6c(-c7ccccn7)nsc6c5c4c3)cc2)cc1]

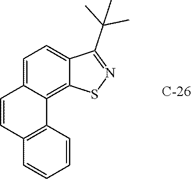 [SMILES: CC(C)(C)c1nsc2c1ccc1ccc3ccccc3c12]

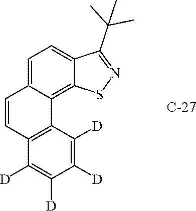 [SMILES: [2H]c1c([2H])c([2H])c2c(ccc3ccc4c(C(C)(C)C)nsc4c32)c1[2H]]

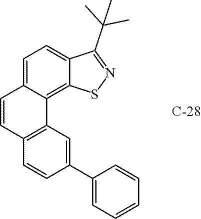 [SMILES: CC(C)(C)c1nsc2c1ccc1ccc3ccc(-c4ccccc4)cc3c12]

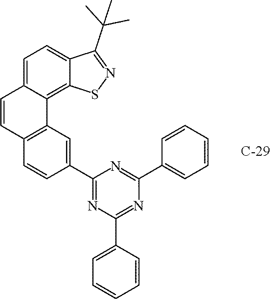 [SMILES: CC(C)(C)c1nsc2c1ccc1ccc3ccc(-c4nc(-c5ccccc5)nc(-c5ccccc5)n4)cc3c12]

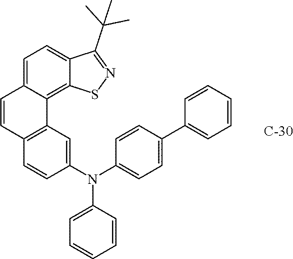 [SMILES: CC(C)(C)c1nsc2c1ccc1ccc3ccc(N(c4ccccc4)c4ccc(-c5ccccc5)cc4)cc3c12]

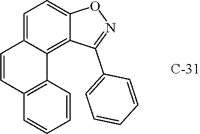 [SMILES: c1ccc(-c2noc3ccc4ccc5ccccc5c4c23)cc1]

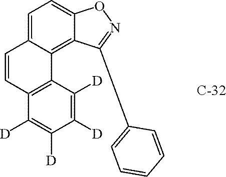 [SMILES: [2H]c1c([2H])c([2H])c2c(ccc3ccc4onc(-c5ccccc5)c4c32)c1[2H]]

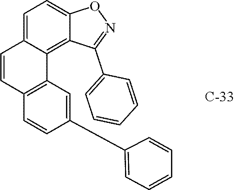 [SMILES: c1ccc(-c2ccc3ccc4ccc5onc(-c6ccccc6)c5c4c3c2)cc1]

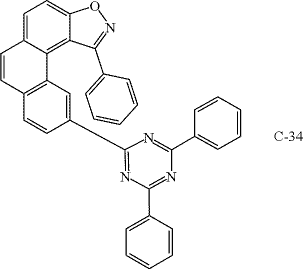 [SMILES: c1ccc(-c2nc(-c3ccccc3)nc(-c3ccc4ccc5ccc6onc(-c7ccccc7)c6c5c4c3)n2)cc1]

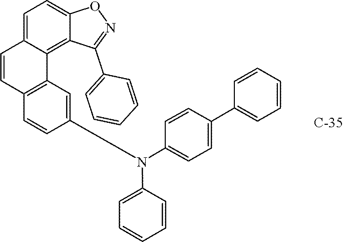 [SMILES: c1ccc(-c2ccc(N(c3ccccc3)c3ccc4ccc5ccc6onc(-c7ccccc7)c6c5c4c3)cc2)cc1]

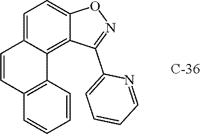 [SMILES: c1ccc(-c2noc3ccc4ccc5ccccc5c4c23)nc1]

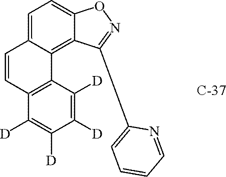 [SMILES: [2H]c1c([2H])c([2H])c2c(ccc3ccc4onc(-c5ccccn5)c4c32)c1[2H]]

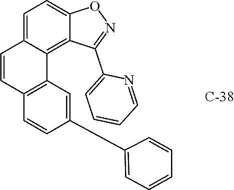 [SMILES: c1ccc(-c2ccc3ccc4ccc5onc(-c6ccccn6)c5c4c3c2)cc1]

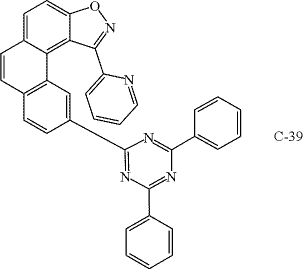 [SMILES: c1ccc(-c2nc(-c3ccccc3)nc(-c3ccc4ccc5ccc6onc(-c7ccccn7)c6c5c4c3)n2)cc1]

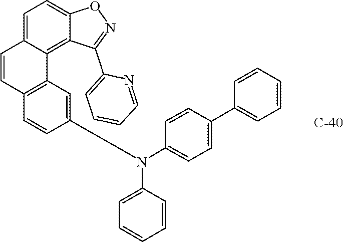 [SMILES: c1ccc(-c2ccc(N(c3ccccc3)c3ccc4ccc5ccc6onc(-c7ccccn7)c6c5c4c3)cc2)cc1]

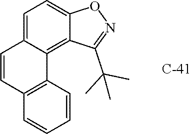 [SMILES: CC(C)(C)c1noc2ccc3ccc4ccccc4c3c12]

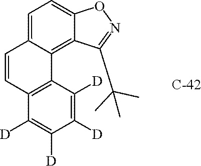 [SMILES: [2H]c1c([2H])c([2H])c2c(ccc3ccc4onc(C(C)(C)C)c4c32)c1[2H]]

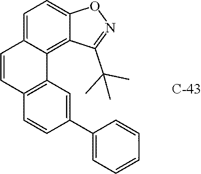 [SMILES: CC(C)(C)c1noc2ccc3ccc4ccc(-c5ccccc5)cc4c3c12]

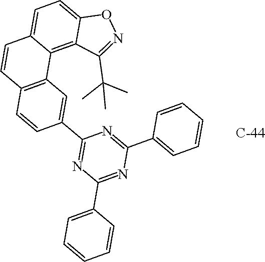 [SMILES: CC(C)(C)c1noc2ccc3ccc4ccc(-c5nc(-c6ccccc6)nc(-c6ccccc6)n5)cc4c3c12]

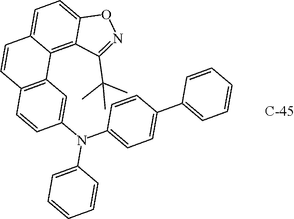 [SMILES: CC(C)(C)c1noc2ccc3ccc4ccc(N(c5ccccc5)c5ccc(-c6ccccc6)cc5)cc4c3c12]

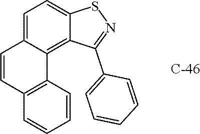 [SMILES: c1ccc(-c2nsc3ccc4ccc5ccccc5c4c23)cc1]

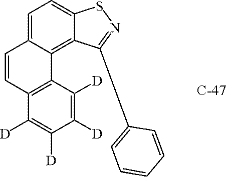 [SMILES: [2H]c1c([2H])c([2H])c2c(ccc3ccc4snc(-c5ccccc5)c4c32)c1[2H]]

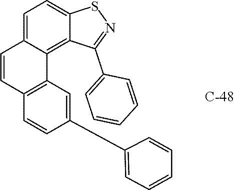 [SMILES: c1ccc(-c2ccc3ccc4ccc5snc(-c6ccccc6)c5c4c3c2)cc1]

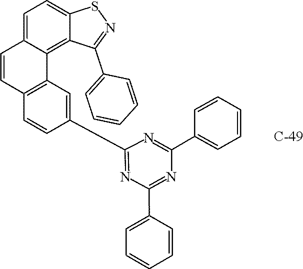 [SMILES: c1ccc(-c2nc(-c3ccccc3)nc(-c3ccc4ccc5ccc6snc(-c7ccccc7)c6c5c4c3)n2)cc1]

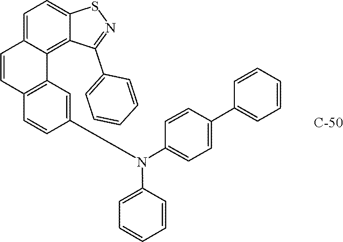 [SMILES: c1ccc(-c2ccc(N(c3ccccc3)c3ccc4ccc5ccc6snc(-c7ccccc7)c6c5c4c3)cc2)cc1]

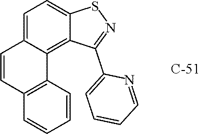 [SMILES: c1ccc(-c2nsc3ccc4ccc5ccccc5c4c23)nc1]

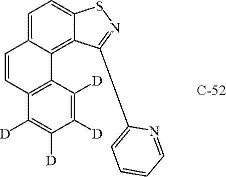 [SMILES: [2H]c1c([2H])c([2H])c2c(ccc3ccc4snc(-c5ccccn5)c4c32)c1[2H]]

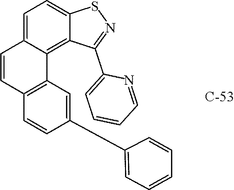 [SMILES: c1ccc(-c2ccc3ccc4ccc5snc(-c6ccccn6)c5c4c3c2)cc1]

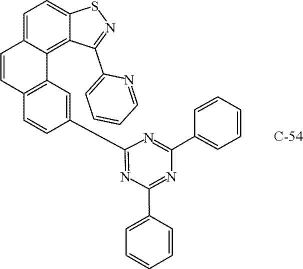 [SMILES: c1ccc(-c2nc(-c3ccccc3)nc(-c3ccc4ccc5ccc6snc(-c7ccccn7)c6c5c4c3)n2)cc1]

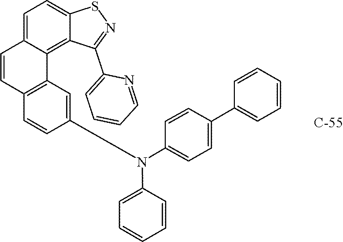 [SMILES: c1ccc(-c2ccc(N(c3ccccc3)c3ccc4ccc5ccc6snc(-c7ccccn7)c6c5c4c3)cc2)cc1]

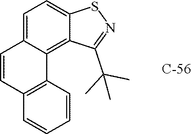 [SMILES: CC(C)(C)c1nsc2ccc3ccc4ccccc4c3c12]

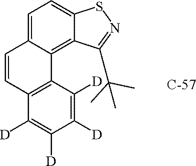 [SMILES: [2H]c1c([2H])c([2H])c2c(ccc3ccc4snc(C(C)(C)C)c4c32)c1[2H]]

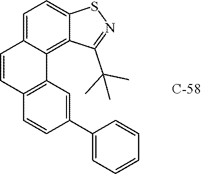 [SMILES: CC(C)(C)c1nsc2ccc3ccc4ccc(-c5ccccc5)cc4c3c12]

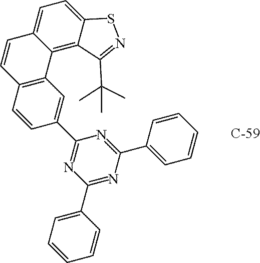 [SMILES: CC(C)(C)c1nsc2ccc3ccc4ccc(-c5nc(-c6ccccc6)nc(-c6ccccc6)n5)cc4c3c12]

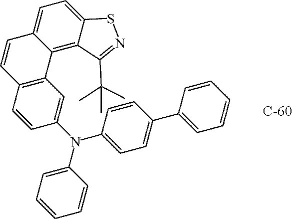 [SMILES: CC(C)(C)c1nsc2ccc3ccc4ccc(N(c5ccccc5)c5ccc(-c6ccccc6)cc5)cc4c3c12]

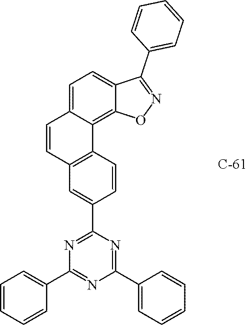 [SMILES: c1ccc(-c2nc(-c3ccccc3)nc(-c3ccc4c(ccc5ccc6c(-c7ccccc7)noc6c54)c3)n2)cc1]

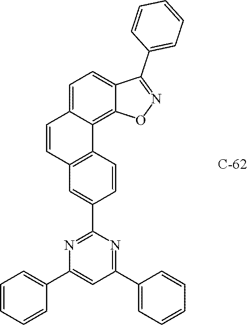 [SMILES: c1ccc(-c2cc(-c3ccccc3)nc(-c3ccc4c(ccc5ccc6c(-c7ccccc7)noc6c54)c3)n2)cc1]

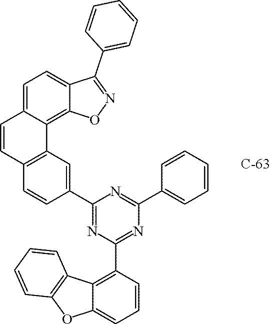 [SMILES: c1ccc(-c2nc(-c3ccc4ccc5ccc6c(-c7ccccc7)noc6c5c4c3)nc(-c3cccc4oc5ccccc5c34)n2)cc1]

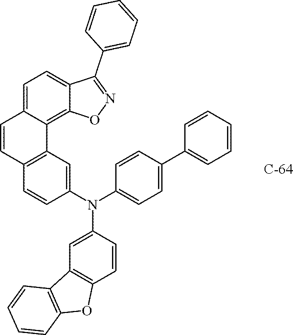 [SMILES: c1ccc(-c2ccc(N(c3ccc4oc5ccccc5c4c3)c3ccc4ccc5ccc6c(-c7ccccc7)noc6c5c4c3)cc2)cc1]

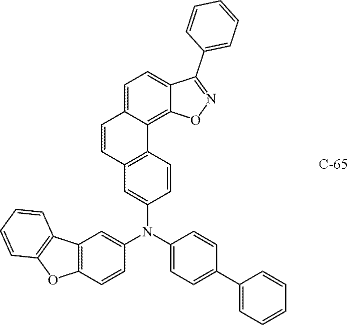 [SMILES: c1ccc(-c2ccc(N(c3ccc4c(ccc5ccc6c(-c7ccccc7)noc6c54)c3)c3ccc4oc5ccccc5c4c3)cc2)cc1]

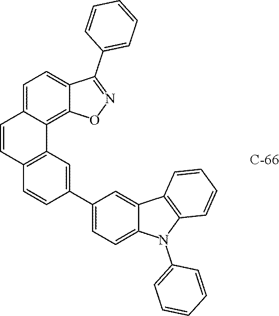 [SMILES: c1ccc(-c2noc3c2ccc2ccc4ccc(-c5ccc6c(c5)c5ccccc5n6-c5ccccc5)cc4c23)cc1]

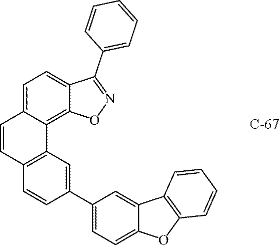 [SMILES: c1ccc(-c2noc3c2ccc2ccc4ccc(-c5ccc6oc7ccccc7c6c5)cc4c23)cc1]

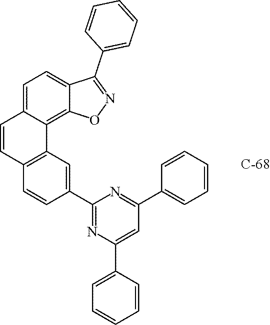 [SMILES: c1ccc(-c2cc(-c3ccccc3)nc(-c3ccc4ccc5ccc6c(-c7ccccc7)noc6c5c4c3)n2)cc1]

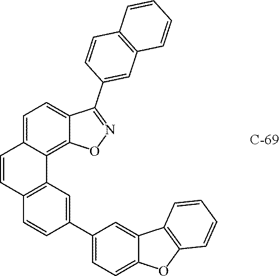 [SMILES: c1ccc2cc(-c3noc4c3ccc3ccc5ccc(-c6ccc7oc8ccccc8c7c6)cc5c34)ccc2c1]

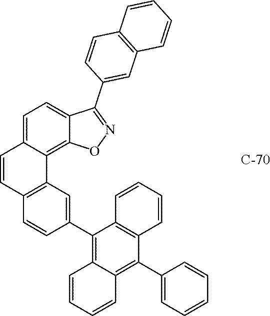 [SMILES: c1ccc(-c2c3ccccc3c(-c3ccc4ccc5ccc6c(-c7ccc8ccccc8c7)noc6c5c4c3)c3ccccc23)cc1]

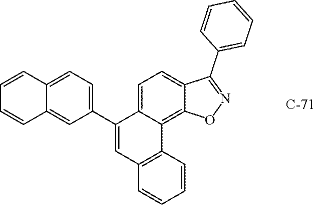 [SMILES: c1ccc(-c2noc3c2ccc2c(-c4ccc5ccccc5c4)cc4ccccc4c23)cc1]

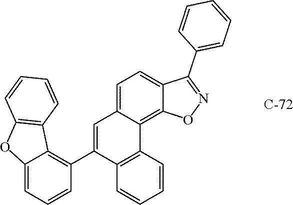 [SMILES: c1ccc(-c2noc3c2ccc2cc(-c4cccc5oc6ccccc6c45)c4ccccc4c23)cc1]

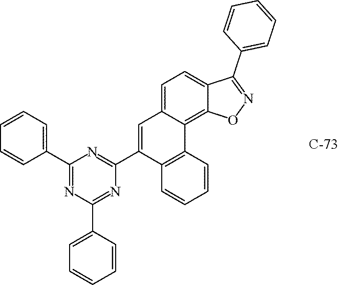 [SMILES: c1ccc(-c2nc(-c3ccccc3)nc(-c3cc4ccc5c(-c6ccccc6)noc5c4c4ccccc34)n2)cc1]

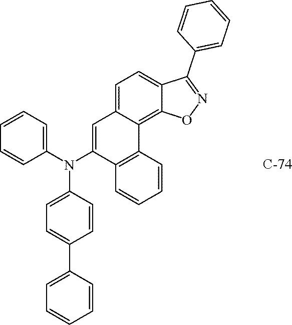 [SMILES: c1ccc(-c2ccc(N(c3ccccc3)c3cc4ccc5c(-c6ccccc6)noc5c4c4ccccc34)cc2)cc1]

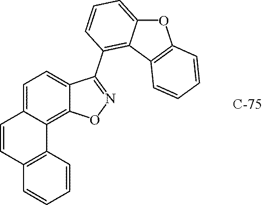 [SMILES: c1ccc2c(c1)ccc1ccc3c(-c4cccc5oc6ccccc6c45)noc3c12]

The present disclosure provides an organic electroluminescent material comprising the organic electroluminescent compound represented by formula 1, and an organic electroluminescent device comprising the same.

An organic electroluminescent device according to the present disclosure comprises an anode, a cathode, and at least one organic layer between the anode and the cathode, wherein the organic layer may comprise an organic electroluminescent material comprising the compound represented by formula 1.

The organic electroluminescent material may be comprised in at least one layer selected from a light-emitting layer, a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. According to one embodiment of the present disclosure, the organic electroluminescent device of the present disclosure comprises an anode, a cathode, and at least one light-emitting layer between the anode and the cathode, wherein the light-emitting layer may comprise the compound represented by formula 1.

The organic electroluminescent material may solely consist of the organic electroluminescent compound of the present disclosure, or may further comprise conventional materials included in the organic electroluminescent material.

The plurality of host materials according to the present disclosure comprises a first host material comprising the compound represented by formula 1 and a second host material comprising the compound represented by formula 2, and may be comprised in a light-emitting layer of the organic electroluminescent device according to the present disclosure.

Hereinafter, the compound represented by formula 2 will be described in more detail.

In formula 2, at least one of T₅ and T₆, and T₇ and T₈ is linked to each other to form a ring(s) together with formula 3. According to one embodiment of the present disclosure, in formula 2, T₅ and T₆, or T₇ and T₈ may form a ring(s) together with formula 3.

In formula 3,

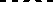

represents a fusion site with formula 2.

In formulas 2 and 3, T₁ to T₄, T₉ to T₁₄, and T₅ to T₈ that do not form a ring(s) together with formula 3 each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono-or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted mono- or di- (C6-C30)arylamino, a substituted or unsubstituted (C1-C30)alkyl(C6-C30)arylamino, a substituted or unsubstituted mono- or di- (3- to 30-membered)heteroarylamino, a substituted or unsubstituted (C1-C30)alkyl(3- to 30-membered)heteroarylamino, a substituted or unsubstituted (C2-C30)alkenyl(C6-C30) arylamino, a substituted or unsubstituted (C2-C30)alkenyl(3- to 30-membered)heteroarylamino, a substituted or unsubstituted (C6-C30)aryl(3- to 30-membered)heteroarylamino, or -L₂-Ar₃, provided that at least one of T₁ to T₁₄ represents -L₂-Ar₃. According to one embodiment of the present disclosure, T₁ to T₄, T₉ to T₁₄, and T₅ to T₈ that do not form a ring(s) together with formula 3, each independently, may be a substituted or unsubstituted (C6-C30)aryl, or -L₂-Ar₃. According to another embodiment of the present disclosure, T₁ to T₄, T₉ to T₁₄, and T₅ to T₈ that do not form a ring(s) together with formula 3, each independently, may be an unsubstituted (C6-C18)aryl, or -L₂-Ar₃. For example, T₁ to T₄, T₉ to T₁₄, and T₅ to T₈ that do not form a ring(s) together with formula 3, each independently, may be hydrogen, a phenyl, a biphenyl, a naphthyl, -L₂-Ar₃, etc.

L₂ each independently represents a single bond, a substituted or unsubstituted (C6-C30)arylene, or a substituted or unsubstituted (3- to 30-membered)heteroarylene. According to one embodiment of the present disclosure, L₂, each independently, may represent a single bond or a substituted or unsubstituted (C6-C18)arylene. According to another embodiment of the present disclosure, L₂, each independently, may represent a single bond, or a (C6-C15)arylene unsubstituted or substituted with (C6-C15)aryl(s). For example, L₂, each independently, may represent a single bond, a phenylene unsubstituted or substituted with a phenyl(s), a biphenylene, a naphthylene, etc.

Ar₃ each independently represents a substituted or unsubstituted (C6-C30)aryl, or a substituted or unsubstituted (3- to 30-membered)heteroaryl, wherein the heteroaryl comprises at least one heteroatom(s) selected from B, N, O, S, Si and P. According to one embodiment of the present disclosure, Ar₃, each independently, may represent an unsubstituted (C6-C25)aryl, or a substituted or unsubstituted (3- to 28-membered)heteroaryl, wherein the heteroaryl may comprise at least one heteroatom(s) selected from N, O, and S, and the substituent(s) of the substituted (3- to 28-membered)heteroaryl may be at least one selected from the group consisting of a (C6-C30)aryl unsubstituted or substituted with at least one of a cyano(s), a halogen(s), a (C1-C30)alkyl(s), a (C3-C30)cycloalkyl(s), a tri(C1-C30)alkylsilyl(s), a tri(C6-C30)arylsilyl(s), a (C6-C30)aryl(s), and a (3- to 30-membered)heteroaryl(s); and a (6- to 20-membered)heteroaryl unsubstituted or substituted with a (C6-C25)aryl(s). According to another embodiment of the present disclosure, Ar₃, each independently, may represent an unsubstituted (C6-C18)aryl, or a substituted or unsubstituted (3- to 27-membered)heteroaryl, wherein the heteroaryl may contain at least one N, and further may contain at least one heteroatom(s) selected from O and S; and the substituent(s) of the substituted (3- to 27-membered)heteroaryl may be at least one selected from the group consisting of a (C6-C30)aryl unsubstituted or substituted with at least one of a cyano(s), a halogen(s), a (C1-C20)alkyl(s), a (C3-C15)cycloalkyl(s), a tri(C1-C20)alkylsilyl(s), a tri(C6-C25)arylsilyl(s), a (C6-C28)aryl(s), and a (3- to 25-membered)heteroaryl(s); and a (6- to 18-membered)heteroaryl unsubstituted or substituted with a (C6-C18)aryl(s). For example, Ar₃, each independently, may represent a phenyl, a biphenyl, a naphthyl, a substituted or unsubstituted pyrimidinyl, a substituted or unsubstituted triazinyl, a substituted or unsubstituted phenanthroimidazolyl, a substituted or unsubstituted quinazolinyl, a substituted or unsubstituted quinoxalinyl, a substituted or unsubstituted benzoquinoxalinyl, a substituted or unsubstituted dibenzoquinoxalinyl, a substituted or unsubstituted benzofuropyrimidinyl, a substituted or unsubstituted benzoimidazolyl, a substituted or unsubstituted acenaphthopyrimidinyl, a substituted or unsubstituted naphthyridinyl, a substituted or unsubstituted quinolyl, a substituted or unsubstituted isoquinolyl, a substituted or unsubstituted benzothienoquinolyl, or a substituted or unsubstituted (16- to 25-membered)heteroaryl, wherein the (16- to 25-membered)heteroaryl includes at least one N, and may further contain at least one heteroatom(s) selected from O and S. Specifically, the substituent(s) of the substituted pyrimidinyl, the substituted triazinyl, the substituted phenanthroimidazolyl, the substituted quinazolinyl, the substituted quinoxalinyl, the substituted benzoquinoxalinyl, the substituted dibenzoquinoxalinyl, the substituted benzofuropyrimidinyl, the substituted benzoimidazolyl, the substituted acenaphthopyrimidinyl, the substituted naphthyridinyl, the substituted or unsubstituted quinolyl, the substituted isoquinolyl, the substituted benzothienoquinolyl, and the substituted (16- to 25-membered)heteroaryl each independently may be at least one selected from the group consisting of a phenyl unsubstituted or substituted with a methyl(s), a t-butyl(s), a cyclohexane(s), a naphthyl(s), a carbazolyl(s), a trimethylsilyl(s), a triphenylsilyl(s), a cyano(s), or a fluoro(s); a biphenyl; a terphenyl; a naphthyl unsubstituted or substituted with a phenyl(s); a phenanthrenyl; a chrysenyl; an anthracenyl; a triphenylenyl; a dimethylfluorenyl; a diphenylfluorenyl; a spirobifluorenyl; a (C22)aryl; a dibenzofuranyl unsubstituted or substituted with a phenyl(s) or a biphenyl(s); a dibenzothiophenyl; a carbazolyl unsubstituted or substituted with a phenyl(s); a phenoxazinyl; a benzothiophenyl; a naphthooxazolyl unsubstituted or substituted with a phenyl(s); and a pyridyl unsubstituted or substituted with a phenyl(s).

According to another embodiment of the present disclosure, Ar₃, each independently, may represent a substituted or unsubstituted phenyl, a substituted or unsubstituted naphthyl, a substituted or unsubstituted biphenyl, a substituted or unsubstituted terphenyl, a substituted or unsubstituted phenanthrenyl, a substituted or unsubstituted phenanthroimidazolyl, a substituted or unsubstituted anthracenyl, a substituted or unsubstituted fluorenyl, a substituted or unsubstituted benzofluorenyl, a substituted or unsubstituted triphenylenyl, a substituted or unsubstituted spirobifluorenyl, a substituted or unsubstituted pyridyl, a substituted or unsubstituted triazinyl, a substituted or unsubstituted pyrimidinyl, a substituted or unsubstituted quinolyl, a substituted or unsubstituted isoquinolyl, a substituted or unsubstituted benzothienoquinolyl, a substituted or unsubstituted quinazolinyl, a substituted or unsubstituted quinoxalinyl, a substituted or unsubstituted dibenzoquinoxalinyl, a substituted or unsubstituted benzoquinazolinyl, a substituted or unsubstituted benzoquinoxalinyl, a substituted or unsubstituted benzofuropyrimidinyl, a substituted or unsubstituted benzoimidazolyl, a substituted or unsubstituted acenaphthopyrimidinyl, a substituted or unsubstituted carbazolyl, a substituted or unsubstituted dibenzothiophenyl, a substituted or unsubstituted benzothiophenyl, a substituted or unsubstituted dibenzofuranyl, a substituted or unsubstituted benzofuranyl, a substituted or unsubstituted naphthyridinyl, a substituted or unsubstituted benzonaphthofuranyl, a substituted or unsubstituted benzonaphthothiophenyl, or a substituted or unsubstituted (16- to 25-membered)heteroaryl comprising at least one heteroatom(s) selected from N, O, and S.

According to one embodiment of the present disclosure, formula 2 may be represented by at least one of the following formulas 2-1 and 2-2.

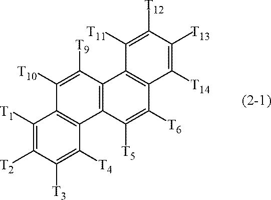

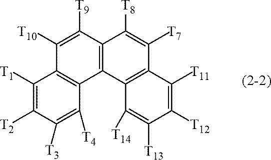

In formulas 2-1 and 2-2, T₁ to T₄, and T₉ to T₁₄ are as defined in formula 2, and T₅ to T₈ are as defined for T₁ to T₄, and T₉ to T₁₄.

According to one embodiment of the present disclosure, formula 2 may be represented by at least one of the following formulas.

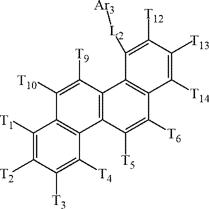

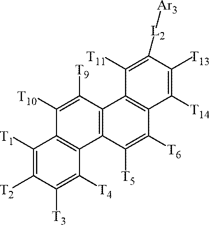

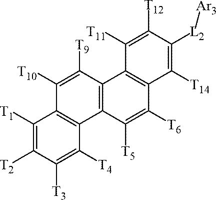

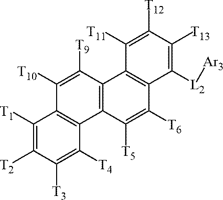

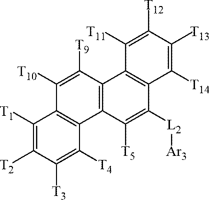

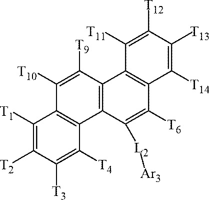

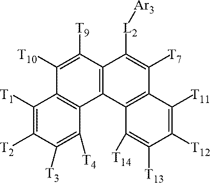

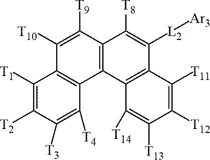

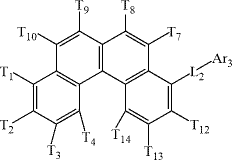

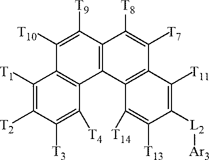

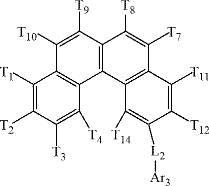

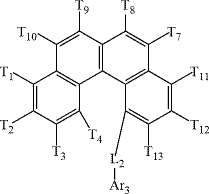

In the above formulas, T₁ to T₁₄ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted mono- or di- (C6-C30)arylamino, a substituted or unsubstituted (C1-C30)alkyl(C6-C30)arylamino, a substituted or unsubstituted mono- or di- (3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino, and L₂ and Ar₃ are as defined in formula 2.

The compound represented by formula 2 may be at least one selected from the following compounds, but is not limited thereto.

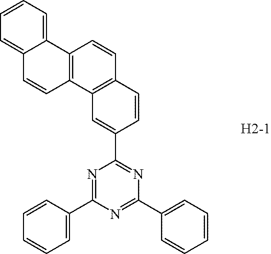 [SMILES: c1ccc(-c2nc(-c3ccccc3)nc(-c3ccc4ccc5c6ccccc6ccc5c4c3)n2)cc1]

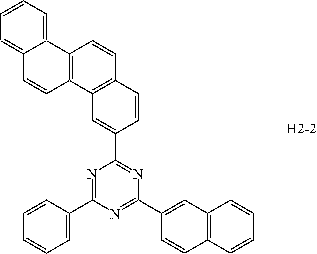 [SMILES: c1ccc(-c2nc(-c3ccc4ccccc4c3)nc(-c3ccc4ccc5c6ccccc6ccc5c4c3)n2)cc1]

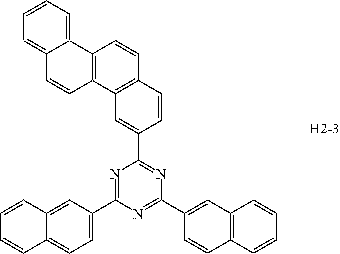 [SMILES: c1ccc2cc(-c3nc(-c4ccc5ccccc5c4)nc(-c4ccc5ccc6c7ccccc7ccc6c5c4)n3)ccc2c1]

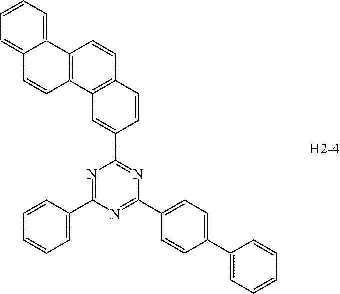 [SMILES: c1ccc(-c2ccc(-c3nc(-c4ccccc4)nc(-c4ccc5ccc6c7ccccc7ccc6c5c4)n3)cc2)cc1]

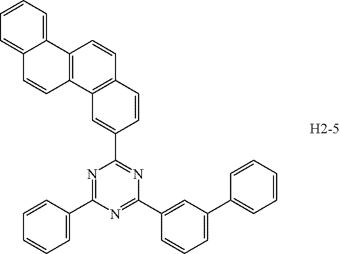 [SMILES: c1ccc(-c2cccc(-c3nc(-c4ccccc4)nc(-c4ccc5ccc6c7ccccc7ccc6c5c4)n3)c2)cc1]

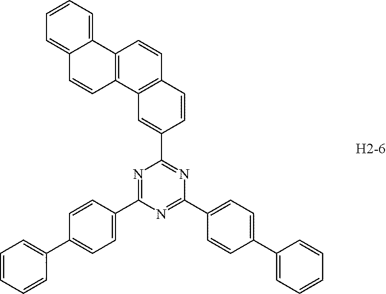 [SMILES: c1ccc(-c2ccc(-c3nc(-c4ccc(-c5ccccc5)cc4)nc(-c4ccc5ccc6c7ccccc7ccc6c5c4)n3)cc2)cc1]

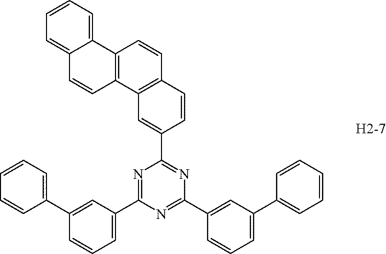 [SMILES: c1ccc(-c2cccc(-c3nc(-c4cccc(-c5ccccc5)c4)nc(-c4ccc5ccc6c7ccccc7ccc6c5c4)n3)c2)cc1]

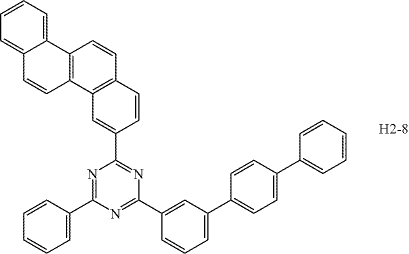 [SMILES: c1ccc(-c2ccc(-c3cccc(-c4nc(-c5ccccc5)nc(-c5ccc6ccc7c8ccccc8ccc7c6c5)n4)c3)cc2)cc1]

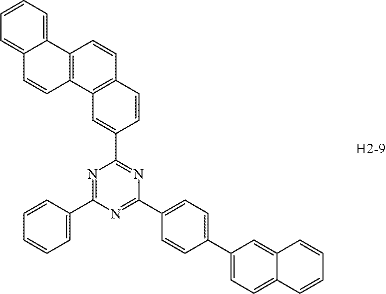 [SMILES: c1ccc(-c2nc(-c3ccc(-c4ccc5ccccc5c4)cc3)nc(-c3ccc4ccc5c6ccccc6ccc5c4c3)n2)cc1]

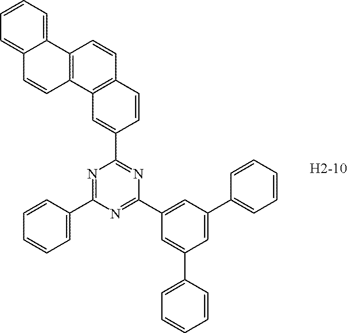 [SMILES: c1ccc(-c2cc(-c3ccccc3)cc(-c3nc(-c4ccccc4)nc(-c4ccc5ccc6c7ccccc7ccc6c5c4)n3)c2)cc1]

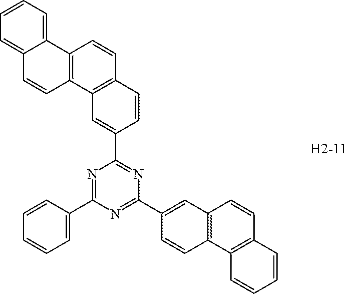 [SMILES: c1ccc(-c2nc(-c3ccc4c(ccc5ccccc54)c3)nc(-c3ccc4ccc5c6ccccc6ccc5c4c3)n2)cc1]

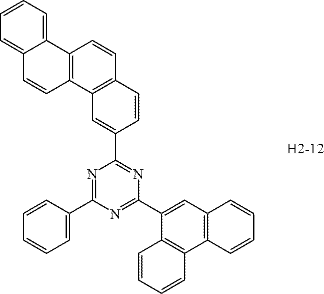 [SMILES: c1ccc(-c2nc(-c3ccc4ccc5c6ccccc6ccc5c4c3)nc(-c3cc4ccccc4c4ccccc34)n2)cc1]

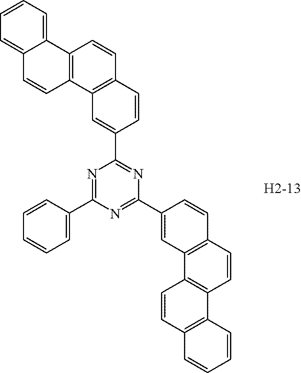 [SMILES: c1ccc(-c2nc(-c3ccc4ccc5c6ccccc6ccc5c4c3)nc(-c3ccc4ccc5c6ccccc6ccc5c4c3)n2)cc1]

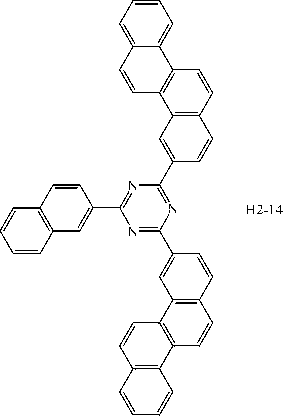 [SMILES: c1ccc2cc(-c3nc(-c4ccc5ccc6c7ccccc7ccc6c5c4)nc(-c4ccc5ccc6c7ccccc7ccc6c5c4)n3)ccc2c1]

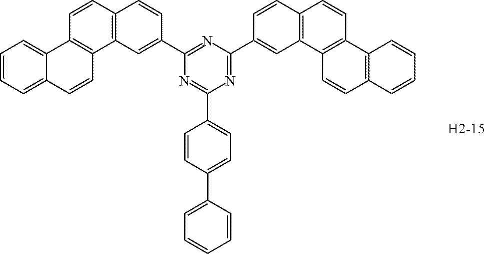 [SMILES: c1ccc(-c2ccc(-c3nc(-c4ccc5ccc6c7ccccc7ccc6c5c4)nc(-c4ccc5ccc6c7ccccc7ccc6c5c4)n3)cc2)cc1]

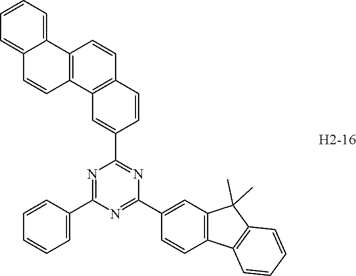 [SMILES: CC1(C)c2ccccc2-c2ccc(-c3nc(-c4ccccc4)nc(-c4ccc5ccc6c7ccccc7ccc6c5c4)n3)cc21]

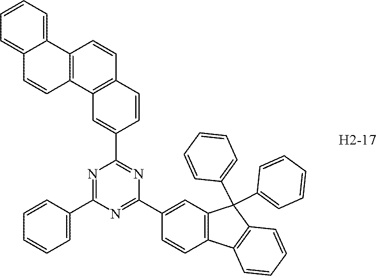 [SMILES: c1ccc(-c2nc(-c3ccc4c(c3)C(c3ccccc3)(c3ccccc3)c3ccccc3-4)nc(-c3ccc4ccc5c6ccccc6ccc5c4c3)n2)cc1]

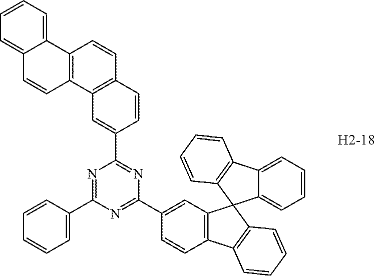 [SMILES: c1ccc(-c2nc(-c3ccc4c(c3)C3(c5ccccc5-c5ccccc53)c3ccccc3-4)nc(-c3ccc4ccc5c6ccccc6ccc5c4c3)n2)cc1]

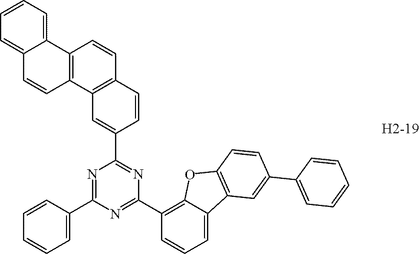 [SMILES: c1ccc(-c2ccc3oc4c(-c5nc(-c6ccccc6)nc(-c6ccc7ccc8c9ccccc9ccc8c7c6)n5)cccc4c3c2)cc1]

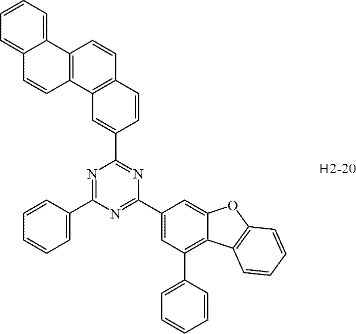 [SMILES: c1ccc(-c2nc(-c3cc(-c4ccccc4)c4c(c3)oc3ccccc34)nc(-c3ccc4ccc5c6ccccc6ccc5c4c3)n2)cc1]

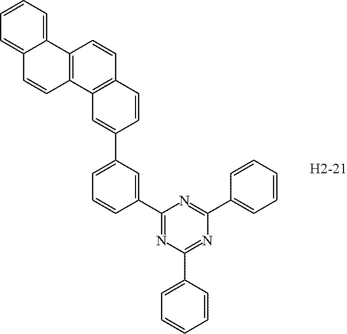 [SMILES: c1ccc(-c2nc(-c3ccccc3)nc(-c3cccc(-c4ccc5ccc6c7ccccc7ccc6c5c4)c3)n2)cc1]

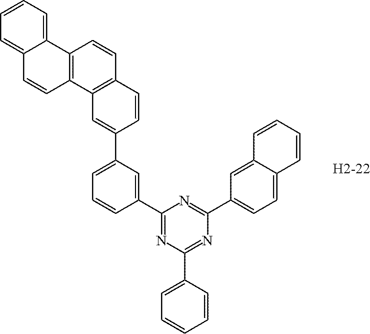 [SMILES: c1ccc(-c2nc(-c3cccc(-c4ccc5ccc6c7ccccc7ccc6c5c4)c3)nc(-c3ccc4ccccc4c3)n2)cc1]

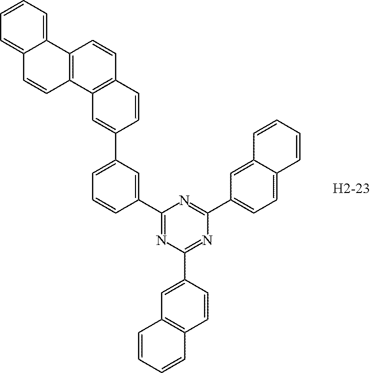 [SMILES: c1cc(-c2ccc3ccc4c5ccccc5ccc4c3c2)cc(-c2nc(-c3ccc4ccccc4c3)nc(-c3ccc4ccccc4c3)n2)c1]

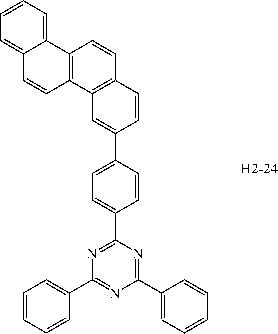 [SMILES: c1ccc(-c2nc(-c3ccccc3)nc(-c3ccc(-c4ccc5ccc6c7ccccc7ccc6c5c4)cc3)n2)cc1]

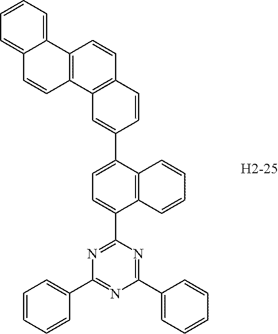 [SMILES: c1ccc(-c2nc(-c3ccccc3)nc(-c3ccc(-c4ccc5ccc6c7ccccc7ccc6c5c4)c4ccccc34)n2)cc1]

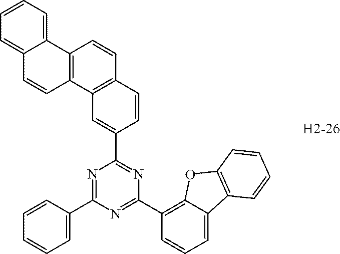 [SMILES: c1ccc(-c2nc(-c3ccc4ccc5c6ccccc6ccc5c4c3)nc(-c3cccc4c3oc3ccccc34)n2)cc1]

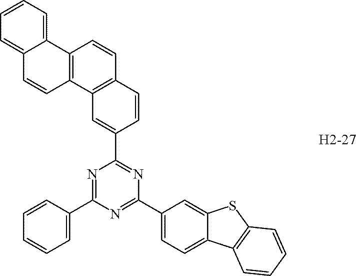 [SMILES: c1ccc(-c2nc(-c3ccc4c(c3)sc3ccccc34)nc(-c3ccc4ccc5c6ccccc6ccc5c4c3)n2)cc1]

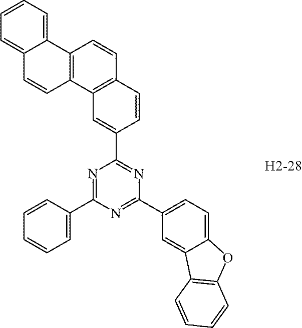 [SMILES: c1ccc(-c2nc(-c3ccc4ccc5c6ccccc6ccc5c4c3)nc(-c3ccc4oc5ccccc5c4c3)n2)cc1]

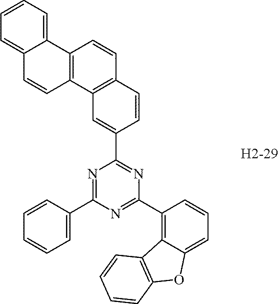 [SMILES: c1ccc(-c2nc(-c3ccc4ccc5c6ccccc6ccc5c4c3)nc(-c3cccc4oc5ccccc5c34)n2)cc1]

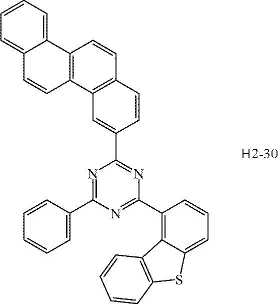 [SMILES: c1ccc(-c2nc(-c3ccc4ccc5c6ccccc6ccc5c4c3)nc(-c3cccc4sc5ccccc5c34)n2)cc1]

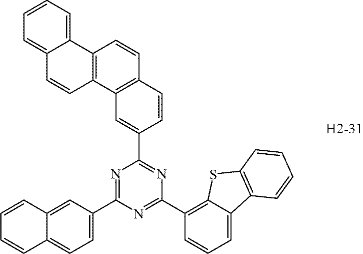 [SMILES: c1ccc2cc(-c3nc(-c4ccc5ccc6c7ccccc7ccc6c5c4)nc(-c4cccc5c4sc4ccccc45)n3)ccc2c1]

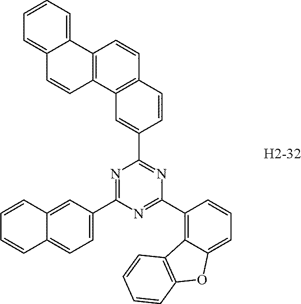 [SMILES: c1ccc2cc(-c3nc(-c4ccc5ccc6c7ccccc7ccc6c5c4)nc(-c4cccc5oc6ccccc6c45)n3)ccc2c1]

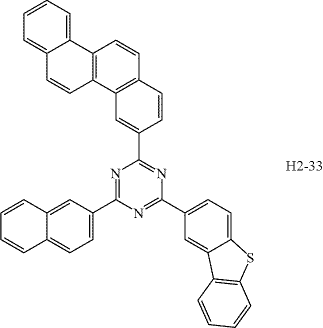 [SMILES: c1ccc2cc(-c3nc(-c4ccc5ccc6c7ccccc7ccc6c5c4)nc(-c4ccc5sc6ccccc6c5c4)n3)ccc2c1]

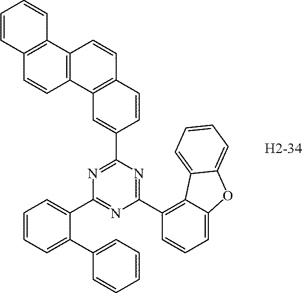 [SMILES: c1ccc(-c2ccccc2-c2nc(-c3ccc4ccc5c6ccccc6ccc5c4c3)nc(-c3cccc4oc5ccccc5c34)n2)cc1]

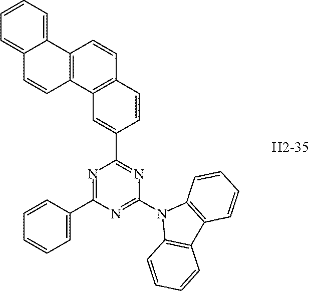 [SMILES: c1ccc(-c2nc(-c3ccc4ccc5c6ccccc6ccc5c4c3)nc(-n3c4ccccc4c4ccccc43)n2)cc1]

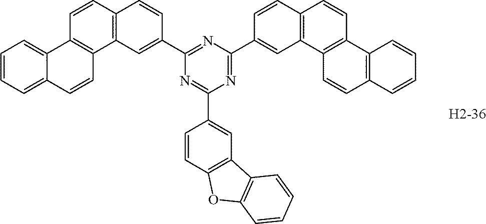 [SMILES: c1ccc2c(c1)ccc1c3cc(-c4nc(-c5ccc6ccc7c8ccccc8ccc7c6c5)nc(-c5ccc6oc7ccccc7c6c5)n4)ccc3ccc21]

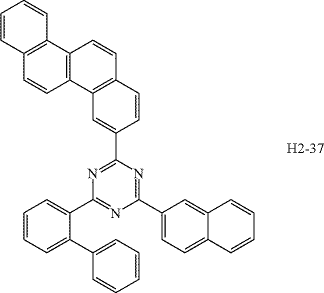 [SMILES: c1ccc(-c2ccccc2-c2nc(-c3ccc4ccccc4c3)nc(-c3ccc4ccc5c6ccccc6ccc5c4c3)n2)cc1]

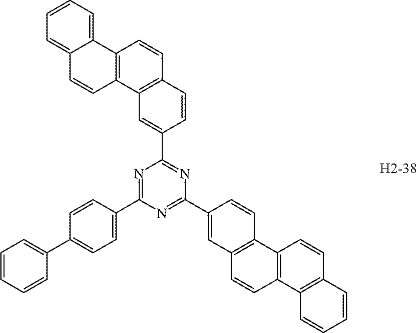 [SMILES: c1ccc(-c2ccc(-c3nc(-c4ccc5c(ccc6c7ccccc7ccc56)c4)nc(-c4ccc5ccc6c7ccccc7ccc6c5c4)n3)cc2)cc1]

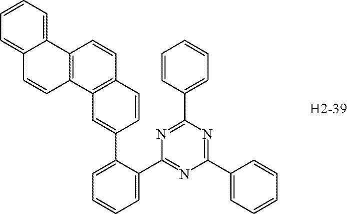 [SMILES: c1ccc(-c2nc(-c3ccccc3)nc(-c3ccccc3-c3ccc4ccc5c6ccccc6ccc5c4c3)n2)cc1]

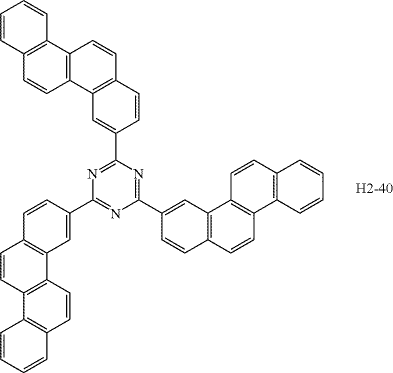 [SMILES: c1ccc2c(c1)ccc1c3cc(-c4nc(-c5ccc6ccc7c8ccccc8ccc7c6c5)nc(-c5ccc6ccc7c8ccccc8ccc7c6c5)n4)ccc3ccc21]

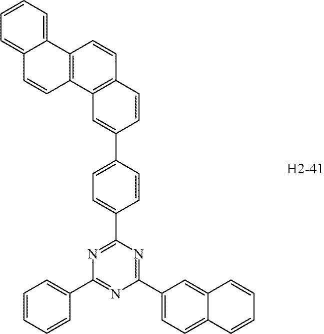 [SMILES: c1ccc(-c2nc(-c3ccc(-c4ccc5ccc6c7ccccc7ccc6c5c4)cc3)nc(-c3ccc4ccccc4c3)n2)cc1]

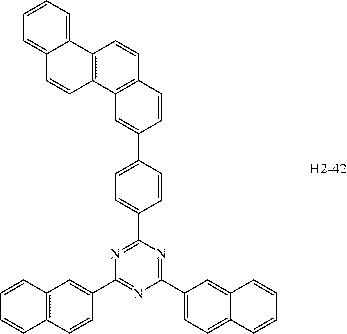 [SMILES: c1ccc2cc(-c3nc(-c4ccc(-c5ccc6ccc7c8ccccc8ccc7c6c5)cc4)nc(-c4ccc5ccccc5c4)n3)ccc2c1]

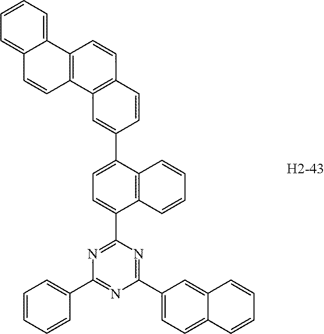 [SMILES: c1ccc(-c2nc(-c3ccc4ccccc4c3)nc(-c3ccc(-c4ccc5ccc6c7ccccc7ccc6c5c4)c4ccccc34)n2)cc1]

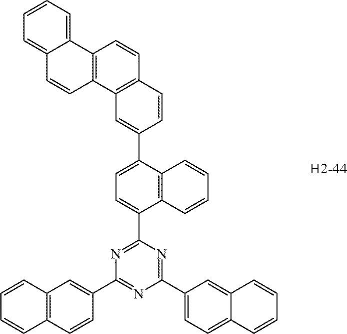 [SMILES: c1ccc2cc(-c3nc(-c4ccc5ccccc5c4)nc(-c4ccc(-c5ccc6ccc7c8ccccc8ccc7c6c5)c5ccccc45)n3)ccc2c1]

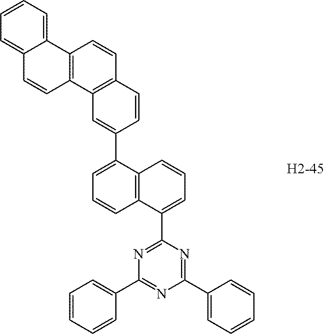 [SMILES: c1ccc(-c2nc(-c3ccccc3)nc(-c3cccc4c(-c5ccc6ccc7c8ccccc8ccc7c6c5)cccc34)n2)cc1]

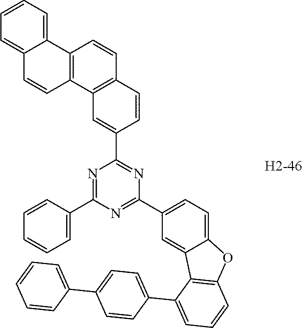 [SMILES: c1ccc(-c2ccc(-c3cccc4oc5ccc(-c6nc(-c7ccccc7)nc(-c7ccc8ccc9c%10ccccc%10ccc9c8c7)n6)cc5c34)cc2)cc1]

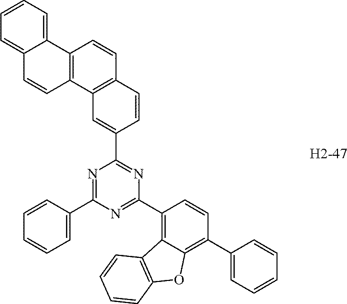 [SMILES: c1ccc(-c2nc(-c3ccc4ccc5c6ccccc6ccc5c4c3)nc(-c3ccc(-c4ccccc4)c4oc5ccccc5c34)n2)cc1]

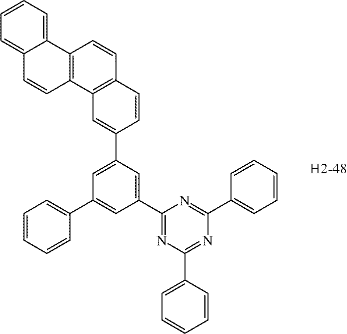 [SMILES: c1ccc(-c2cc(-c3ccc4ccc5c6ccccc6ccc5c4c3)cc(-c3nc(-c4ccccc4)nc(-c4ccccc4)n3)c2)cc1]

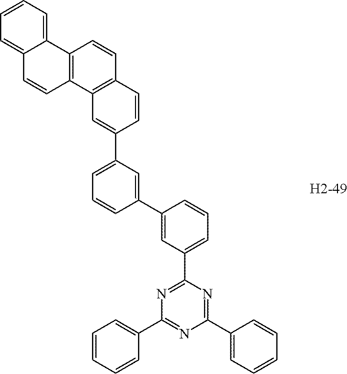 [SMILES: c1ccc(-c2nc(-c3ccccc3)nc(-c3cccc(-c4cccc(-c5ccc6ccc7c8ccccc8ccc7c6c5)c4)c3)n2)cc1]

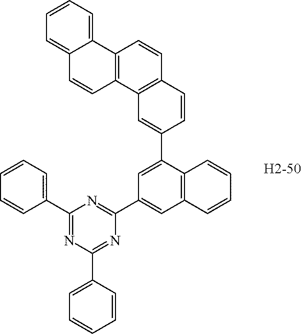 [SMILES: c1ccc(-c2nc(-c3ccccc3)nc(-c3cc(-c4ccc5ccc6c7ccccc7ccc6c5c4)c4ccccc4c3)n2)cc1]

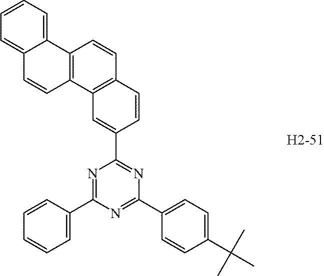 [SMILES: CC(C)(C)c1ccc(-c2nc(-c3ccccc3)nc(-c3ccc4ccc5c6ccccc6ccc5c4c3)n2)cc1]

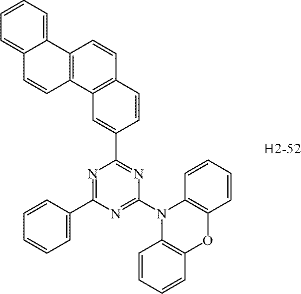 [SMILES: c1ccc(-c2nc(-c3ccc4ccc5c6ccccc6ccc5c4c3)nc(N3c4ccccc4Oc4ccccc43)n2)cc1]

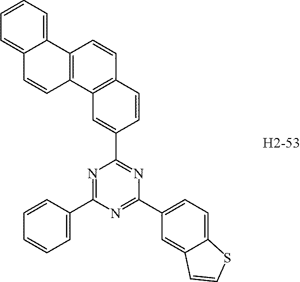 [SMILES: c1ccc(-c2nc(-c3ccc4sccc4c3)nc(-c3ccc4ccc5c6ccccc6ccc5c4c3)n2)cc1]

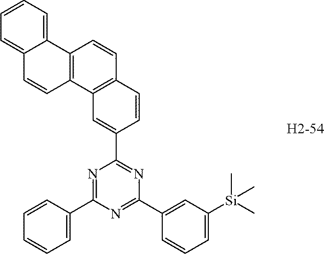 [SMILES: C[Si](C)(C)c1cccc(-c2nc(-c3ccccc3)nc(-c3ccc4ccc5c6ccccc6ccc5c4c3)n2)c1]

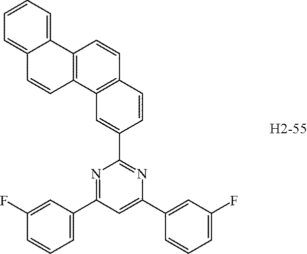 [SMILES: Fc1cccc(-c2cc(-c3cccc(F)c3)nc(-c3ccc4ccc5c6ccccc6ccc5c4c3)n2)c1]

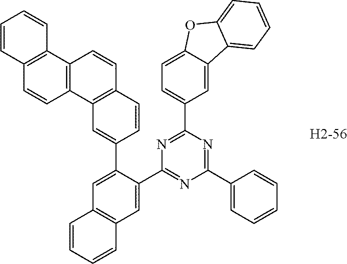 [SMILES: c1ccc(-c2nc(-c3ccc4oc5ccccc5c4c3)nc(-c3cc4ccccc4cc3-c3ccc4ccc5c6ccccc6ccc5c4c3)n2)cc1]

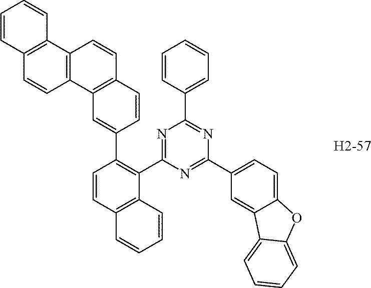 [SMILES: c1ccc(-c2nc(-c3ccc4oc5ccccc5c4c3)nc(-c3c(-c4ccc5ccc6c7ccccc7ccc6c5c4)ccc4ccccc34)n2)cc1]

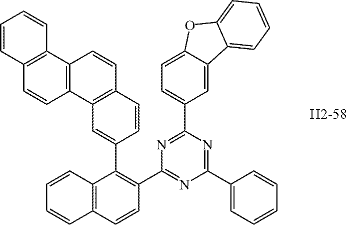 [SMILES: c1ccc(-c2nc(-c3ccc4oc5ccccc5c4c3)nc(-c3ccc4ccccc4c3-c3ccc4ccc5c6ccccc6ccc5c4c3)n2)cc1]

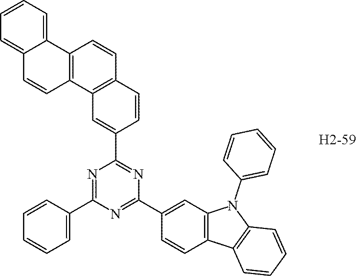 [SMILES: c1ccc(-c2nc(-c3ccc4ccc5c6ccccc6ccc5c4c3)nc(-c3ccc4c5ccccc5n(-c5ccccc5)c4c3)n2)cc1]

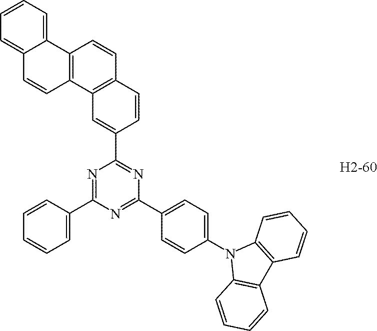 [SMILES: c1ccc(-c2nc(-c3ccc(-n4c5ccccc5c5ccccc54)cc3)nc(-c3ccc4ccc5c6ccccc6ccc5c4c3)n2)cc1]

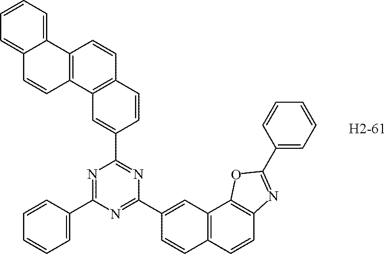 [SMILES: c1ccc(-c2nc(-c3ccc4ccc5c6ccccc6ccc5c4c3)nc(-c3ccc4ccc5nc(-c6ccccc6)oc5c4c3)n2)cc1]

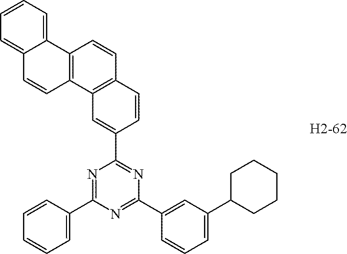 [SMILES: c1ccc(-c2nc(-c3cccc(C4CCCCC4)c3)nc(-c3ccc4ccc5c6ccccc6ccc5c4c3)n2)cc1]

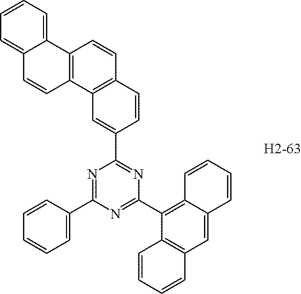 [SMILES: c1ccc(-c2nc(-c3ccc4ccc5c6ccccc6ccc5c4c3)nc(-c3c4ccccc4cc4ccccc34)n2)cc1]

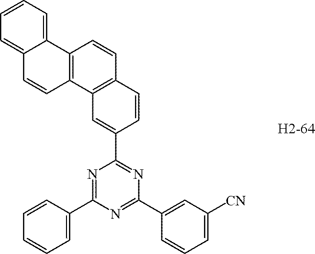 [SMILES: N#Cc1cccc(-c2nc(-c3ccccc3)nc(-c3ccc4ccc5c6ccccc6ccc5c4c3)n2)c1]

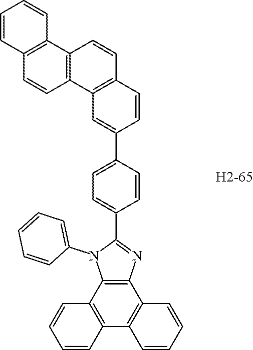 [SMILES: c1ccc(-n2c(-c3ccc(-c4ccc5ccc6c7ccccc7ccc6c5c4)cc3)nc3c4ccccc4c4ccccc4c32)cc1]

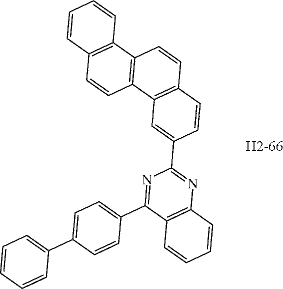 [SMILES: c1ccc(-c2ccc(-c3nc(-c4ccc5ccc6c7ccccc7ccc6c5c4)nc4ccccc34)cc2)cc1]

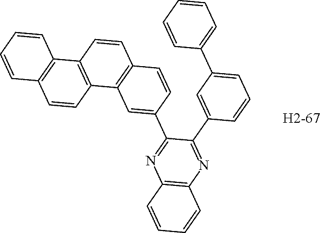 [SMILES: c1ccc(-c2cccc(-c3nc4ccccc4nc3-c3ccc4ccc5c6ccccc6ccc5c4c3)c2)cc1]

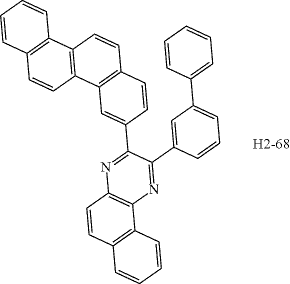 [SMILES: c1ccc(-c2cccc(-c3nc4c(ccc5ccccc54)nc3-c3ccc4ccc5c6ccccc6ccc5c4c3)c2)cc1]

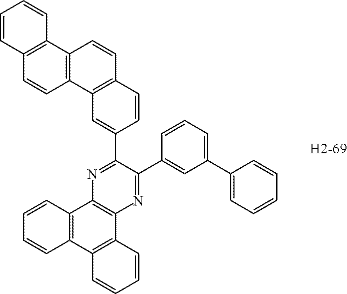 [SMILES: c1ccc(-c2cccc(-c3nc4c5ccccc5c5ccccc5c4nc3-c3ccc4ccc5c6ccccc6ccc5c4c3)c2)cc1]

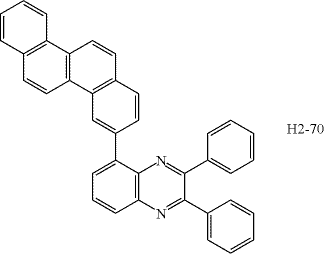 [SMILES: c1ccc(-c2nc3cccc(-c4ccc5ccc6c7ccccc7ccc6c5c4)c3nc2-c2ccccc2)cc1]

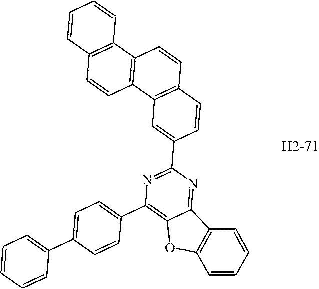 [SMILES: c1ccc(-c2ccc(-c3nc(-c4ccc5ccc6c7ccccc7ccc6c5c4)nc4c3oc3ccccc34)cc2)cc1]

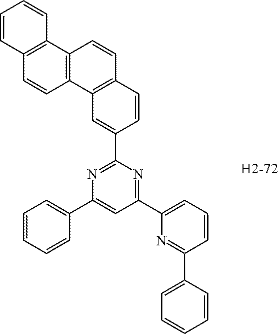 [SMILES: c1ccc(-c2cccc(-c3cc(-c4ccccc4)nc(-c4ccc5ccc6c7ccccc7ccc6c5c4)n3)n2)cc1]

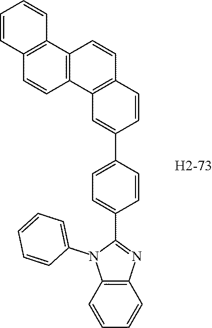 [SMILES: c1ccc(-n2c(-c3ccc(-c4ccc5ccc6c7ccccc7ccc6c5c4)cc3)nc3ccccc32)cc1]

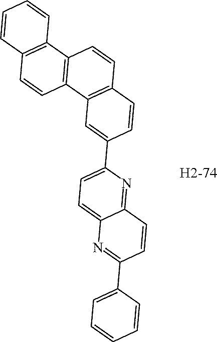 [SMILES: c1ccc(-c2ccc3nc(-c4ccc5ccc6c7ccccc7ccc6c5c4)ccc3n2)cc1]

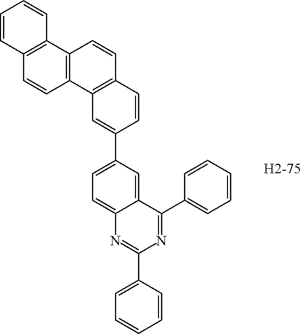 [SMILES: c1ccc(-c2nc(-c3ccccc3)c3cc(-c4ccc5ccc6c7ccccc7ccc6c5c4)ccc3n2)cc1]

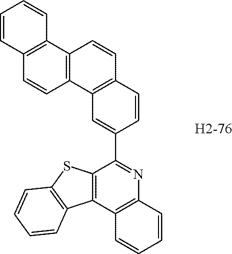 [SMILES: c1ccc2c(c1)ccc1c3cc(-c4nc5ccccc5c5c4sc4ccccc45)ccc3ccc21]

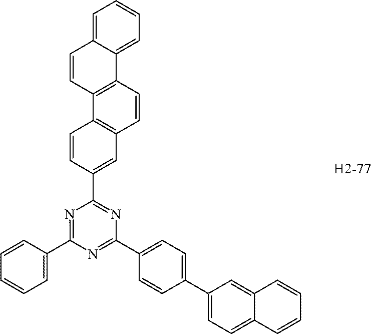 [SMILES: c1ccc(-c2nc(-c3ccc(-c4ccc5ccccc5c4)cc3)nc(-c3ccc4c(ccc5c6ccccc6ccc45)c3)n2)cc1]

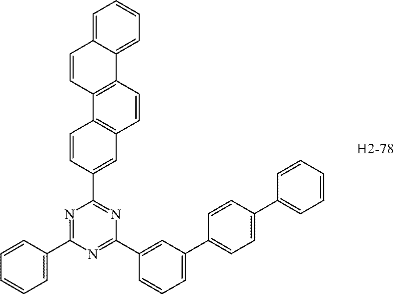 [SMILES: c1ccc(-c2ccc(-c3cccc(-c4nc(-c5ccccc5)nc(-c5ccc6c(ccc7c8ccccc8ccc67)c5)n4)c3)cc2)cc1]

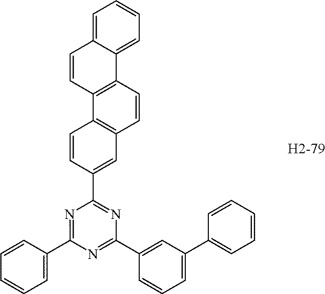 [SMILES: c1ccc(-c2cccc(-c3nc(-c4ccccc4)nc(-c4ccc5c(ccc6c7ccccc7ccc56)c4)n3)c2)cc1]

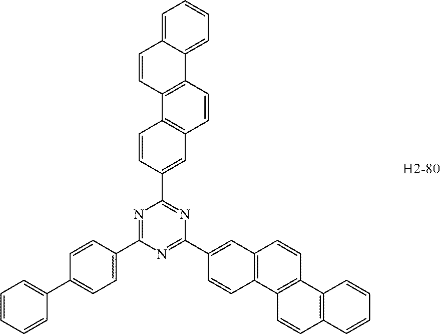 [SMILES: c1ccc(-c2ccc(-c3nc(-c4ccc5c(ccc6c7ccccc7ccc56)c4)nc(-c4ccc5c(ccc6c7ccccc7ccc56)c4)n3)cc2)cc1]

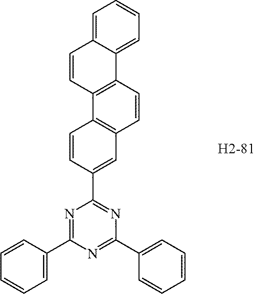 [SMILES: c1ccc(-c2nc(-c3ccccc3)nc(-c3ccc4c(ccc5c6ccccc6ccc45)c3)n2)cc1]

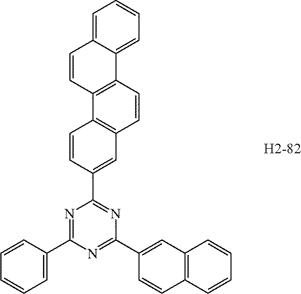 [SMILES: c1ccc(-c2nc(-c3ccc4ccccc4c3)nc(-c3ccc4c(ccc5c6ccccc6ccc45)c3)n2)cc1]

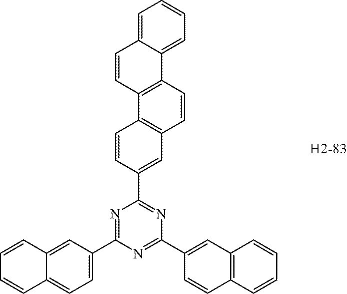 [SMILES: c1ccc2cc(-c3nc(-c4ccc5ccccc5c4)nc(-c4ccc5c(ccc6c7ccccc7ccc56)c4)n3)ccc2c1]

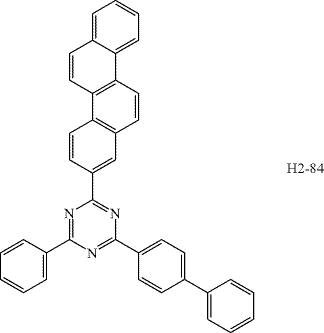 [SMILES: c1ccc(-c2ccc(-c3nc(-c4ccccc4)nc(-c4ccc5c(ccc6c7ccccc7ccc56)c4)n3)cc2)cc1]

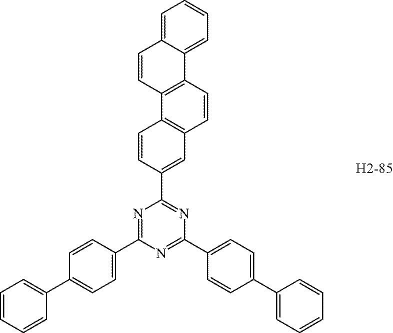 [SMILES: c1ccc(-c2ccc(-c3nc(-c4ccc(-c5ccccc5)cc4)nc(-c4ccc5c(ccc6c7ccccc7ccc56)c4)n3)cc2)cc1]

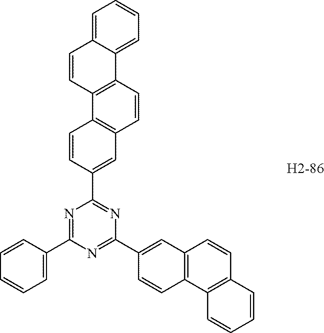 [SMILES: c1ccc(-c2nc(-c3ccc4c(ccc5ccccc54)c3)nc(-c3ccc4c(ccc5c6ccccc6ccc45)c3)n2)cc1]

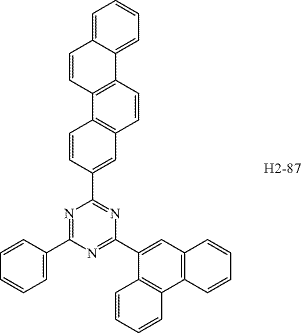 [SMILES: c1ccc(-c2nc(-c3ccc4c(ccc5c6ccccc6ccc45)c3)nc(-c3cc4ccccc4c4ccccc34)n2)cc1]

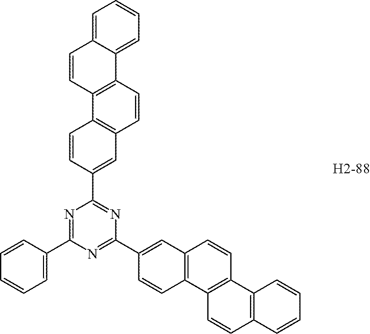 [SMILES: c1ccc(-c2nc(-c3ccc4c(ccc5c6ccccc6ccc45)c3)nc(-c3ccc4c(ccc5c6ccccc6ccc45)c3)n2)cc1]

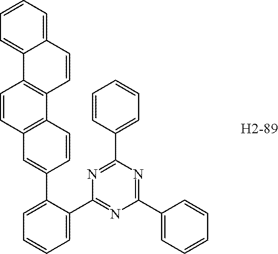 [SMILES: c1ccc(-c2nc(-c3ccccc3)nc(-c3ccccc3-c3ccc4c(ccc5c6ccccc6ccc45)c3)n2)cc1]

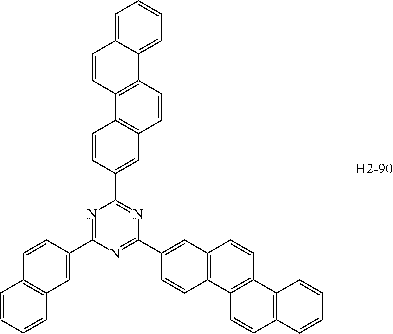 [SMILES: c1ccc2cc(-c3nc(-c4ccc5c(ccc6c7ccccc7ccc56)c4)nc(-c4ccc5c(ccc6c7ccccc7ccc56)c4)n3)ccc2c1]

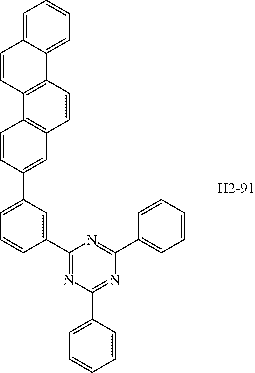 [SMILES: c1ccc(-c2nc(-c3ccccc3)nc(-c3cccc(-c4ccc5c(ccc6c7ccccc7ccc56)c4)c3)n2)cc1]

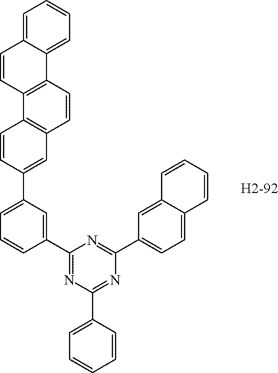 [SMILES: c1ccc(-c2nc(-c3cccc(-c4ccc5c(ccc6c7ccccc7ccc56)c4)c3)nc(-c3ccc4ccccc4c3)n2)cc1]

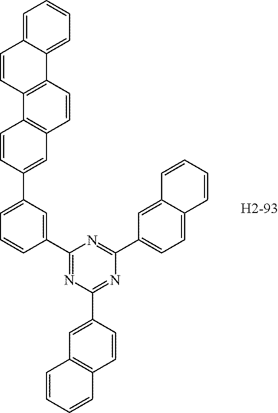 [SMILES: c1cc(-c2ccc3c(ccc4c5ccccc5ccc34)c2)cc(-c2nc(-c3ccc4ccccc4c3)nc(-c3ccc4ccccc4c3)n2)c1]

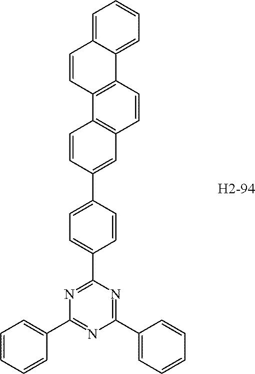 [SMILES: c1ccc(-c2nc(-c3ccccc3)nc(-c3ccc(-c4ccc5c(ccc6c7ccccc7ccc56)c4)cc3)n2)cc1]

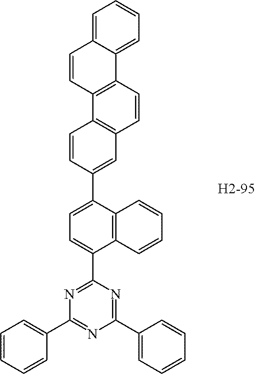 [SMILES: c1ccc(-c2nc(-c3ccccc3)nc(-c3ccc(-c4ccc5c(ccc6c7ccccc7ccc56)c4)c4ccccc34)n2)cc1]

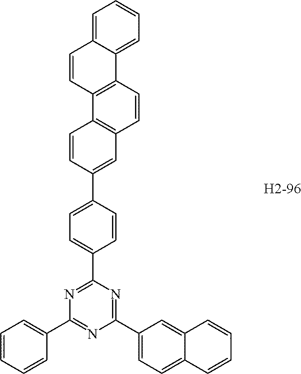 [SMILES: c1ccc(-c2nc(-c3ccc(-c4ccc5c(ccc6c7ccccc7ccc56)c4)cc3)nc(-c3ccc4ccccc4c3)n2)cc1]

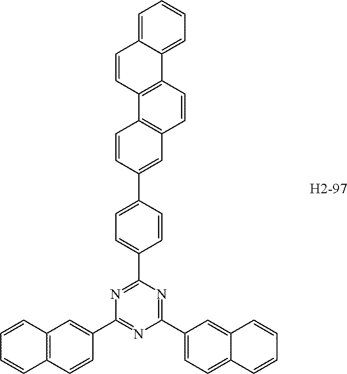 [SMILES: c1ccc2cc(-c3nc(-c4ccc(-c5ccc6c(ccc7c8ccccc8ccc67)c5)cc4)nc(-c4ccc5ccccc5c4)n3)ccc2c1]

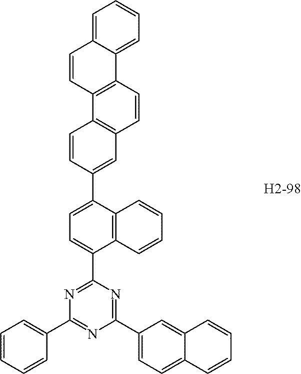 [SMILES: c1ccc(-c2nc(-c3ccc4ccccc4c3)nc(-c3ccc(-c4ccc5c(ccc6c7ccccc7ccc56)c4)c4ccccc34)n2)cc1]

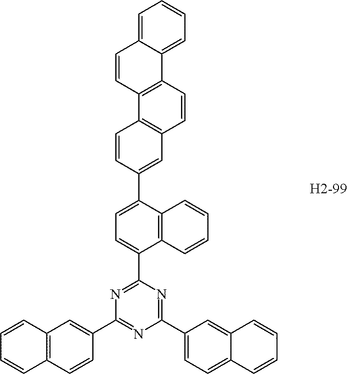 [SMILES: c1ccc2cc(-c3nc(-c4ccc5ccccc5c4)nc(-c4ccc(-c5ccc6c(ccc7c8ccccc8ccc67)c5)c5ccccc45)n3)ccc2c1]

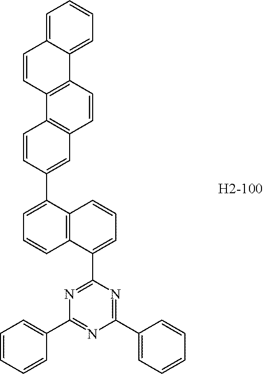 [SMILES: c1ccc(-c2nc(-c3ccccc3)nc(-c3cccc4c(-c5ccc6c(ccc7c8ccccc8ccc67)c5)cccc34)n2)cc1]

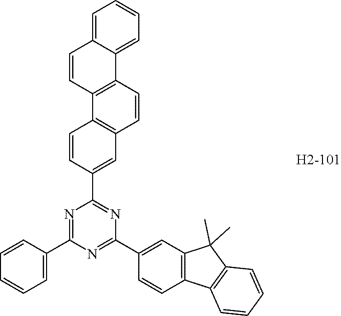 [SMILES: CC1(C)c2ccccc2-c2ccc(-c3nc(-c4ccccc4)nc(-c4ccc5c(ccc6c7ccccc7ccc56)c4)n3)cc21]

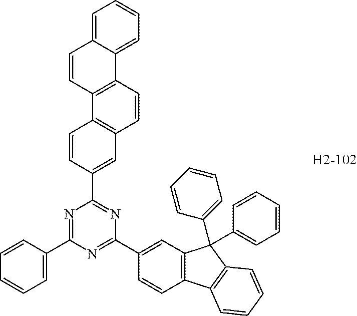 [SMILES: c1ccc(-c2nc(-c3ccc4c(c3)C(c3ccccc3)(c3ccccc3)c3ccccc3-4)nc(-c3ccc4c(ccc5c6ccccc6ccc45)c3)n2)cc1]

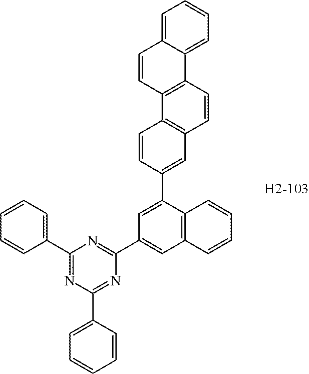 [SMILES: c1ccc(-c2nc(-c3ccccc3)nc(-c3cc(-c4ccc5c(ccc6c7ccccc7ccc56)c4)c4ccccc4c3)n2)cc1]

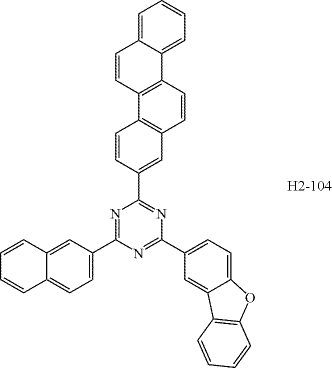 [SMILES: c1ccc2cc(-c3nc(-c4ccc5c(ccc6c7ccccc7ccc56)c4)nc(-c4ccc5oc6ccccc6c5c4)n3)ccc2c1]

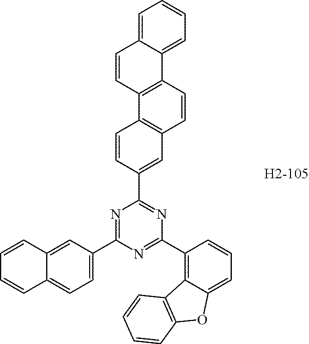 [SMILES: c1ccc2cc(-c3nc(-c4ccc5c(ccc6c7ccccc7ccc56)c4)nc(-c4cccc5oc6ccccc6c45)n3)ccc2c1]

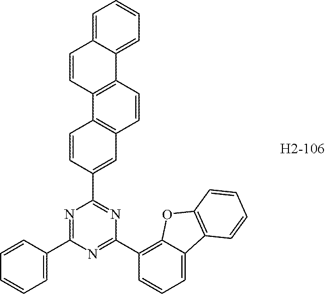 [SMILES: c1ccc(-c2nc(-c3ccc4c(ccc5c6ccccc6ccc45)c3)nc(-c3cccc4c3oc3ccccc34)n2)cc1]

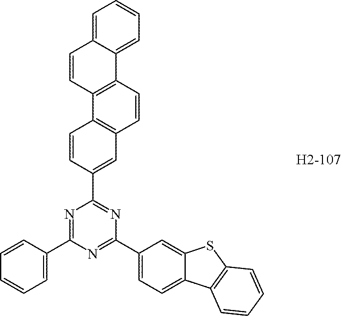 [SMILES: c1ccc(-c2nc(-c3ccc4c(ccc5c6ccccc6ccc45)c3)nc(-c3ccc4c(c3)sc3ccccc34)n2)cc1]

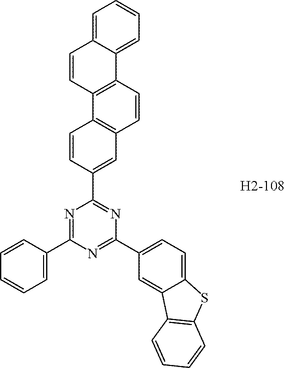 [SMILES: c1ccc(-c2nc(-c3ccc4c(ccc5c6ccccc6ccc45)c3)nc(-c3ccc4sc5ccccc5c4c3)n2)cc1]

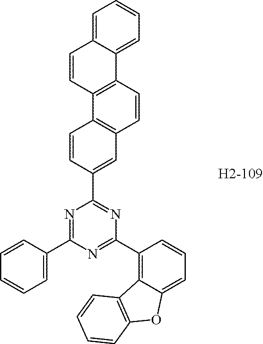 [SMILES: c1ccc(-c2nc(-c3ccc4c(ccc5c6ccccc6ccc45)c3)nc(-c3cccc4oc5ccccc5c34)n2)cc1]

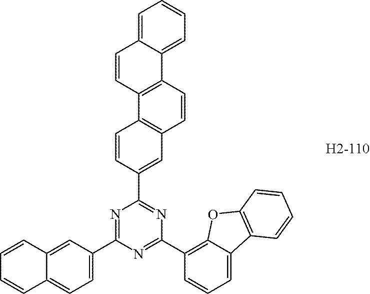 [SMILES: c1ccc2cc(-c3nc(-c4ccc5c(ccc6c7ccccc7ccc56)c4)nc(-c4cccc5c4oc4ccccc45)n3)ccc2c1]

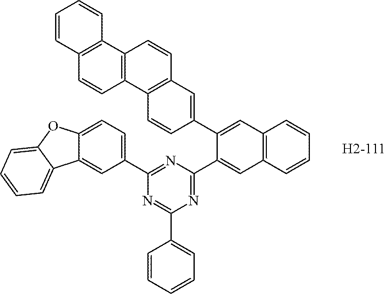 [SMILES: c1ccc(-c2nc(-c3ccc4oc5ccccc5c4c3)nc(-c3cc4ccccc4cc3-c3ccc4c(ccc5c6ccccc6ccc45)c3)n2)cc1]

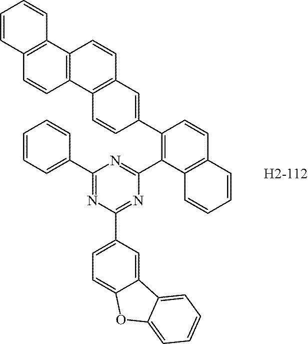 [SMILES: c1ccc(-c2nc(-c3ccc4oc5ccccc5c4c3)nc(-c3c(-c4ccc5c(ccc6c7ccccc7ccc56)c4)ccc4ccccc34)n2)cc1]

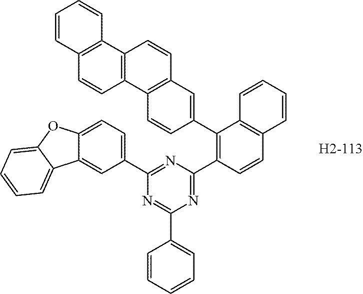 [SMILES: c1ccc(-c2nc(-c3ccc4oc5ccccc5c4c3)nc(-c3ccc4ccccc4c3-c3ccc4c(ccc5c6ccccc6ccc45)c3)n2)cc1]

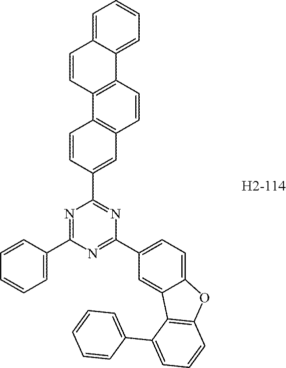 [SMILES: c1ccc(-c2nc(-c3ccc4c(ccc5c6ccccc6ccc45)c3)nc(-c3ccc4oc5cccc(-c6ccccc6)c5c4c3)n2)cc1]

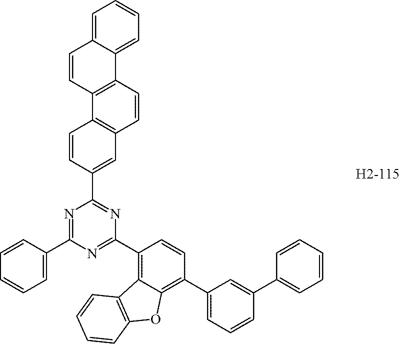 [SMILES: c1ccc(-c2cccc(-c3ccc(-c4nc(-c5ccccc5)nc(-c5ccc6c(ccc7c8ccccc8ccc67)c5)n4)c4c3oc3ccccc34)c2)cc1]

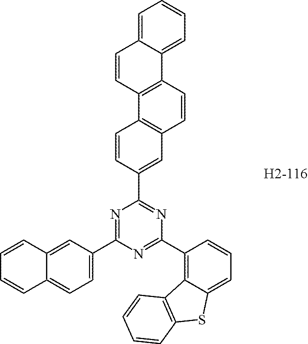 [SMILES: c1ccc2cc(-c3nc(-c4ccc5c(ccc6c7ccccc7ccc56)c4)nc(-c4cccc5sc6ccccc6c45)n3)ccc2c1]

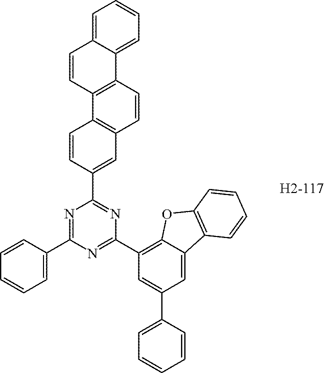 [SMILES: c1ccc(-c2cc(-c3nc(-c4ccccc4)nc(-c4ccc5c(ccc6c7ccccc7ccc56)c4)n3)c3oc4ccccc4c3c2)cc1]

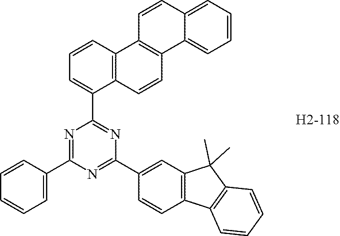 [SMILES: CC1(C)c2ccccc2-c2ccc(-c3nc(-c4ccccc4)nc(-c4cccc5c4ccc4c6ccccc6ccc54)n3)cc21]

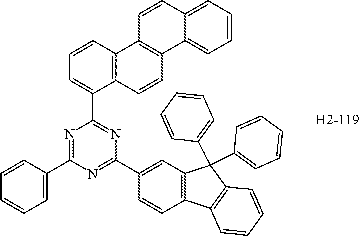 [SMILES: c1ccc(-c2nc(-c3ccc4c(c3)C(c3ccccc3)(c3ccccc3)c3ccccc3-4)nc(-c3cccc4c3ccc3c5ccccc5ccc43)n2)cc1]

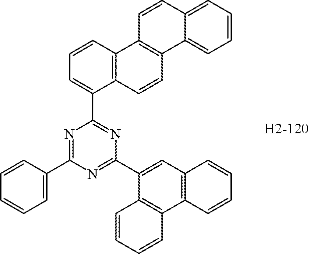 [SMILES: c1ccc(-c2nc(-c3cc4ccccc4c4ccccc34)nc(-c3cccc4c3ccc3c5ccccc5ccc43)n2)cc1]

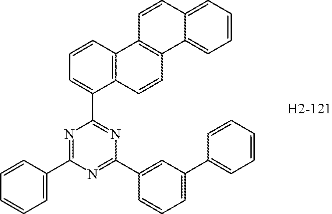 [SMILES: c1ccc(-c2cccc(-c3nc(-c4ccccc4)nc(-c4cccc5c4ccc4c6ccccc6ccc54)n3)c2)cc1]

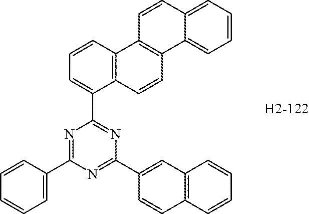 [SMILES: c1ccc(-c2nc(-c3ccc4ccccc4c3)nc(-c3cccc4c3ccc3c5ccccc5ccc43)n2)cc1]

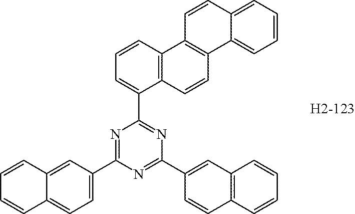 [SMILES: c1ccc2cc(-c3nc(-c4ccc5ccccc5c4)nc(-c4cccc5c4ccc4c6ccccc6ccc54)n3)ccc2c1]

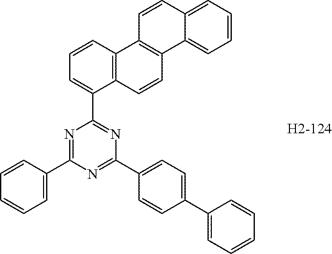 [SMILES: c1ccc(-c2ccc(-c3nc(-c4ccccc4)nc(-c4cccc5c4ccc4c6ccccc6ccc54)n3)cc2)cc1]

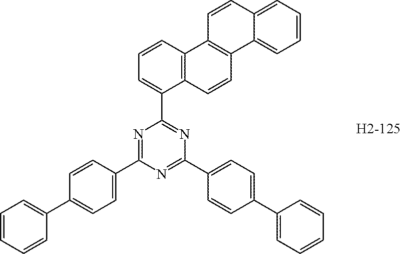 [SMILES: c1ccc(-c2ccc(-c3nc(-c4ccc(-c5ccccc5)cc4)nc(-c4cccc5c4ccc4c6ccccc6ccc54)n3)cc2)cc1]

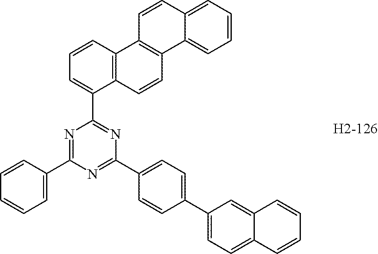 [SMILES: c1ccc(-c2nc(-c3ccc(-c4ccc5ccccc5c4)cc3)nc(-c3cccc4c3ccc3c5ccccc5ccc43)n2)cc1]

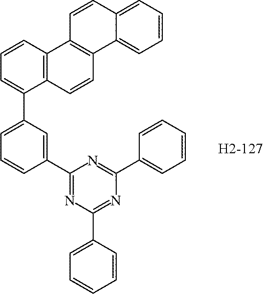 [SMILES: c1ccc(-c2nc(-c3ccccc3)nc(-c3cccc(-c4cccc5c4ccc4c6ccccc6ccc54)c3)n2)cc1]

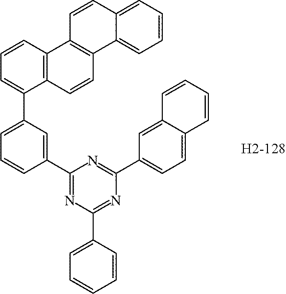 [SMILES: c1ccc(-c2nc(-c3cccc(-c4cccc5c4ccc4c6ccccc6ccc54)c3)nc(-c3ccc4ccccc4c3)n2)cc1]

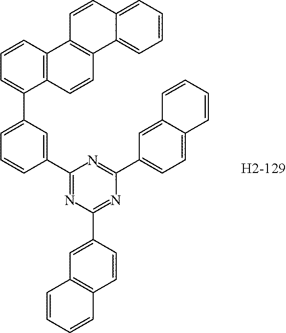 [SMILES: c1cc(-c2nc(-c3ccc4ccccc4c3)nc(-c3ccc4ccccc4c3)n2)cc(-c2cccc3c2ccc2c4ccccc4ccc32)c1]

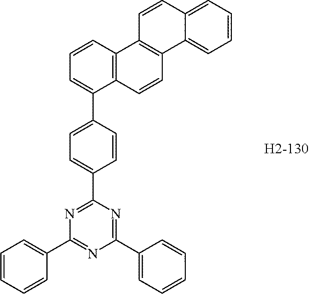 [SMILES: c1ccc(-c2nc(-c3ccccc3)nc(-c3ccc(-c4cccc5c4ccc4c6ccccc6ccc54)cc3)n2)cc1]

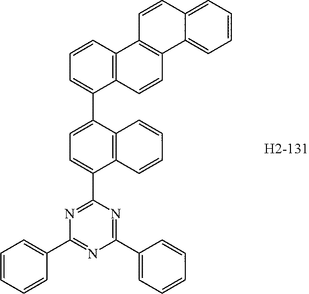 [SMILES: c1ccc(-c2nc(-c3ccccc3)nc(-c3ccc(-c4cccc5c4ccc4c6ccccc6ccc54)c4ccccc34)n2)cc1]

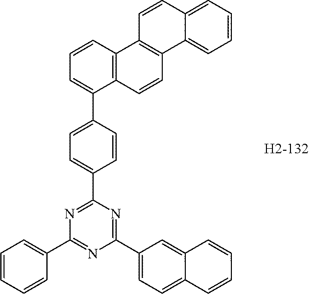 [SMILES: c1ccc(-c2nc(-c3ccc(-c4cccc5c4ccc4c6ccccc6ccc54)cc3)nc(-c3ccc4ccccc4c3)n2)cc1]

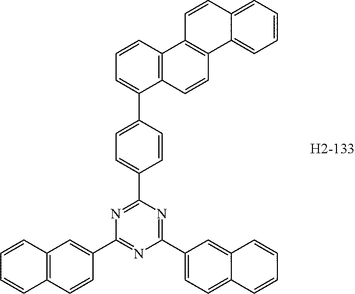 [SMILES: c1ccc2cc(-c3nc(-c4ccc(-c5cccc6c5ccc5c7ccccc7ccc65)cc4)nc(-c4ccc5ccccc5c4)n3)ccc2c1]

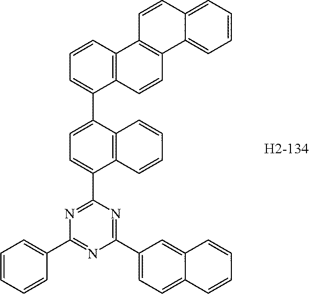 [SMILES: c1ccc(-c2nc(-c3ccc4ccccc4c3)nc(-c3ccc(-c4cccc5c4ccc4c6ccccc6ccc54)c4ccccc34)n2)cc1]

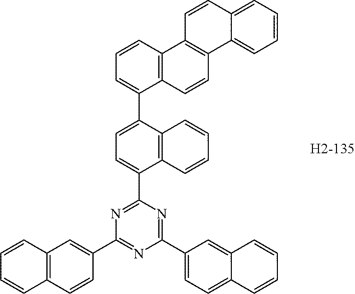 [SMILES: c1ccc2cc(-c3nc(-c4ccc5ccccc5c4)nc(-c4ccc(-c5cccc6c5ccc5c7ccccc7ccc65)c5ccccc45)n3)ccc2c1]

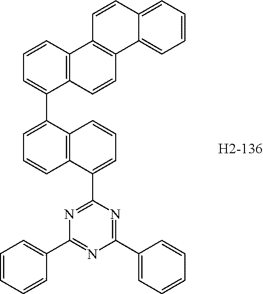 [SMILES: c1ccc(-c2nc(-c3ccccc3)nc(-c3cccc4c(-c5cccc6c5ccc5c7ccccc7ccc65)cccc34)n2)cc1]

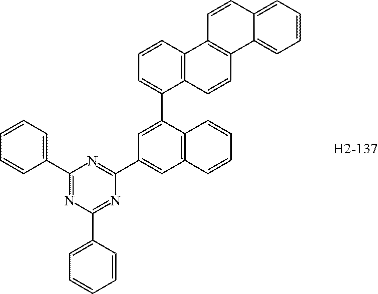 [SMILES: c1ccc(-c2nc(-c3ccccc3)nc(-c3cc(-c4cccc5c4ccc4c6ccccc6ccc54)c4ccccc4c3)n2)cc1]

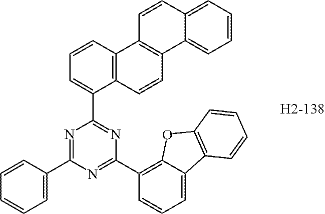 [SMILES: c1ccc(-c2nc(-c3cccc4c3ccc3c5ccccc5ccc43)nc(-c3cccc4c3oc3ccccc34)n2)cc1]

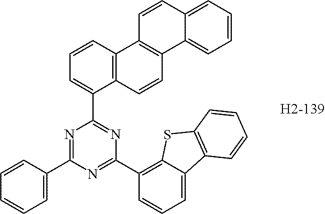 [SMILES: c1ccc(-c2nc(-c3cccc4c3ccc3c5ccccc5ccc43)nc(-c3cccc4c3sc3ccccc34)n2)cc1]

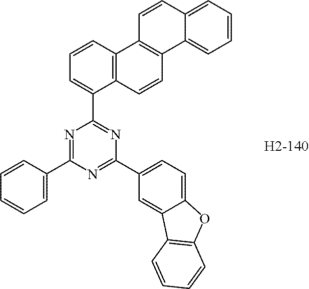 [SMILES: c1ccc(-c2nc(-c3ccc4oc5ccccc5c4c3)nc(-c3cccc4c3ccc3c5ccccc5ccc43)n2)cc1]

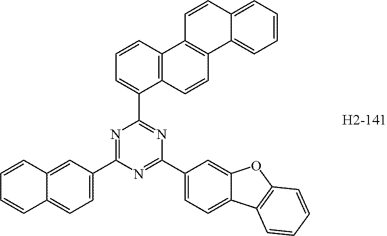 [SMILES: c1ccc2cc(-c3nc(-c4ccc5c(c4)oc4ccccc45)nc(-c4cccc5c4ccc4c6ccccc6ccc54)n3)ccc2c1]

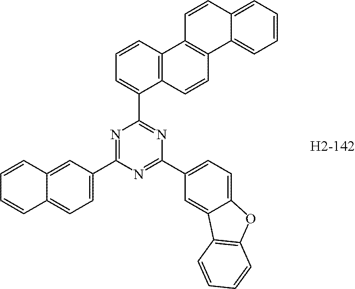 [SMILES: c1ccc2cc(-c3nc(-c4ccc5oc6ccccc6c5c4)nc(-c4cccc5c4ccc4c6ccccc6ccc54)n3)ccc2c1]

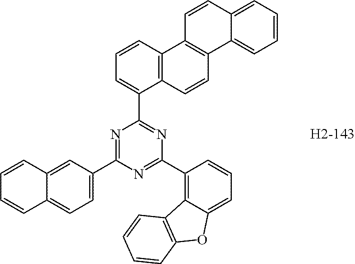 [SMILES: c1ccc2cc(-c3nc(-c4cccc5c4ccc4c6ccccc6ccc54)nc(-c4cccc5oc6ccccc6c45)n3)ccc2c1]

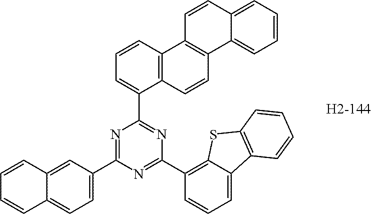 [SMILES: c1ccc2cc(-c3nc(-c4cccc5c4ccc4c6ccccc6ccc54)nc(-c4cccc5c4sc4ccccc45)n3)ccc2c1]

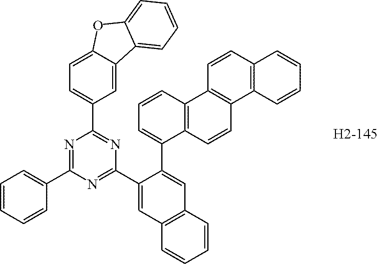 [SMILES: c1ccc(-c2nc(-c3ccc4oc5ccccc5c4c3)nc(-c3cc4ccccc4cc3-c3cccc4c3ccc3c5ccccc5ccc43)n2)cc1]

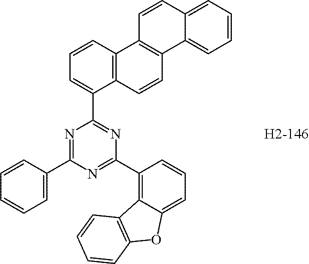 [SMILES: c1ccc(-c2nc(-c3cccc4c3ccc3c5ccccc5ccc43)nc(-c3cccc4oc5ccccc5c34)n2)cc1]

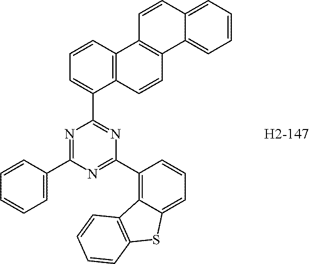 [SMILES: c1ccc(-c2nc(-c3cccc4c3ccc3c5ccccc5ccc43)nc(-c3cccc4sc5ccccc5c34)n2)cc1]

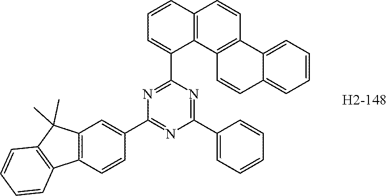 [SMILES: CC1(C)c2ccccc2-c2ccc(-c3nc(-c4ccccc4)nc(-c4cccc5ccc6c7ccccc7ccc6c45)n3)cc21]

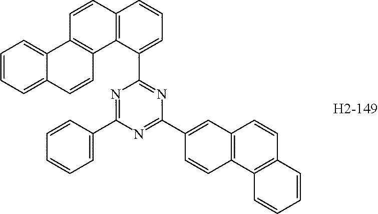 [SMILES: c1ccc(-c2nc(-c3ccc4c(ccc5ccccc54)c3)nc(-c3cccc4ccc5c6ccccc6ccc5c34)n2)cc1]

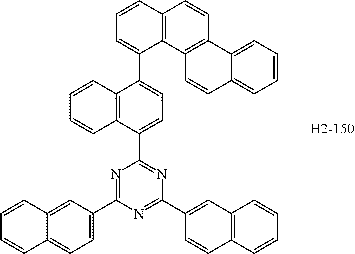 [SMILES: c1ccc2cc(-c3nc(-c4ccc5ccccc5c4)nc(-c4ccc(-c5cccc6ccc7c8ccccc8ccc7c56)c5ccccc45)n3)ccc2c1]

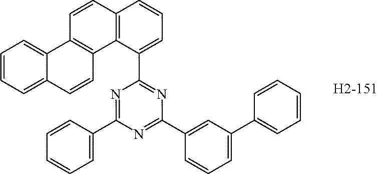 [SMILES: c1ccc(-c2cccc(-c3nc(-c4ccccc4)nc(-c4cccc5ccc6c7ccccc7ccc6c45)n3)c2)cc1]

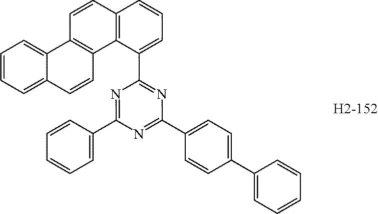 [SMILES: c1ccc(-c2ccc(-c3nc(-c4ccccc4)nc(-c4cccc5ccc6c7ccccc7ccc6c45)n3)cc2)cc1]

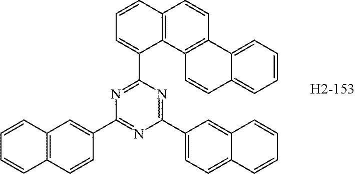 [SMILES: c1ccc2cc(-c3nc(-c4ccc5ccccc5c4)nc(-c4cccc5ccc6c7ccccc7ccc6c45)n3)ccc2c1]

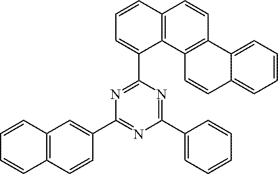 [SMILES: c1ccc(-c2nc(-c3ccc4ccccc4c3)nc(-c3cccc4ccc5c6ccccc6ccc5c34)n2)cc1]

H2-154

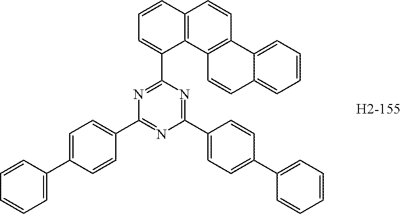 [SMILES: c1ccc(-c2ccc(-c3nc(-c4ccc(-c5ccccc5)cc4)nc(-c4cccc5ccc6c7ccccc7ccc6c45)n3)cc2)cc1]

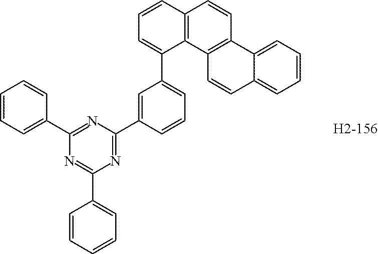 [SMILES: c1ccc(-c2nc(-c3ccccc3)nc(-c3cccc(-c4cccc5ccc6c7ccccc7ccc6c45)c3)n2)cc1]

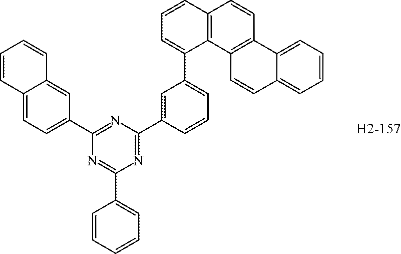 [SMILES: c1ccc(-c2nc(-c3cccc(-c4cccc5ccc6c7ccccc7ccc6c45)c3)nc(-c3ccc4ccccc4c3)n2)cc1]

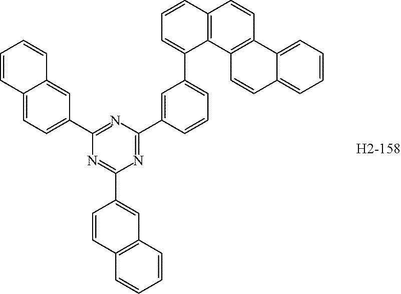 [SMILES: c1cc(-c2nc(-c3ccc4ccccc4c3)nc(-c3ccc4ccccc4c3)n2)cc(-c2cccc3ccc4c5ccccc5ccc4c23)c1]

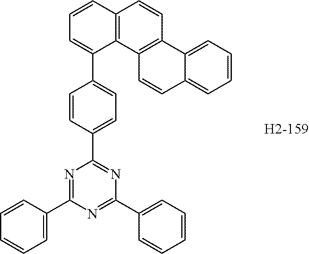 [SMILES: c1ccc(-c2nc(-c3ccccc3)nc(-c3ccc(-c4cccc5ccc6c7ccccc7ccc6c45)cc3)n2)cc1]

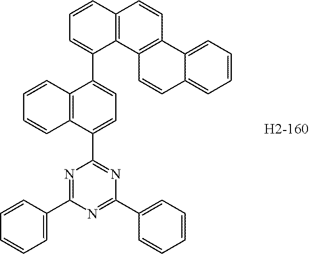 [SMILES: c1ccc(-c2nc(-c3ccccc3)nc(-c3ccc(-c4cccc5ccc6c7ccccc7ccc6c45)c4ccccc34)n2)cc1]

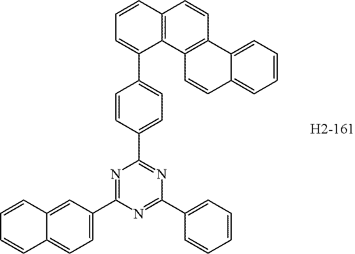 [SMILES: c1ccc(-c2nc(-c3ccc(-c4cccc5ccc6c7ccccc7ccc6c45)cc3)nc(-c3ccc4ccccc4c3)n2)cc1]

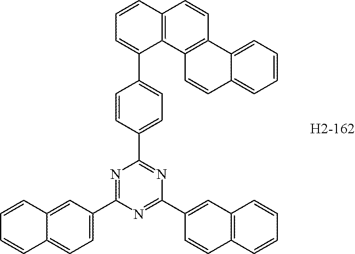 [SMILES: c1ccc2cc(-c3nc(-c4ccc(-c5cccc6ccc7c8ccccc8ccc7c56)cc4)nc(-c4ccc5ccccc5c4)n3)ccc2c1]

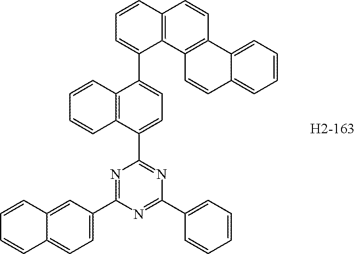 [SMILES: c1ccc(-c2nc(-c3ccc4ccccc4c3)nc(-c3ccc(-c4cccc5ccc6c7ccccc7ccc6c45)c4ccccc34)n2)cc1]

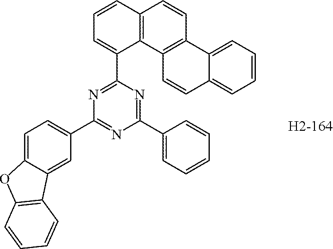 [SMILES: c1ccc(-c2nc(-c3ccc4oc5ccccc5c4c3)nc(-c3cccc4ccc5c6ccccc6ccc5c34)n2)cc1]

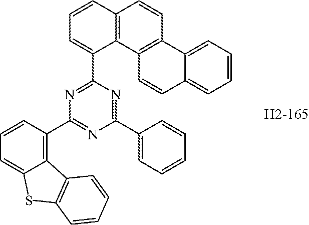 [SMILES: c1ccc(-c2nc(-c3cccc4ccc5c6ccccc6ccc5c34)nc(-c3cccc4sc5ccccc5c34)n2)cc1]

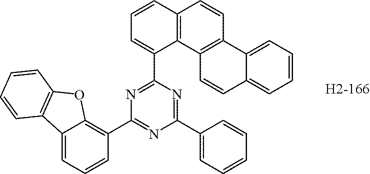 [SMILES: c1ccc(-c2nc(-c3cccc4c3oc3ccccc34)nc(-c3cccc4ccc5c6ccccc6ccc5c34)n2)cc1]

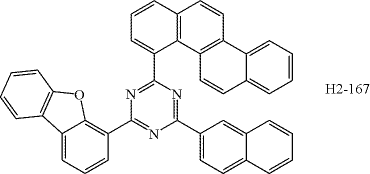 [SMILES: c1ccc2cc(-c3nc(-c4cccc5c4oc4ccccc45)nc(-c4cccc5ccc6c7ccccc7ccc6c45)n3)ccc2c1]

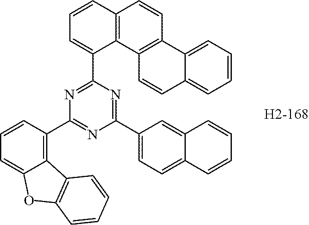 [SMILES: c1ccc2cc(-c3nc(-c4cccc5ccc6c7ccccc7ccc6c45)nc(-c4cccc5oc6ccccc6c45)n3)ccc2c1]

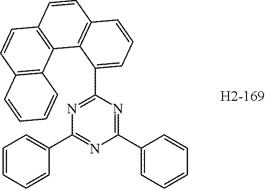 [SMILES: c1ccc(-c2nc(-c3ccccc3)nc(-c3cccc4ccc5ccc6ccccc6c5c34)n2)cc1]

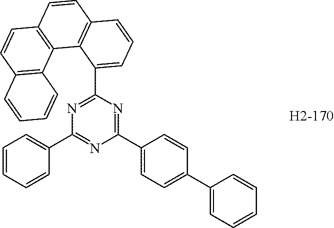 [SMILES: c1ccc(-c2ccc(-c3nc(-c4ccccc4)nc(-c4cccc5ccc6ccc7ccccc7c6c45)n3)cc2)cc1]

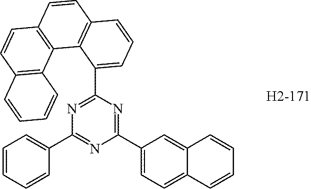 [SMILES: c1ccc(-c2nc(-c3ccc4ccccc4c3)nc(-c3cccc4ccc5ccc6ccccc6c5c34)n2)cc1]

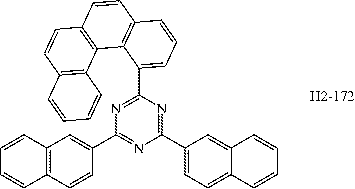 [SMILES: c1ccc2cc(-c3nc(-c4ccc5ccccc5c4)nc(-c4cccc5ccc6ccc7ccccc7c6c45)n3)ccc2c1]

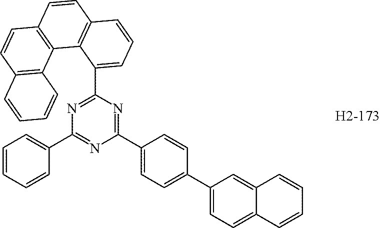 [SMILES: c1ccc(-c2nc(-c3ccc(-c4ccc5ccccc5c4)cc3)nc(-c3cccc4ccc5ccc6ccccc6c5c34)n2)cc1]

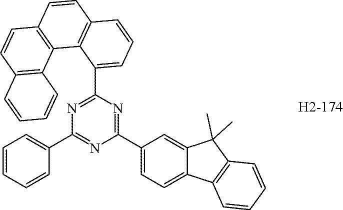 [SMILES: CC1(C)c2ccccc2-c2ccc(-c3nc(-c4ccccc4)nc(-c4cccc5ccc6ccc7ccccc7c6c45)n3)cc21]

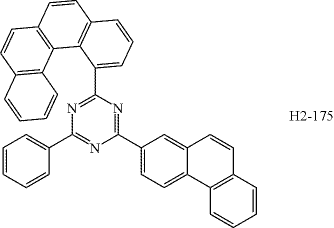 [SMILES: c1ccc(-c2nc(-c3ccc4c(ccc5ccccc54)c3)nc(-c3cccc4ccc5ccc6ccccc6c5c34)n2)cc1]

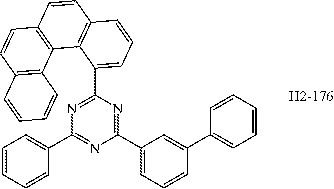 [SMILES: c1ccc(-c2cccc(-c3nc(-c4ccccc4)nc(-c4cccc5ccc6ccc7ccccc7c6c45)n3)c2)cc1]

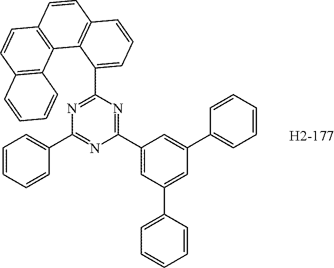 [SMILES: c1ccc(-c2cc(-c3ccccc3)cc(-c3nc(-c4ccccc4)nc(-c4cccc5ccc6ccc7ccccc7c6c45)n3)c2)cc1]

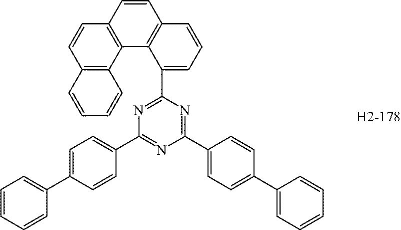 [SMILES: c1ccc(-c2ccc(-c3nc(-c4ccc(-c5ccccc5)cc4)nc(-c4cccc5ccc6ccc7ccccc7c6c45)n3)cc2)cc1]

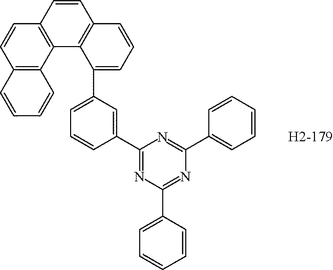 [SMILES: c1ccc(-c2nc(-c3ccccc3)nc(-c3cccc(-c4cccc5ccc6ccc7ccccc7c6c45)c3)n2)cc1]

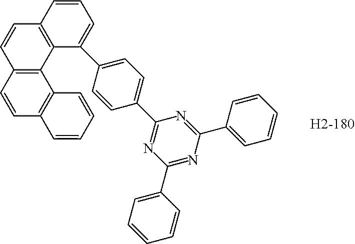 [SMILES: c1ccc(-c2nc(-c3ccccc3)nc(-c3ccc(-c4cccc5ccc6ccc7ccccc7c6c45)cc3)n2)cc1]

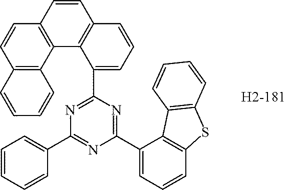 [SMILES: c1ccc(-c2nc(-c3cccc4sc5ccccc5c34)nc(-c3cccc4ccc5ccc6ccccc6c5c34)n2)cc1]

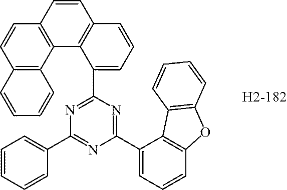 [SMILES: c1ccc(-c2nc(-c3cccc4oc5ccccc5c34)nc(-c3cccc4ccc5ccc6ccccc6c5c34)n2)cc1]

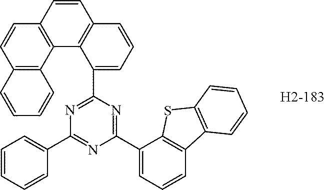 [SMILES: c1ccc(-c2nc(-c3cccc4c3sc3ccccc34)nc(-c3cccc4ccc5ccc6ccccc6c5c34)n2)cc1]

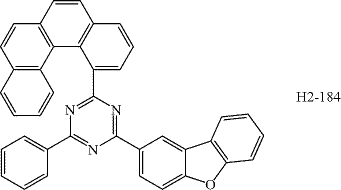 [SMILES: c1ccc(-c2nc(-c3ccc4oc5ccccc5c4c3)nc(-c3cccc4ccc5ccc6ccccc6c5c34)n2)cc1]

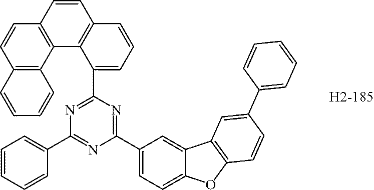 [SMILES: c1ccc(-c2ccc3oc4ccc(-c5nc(-c6ccccc6)nc(-c6cccc7ccc8ccc9ccccc9c8c67)n5)cc4c3c2)cc1]

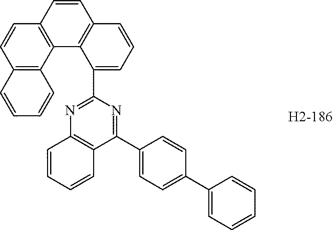 [SMILES: c1ccc(-c2ccc(-c3nc(-c4cccc5ccc6ccc7ccccc7c6c45)nc4ccccc34)cc2)cc1]

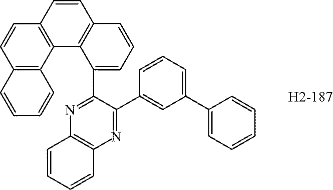 [SMILES: c1ccc(-c2cccc(-c3nc4ccccc4nc3-c3cccc4ccc5ccc6ccccc6c5c34)c2)cc1]

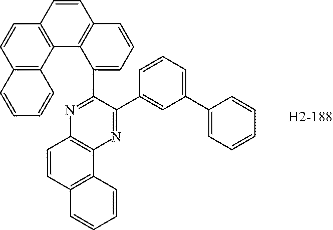 [SMILES: c1ccc(-c2cccc(-c3nc4c(ccc5ccccc54)nc3-c3cccc4ccc5ccc6ccccc6c5c34)c2)cc1]

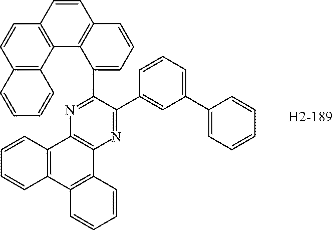 [SMILES: c1ccc(-c2cccc(-c3nc4c5ccccc5c5ccccc5c4nc3-c3cccc4ccc5ccc6ccccc6c5c34)c2)cc1]

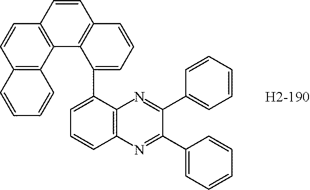 [SMILES: c1ccc(-c2nc3cccc(-c4cccc5ccc6ccc7ccccc7c6c45)c3nc2-c2ccccc2)cc1]

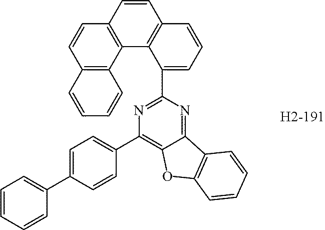 [SMILES: c1ccc(-c2ccc(-c3nc(-c4cccc5ccc6ccc7ccccc7c6c45)nc4c3oc3ccccc34)cc2)cc1]

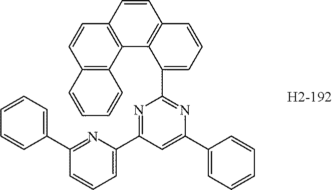 [SMILES: c1ccc(-c2cccc(-c3cc(-c4ccccc4)nc(-c4cccc5ccc6ccc7ccccc7c6c45)n3)n2)cc1]

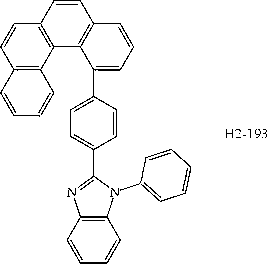 [SMILES: c1ccc(-n2c(-c3ccc(-c4cccc5ccc6ccc7ccccc7c6c45)cc3)nc3ccccc32)cc1]

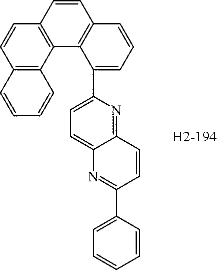 [SMILES: c1ccc(-c2ccc3nc(-c4cccc5ccc6ccc7ccccc7c6c45)ccc3n2)cc1]

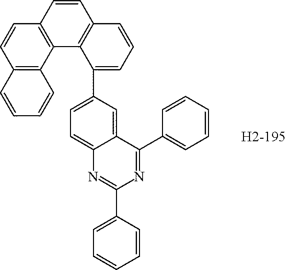 [SMILES: c1ccc(-c2nc(-c3ccccc3)c3cc(-c4cccc5ccc6ccc7ccccc7c6c45)ccc3n2)cc1]

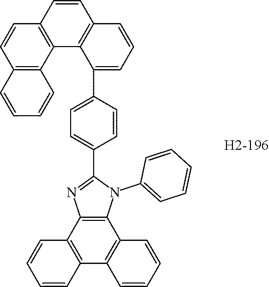 [SMILES: c1ccc(-n2c(-c3ccc(-c4cccc5ccc6ccc7ccccc7c6c45)cc3)nc3c4ccccc4c4ccccc4c32)cc1]

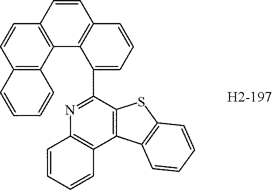 [SMILES: c1ccc2c(c1)ccc1ccc3cccc(-c4nc5ccccc5c5c4sc4ccccc45)c3c12]

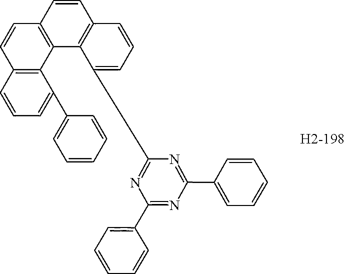 [SMILES: c1ccc(-c2nc(-c3ccccc3)nc(-c3cccc4ccc5ccc6cccc(-c7ccccc7)c6c5c34)n2)cc1]

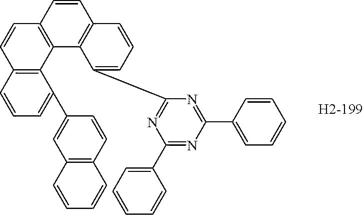 [SMILES: c1ccc(-c2nc(-c3ccccc3)nc(-c3cccc4ccc5ccc6cccc(-c7ccc8ccccc8c7)c6c5c34)n2)cc1]

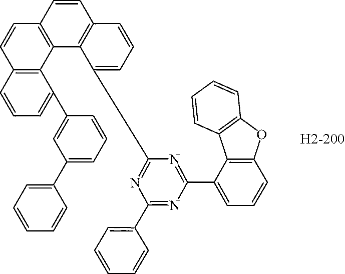 [SMILES: c1ccc(-c2cccc(-c3cccc4ccc5ccc6cccc(-c7nc(-c8ccccc8)nc(-c8cccc9oc%10ccccc%10c89)n7)c6c5c34)c2)cc1]

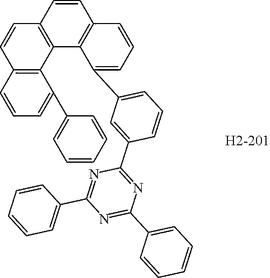 [SMILES: c1ccc(-c2nc(-c3ccccc3)nc(-c3cccc(-c4cccc5ccc6ccc7cccc(-c8ccccc8)c7c6c45)c3)n2)cc1]

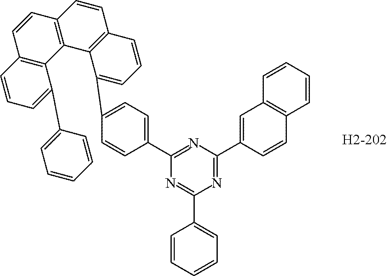 [SMILES: c1ccc(-c2nc(-c3ccc(-c4cccc5ccc6ccc7cccc(-c8ccccc8)c7c6c45)cc3)nc(-c3ccc4ccccc4c3)n2)cc1]

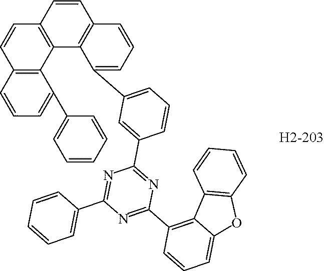 [SMILES: c1ccc(-c2nc(-c3cccc(-c4cccc5ccc6ccc7cccc(-c8ccccc8)c7c6c45)c3)nc(-c3cccc4oc5ccccc5c34)n2)cc1]

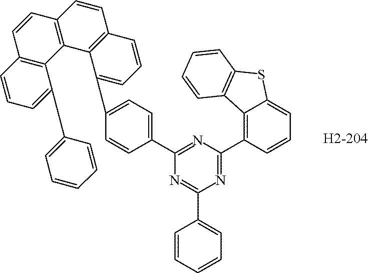 [SMILES: c1ccc(-c2nc(-c3ccc(-c4cccc5ccc6ccc7cccc(-c8ccccc8)c7c6c45)cc3)nc(-c3cccc4sc5ccccc5c34)n2)cc1]

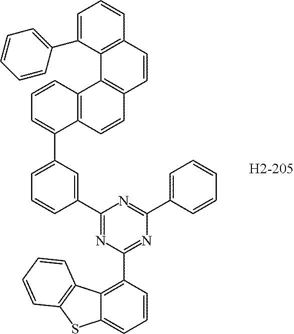 [SMILES: c1ccc(-c2nc(-c3cccc(-c4cccc5c4ccc4ccc6cccc(-c7ccccc7)c6c45)c3)nc(-c3cccc4sc5ccccc5c34)n2)cc1]

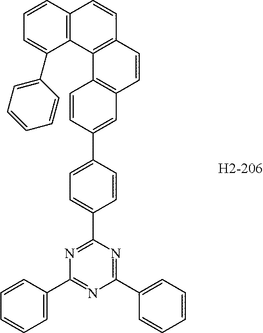 [SMILES: c1ccc(-c2nc(-c3ccccc3)nc(-c3ccc(-c4ccc5c(ccc6ccc7cccc(-c8ccccc8)c7c65)c4)cc3)n2)cc1]

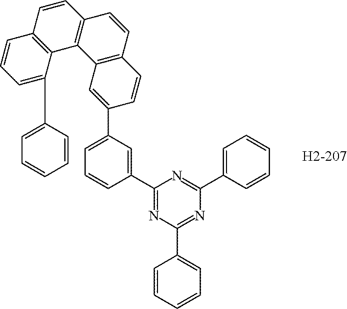 [SMILES: c1ccc(-c2nc(-c3ccccc3)nc(-c3cccc(-c4ccc5ccc6ccc7cccc(-c8ccccc8)c7c6c5c4)c3)n2)cc1]

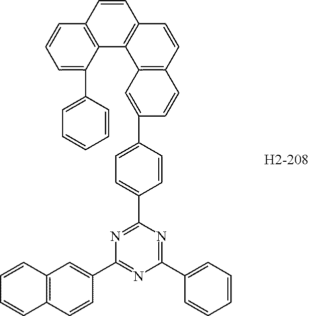 [SMILES: c1ccc(-c2nc(-c3ccc(-c4ccc5ccc6ccc7cccc(-c8ccccc8)c7c6c5c4)cc3)nc(-c3ccc4ccccc4c3)n2)cc1]

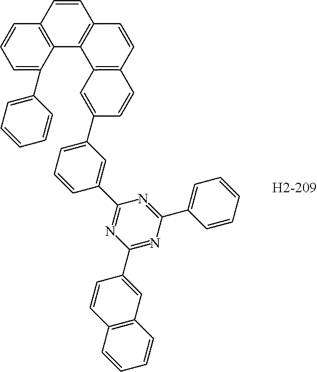 [SMILES: c1ccc(-c2nc(-c3cccc(-c4ccc5ccc6ccc7cccc(-c8ccccc8)c7c6c5c4)c3)nc(-c3ccc4ccccc4c3)n2)cc1]

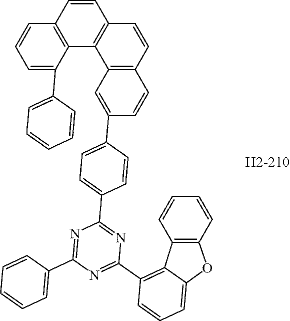 [SMILES: c1ccc(-c2nc(-c3ccc(-c4ccc5ccc6ccc7cccc(-c8ccccc8)c7c6c5c4)cc3)nc(-c3cccc4oc5ccccc5c34)n2)cc1]

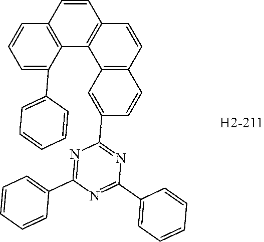 [SMILES: c1ccc(-c2nc(-c3ccccc3)nc(-c3ccc4ccc5ccc6cccc(-c7ccccc7)c6c5c4c3)n2)cc1]

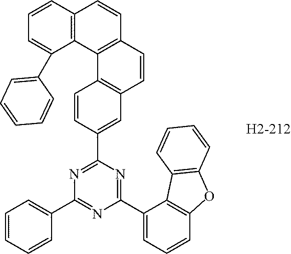 [SMILES: c1ccc(-c2nc(-c3ccc4c(ccc5ccc6cccc(-c7ccccc7)c6c54)c3)nc(-c3cccc4oc5ccccc5c34)n2)cc1]

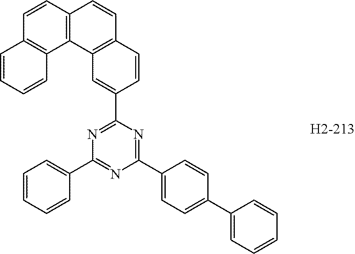 [SMILES: c1ccc(-c2ccc(-c3nc(-c4ccccc4)nc(-c4ccc5ccc6ccc7ccccc7c6c5c4)n3)cc2)cc1]

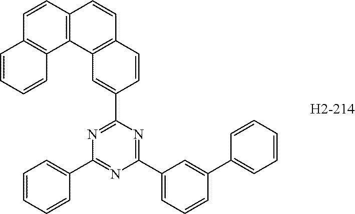 [SMILES: c1ccc(-c2cccc(-c3nc(-c4ccccc4)nc(-c4ccc5ccc6ccc7ccccc7c6c5c4)n3)c2)cc1]

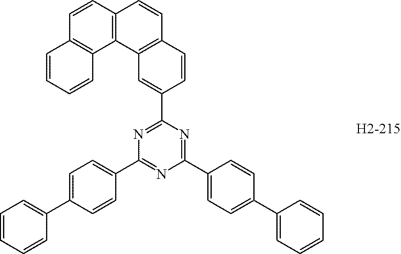 [SMILES: c1ccc(-c2ccc(-c3nc(-c4ccc(-c5ccccc5)cc4)nc(-c4ccc5ccc6ccc7ccccc7c6c5c4)n3)cc2)cc1]

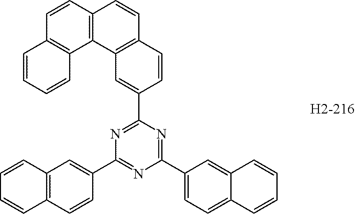 [SMILES: c1ccc2cc(-c3nc(-c4ccc5ccccc5c4)nc(-c4ccc5ccc6ccc7ccccc7c6c5c4)n3)ccc2c1]

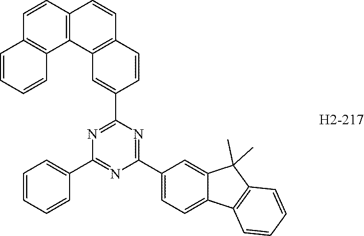 [SMILES: CC1(C)c2ccccc2-c2ccc(-c3nc(-c4ccccc4)nc(-c4ccc5ccc6ccc7ccccc7c6c5c4)n3)cc21]

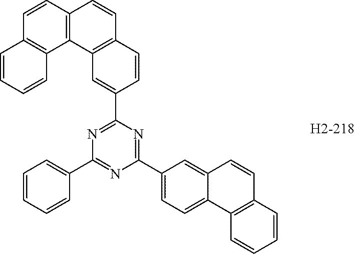 [SMILES: c1ccc(-c2nc(-c3ccc4c(ccc5ccccc54)c3)nc(-c3ccc4ccc5ccc6ccccc6c5c4c3)n2)cc1]

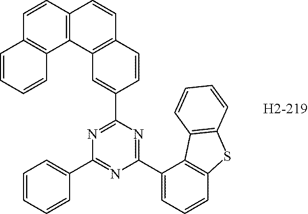 [SMILES: c1ccc(-c2nc(-c3ccc4ccc5ccc6ccccc6c5c4c3)nc(-c3cccc4sc5ccccc5c34)n2)cc1]

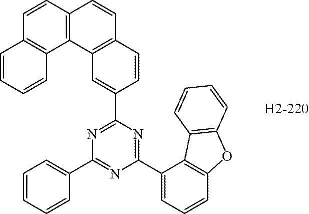 [SMILES: c1ccc(-c2nc(-c3ccc4ccc5ccc6ccccc6c5c4c3)nc(-c3cccc4oc5ccccc5c34)n2)cc1]

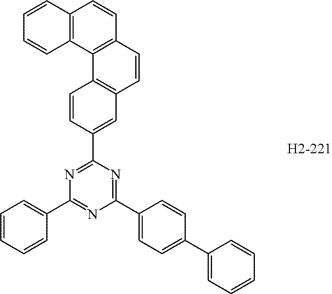 [SMILES: c1ccc(-c2ccc(-c3nc(-c4ccccc4)nc(-c4ccc5c(ccc6ccc7ccccc7c65)c4)n3)cc2)cc1]

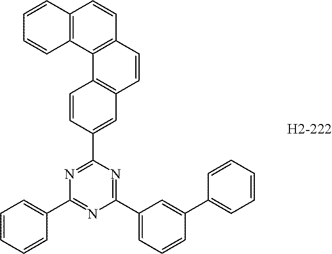 [SMILES: c1ccc(-c2cccc(-c3nc(-c4ccccc4)nc(-c4ccc5c(ccc6ccc7ccccc7c65)c4)n3)c2)cc1]

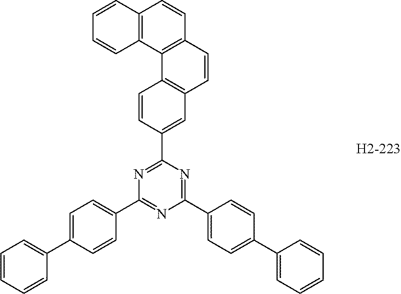 [SMILES: c1ccc(-c2ccc(-c3nc(-c4ccc(-c5ccccc5)cc4)nc(-c4ccc5c(ccc6ccc7ccccc7c65)c4)n3)cc2)cc1]

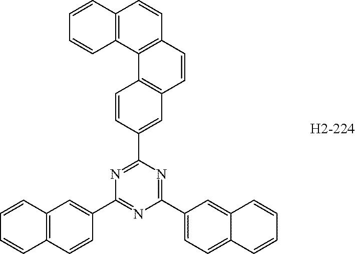 [SMILES: c1ccc2cc(-c3nc(-c4ccc5ccccc5c4)nc(-c4ccc5c(ccc6ccc7ccccc7c65)c4)n3)ccc2c1]

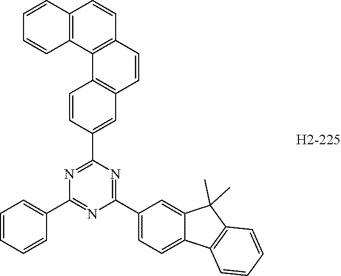 [SMILES: CC1(C)c2ccccc2-c2ccc(-c3nc(-c4ccccc4)nc(-c4ccc5c(ccc6ccc7ccccc7c65)c4)n3)cc21]

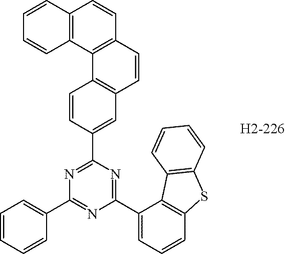 [SMILES: c1ccc(-c2nc(-c3ccc4c(ccc5ccc6ccccc6c54)c3)nc(-c3cccc4sc5ccccc5c34)n2)cc1]

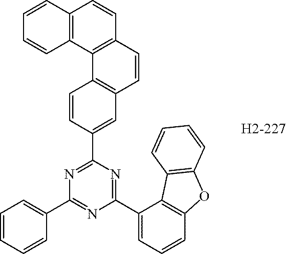 [SMILES: c1ccc(-c2nc(-c3ccc4c(ccc5ccc6ccccc6c54)c3)nc(-c3cccc4oc5ccccc5c34)n2)cc1]

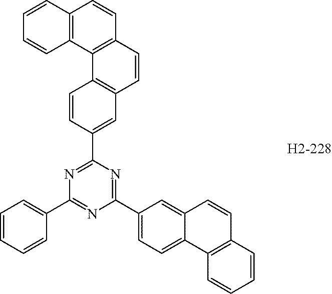 [SMILES: c1ccc(-c2nc(-c3ccc4c(ccc5ccccc54)c3)nc(-c3ccc4c(ccc5ccc6ccccc6c54)c3)n2)cc1]

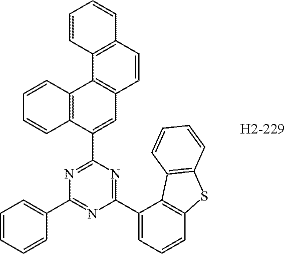 [SMILES: c1ccc(-c2nc(-c3cc4ccc5ccccc5c4c4ccccc34)nc(-c3cccc4sc5ccccc5c34)n2)cc1]

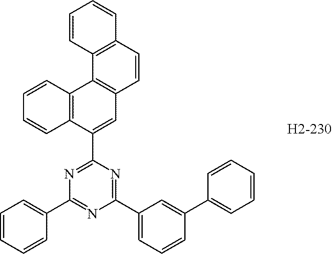 [SMILES: c1ccc(-c2cccc(-c3nc(-c4ccccc4)nc(-c4cc5ccc6ccccc6c5c5ccccc45)n3)c2)cc1]

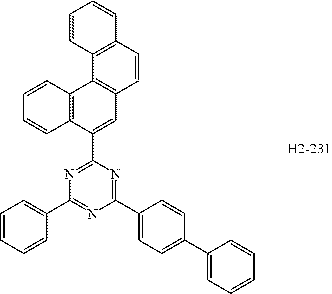 [SMILES: c1ccc(-c2ccc(-c3nc(-c4ccccc4)nc(-c4cc5ccc6ccccc6c5c5ccccc45)n3)cc2)cc1]

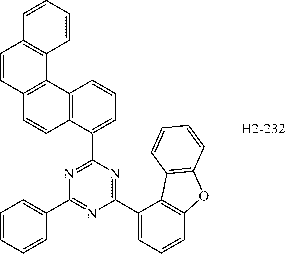 [SMILES: c1ccc(-c2nc(-c3cccc4c3ccc3ccc5ccccc5c34)nc(-c3cccc4oc5ccccc5c34)n2)cc1]

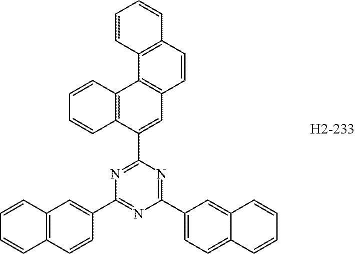 [SMILES: c1ccc2cc(-c3nc(-c4ccc5ccccc5c4)nc(-c4cc5ccc6ccccc6c5c5ccccc45)n3)ccc2c1]

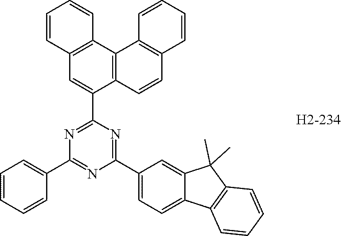 [SMILES: CC1(C)c2ccccc2-c2ccc(-c3nc(-c4ccccc4)nc(-c4cc5ccccc5c5c4ccc4ccccc45)n3)cc21]

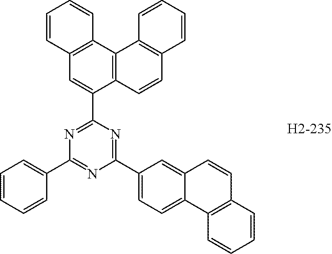 [SMILES: c1ccc(-c2nc(-c3ccc4c(ccc5ccccc54)c3)nc(-c3cc4ccccc4c4c3ccc3ccccc34)n2)cc1]

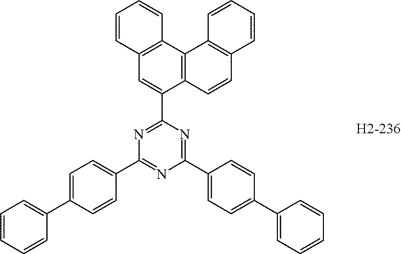 [SMILES: c1ccc(-c2ccc(-c3nc(-c4ccc(-c5ccccc5)cc4)nc(-c4cc5ccccc5c5c4ccc4ccccc45)n3)cc2)cc1]

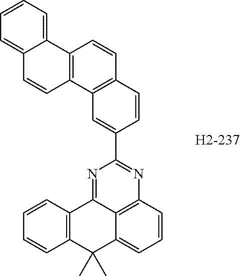 [SMILES: CC1(C)c2ccccc2-c2nc(-c3ccc4ccc5c6ccccc6ccc5c4c3)nc3cccc1c23]

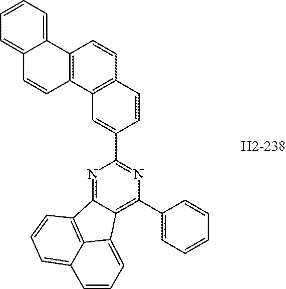 [SMILES: c1ccc(-c2nc(-c3ccc4ccc5c6ccccc6ccc5c4c3)nc3c2-c2cccc4cccc-3c24)cc1]

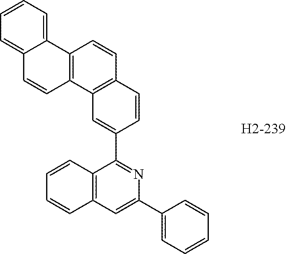 [SMILES: c1ccc(-c2cc3ccccc3c(-c3ccc4ccc5c6ccccc6ccc5c4c3)n2)cc1]

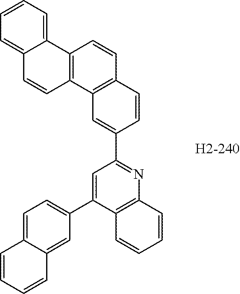 [SMILES: c1ccc2cc(-c3cc(-c4ccc5ccc6c7ccccc7ccc6c5c4)nc4ccccc34)ccc2c1]

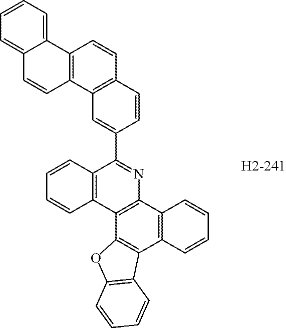 [SMILES: c1ccc2c(c1)ccc1c3cc(-c4nc5c6ccccc6c6c7ccccc7oc6c5c5ccccc45)ccc3ccc21]

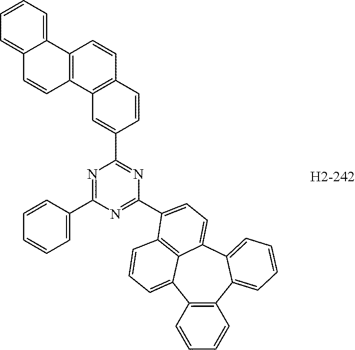 [SMILES: c1ccc(-c2nc(-c3ccc4ccc5c6ccccc6ccc5c4c3)nc(-c3ccc4c5c(cccc35)-c3ccccc3-c3ccccc3-4)n2)cc1]

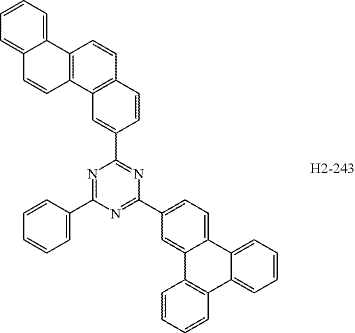 [SMILES: c1ccc(-c2nc(-c3ccc4ccc5c6ccccc6ccc5c4c3)nc(-c3ccc4c5ccccc5c5ccccc5c4c3)n2)cc1]

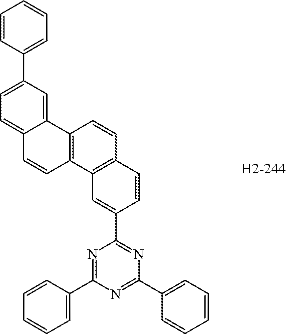 [SMILES: c1ccc(-c2ccc3ccc4c5cc(-c6nc(-c7ccccc7)nc(-c7ccccc7)n6)ccc5ccc4c3c2)cc1]

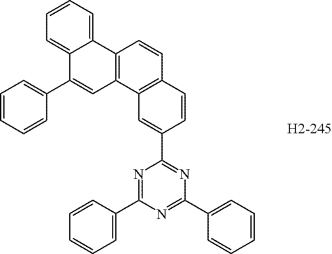 [SMILES: c1ccc(-c2nc(-c3ccccc3)nc(-c3ccc4ccc5c6ccccc6c(-c6ccccc6)cc5c4c3)n2)cc1]

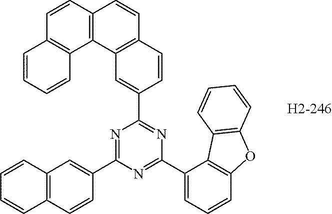 [SMILES: c1ccc2cc(-c3nc(-c4ccc5ccc6ccc7ccccc7c6c5c4)nc(-c4cccc5oc6ccccc6c45)n3)ccc2c1]

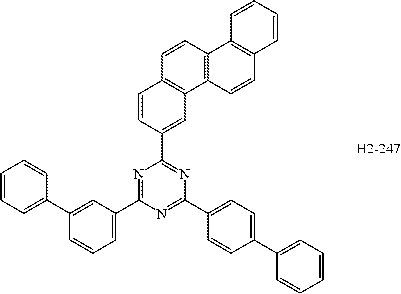 [SMILES: c1ccc(-c2ccc(-c3nc(-c4cccc(-c5ccccc5)c4)nc(-c4ccc5ccc6c7ccccc7ccc6c5c4)n3)cc2)cc1]

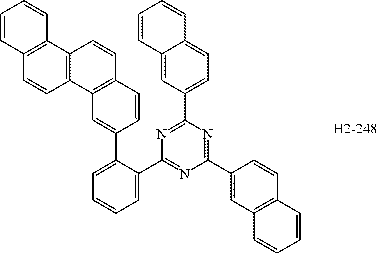 [SMILES: c1ccc(-c2nc(-c3ccc4ccccc4c3)nc(-c3ccc4ccccc4c3)n2)c(-c2ccc3ccc4c5ccccc5ccc4c3c2)c1]

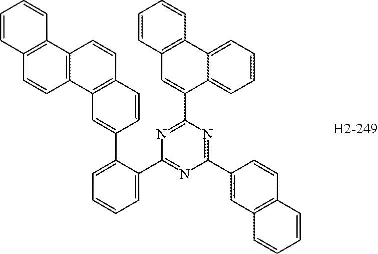 [SMILES: c1ccc(-c2nc(-c3ccc4ccccc4c3)nc(-c3cc4ccccc4c4ccccc34)n2)c(-c2ccc3ccc4c5ccccc5ccc4c3c2)c1]

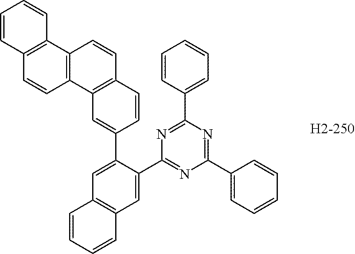 [SMILES: c1ccc(-c2nc(-c3ccccc3)nc(-c3cc4ccccc4cc3-c3ccc4ccc5c6ccccc6ccc5c4c3)n2)cc1]

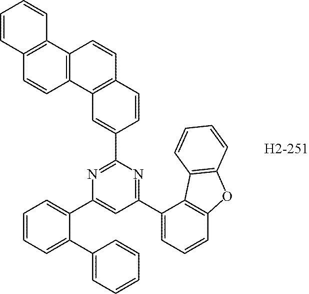 [SMILES: c1ccc(-c2ccccc2-c2cc(-c3cccc4oc5ccccc5c34)nc(-c3ccc4ccc5c6ccccc6ccc5c4c3)n2)cc1]

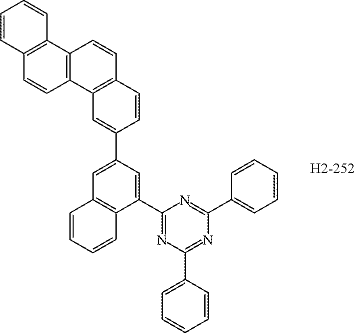 [SMILES: c1ccc(-c2nc(-c3ccccc3)nc(-c3cc(-c4ccc5ccc6c7ccccc7ccc6c5c4)cc4ccccc34)n2)cc1]

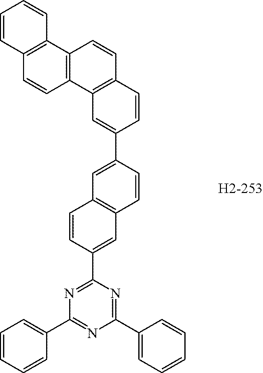 [SMILES: c1ccc(-c2nc(-c3ccccc3)nc(-c3ccc4cc(-c5ccc6ccc7c8ccccc8ccc7c6c5)ccc4c3)n2)cc1]

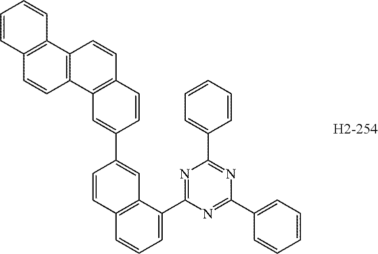 [SMILES: c1ccc(-c2nc(-c3ccccc3)nc(-c3cccc4ccc(-c5ccc6ccc7c8ccccc8ccc7c6c5)cc34)n2)cc1]

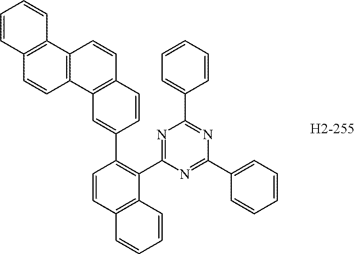 [SMILES: c1ccc(-c2nc(-c3ccccc3)nc(-c3c(-c4ccc5ccc6c7ccccc7ccc6c5c4)ccc4ccccc34)n2)cc1]

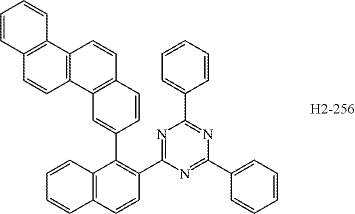 [SMILES: c1ccc(-c2nc(-c3ccccc3)nc(-c3ccc4ccccc4c3-c3ccc4ccc5c6ccccc6ccc5c4c3)n2)cc1]

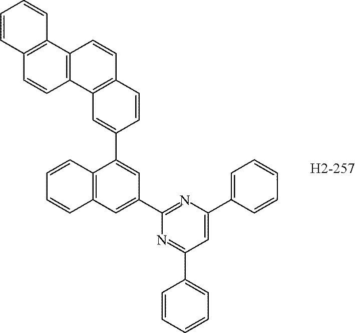 [SMILES: c1ccc(-c2cc(-c3ccccc3)nc(-c3cc(-c4ccc5ccc6c7ccccc7ccc6c5c4)c4ccccc4c3)n2)cc1]

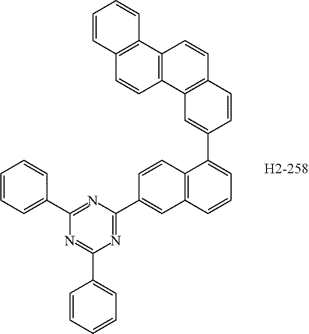 [SMILES: c1ccc(-c2nc(-c3ccccc3)nc(-c3ccc4c(-c5ccc6ccc7c8ccccc8ccc7c6c5)cccc4c3)n2)cc1]

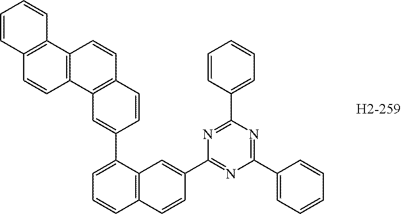 [SMILES: c1ccc(-c2nc(-c3ccccc3)nc(-c3ccc4cccc(-c5ccc6ccc7c8ccccc8ccc7c6c5)c4c3)n2)cc1]

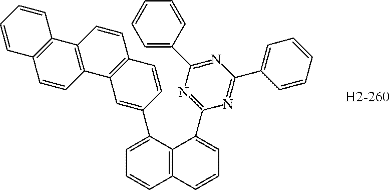 [SMILES: c1ccc(-c2nc(-c3ccccc3)nc(-c3cccc4cccc(-c5ccc6ccc7c8ccccc8ccc7c6c5)c34)n2)cc1]

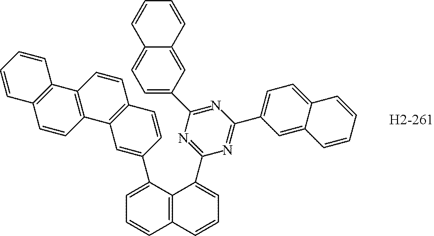 [SMILES: c1ccc2cc(-c3nc(-c4ccc5ccccc5c4)nc(-c4cccc5cccc(-c6ccc7ccc8c9ccccc9ccc8c7c6)c45)n3)ccc2c1]

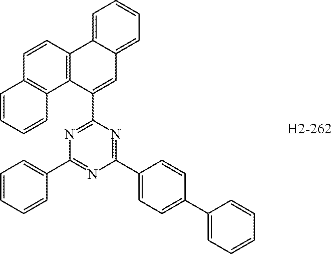 [SMILES: c1ccc(-c2ccc(-c3nc(-c4ccccc4)nc(-c4cc5ccccc5c5ccc6ccccc6c45)n3)cc2)cc1]

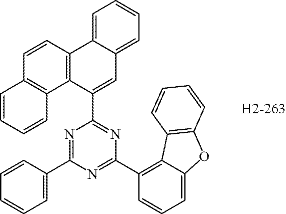 [SMILES: c1ccc(-c2nc(-c3cccc4oc5ccccc5c34)nc(-c3cc4ccccc4c4ccc5ccccc5c34)n2)cc1]

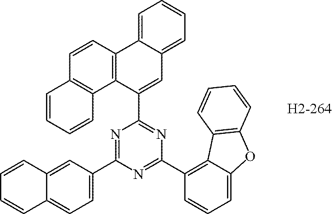 [SMILES: c1ccc2cc(-c3nc(-c4cccc5oc6ccccc6c45)nc(-c4cc5ccccc5c5ccc6ccccc6c45)n3)ccc2c1]

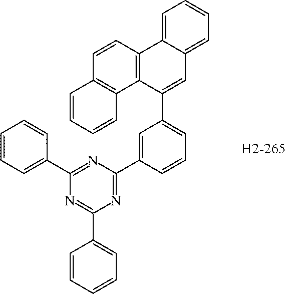 [SMILES: c1ccc(-c2nc(-c3ccccc3)nc(-c3cccc(-c4cc5ccccc5c5ccc6ccccc6c45)c3)n2)cc1]

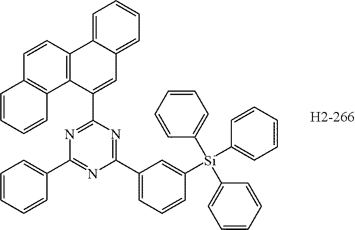 [SMILES: c1ccc(-c2nc(-c3cccc([Si](c4ccccc4)(c4ccccc4)c4ccccc4)c3)nc(-c3cc4ccccc4c4ccc5ccccc5c34)n2)cc1]

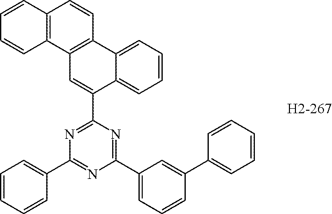 [SMILES: c1ccc(-c2cccc(-c3nc(-c4ccccc4)nc(-c4cc5c6ccccc6ccc5c5ccccc45)n3)c2)cc1]

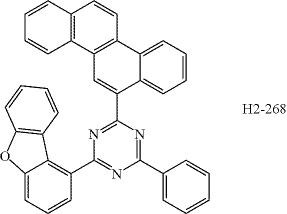 [SMILES: c1ccc(-c2nc(-c3cc4c5ccccc5ccc4c4ccccc34)nc(-c3cccc4oc5ccccc5c34)n2)cc1]

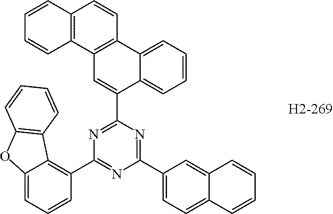 [SMILES: c1ccc2cc(-c3nc(-c4cc5c6ccccc6ccc5c5ccccc45)nc(-c4cccc5oc6ccccc6c45)n3)ccc2c1]

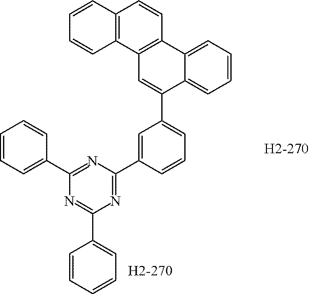

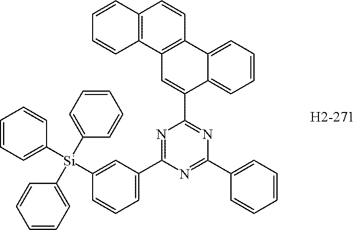 [SMILES: c1ccc(-c2nc(-c3cccc([Si](c4ccccc4)(c4ccccc4)c4ccccc4)c3)nc(-c3cc4c5ccccc5ccc4c4ccccc34)n2)cc1]

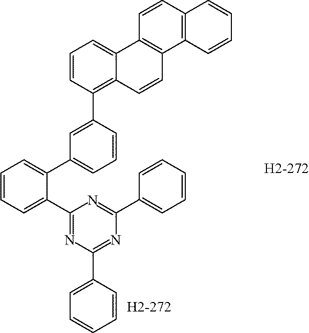

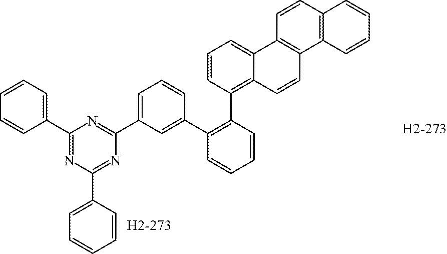

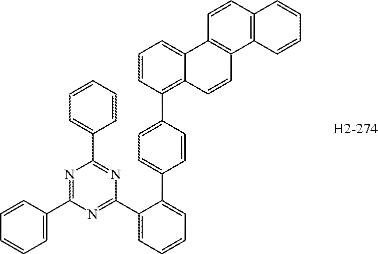 [SMILES: c1ccc(-c2nc(-c3ccccc3)nc(-c3ccccc3-c3ccc(-c4cccc5c4ccc4c6ccccc6ccc54)cc3)n2)cc1]

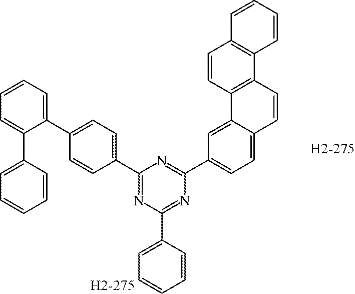

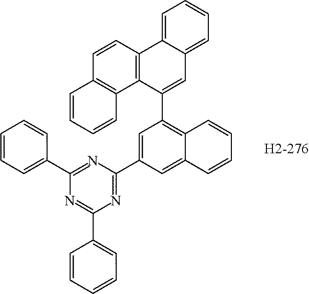 [SMILES: c1ccc(-c2nc(-c3ccccc3)nc(-c3cc(-c4cc5ccccc5c5ccc6ccccc6c45)c4ccccc4c3)n2)cc1]

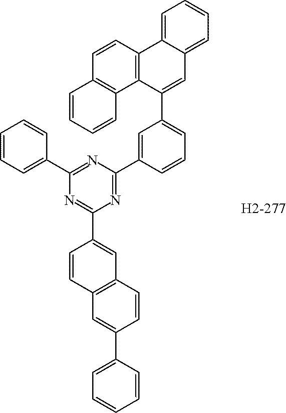 [SMILES: c1ccc(-c2ccc3cc(-c4nc(-c5ccccc5)nc(-c5cccc(-c6cc7ccccc7c7ccc8ccccc8c67)c5)n4)ccc3c2)cc1]

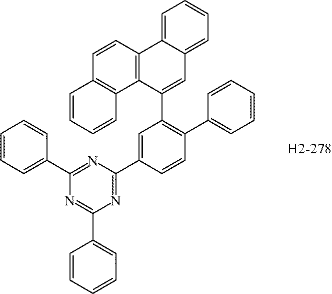 [SMILES: c1ccc(-c2nc(-c3ccccc3)nc(-c3ccc(-c4ccccc4)c(-c4cc5ccccc5c5ccc6ccccc6c45)c3)n2)cc1]

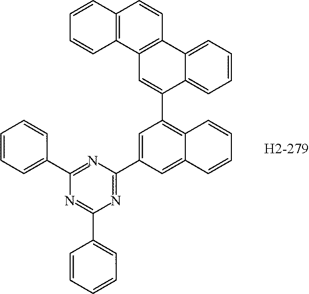 [SMILES: c1ccc(-c2nc(-c3ccccc3)nc(-c3cc(-c4cc5c6ccccc6ccc5c5ccccc45)c4ccccc4c3)n2)cc1]

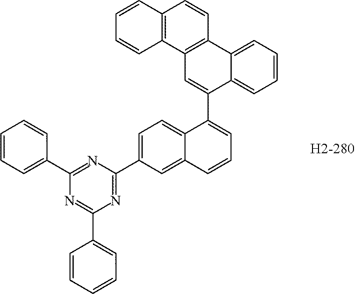 [SMILES: c1ccc(-c2nc(-c3ccccc3)nc(-c3ccc4c(-c5cc6c7ccccc7ccc6c6ccccc56)cccc4c3)n2)cc1]

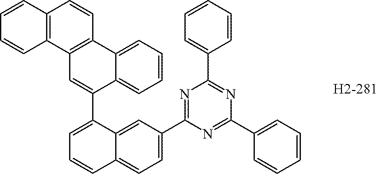 [SMILES: c1ccc(-c2nc(-c3ccccc3)nc(-c3ccc4cccc(-c5cc6c7ccccc7ccc6c6ccccc56)c4c3)n2)cc1]

A combination of at least one of compounds C-1 to C-75 and at least one of compounds H2-1 to H2-281 may be used in an organic electroluminescent device.

The compound represented by formula 1 according to the present disclosure may be produced by referring to the following Reaction Scheme 1, but is not limited thereto.

The compound represented by formula 2 according to the present disclosure may be produced by synthetic methods known to a person skilled in the art, for example, by referring to the following Reaction Scheme 2, but is not limited thereto.

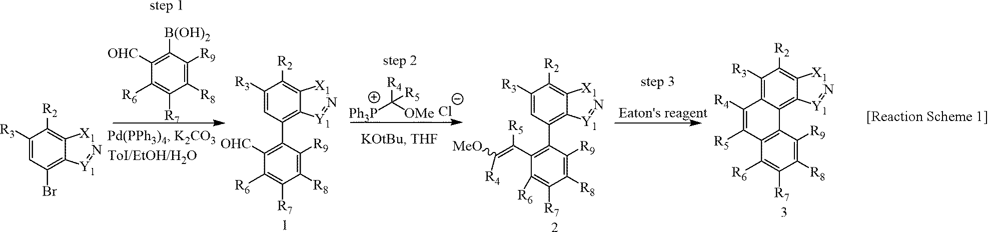

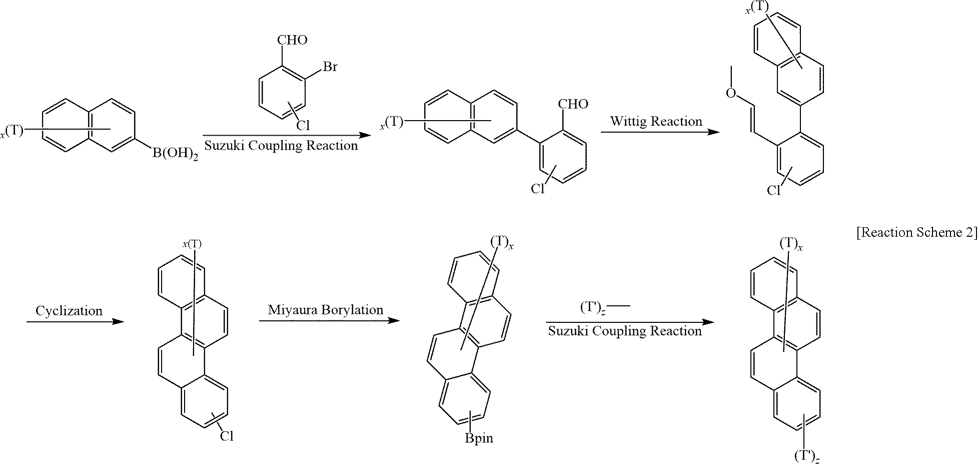

In Reaction Scheme 1, X₁, Y₁, and R₂ to R₉ are as defined for formula 1, and in Reaction Scheme 2, T and T′ are as defined for T₁ to T₄, and T₉ to T₁₄ in formula 2, x represents an integer of 1 to 7, and z represents an integer of 1 to 4, where if x and z are 2 or more, each of T and each of T′ may be the same as or different from each other.

Although illustrative synthesis examples of the compounds represented by formulas 1 and 2 are described above, one skilled in the art will be able to readily understand that all of them are based on a Buchwald-Hartwig cross coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, and a Phosphine-mediated reductive cyclization reaction, etc., and the above reactions proceed even when substituents defined in formulas 1 and 2 other than the substituents specified in the specific synthesis examples, are bonded.

An organic electroluminescent device according to the present disclosure comprises an anode, a cathode, and at least one organic layer between the anode and the cathode, wherein the organic layer may comprise a plurality of organic electroluminescent materials comprising the compound represented by formula 1 as a first organic electroluminescent material, and the compound represented by formula 2 as a second organic electroluminescent material. According to one embodiment of the present disclosure, the organic electroluminescent device according to the present disclosure comprises an anode, a cathode, and at least one light-emitting layer between the anode and the cathode, wherein the light-emitting layer may comprise the compound represented by formula 1 and the compound represented by formula 2.

The light-emitting layer comprises a host and a dopant, wherein the host comprises a plurality of host materials, the compound represented by formula 1 may be comprised as a first host compound of the plurality of host materials, and the compound represented by formula 2 may be comprised as a second host compound of the plurality of host materials. Herein, the weight ratio of the first host compound to the second host compound is about 1:99 to about 99:1, preferably about 10:90 to about 90:10, more preferably about 30:70 to about 70:30, still more preferably about 40:60 to about 60:40, even more preferably about 50:50.

In the present disclosure, the light-emitting layer is a layer from which light is emitted, and can be a single layer or a multi-layer of which two or more layers are stacked. In the plurality of host materials of the present disclosure, both the first and second host materials may be comprised in one layer, or the first and second host materials may be respectively comprised in different light-emitting layers. According to one embodiment of the present disclosure, the doping concentration of the dopant compound with respect to the host compound of the light-emitting layer may be less than 20 wt%.

The organic electroluminescent device of the present disclosure may further comprise at least one layer selected from a hole injection layer, a hole transport layer, a hole auxiliary layer, a light-emitting auxiliary layer, an electron transport layer, an electron injection layer, an interlayer, an electron buffer layer, a hole blocking layer, and an electron blocking layer. According to one embodiment of the present disclosure, the organic electroluminescent device of the present disclosure may further comprise an amine-based compound as at least one of a hole injection material, a hole transport material, a hole auxiliary material, a light-emitting material, a light-emitting auxiliary material, and an electron blocking material besides the plurality of host materials of the present disclosure. Also, according to one embodiment of the present disclosure, the organic electroluminescent device of the present disclosure may further comprise an azine-based compound as at least one of an electron transport material, an electron injection material, an electron buffer material and a hole blocking material besides the plurality of host materials of the present disclosure.

The plurality of host materials according to one embodiment may be used as light-emitting materials for a white organic light-emitting device. The white organic light-emitting device has been suggested to have various structures such as a side-by-side structure or a stacking structure depending on the arrangement of R (Red), G (Green) or YG (yellowish green), and B (blue) light-emitting parts, or color conversion material (CCM) method, etc. In addition, the plurality of host materials according to one embodiment of the present disclosure may also be used in an organic electroluminescent device comprising a quantum dot (QD).

A hole injection layer, a hole transport layer, an electron blocking layer, or a combination thereof may be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. In addition, the hole injection layer may be doped with a p-dopant. The electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron blocking layer may be multi-layers, wherein a plurality of compounds may be used in each of the multi-layers.

An electron buffer layer, a hole blocking layer, an electron transport layer, an electron injection layer, or a combination thereof may be used between the light-emitting layer and the cathode. The electron buffer layer may be multi-layers in order to control electron injection and improve interfacial properties between the light-emitting layer and the electron injection layer, wherein two compounds may be simultaneously used in each of the multilayers. The hole blocking layer or the electron transport layer may also be multi-layers, wherein a plurality of compounds may be used in each of the multi-layers. In addition, the electron injection layer may be doped with an n-dopant.

The dopants comprised in the organic electroluminescent device of the present disclosure may be at least one phosphorescent or fluorescent dopant, and is preferably a phosphorescent dopant. The phosphorescent dopant materials applied to the organic electroluminescent device according to the present disclosure are not particularly limited, but may be a complex compound of a metal atom selected from iridium (Ir), osmium (Os), copper (Cu), and platinum (Pt), preferably ortho-metallated complex compounds of a metal atom selected from iridium (Ir), osmium (Os), copper (Cu), and platinum (Pt), and more preferably ortho-metallated iridium complex compounds.

The dopant comprised in the organic electroluminescent device of the present disclosure may be a compound represented by the following formula 101, but is not limited thereto.

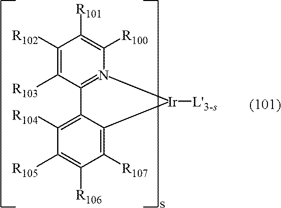

In formula 101,
L′ is selected from the following structures 1 to 3:

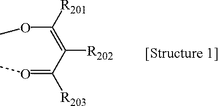

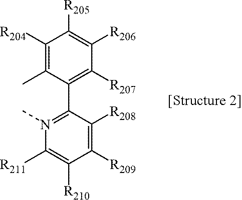

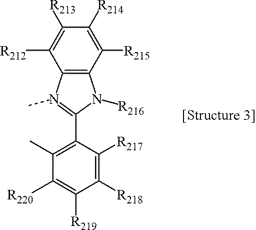

R₁₀₀ to R₁₀₃ each independently represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent(s) to form a ring(s), for example, a substituted or unsubstituted quinoline, a substituted or unsubstituted isoquinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline, together with pyridine;
R₁₀₄ to R₁₀₇ each independently represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent(s) to form a ring(s), for example, a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine, together with benzene;
R₂₀₁ to R₂₂₀ each independently represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent(s) to form a ring(s); and
s is an integer from 1 to 3.

The specific examples of the dopant compound are as follows, but are not limited thereto.

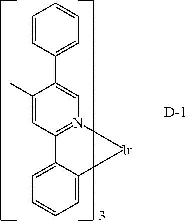

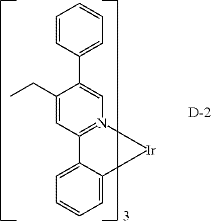

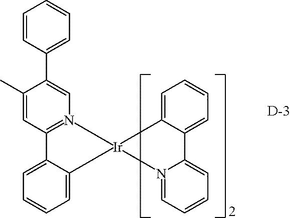

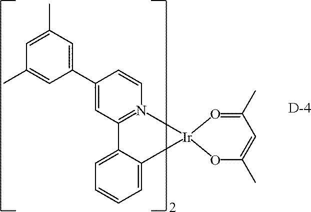

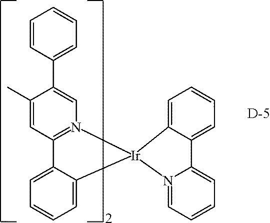

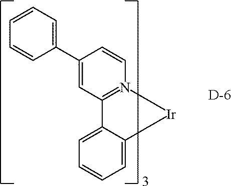

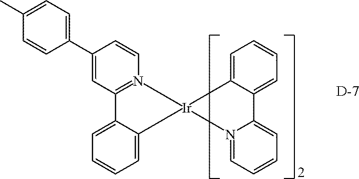

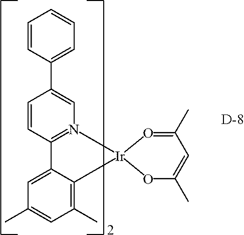

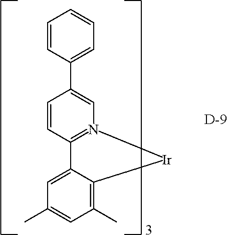

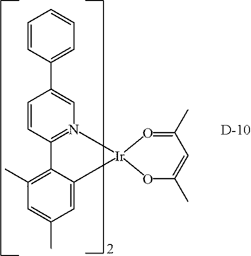

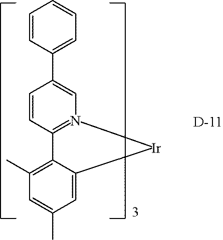

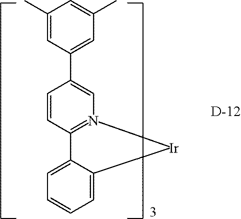

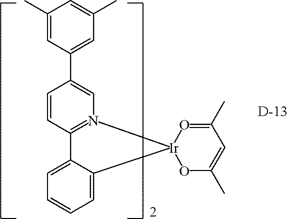

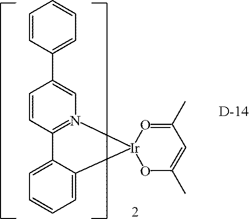

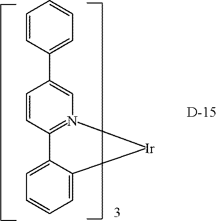

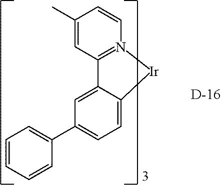

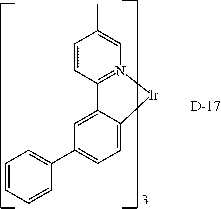

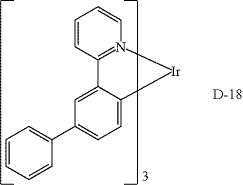

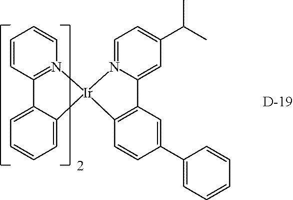

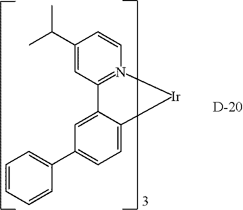

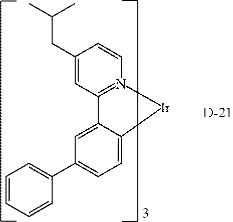

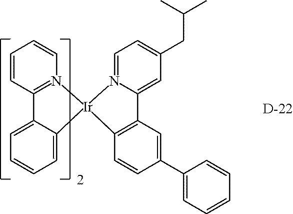

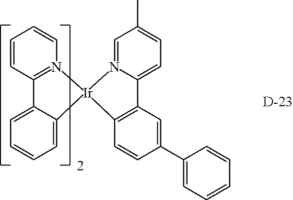

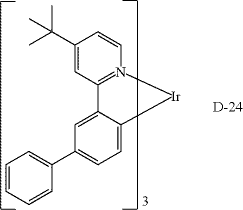

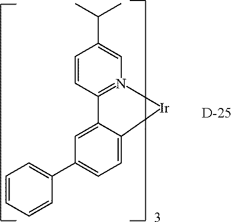

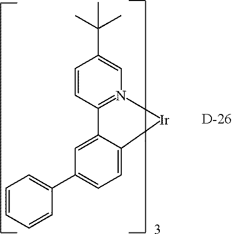

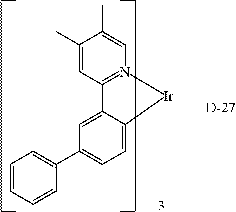

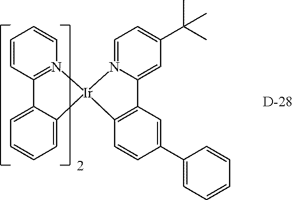

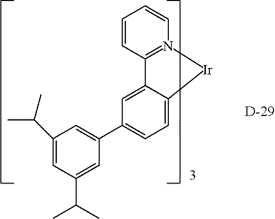

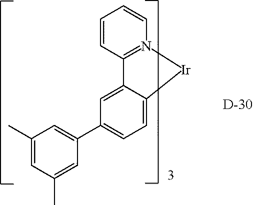

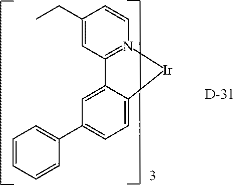

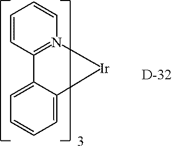

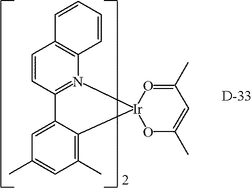

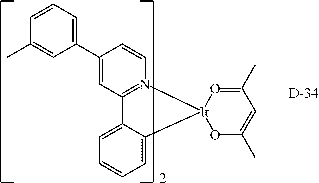

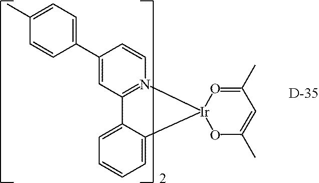

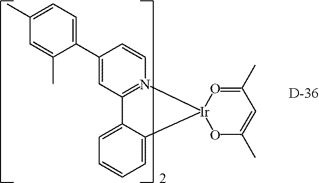

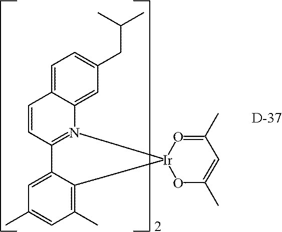

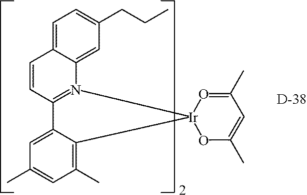

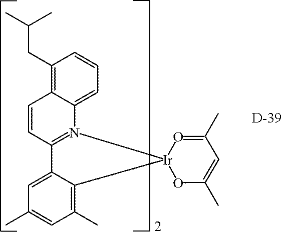

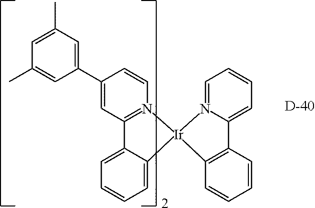

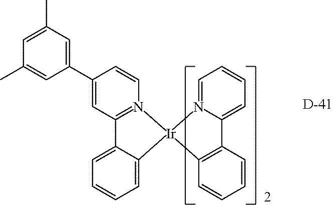

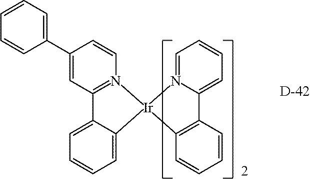

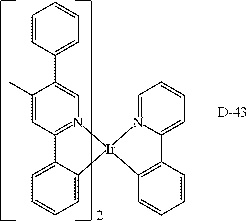

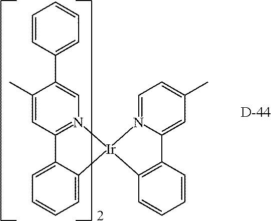

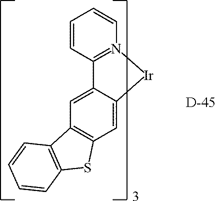

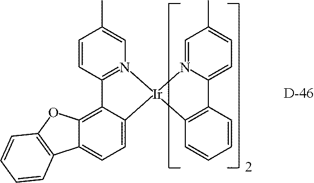

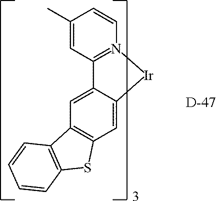

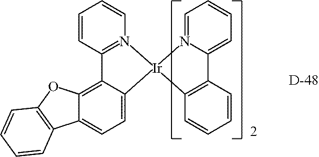

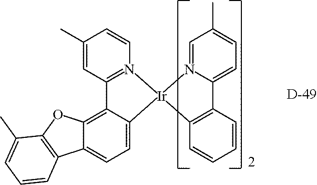

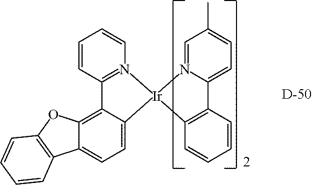

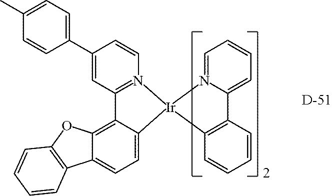

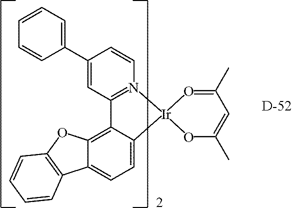

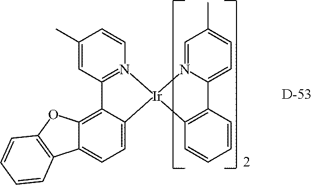

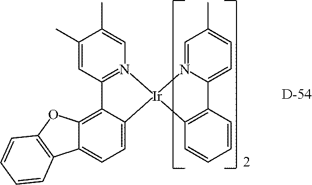

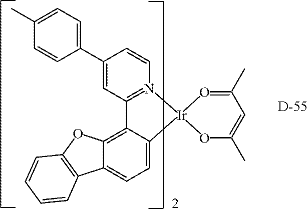

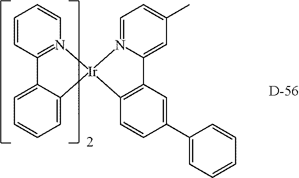

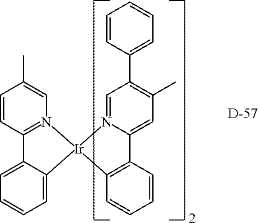

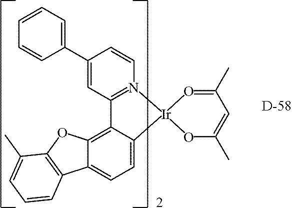

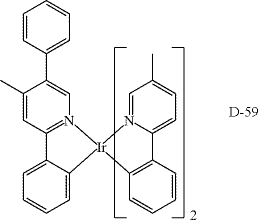

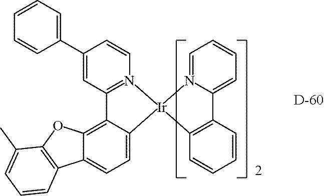

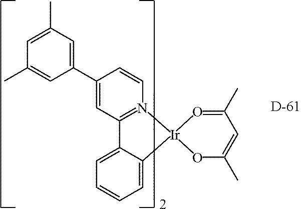

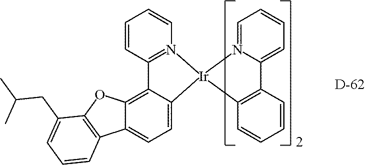

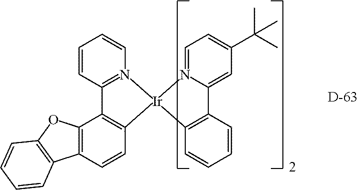

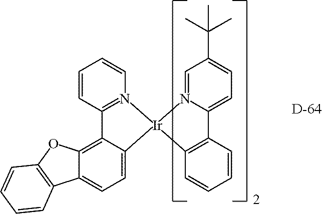

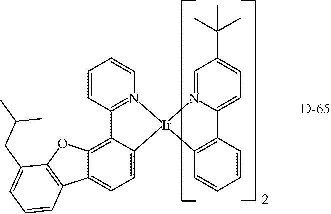

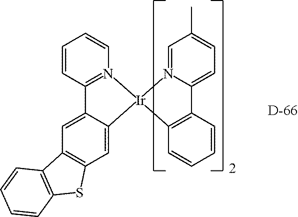

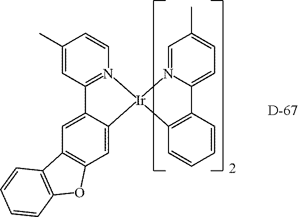

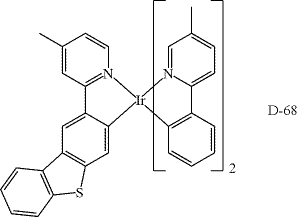

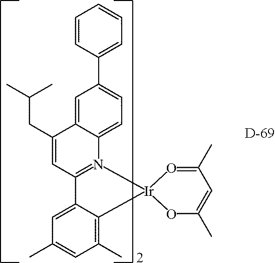

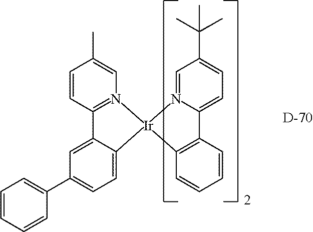

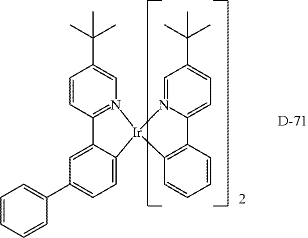

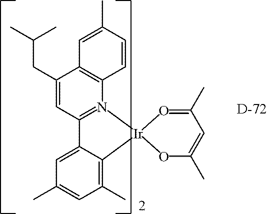

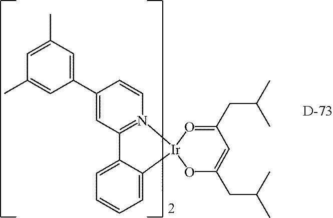

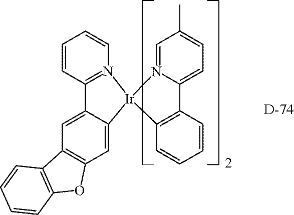

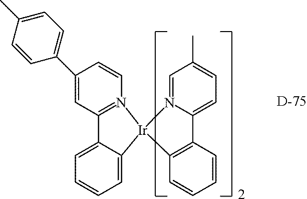

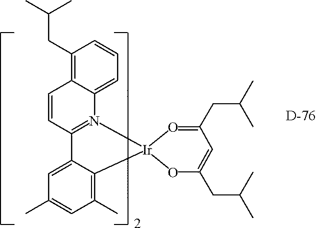

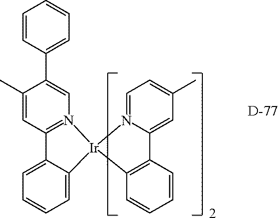

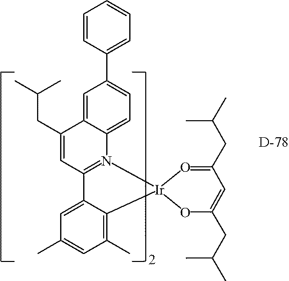

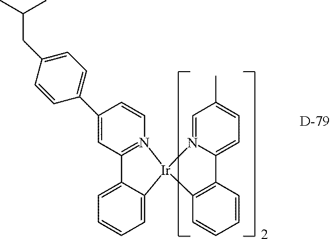

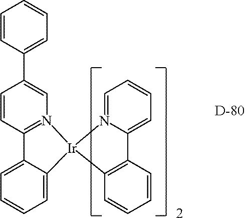

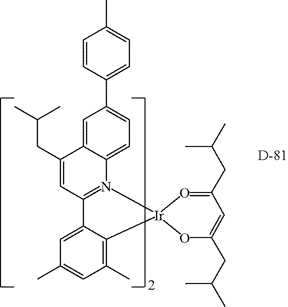

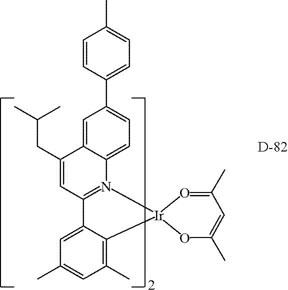

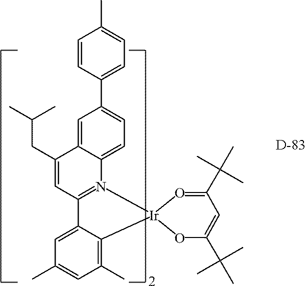

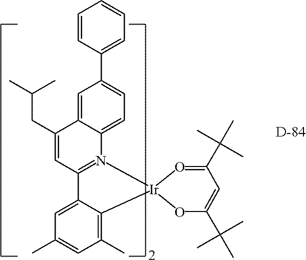

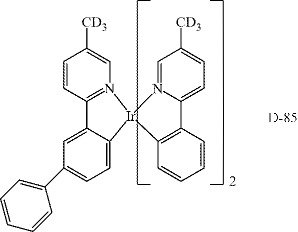

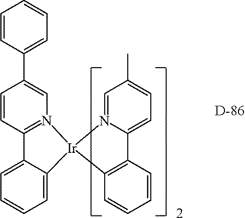

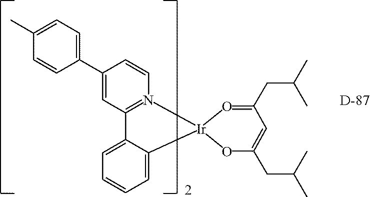

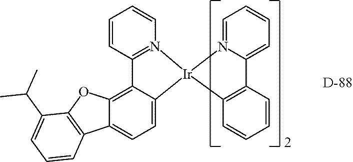

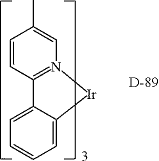

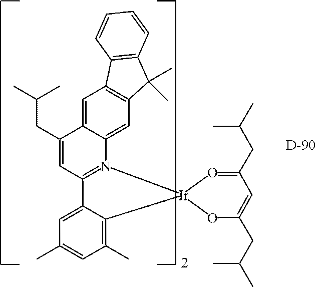

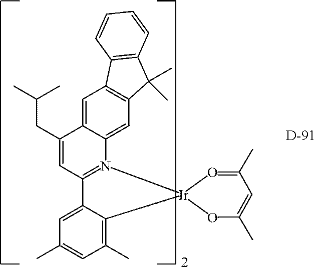

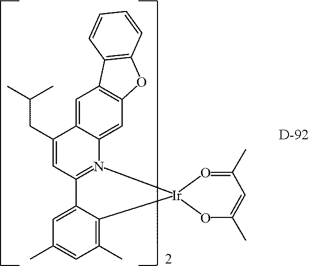

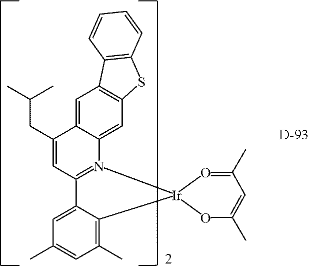

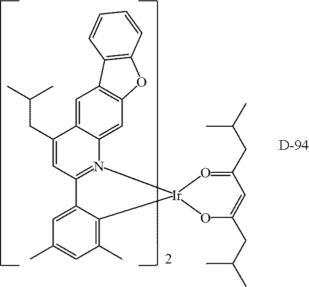

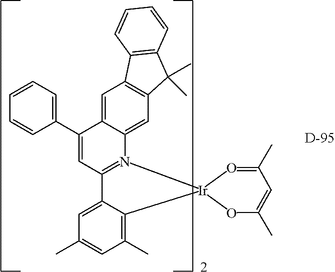

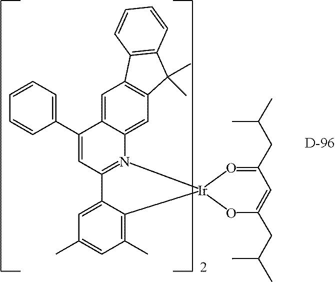

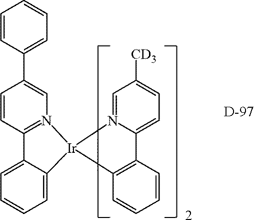

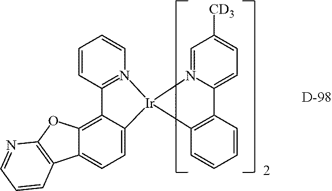

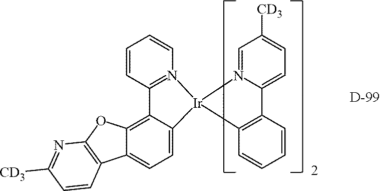

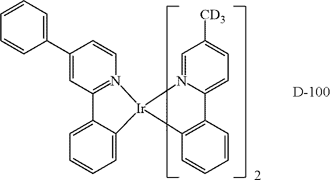

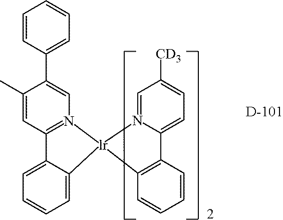

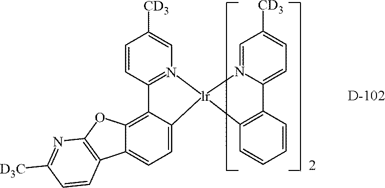

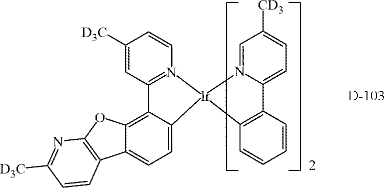

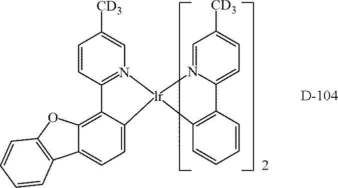

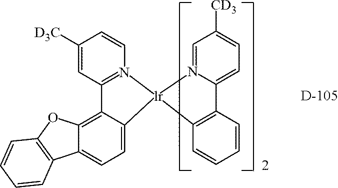

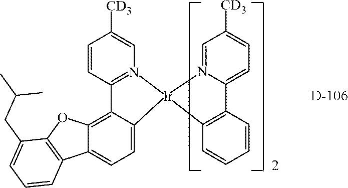

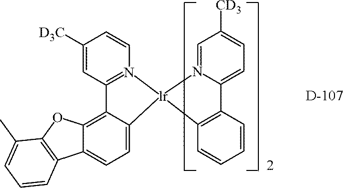

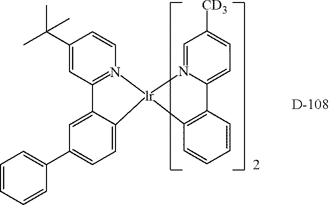

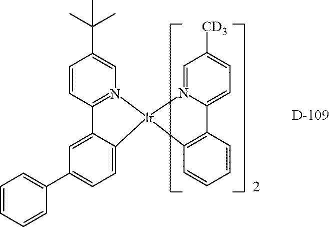

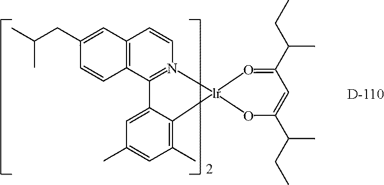

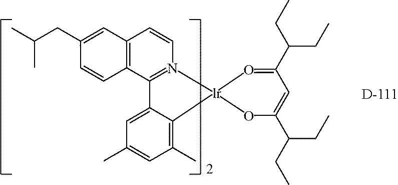

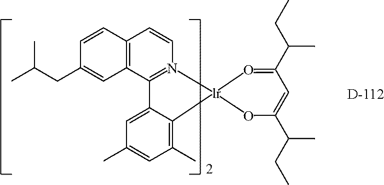

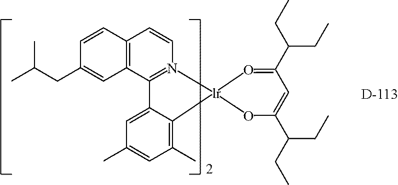

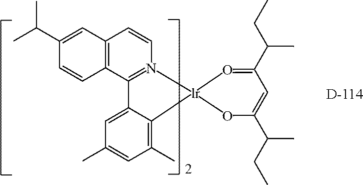

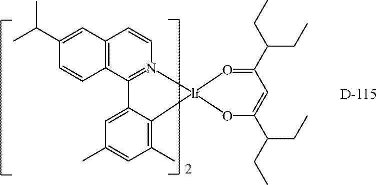

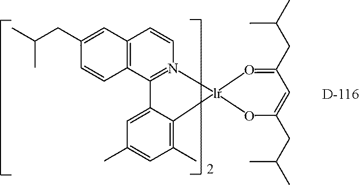

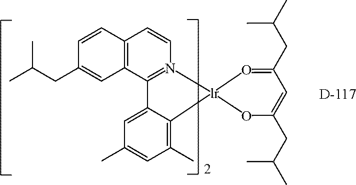

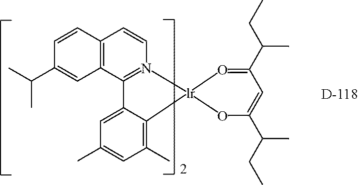

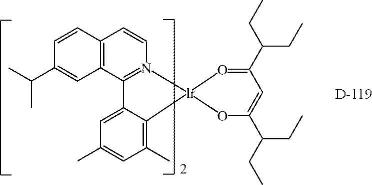

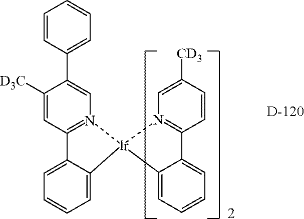

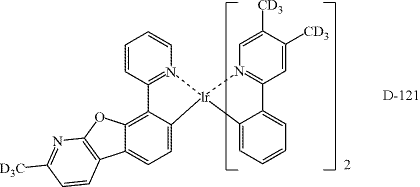

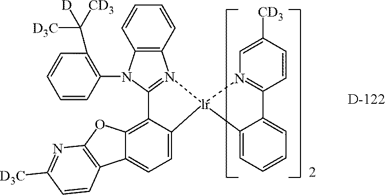

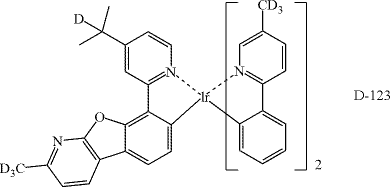

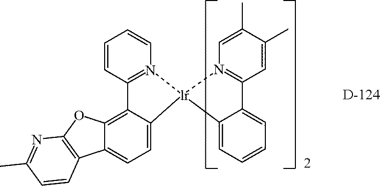

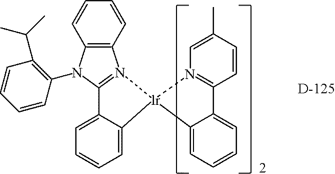

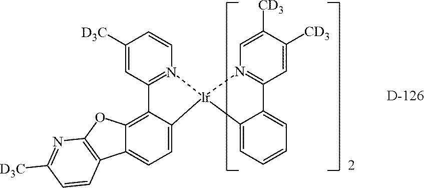

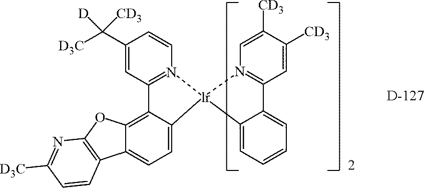

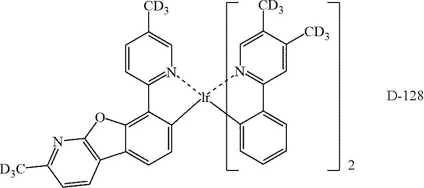

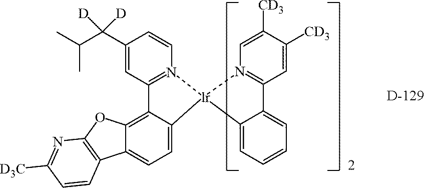

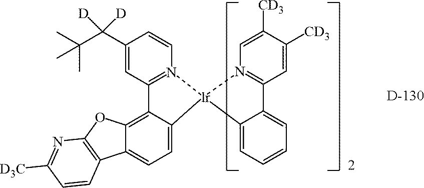

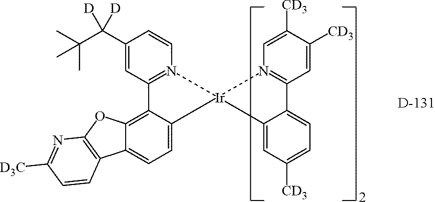

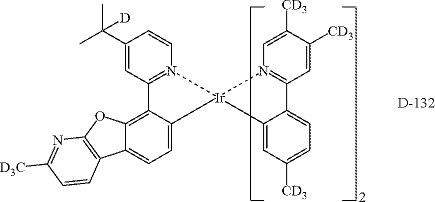

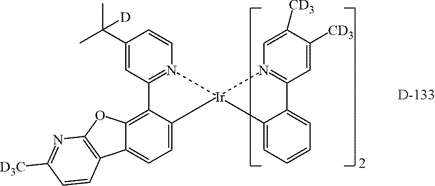

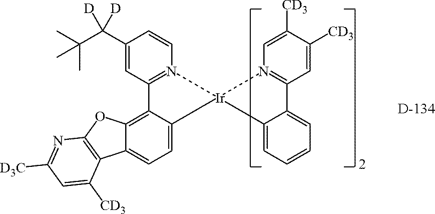

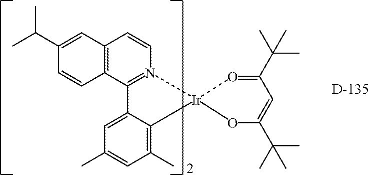

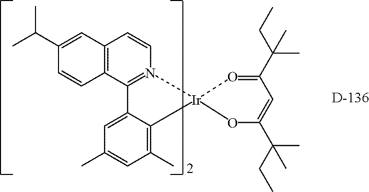

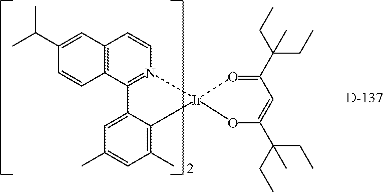

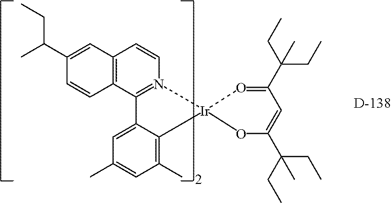

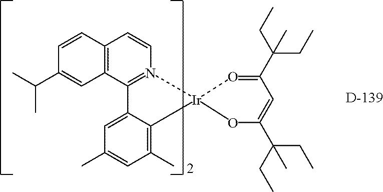

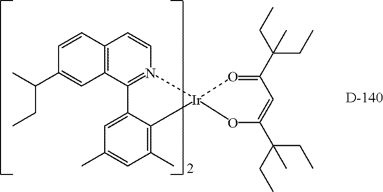

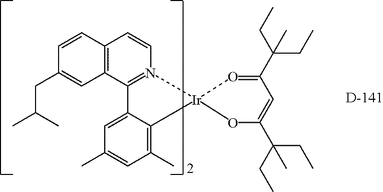

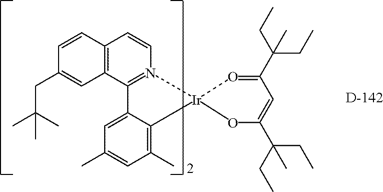

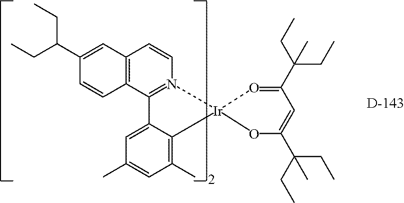

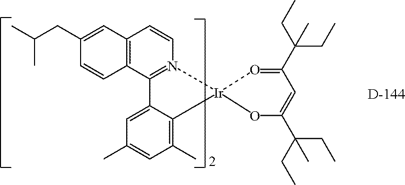

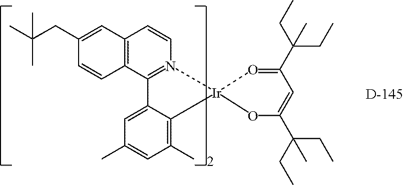

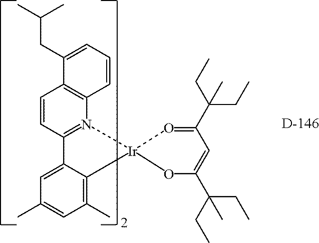

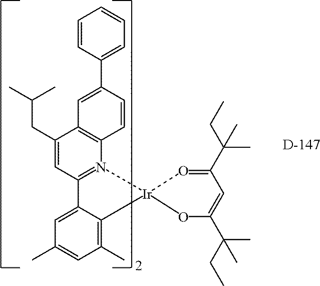

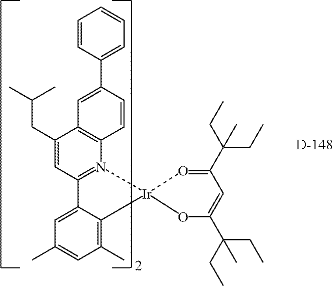

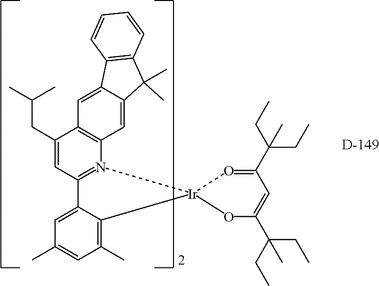

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

In addition, the first and second host compounds of the present disclosure may be film-formed by the methods listed above, commonly by a co-evaporation process or a mixture-evaporation process. The co-deposition is a mixed deposition method in which two or more materials are put into respective individual crucible sources and a current is applied to both cells simultaneously to evaporate the materials. The mixture-deposition is a mixed deposition method in which two or more materials are mixed in one crucible source before evaporating them, and a current is applied to one cell to evaporate the materials. In addition, when the first host compound and the second host compound are present in the same layer or different layers in an organic electroluminescent device, each of the two host compounds may individually form films. For example, the second host compound may be deposited after depositing the first host compound.

The present disclosure can provide a display device by using the organic electroluminescent material comprising the compound represented by formula 1, or the plurality of host materials comprising the compound represented by formula 1 and the compound represented by formula 2. That is, it is possible to manufacture a display system or a lighting system using the organic electroluminescent material of the present disclosure or the plurality of types of host materials of the present disclosure. Specifically, a display system, for example, a display system for white organic light-emitting devices, smartphones, tablets, notebooks, PCs, TVs, or cars; or a lighting system, for example, an outdoor or indoor lighting system, can be produced by using the plurality of host materials of the present disclosure.

Hereinafter, the preparation method of the compound according to the present disclosure and the physical properties thereof, and the properties of the organic electroluminescent device (OLED) device comprising the plurality of host materials of the present disclosure will be explained with reference to the representative compounds of the present disclosure. However, the following examples are only to describe the characteristics of the OLED device comprising the compound according to the present disclosure and the plurality of host materials according to the present disclosure, but the present disclosure is not limited to the following examples.

### Example 1: Preparation of Compound C-64

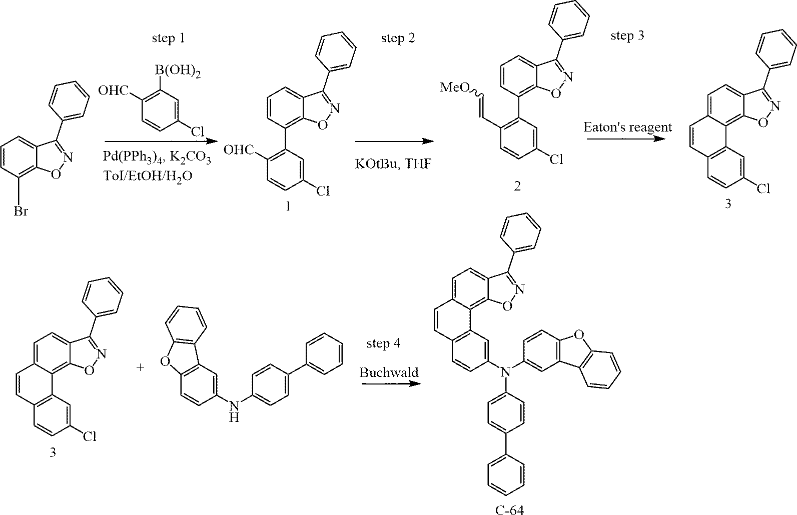

### 1) Synthesis of Compound 1

7-bromophenylbenzo[d]isoxazole (20 g, 73 mmol), (5-chloro-2-formylphenyl)boronic acid (27 g, 145 mmol), Pd(PPh₃)₄ (4.2 g, 3.6 mmol), K₂CO₃ (25 g, 182 mmol), 360 mL of toluene, 90 mL of ethanol, and 90 mL of water were added into a flask, dissolved, and then stirred for 18 hours at 120° C. After completion of the reaction, the organic layer was extracted with ethyl acetate, and the remaining moisture was removed with magnesium sulfate. Thereafter, the reaction mixture was dried, and separated by column chromatography to obtain compound 1 (10 g, yield: 41%).

### 2) Synthesis of Compound 2

Compound 1 (9.5 g, 28 mmol), (methoxymethyl)triphenyl phosphonium chloride (14.6 g, 43 mmol), and 280 mL of tetrahydrofuran (THF) were added into a flask and dissolved. Next, potassium t-butoxide (1 M, 43 mL, 43 mmol) dissolved in THF was slowly added dropwise at 0° C., and the mixture was stirred for 2 hours at room temperature. After completion of the reaction, the organic layer was extracted with ethyl acetate, and the remaining moisture was removed with magnesium sulfate. Thereafter, the reaction mixture was dried, and separated by column chromatography to obtain compound 2 (8.3 g, yield: 80%).

### 3) Synthesis of Compound 3

Compound 2 (11.2 g, 31 mmol) and 100 mL of chlorobenzene were added into a flask and dissolved. 0.8 mL of Eaton’s reagent was slowly added dropwise, and then the mixture was heated under reflux for 1 hour. After completion of the reaction, the organic layer was extracted with ethyl acetate, and the remaining moisture was removed with magnesium sulfate. Thereafter, the reaction mixture was dried, and separated by column chromatography to obtain compound 3 (1.0 g, yield: 10%).

### 4) Synthesis of Compound C-64

Compound 3 (1.0 g, 3 mmol), N-([1,1′-biphenyl]-4-yl)dibenzo[b,d]furan-2-amine (1.0 g, 3 mmol), Pd₂dba₃ (139 mg, 0.15 mmol), s-phos (124 mg, 0.3 mmol), sodium t-butoxide (582 mg, 6 mmol), and 15 mL of o-xylene were added into a flask and dissolved. Next, the mixture was heated under reflux for 18 hours. After completion of the reaction, the organic layer was extracted with ethyl acetate, and the remaining moisture was removed with magnesium sulfate. Thereafter, the reaction mixture was dried, and separated by column chromatography to obtain compound C-64 (1.2 g, yield: 63%).

| Compound | MW | M.P. | Tg |
|---|---|---|---|
| C-64 | 628.73 | 254° C. | 124° C. |

### Device Example 1: Producing OLEDs Co-deposited With the Plurality of Host materials according to the present disclosure

An OLED according to the present disclosure was produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was mounted on a substrate holder of a vacuum vapor deposition apparatus. Compound HI-1 shown in Table 2 was introduced into a cell of the vacuum vapor deposition apparatus, and compound HT-1 was introduced into another cell. The two materials were evaporated at different rates, and compound HI-1 was deposited in a doping amount of 3 wt% based to the total amount of compound HI-1 and compound HT-1 to form a hole injection layer with a thickness of 10 nm. Subsequently, compound HT-1 was deposited on the hole injection layer to form a first hole transport layer with a thickness of 80 nm. Next, compound HT-2 was introduced into another cell of the vacuum vapor deposition apparatus and was evaporated by applying an electric current to the cell, thereby depositing a second hole transport layer with a thickness of 60 nm on the first hole transport layer. After forming the hole injection layer and the hole transport layers, a light-emitting layer was deposited thereon as follows: Each of the compound C-64 and compound H2-146 shown in Table 1 below were introduced into two cells of the vacuum vapor deposition apparatus as hosts, and compound D-39 was introduced into another cell as a dopant. The two host materials were evaporated at a rate of 1:1 and the dopant material was simultaneously evaporated at a different rate, and the dopant was deposited in a doping amount of 3 wt% based on the total amount of the hosts and dopant to form a light-emitting layer with a thickness of 40 nm on the second hole transport layer. Then, compound ET-1 and compound EI-1 were evaporated at a weight ratio of 50:50 as an electron transport material to form an electron transport layer having a thickness of 35 nm on the light-emitting layer. After depositing compound EI-1 as an electron injection layer with a thickness of 2 nm on the electron transport layer, an Al cathode was deposited with a thickness of 80 nm on the electron injection layer by using another vacuum vapor deposition apparatus, thereby producing an OLED. All the materials used for producing the OLED were purified by vacuum sublimation at 10⁻⁶ torr.

### Comparative Example 1: Producing an OLED Comprising a Host Combination Not according to the present disclosure as hosts

An OLED was produced in the same manner as in Device Example 1, except that compound A-1 shown in Table 1 below was used as the first host of the light-emitting layer instead of compound C-64.

The driving voltage, luminous efficiency, and light-emitting color at a luminance of 1,000 nit of the OLEDs produced in Device Example 1 and Comparative Example 1 produced as described above are provided in Table 1 below.

TABLE 1

| | First Host | Second Host | Driving Voltage (V) | Luminous Efficiency (cd/A) | Light-Emitting Color |
|---|---|---|---|---|---|
| Device Example 1 | C-64 | H2-146 | 3.1 | 35.4 | Red |
| Comparative Example 1 | A-1 | H2-146 | 3.4 | 34.9 | Red |

From Table 1 above, it can be confirmed that the OLED (Device Example 1) comprising the plurality of host materials according to the present disclosure, exhibit lower driving voltage and higher luminous efficiency, compared to the OLED (Comparative Example 1) comprising a host combination not according to the present disclosure.

TABLE 2

| | | | |
|---|---|---|---|
| Hole Injection Layer / Hole Transport Layer | 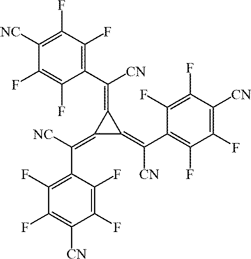 | 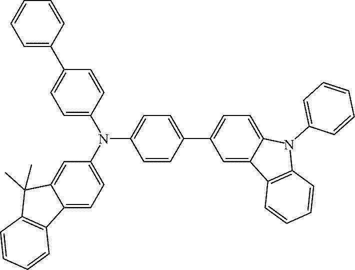 | 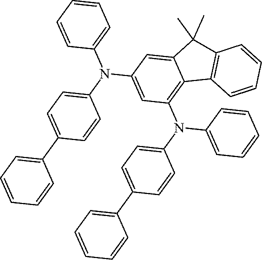 [SMILES: CC1(C)c2ccccc2-c2c(N(c3ccccc3)c3ccc(-c4ccccc4)cc3)cc(N(c3ccccc3)c3ccc(-c4ccccc4)cc3)cc21] |
| | HI-1 | HT-1 | HT-2 |
| Light-Emitting Layer | 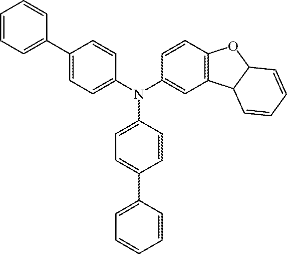 | 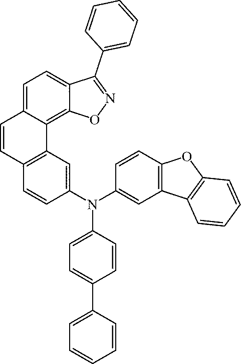 | 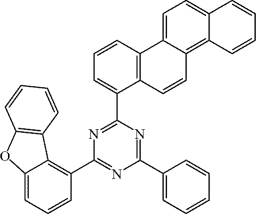 [SMILES: c1ccc(-c2nc(-c3cccc4c3ccc3c5ccccc5ccc43)nc(-c3cccc4oc5ccccc5c34)n2)cc1] |
| | A-1 | C-64 | H2-146 |
| | 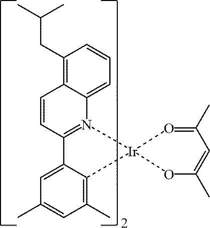 D-39 | | |
| Electron Transport Layer / Electron Injection Layer | 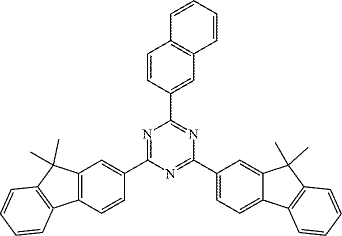 | 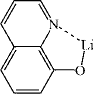 | |
| | ET-1 | EI-1 | |

1. A plurality of host materials comprising at least one first host compound and at least one second host compound, wherein the first host compound is represented by the following formula 1, and the second host compound is represented by the following formula 2: 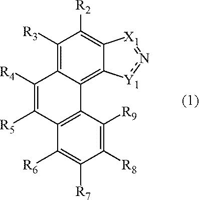
in formula 1,
X₁ and Y₁ each independently represent —O—, —S— or -CR₁=;
provided that one of X₁ and Y₁ represents -CR₁=, and the other of X₁ and Y₁ represents —O— or —S—;
R₁ represents a substituted or unsubstituted (C1-C30)alkyl, a substituted or unsubstituted (C6-C30)aryl, or a substituted or unsubstituted (3- to 30-membered)heteroaryl;
R₂ to R₉ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono-or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted mono- or di- (C6-C30)arylamino, a substituted or unsubstituted (C1-C30)alkyl(C6-C30)arylamino, a substituted or unsubstituted mono- or di- (3- to 30-membered)heteroarylamino, a substituted or unsubstituted (C1-C30)alkyl(3- to 30-membered)heteroarylamino, a substituted or unsubstituted (C2-C30)alkenyl(C6-C30) arylamino, a substituted or unsubstituted (C2-C30)alkenyl(3- to 30-membered)heteroarylamino, a substituted or unsubstituted (C6-C30)aryl(3- to 30-membered)heteroarylamino, or -L₁-N(Ar₁)(Ar₂); or may be linked to an adjacent substituent(s) to form a ring(s);
L₁ each independently represents a single bond, a substituted or unsubstituted (C6-C30)arylene, or a substituted or unsubstituted (3- to 30-membered)heteroarylene; and
Ar₁ and Ar₂ each independently represent a substituted or unsubstituted (C6-C30) aryl, or a substituted or unsubstituted (3- to 30-membered)heteroaryl; 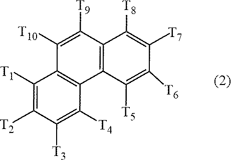
in formula 2,
at least one of T₅ and T₆, and T₇ and T₈ is linked to each other to form a ring(s) together with the following formula 3; 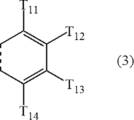
in formulas 2 and 3,
T₁ to T₄, T₉ to T₁₄, and T₅ to T₈ that do not form a ring(s) together with formula 3 each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono- or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted mono- or di- (C6-C30)arylamino, a substituted or unsubstituted (C1-C30)alkyl(C6-C30)arylamino, a substituted or unsubstituted mono- or di- (3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, a substituted or unsubstituted (C6-C30)aryl(3- to 30-membered)heteroarylamino, or -L₂-Ar₃, provided that at least one of T₁ to T₁₄ represents -L₂-Ar₃;
L₂ each independently represents a single bond, a substituted or unsubstituted (C6-C30)arylene, or a substituted or unsubstituted (3- to 30-membered)heteroarylene;
Ar₃ each independently represents a substituted or unsubstituted (C6-C30)aryl, or a substituted or unsubstituted (3- to 30-membered)heteroaryl; 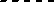
represents a fusion site with formula 2; and
the heteroaryl and the heteroarylene contain one or more heteroatoms selected from B, N, O, S, Si and P.

2. The plurality of host materials according to claim 1, wherein the substituent(s) of the substituted alkyl, the substituted aryl, the substituted arylene, the substituted heteroaryl, the substituted heteroarylene, the substituted cycloalkyl, the substituted alkoxy, the substituted trialkylsilyl, the substituted dialkylarylsilyl, the substituted alkyldiarylsilyl, the substituted triarylsilyl, the substituted fused ring group of an aliphatic ring(s) and an aromatic ring(s), the substituted mono- or di- alkylamino, the substituted mono- or di- alkenylamino, the substituted alkylalkenylamino, the substituted mono- or di- arylamino, the substituted alkylarylamino, the substituted mono- or di- heteroarylamino, the substituted alkylheteroarylamino, the substituted alkenylarylamino, the substituted alkenylheteroarylamino, and the substituted arylheteroarylamino each independently are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of a (C1-C30)alkyl(s) and a (C6-C30)aryl(s); a (C6-C30)aryl unsubstituted or substituted with at least one of deuterium, a cyano(s), a halogen(s), a (C1-C30)alkyl(s), a (C3-C30)cycloalkyl(s), a tri(C1-C30)alkylsilyl(s), a tri(C6-C30)arylsilyl(s), a (C6-C30)aryl(s) and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di- (C1-C30)alkylamino; a mono- or di- (C2-C30)alkenylamino; a (C1-C30)alkyl(C2-C30)alkenylamino; a mono- or di- (C6-C30)arylamino; a (C1-C30)alkyl(C6-C30)arylamino; a mono- or di- (3- to 30-membered)heteroarylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

3. The plurality of host materials according to claim 1, wherein formula 1 is represented by the following formula 4: 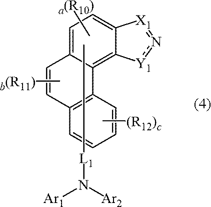
in formula 4,
R₁₀ to R₁₂ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono-or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted mono- or di- (C6-C30)arylamino, a substituted or unsubstituted (C1-C30)alkyl(C6-C30)arylamino, a substituted or unsubstituted mono- or di- (3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or may be linked to an adjacent substituent(s) to form a ring(s);
a and b each independently represent an integer of 1 or 2, and c represents an integer of 1 to 4, where if a to c are an interger of 2 or more, each of R₁₀ to each of R₁₂ may be the same or different; and
X₁, Y₁, L₁, Ar₁, and Ar₂ are as defined in claim 1.

4. The plurality of host materials according to claim 1, wherein R₁, Ar₁ and Ar₂ each independently represent a substituted or unsubstituted phenyl, a substituted or unsubstituted naphthyl, a substituted or unsubstituted biphenyl, a substituted or unsubstituted terphenyl, a substituted or unsubstituted phenanthrenyl, a substituted or unsubstituted anthracenyl, a substituted or unsubstituted fluorenyl, a substituted or unsubstituted benzofluorenyl, substituted or unsubstituted triphenylenyl, a substituted or unsubstituted spirobifluorenyl, a substituted or unsubstituted pyridyl, a substituted or unsubstituted triazinyl, a substituted or unsubstituted pyrimidinyl, a substituted or unsubstituted quinolyl, a substituted or unsubstituted quinazolinyl, a substituted or unsubstituted quinoxalinyl, a substituted or unsubstituted benzoquinazolinyl, a substituted or unsubstituted benzoquinoxalinyl, a substituted or unsubstituted benzofuropyrimidinyl, a substituted or unsubstituted carbazolyl, a substituted or unsubstituted dibenzothiophenyl, a substituted or unsubstituted benzothiophenyl, a substituted or unsubstituted dibenzofuranyl, a substituted or unsubstituted benzofuranyl, a substituted or unsubstituted naphthyridinyl, a substituted or unsubstituted benzonaphthofuranyl, or a substituted or unsubstituted benzonaphthothiophenyl.

5. The plurality of host materials according to claim 1, wherein formula 2 is represented by at least one of the following formulas 2-1 and 2-2: 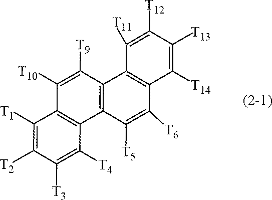 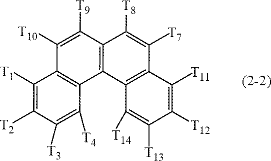
in formulas 2-1 and 2-2,
T₁ to T₄, and T₉ to T₁₄ are as defined in claim 1, and
T₅ to T₈ are as defined for T₁ to T₄, and T₉ to T₁₄.

6. The plurality of host materials according to claim 1, wherein formula 2 is represented by at least one of following formulas: 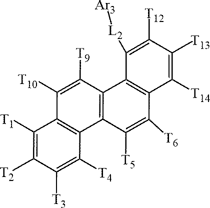 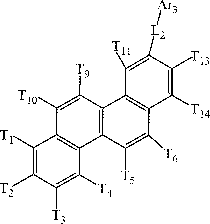 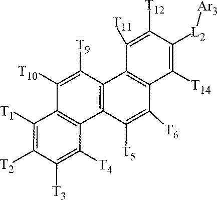 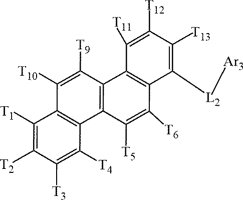 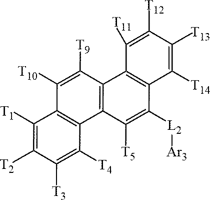 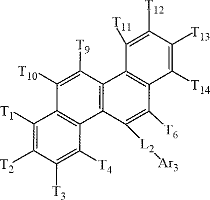 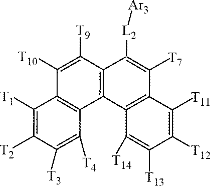 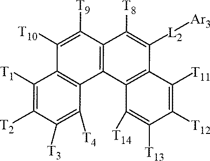 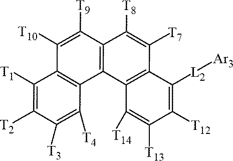 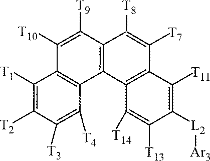 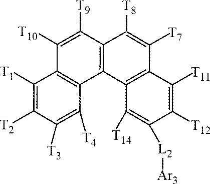 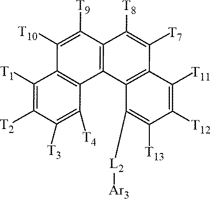
in the above formulas,
T₁ to T₁₄ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono-or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted mono- or di- (C6-C30)arylamino, a substituted or unsubstituted (C1-C30)alkyl(C6-C30)arylamino, a substituted or unsubstituted mono- or di- (3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; and
L₂ and Ar₃ are as defined in claim 1.

7. The plurality of host materials according to claim 1, wherein Ar₃ each independently represents a substituted or unsubstituted phenyl, a substituted or unsubstituted naphthyl, a substituted or unsubstituted biphenyl, a substituted or unsubstituted terphenyl, a substituted or unsubstituted phenanthrenyl, a substituted or unsubstituted phenanthroimidazolyl, a substituted or unsubstituted anthracenyl, a substituted or unsubstituted fluorenyl, a substituted or unsubstituted benzofluorenyl, a substituted or unsubstituted triphenylenyl, a substituted or unsubstituted spirobifluorenyl, a substituted or unsubstituted pyridyl, a substituted or unsubstituted triazinyl, a substituted or unsubstituted pyrimidinyl, a substituted or unsubstituted quinolyl, a substituted or unsubstituted isoquinolyl, a substituted or unsubstituted benzothienoquinolyl, a substituted or unsubstituted quinazolinyl, a substituted or unsubstituted quinoxalinyl, a substituted or unsubstituted dibenzoquinoxalinyl, a substituted or unsubstituted benzoquinazolinyl, a substituted or unsubstituted benzoquinoxalinyl, a substituted or unsubstituted benzofuropyrimidinyl, a substituted or unsubstituted benzoimidazolyl, a substituted or unsubstituted acenaphthopyrimidinyl, a substituted or unsubstituted carbazolyl, a substituted or unsubstituted dibenzothiophenyl, a substituted or unsubstituted benzothiophenyl, a substituted or unsubstituted dibenzofuranyl, a substituted or unsubstituted benzofuranyl, a substituted or unsubstituted naphthyridinyl, a substituted or unsubstituted benzonaphthofuranyl, a substituted or unsubstituted benzonaphthothiophenyl, or a substituted or unsubstituted (16- to 25-membered)heteroaryl comprising at least one heteroatom(s) selected from N, O, and S.

8. The plurality of host materials according to claim 1, wherein the compound represented by formula 1 is at least one selected from the following compounds: 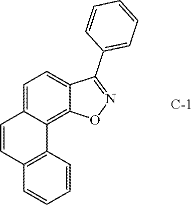 [SMILES: c1ccc(-c2noc3c2ccc2ccc4ccccc4c23)cc1] 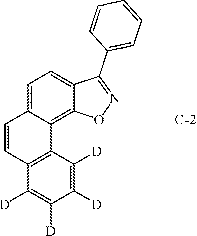 [SMILES: [2H]c1c([2H])c([2H])c2c(ccc3ccc4c(-c5ccccc5)noc4c32)c1[2H]] 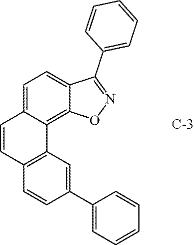 [SMILES: c1ccc(-c2ccc3ccc4ccc5c(-c6ccccc6)noc5c4c3c2)cc1] 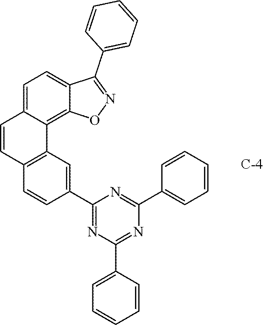 [SMILES: c1ccc(-c2nc(-c3ccccc3)nc(-c3ccc4ccc5ccc6c(-c7ccccc7)noc6c5c4c3)n2)cc1] 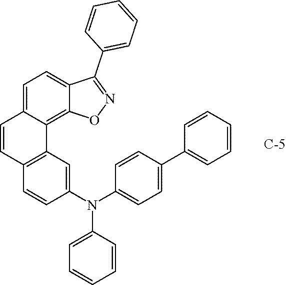 [SMILES: c1ccc(-c2ccc(N(c3ccccc3)c3ccc4ccc5ccc6c(-c7ccccc7)noc6c5c4c3)cc2)cc1] 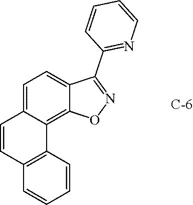 [SMILES: c1ccc(-c2noc3c2ccc2ccc4ccccc4c23)nc1] 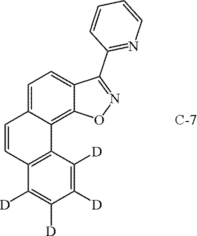 [SMILES: [2H]c1c([2H])c([2H])c2c(ccc3ccc4c(-c5ccccn5)noc4c32)c1[2H]] 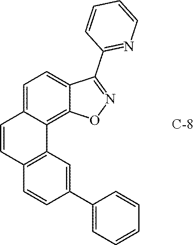 [SMILES: c1ccc(-c2ccc3ccc4ccc5c(-c6ccccn6)noc5c4c3c2)cc1] 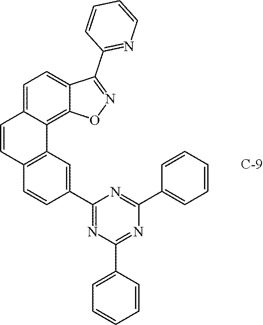 [SMILES: c1ccc(-c2nc(-c3ccccc3)nc(-c3ccc4ccc5ccc6c(-c7ccccn7)noc6c5c4c3)n2)cc1] 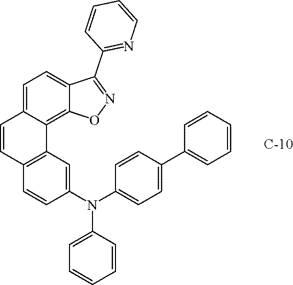 [SMILES: c1ccc(-c2ccc(N(c3ccccc3)c3ccc4ccc5ccc6c(-c7ccccn7)noc6c5c4c3)cc2)cc1] 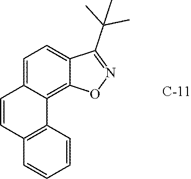 [SMILES: CC(C)(C)c1noc2c1ccc1ccc3ccccc3c12] 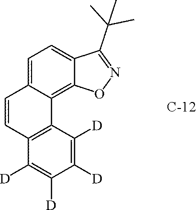 [SMILES: [2H]c1c([2H])c([2H])c2c(ccc3ccc4c(C(C)(C)C)noc4c32)c1[2H]] 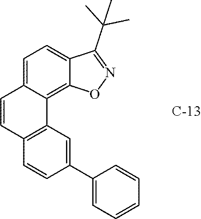 [SMILES: CC(C)(C)c1noc2c1ccc1ccc3ccc(-c4ccccc4)cc3c12] 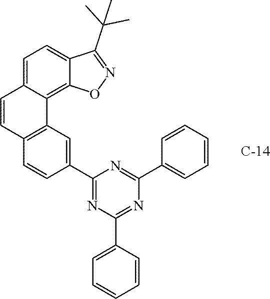 [SMILES: CC(C)(C)c1noc2c1ccc1ccc3ccc(-c4nc(-c5ccccc5)nc(-c5ccccc5)n4)cc3c12] 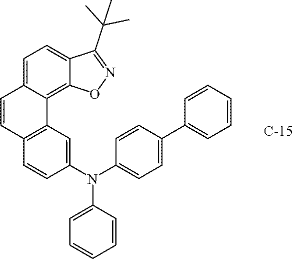 [SMILES: CC(C)(C)c1noc2c1ccc1ccc3ccc(N(c4ccccc4)c4ccc(-c5ccccc5)cc4)cc3c12] 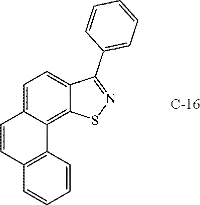 [SMILES: c1ccc(-c2nsc3c2ccc2ccc4ccccc4c23)cc1] 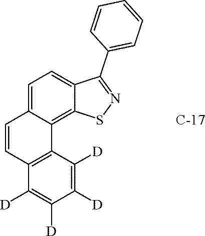 [SMILES: [2H]c1c([2H])c([2H])c2c(ccc3ccc4c(-c5ccccc5)nsc4c32)c1[2H]] 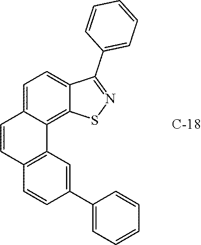 [SMILES: c1ccc(-c2ccc3ccc4ccc5c(-c6ccccc6)nsc5c4c3c2)cc1] 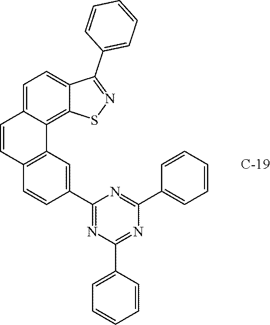 [SMILES: c1ccc(-c2nc(-c3ccccc3)nc(-c3ccc4ccc5ccc6c(-c7ccccc7)nsc6c5c4c3)n2)cc1] 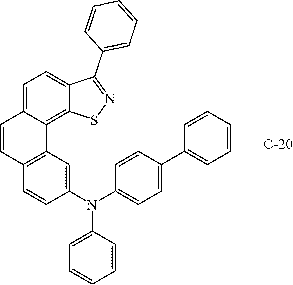 [SMILES: c1ccc(-c2ccc(N(c3ccccc3)c3ccc4ccc5ccc6c(-c7ccccc7)nsc6c5c4c3)cc2)cc1] 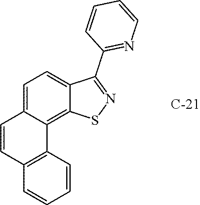 [SMILES: c1ccc(-c2nsc3c2ccc2ccc4ccccc4c23)nc1] 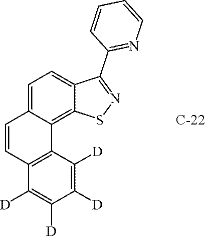 [SMILES: [2H]c1c([2H])c([2H])c2c(ccc3ccc4c(-c5ccccn5)nsc4c32)c1[2H]] 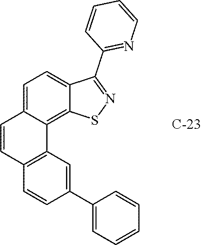 [SMILES: c1ccc(-c2ccc3ccc4ccc5c(-c6ccccn6)nsc5c4c3c2)cc1] 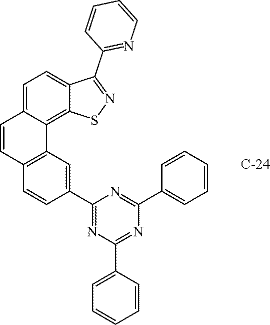 [SMILES: c1ccc(-c2nc(-c3ccccc3)nc(-c3ccc4ccc5ccc6c(-c7ccccn7)nsc6c5c4c3)n2)cc1] 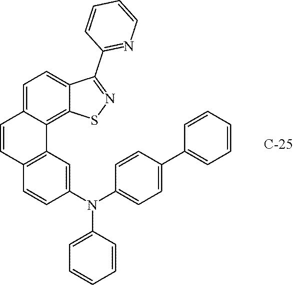 [SMILES: c1ccc(-c2ccc(N(c3ccccc3)c3ccc4ccc5ccc6c(-c7ccccn7)nsc6c5c4c3)cc2)cc1] 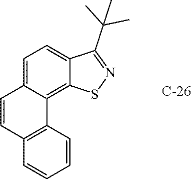 [SMILES: CC(C)(C)c1nsc2c1ccc1ccc3ccccc3c12] 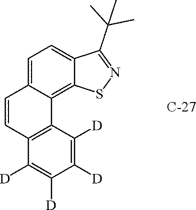 [SMILES: [2H]c1c([2H])c([2H])c2c(ccc3ccc4c(C(C)(C)C)nsc4c32)c1[2H]] 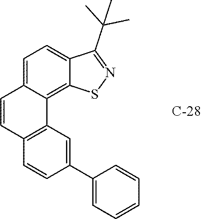 [SMILES: CC(C)(C)c1nsc2c1ccc1ccc3ccc(-c4ccccc4)cc3c12] 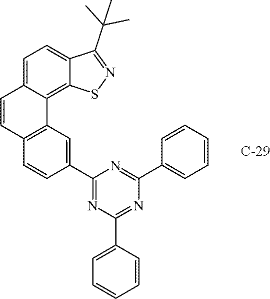 [SMILES: CC(C)(C)c1nsc2c1ccc1ccc3ccc(-c4nc(-c5ccccc5)nc(-c5ccccc5)n4)cc3c12] 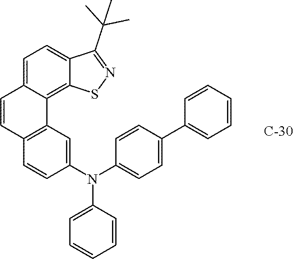 [SMILES: CC(C)(C)c1nsc2c1ccc1ccc3ccc(N(c4ccccc4)c4ccc(-c5ccccc5)cc4)cc3c12] 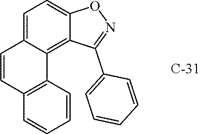 [SMILES: c1ccc(-c2noc3ccc4ccc5ccccc5c4c23)cc1] 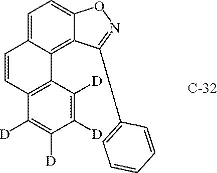 [SMILES: [2H]c1c([2H])c([2H])c2c(ccc3ccc4onc(-c5ccccc5)c4c32)c1[2H]] 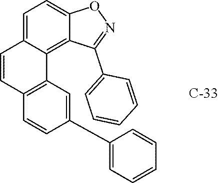 [SMILES: c1ccc(-c2ccc3ccc4ccc5onc(-c6ccccc6)c5c4c3c2)cc1] 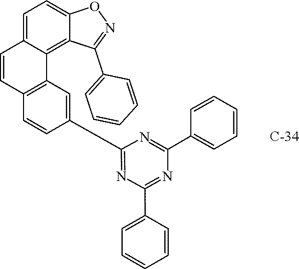 [SMILES: c1ccc(-c2nc(-c3ccccc3)nc(-c3ccc4ccc5ccc6onc(-c7ccccc7)c6c5c4c3)n2)cc1] 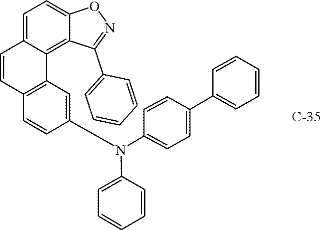 [SMILES: c1ccc(-c2ccc(N(c3ccccc3)c3ccc4ccc5ccc6onc(-c7ccccc7)c6c5c4c3)cc2)cc1] 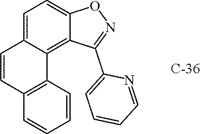 [SMILES: c1ccc(-c2noc3ccc4ccc5ccccc5c4c23)nc1] 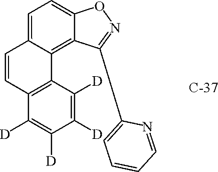 [SMILES: [2H]c1c([2H])c([2H])c2c(ccc3ccc4onc(-c5ccccn5)c4c32)c1[2H]] 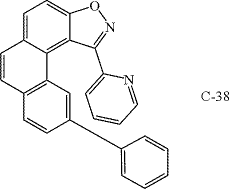 [SMILES: c1ccc(-c2ccc3ccc4ccc5onc(-c6ccccn6)c5c4c3c2)cc1] 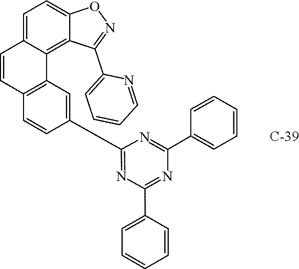 [SMILES: c1ccc(-c2nc(-c3ccccc3)nc(-c3ccc4ccc5ccc6onc(-c7ccccn7)c6c5c4c3)n2)cc1] 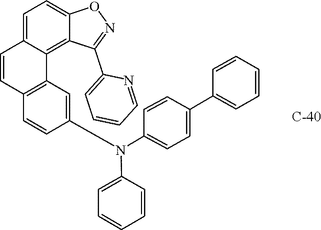 [SMILES: c1ccc(-c2ccc(N(c3ccccc3)c3ccc4ccc5ccc6onc(-c7ccccn7)c6c5c4c3)cc2)cc1] 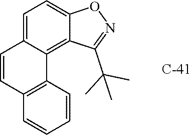 [SMILES: CC(C)(C)c1noc2ccc3ccc4ccccc4c3c12] 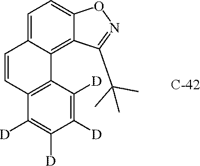 [SMILES: [2H]c1c([2H])c([2H])c2c(ccc3ccc4onc(C(C)(C)C)c4c32)c1[2H]] 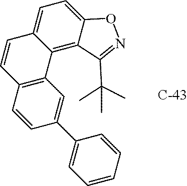 [SMILES: CC(C)(C)c1noc2ccc3ccc4ccc(-c5ccccc5)cc4c3c12] 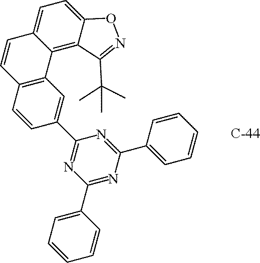 [SMILES: CC(C)(C)c1noc2ccc3ccc4ccc(-c5nc(-c6ccccc6)nc(-c6ccccc6)n5)cc4c3c12] 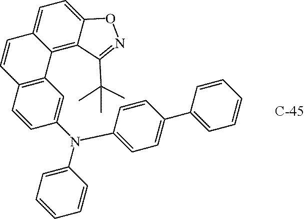 [SMILES: CC(C)(C)c1noc2ccc3ccc4ccc(N(c5ccccc5)c5ccc(-c6ccccc6)cc5)cc4c3c12] 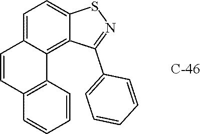 [SMILES: c1ccc(-c2nsc3ccc4ccc5ccccc5c4c23)cc1] 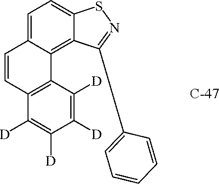 [SMILES: [2H]c1c([2H])c([2H])c2c(ccc3ccc4snc(-c5ccccc5)c4c32)c1[2H]] 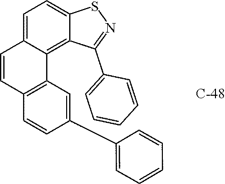 [SMILES: c1ccc(-c2ccc3ccc4ccc5snc(-c6ccccc6)c5c4c3c2)cc1] 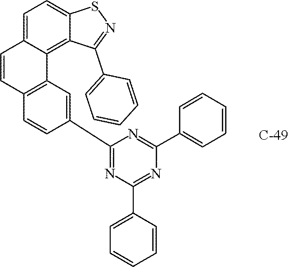 [SMILES: c1ccc(-c2nc(-c3ccccc3)nc(-c3ccc4ccc5ccc6snc(-c7ccccc7)c6c5c4c3)n2)cc1] 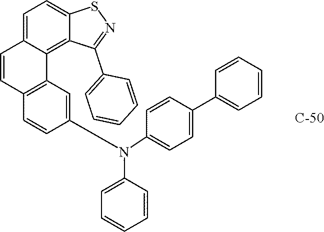 [SMILES: c1ccc(-c2ccc(N(c3ccccc3)c3ccc4ccc5ccc6snc(-c7ccccc7)c6c5c4c3)cc2)cc1] 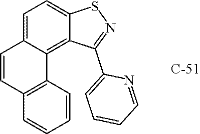 [SMILES: c1ccc(-c2nsc3ccc4ccc5ccccc5c4c23)nc1] 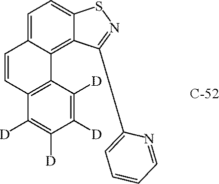 [SMILES: [2H]c1c([2H])c([2H])c2c(ccc3ccc4snc(-c5ccccn5)c4c32)c1[2H]] 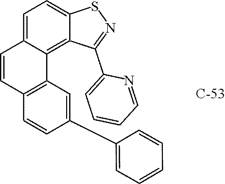 [SMILES: c1ccc(-c2ccc3ccc4ccc5snc(-c6ccccn6)c5c4c3c2)cc1] 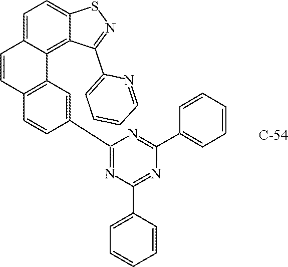 [SMILES: c1ccc(-c2nc(-c3ccccc3)nc(-c3ccc4ccc5ccc6snc(-c7ccccn7)c6c5c4c3)n2)cc1] 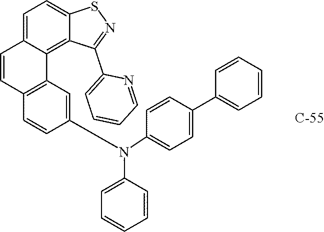 [SMILES: c1ccc(-c2ccc(N(c3ccccc3)c3ccc4ccc5ccc6snc(-c7ccccn7)c6c5c4c3)cc2)cc1] 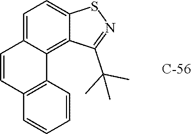 [SMILES: CC(C)(C)c1nsc2ccc3ccc4ccccc4c3c12] 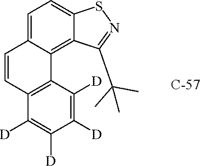 [SMILES: [2H]c1c([2H])c([2H])c2c(ccc3ccc4snc(C(C)(C)C)c4c32)c1[2H]] 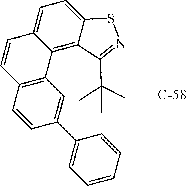 [SMILES: CC(C)(C)c1nsc2ccc3ccc4ccc(-c5ccccc5)cc4c3c12] 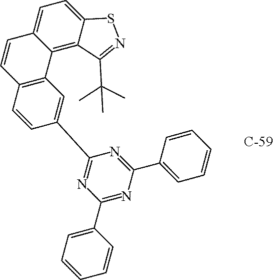 [SMILES: CC(C)(C)c1nsc2ccc3ccc4ccc(-c5nc(-c6ccccc6)nc(-c6ccccc6)n5)cc4c3c12] 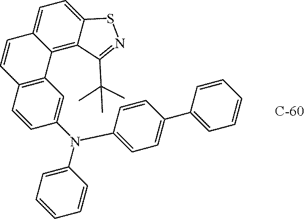 [SMILES: CC(C)(C)c1nsc2ccc3ccc4ccc(N(c5ccccc5)c5ccc(-c6ccccc6)cc5)cc4c3c12] 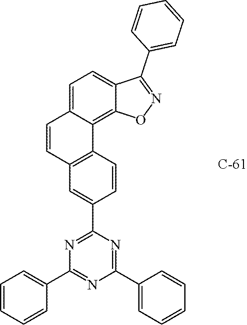 [SMILES: c1ccc(-c2nc(-c3ccccc3)nc(-c3ccc4c(ccc5ccc6c(-c7ccccc7)noc6c54)c3)n2)cc1] 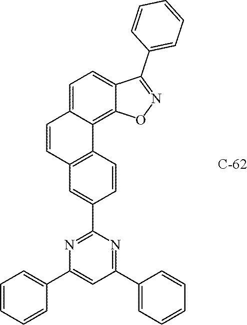 [SMILES: c1ccc(-c2cc(-c3ccccc3)nc(-c3ccc4c(ccc5ccc6c(-c7ccccc7)noc6c54)c3)n2)cc1] 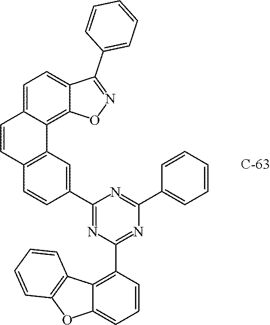 [SMILES: c1ccc(-c2nc(-c3ccc4ccc5ccc6c(-c7ccccc7)noc6c5c4c3)nc(-c3cccc4oc5ccccc5c34)n2)cc1] 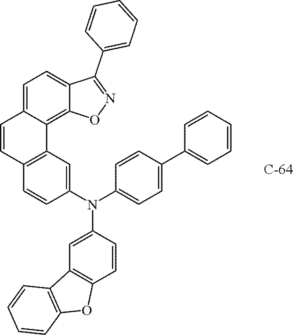 [SMILES: c1ccc(-c2ccc(N(c3ccc4oc5ccccc5c4c3)c3ccc4ccc5ccc6c(-c7ccccc7)noc6c5c4c3)cc2)cc1] 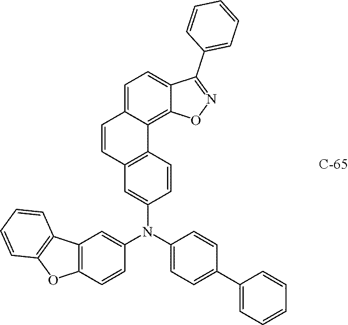 [SMILES: c1ccc(-c2ccc(N(c3ccc4c(ccc5ccc6c(-c7ccccc7)noc6c54)c3)c3ccc4oc5ccccc5c4c3)cc2)cc1] 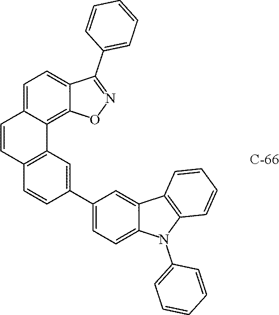 [SMILES: c1ccc(-c2noc3c2ccc2ccc4ccc(-c5ccc6c(c5)c5ccccc5n6-c5ccccc5)cc4c23)cc1] 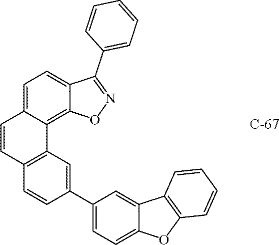 [SMILES: c1ccc(-c2noc3c2ccc2ccc4ccc(-c5ccc6oc7ccccc7c6c5)cc4c23)cc1] 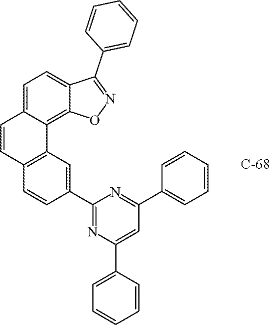 [SMILES: c1ccc(-c2cc(-c3ccccc3)nc(-c3ccc4ccc5ccc6c(-c7ccccc7)noc6c5c4c3)n2)cc1] 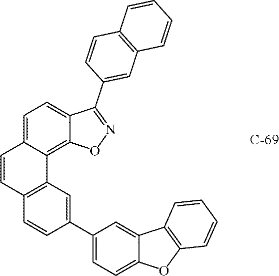 [SMILES: c1ccc2cc(-c3noc4c3ccc3ccc5ccc(-c6ccc7oc8ccccc8c7c6)cc5c34)ccc2c1] 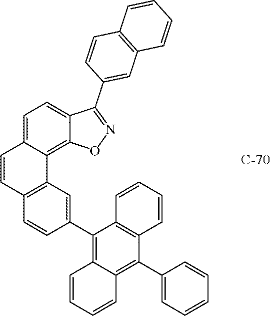 [SMILES: c1ccc(-c2c3ccccc3c(-c3ccc4ccc5ccc6c(-c7ccc8ccccc8c7)noc6c5c4c3)c3ccccc23)cc1] 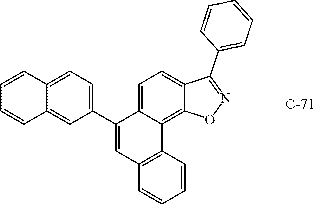 [SMILES: c1ccc(-c2noc3c2ccc2c(-c4ccc5ccccc5c4)cc4ccccc4c23)cc1] 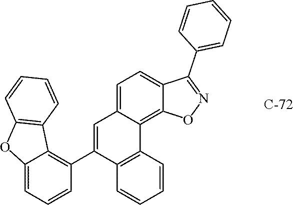 [SMILES: c1ccc(-c2noc3c2ccc2cc(-c4cccc5oc6ccccc6c45)c4ccccc4c23)cc1] 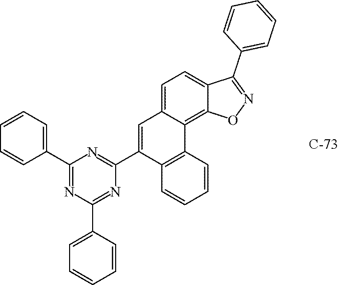 [SMILES: c1ccc(-c2nc(-c3ccccc3)nc(-c3cc4ccc5c(-c6ccccc6)noc5c4c4ccccc34)n2)cc1] 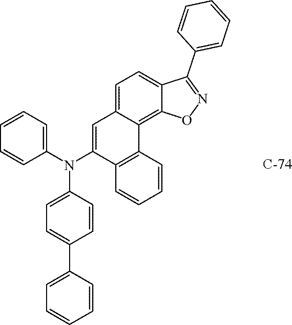 [SMILES: c1ccc(-c2ccc(N(c3ccccc3)c3cc4ccc5c(-c6ccccc6)noc5c4c4ccccc34)cc2)cc1]
and 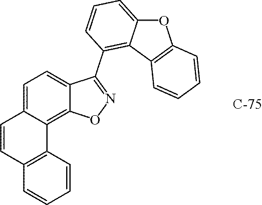 [SMILES: c1ccc2c(c1)ccc1ccc3c(-c4cccc5oc6ccccc6c45)noc3c12]
.

9. The plurality of host materials according to claim 1, wherein the compound represented by formula 2 is at least one selected from the following compounds: 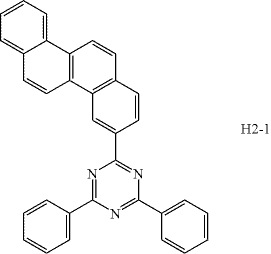 [SMILES: c1ccc(-c2nc(-c3ccccc3)nc(-c3ccc4ccc5c6ccccc6ccc5c4c3)n2)cc1] 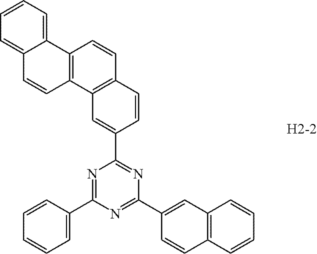 [SMILES: c1ccc(-c2nc(-c3ccc4ccccc4c3)nc(-c3ccc4ccc5c6ccccc6ccc5c4c3)n2)cc1] 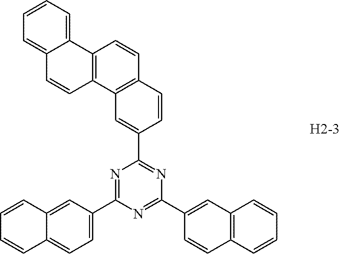 [SMILES: c1ccc2cc(-c3nc(-c4ccc5ccccc5c4)nc(-c4ccc5ccc6c7ccccc7ccc6c5c4)n3)ccc2c1] 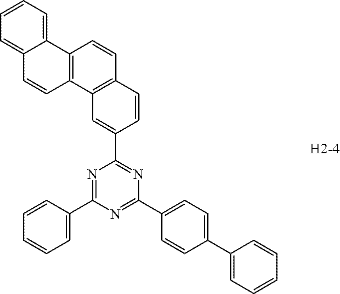 [SMILES: c1ccc(-c2ccc(-c3nc(-c4ccccc4)nc(-c4ccc5ccc6c7ccccc7ccc6c5c4)n3)cc2)cc1] 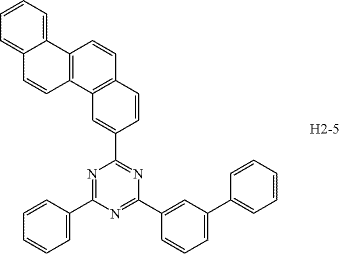 [SMILES: c1ccc(-c2cccc(-c3nc(-c4ccccc4)nc(-c4ccc5ccc6c7ccccc7ccc6c5c4)n3)c2)cc1] 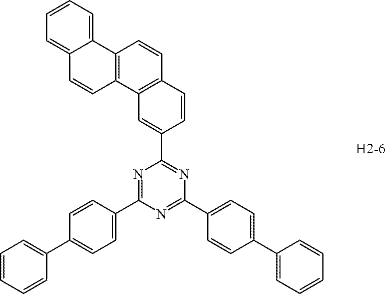 [SMILES: c1ccc(-c2ccc(-c3nc(-c4ccc(-c5ccccc5)cc4)nc(-c4ccc5ccc6c7ccccc7ccc6c5c4)n3)cc2)cc1] 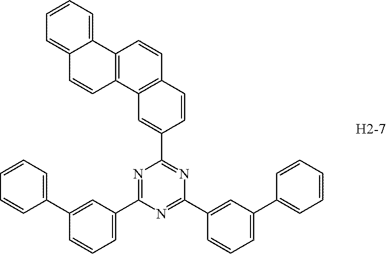 [SMILES: c1ccc(-c2cccc(-c3nc(-c4cccc(-c5ccccc5)c4)nc(-c4ccc5ccc6c7ccccc7ccc6c5c4)n3)c2)cc1] 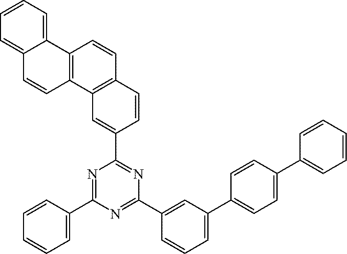 [SMILES: c1ccc(-c2ccc(-c3cccc(-c4nc(-c5ccccc5)nc(-c5ccc6ccc7c8ccccc8ccc7c6c5)n4)c3)cc2)cc1] 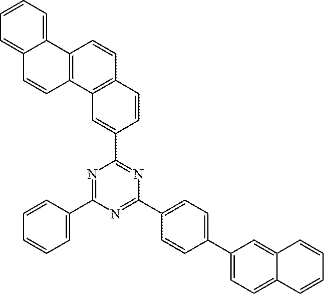 [SMILES: c1ccc(-c2nc(-c3ccc(-c4ccc5ccccc5c4)cc3)nc(-c3ccc4ccc5c6ccccc6ccc5c4c3)n2)cc1] 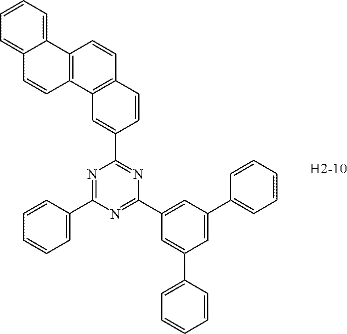 [SMILES: c1ccc(-c2cc(-c3ccccc3)cc(-c3nc(-c4ccccc4)nc(-c4ccc5ccc6c7ccccc7ccc6c5c4)n3)c2)cc1] 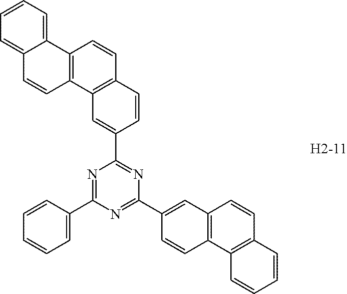 [SMILES: c1ccc(-c2nc(-c3ccc4c(ccc5ccccc54)c3)nc(-c3ccc4ccc5c6ccccc6ccc5c4c3)n2)cc1] 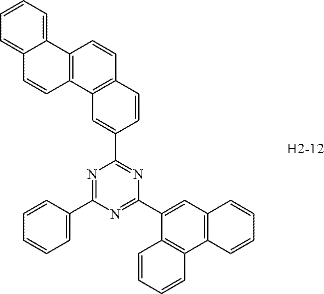 [SMILES: c1ccc(-c2nc(-c3ccc4ccc5c6ccccc6ccc5c4c3)nc(-c3cc4ccccc4c4ccccc34)n2)cc1] 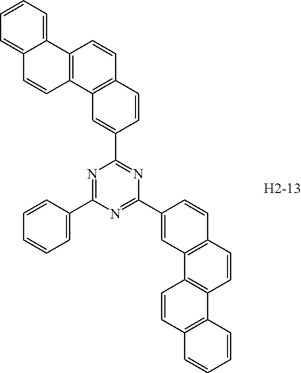 [SMILES: c1ccc(-c2nc(-c3ccc4ccc5c6ccccc6ccc5c4c3)nc(-c3ccc4ccc5c6ccccc6ccc5c4c3)n2)cc1] 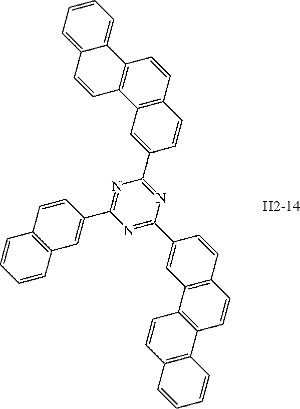 [SMILES: c1ccc2cc(-c3nc(-c4ccc5ccc6c7ccccc7ccc6c5c4)nc(-c4ccc5ccc6c7ccccc7ccc6c5c4)n3)ccc2c1] 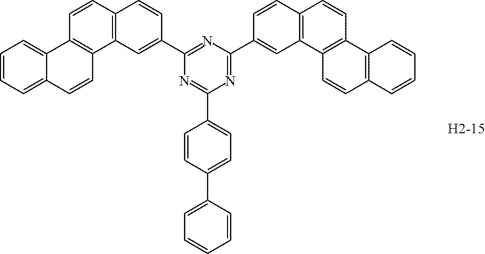 [SMILES: c1ccc(-c2ccc(-c3nc(-c4ccc5ccc6c7ccccc7ccc6c5c4)nc(-c4ccc5ccc6c7ccccc7ccc6c5c4)n3)cc2)cc1] 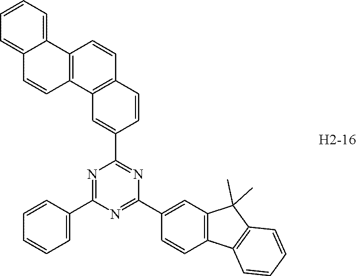 [SMILES: CC1(C)c2ccccc2-c2ccc(-c3nc(-c4ccccc4)nc(-c4ccc5ccc6c7ccccc7ccc6c5c4)n3)cc21] 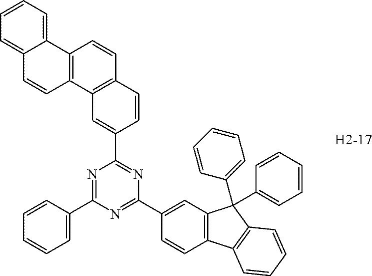 [SMILES: c1ccc(-c2nc(-c3ccc4c(c3)C(c3ccccc3)(c3ccccc3)c3ccccc3-4)nc(-c3ccc4ccc5c6ccccc6ccc5c4c3)n2)cc1] 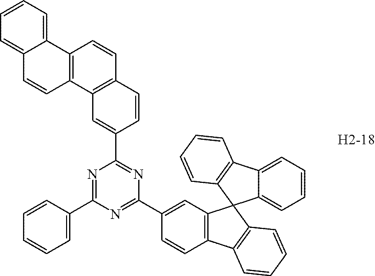 [SMILES: c1ccc(-c2nc(-c3ccc4c(c3)C3(c5ccccc5-c5ccccc53)c3ccccc3-4)nc(-c3ccc4ccc5c6ccccc6ccc5c4c3)n2)cc1] 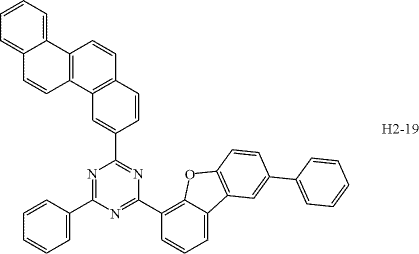 [SMILES: c1ccc(-c2ccc3oc4c(-c5nc(-c6ccccc6)nc(-c6ccc7ccc8c9ccccc9ccc8c7c6)n5)cccc4c3c2)cc1] 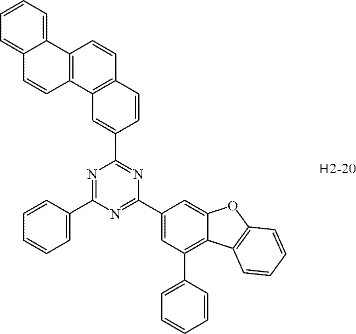 [SMILES: c1ccc(-c2nc(-c3cc(-c4ccccc4)c4c(c3)oc3ccccc34)nc(-c3ccc4ccc5c6ccccc6ccc5c4c3)n2)cc1] 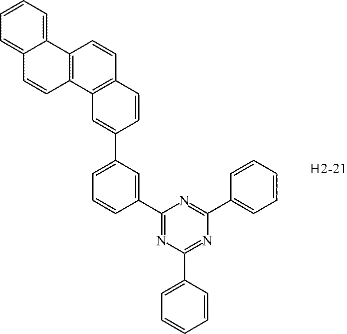 [SMILES: c1ccc(-c2nc(-c3ccccc3)nc(-c3cccc(-c4ccc5ccc6c7ccccc7ccc6c5c4)c3)n2)cc1] 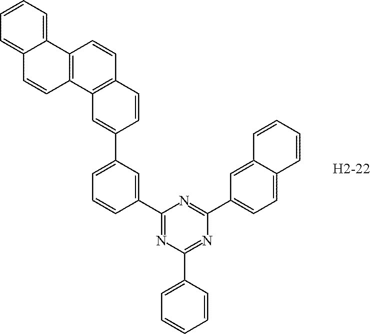 [SMILES: c1ccc(-c2nc(-c3cccc(-c4ccc5ccc6c7ccccc7ccc6c5c4)c3)nc(-c3ccc4ccccc4c3)n2)cc1] 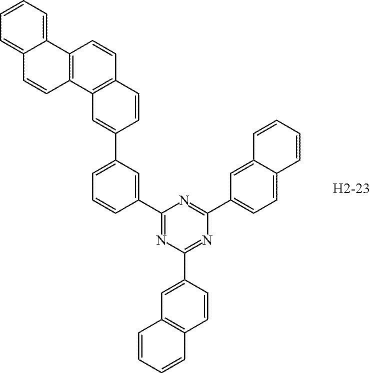 [SMILES: c1cc(-c2ccc3ccc4c5ccccc5ccc4c3c2)cc(-c2nc(-c3ccc4ccccc4c3)nc(-c3ccc4ccccc4c3)n2)c1] 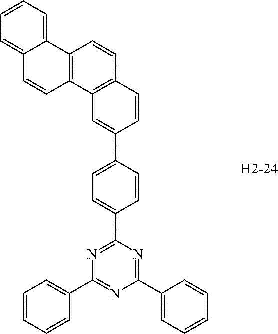 [SMILES: c1ccc(-c2nc(-c3ccccc3)nc(-c3ccc(-c4ccc5ccc6c7ccccc7ccc6c5c4)cc3)n2)cc1] 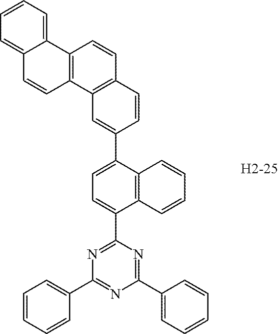 [SMILES: c1ccc(-c2nc(-c3ccccc3)nc(-c3ccc(-c4ccc5ccc6c7ccccc7ccc6c5c4)c4ccccc34)n2)cc1] 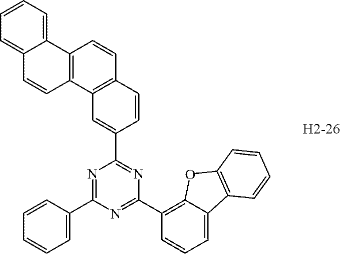 [SMILES: c1ccc(-c2nc(-c3ccc4ccc5c6ccccc6ccc5c4c3)nc(-c3cccc4c3oc3ccccc34)n2)cc1] 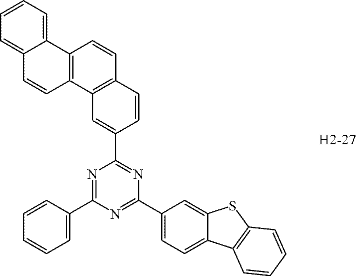 [SMILES: c1ccc(-c2nc(-c3ccc4c(c3)sc3ccccc34)nc(-c3ccc4ccc5c6ccccc6ccc5c4c3)n2)cc1] 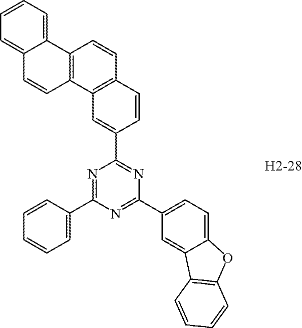 [SMILES: c1ccc(-c2nc(-c3ccc4ccc5c6ccccc6ccc5c4c3)nc(-c3ccc4oc5ccccc5c4c3)n2)cc1] 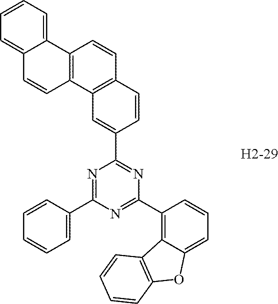 [SMILES: c1ccc(-c2nc(-c3ccc4ccc5c6ccccc6ccc5c4c3)nc(-c3cccc4oc5ccccc5c34)n2)cc1] 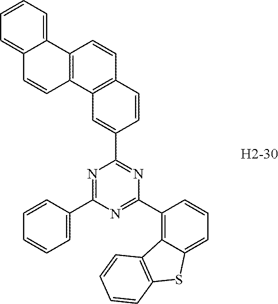 [SMILES: c1ccc(-c2nc(-c3ccc4ccc5c6ccccc6ccc5c4c3)nc(-c3cccc4sc5ccccc5c34)n2)cc1] 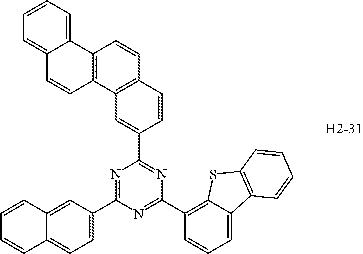 [SMILES: c1ccc2cc(-c3nc(-c4ccc5ccc6c7ccccc7ccc6c5c4)nc(-c4cccc5c4sc4ccccc45)n3)ccc2c1] 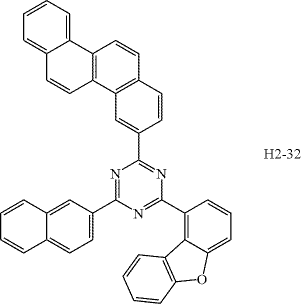 [SMILES: c1ccc2cc(-c3nc(-c4ccc5ccc6c7ccccc7ccc6c5c4)nc(-c4cccc5oc6ccccc6c45)n3)ccc2c1] 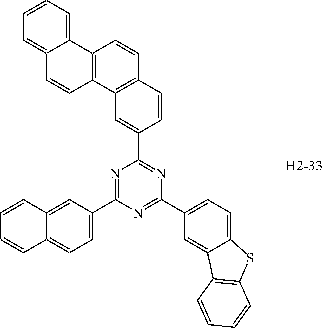 [SMILES: c1ccc2cc(-c3nc(-c4ccc5ccc6c7ccccc7ccc6c5c4)nc(-c4ccc5sc6ccccc6c5c4)n3)ccc2c1] 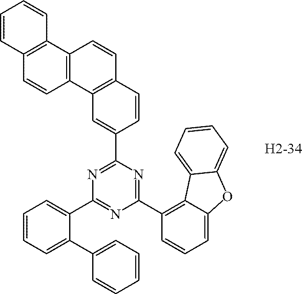 [SMILES: c1ccc(-c2ccccc2-c2nc(-c3ccc4ccc5c6ccccc6ccc5c4c3)nc(-c3cccc4oc5ccccc5c34)n2)cc1] 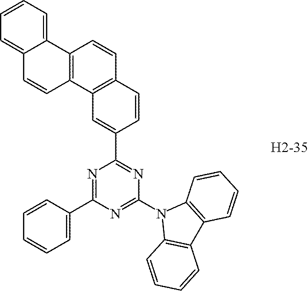 [SMILES: c1ccc(-c2nc(-c3ccc4ccc5c6ccccc6ccc5c4c3)nc(-n3c4ccccc4c4ccccc43)n2)cc1] 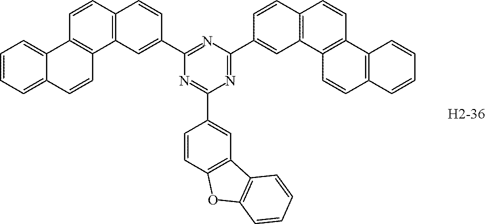 [SMILES: c1ccc2c(c1)ccc1c3cc(-c4nc(-c5ccc6ccc7c8ccccc8ccc7c6c5)nc(-c5ccc6oc7ccccc7c6c5)n4)ccc3ccc21] 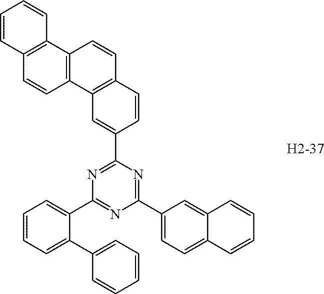 [SMILES: c1ccc(-c2ccccc2-c2nc(-c3ccc4ccccc4c3)nc(-c3ccc4ccc5c6ccccc6ccc5c4c3)n2)cc1] 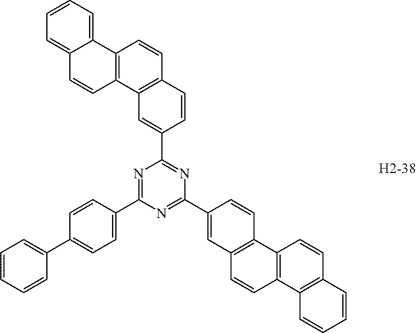 [SMILES: c1ccc(-c2ccc(-c3nc(-c4ccc5c(ccc6c7ccccc7ccc56)c4)nc(-c4ccc5ccc6c7ccccc7ccc6c5c4)n3)cc2)cc1] 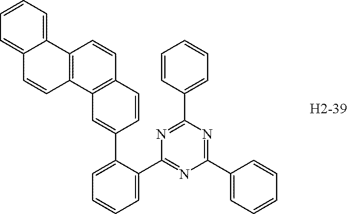 [SMILES: c1ccc(-c2nc(-c3ccccc3)nc(-c3ccccc3-c3ccc4ccc5c6ccccc6ccc5c4c3)n2)cc1] 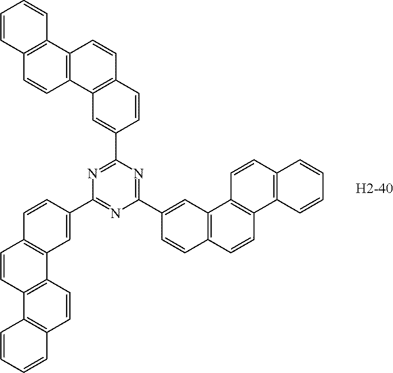 [SMILES: c1ccc2c(c1)ccc1c3cc(-c4nc(-c5ccc6ccc7c8ccccc8ccc7c6c5)nc(-c5ccc6ccc7c8ccccc8ccc7c6c5)n4)ccc3ccc21] 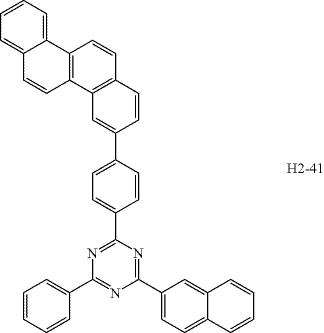 [SMILES: c1ccc(-c2nc(-c3ccc(-c4ccc5ccc6c7ccccc7ccc6c5c4)cc3)nc(-c3ccc4ccccc4c3)n2)cc1] 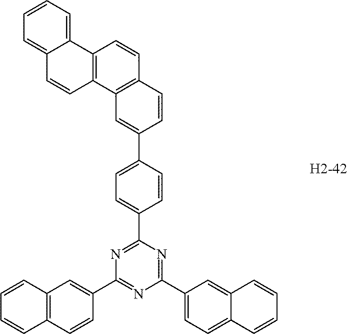 [SMILES: c1ccc2cc(-c3nc(-c4ccc(-c5ccc6ccc7c8ccccc8ccc7c6c5)cc4)nc(-c4ccc5ccccc5c4)n3)ccc2c1] 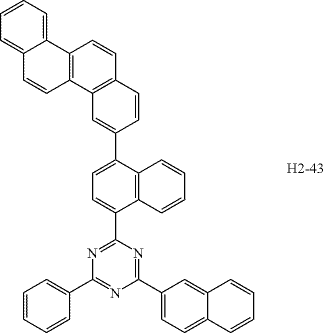 [SMILES: c1ccc(-c2nc(-c3ccc4ccccc4c3)nc(-c3ccc(-c4ccc5ccc6c7ccccc7ccc6c5c4)c4ccccc34)n2)cc1] 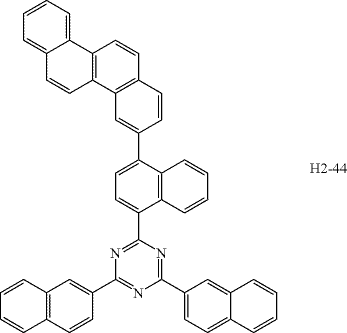 [SMILES: c1ccc2cc(-c3nc(-c4ccc5ccccc5c4)nc(-c4ccc(-c5ccc6ccc7c8ccccc8ccc7c6c5)c5ccccc45)n3)ccc2c1] 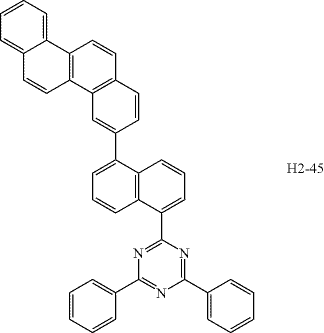 [SMILES: c1ccc(-c2nc(-c3ccccc3)nc(-c3cccc4c(-c5ccc6ccc7c8ccccc8ccc7c6c5)cccc34)n2)cc1] 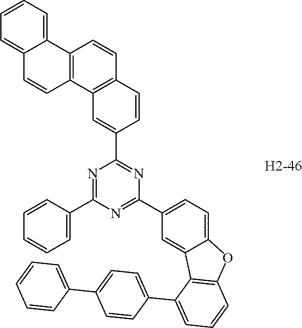 [SMILES: c1ccc(-c2ccc(-c3cccc4oc5ccc(-c6nc(-c7ccccc7)nc(-c7ccc8ccc9c%10ccccc%10ccc9c8c7)n6)cc5c34)cc2)cc1] 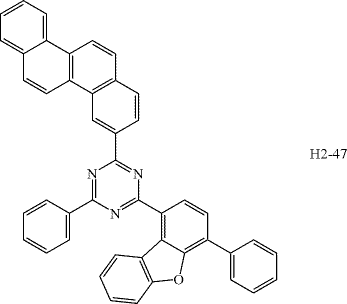 [SMILES: c1ccc(-c2nc(-c3ccc4ccc5c6ccccc6ccc5c4c3)nc(-c3ccc(-c4ccccc4)c4oc5ccccc5c34)n2)cc1] 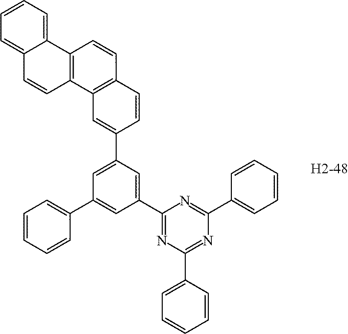 [SMILES: c1ccc(-c2cc(-c3ccc4ccc5c6ccccc6ccc5c4c3)cc(-c3nc(-c4ccccc4)nc(-c4ccccc4)n3)c2)cc1] 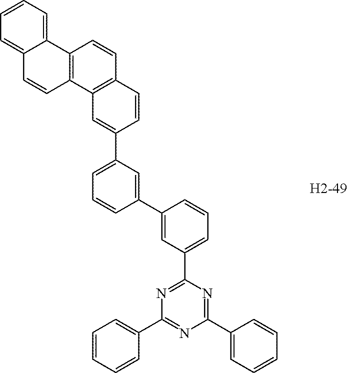 [SMILES: c1ccc(-c2nc(-c3ccccc3)nc(-c3cccc(-c4cccc(-c5ccc6ccc7c8ccccc8ccc7c6c5)c4)c3)n2)cc1] 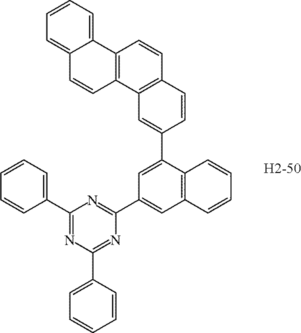 [SMILES: c1ccc(-c2nc(-c3ccccc3)nc(-c3cc(-c4ccc5ccc6c7ccccc7ccc6c5c4)c4ccccc4c3)n2)cc1] 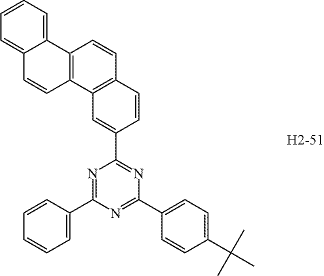 [SMILES: CC(C)(C)c1ccc(-c2nc(-c3ccccc3)nc(-c3ccc4ccc5c6ccccc6ccc5c4c3)n2)cc1] 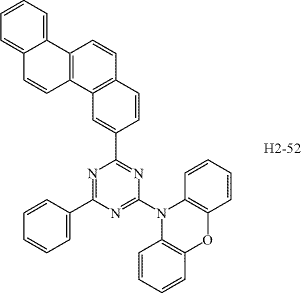 [SMILES: c1ccc(-c2nc(-c3ccc4ccc5c6ccccc6ccc5c4c3)nc(N3c4ccccc4Oc4ccccc43)n2)cc1] 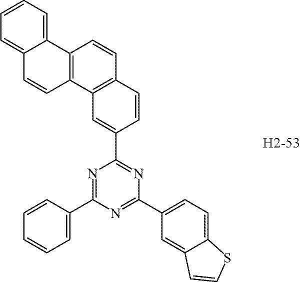 [SMILES: c1ccc(-c2nc(-c3ccc4sccc4c3)nc(-c3ccc4ccc5c6ccccc6ccc5c4c3)n2)cc1] 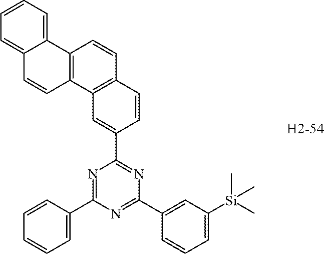 [SMILES: C[Si](C)(C)c1cccc(-c2nc(-c3ccccc3)nc(-c3ccc4ccc5c6ccccc6ccc5c4c3)n2)c1] 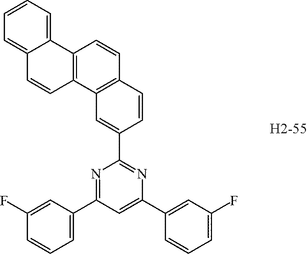 [SMILES: Fc1cccc(-c2cc(-c3cccc(F)c3)nc(-c3ccc4ccc5c6ccccc6ccc5c4c3)n2)c1] 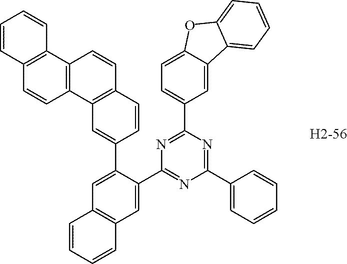 [SMILES: c1ccc(-c2nc(-c3ccc4oc5ccccc5c4c3)nc(-c3cc4ccccc4cc3-c3ccc4ccc5c6ccccc6ccc5c4c3)n2)cc1] 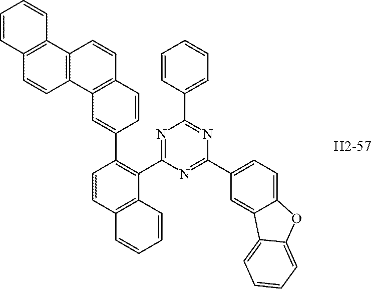 [SMILES: c1ccc(-c2nc(-c3ccc4oc5ccccc5c4c3)nc(-c3c(-c4ccc5ccc6c7ccccc7ccc6c5c4)ccc4ccccc34)n2)cc1] 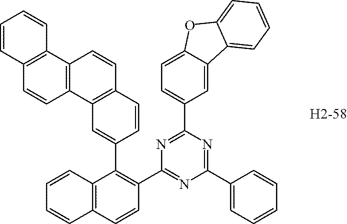 [SMILES: c1ccc(-c2nc(-c3ccc4oc5ccccc5c4c3)nc(-c3ccc4ccccc4c3-c3ccc4ccc5c6ccccc6ccc5c4c3)n2)cc1] 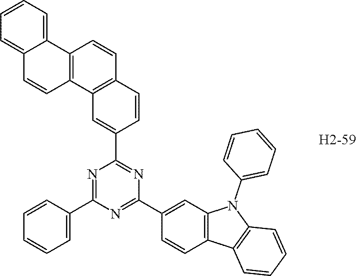 [SMILES: c1ccc(-c2nc(-c3ccc4ccc5c6ccccc6ccc5c4c3)nc(-c3ccc4c5ccccc5n(-c5ccccc5)c4c3)n2)cc1] 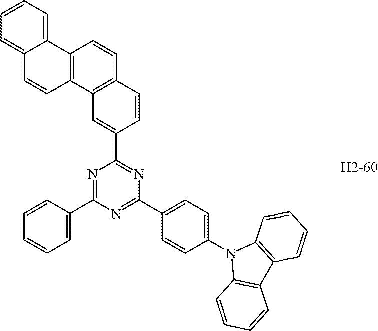 [SMILES: c1ccc(-c2nc(-c3ccc(-n4c5ccccc5c5ccccc54)cc3)nc(-c3ccc4ccc5c6ccccc6ccc5c4c3)n2)cc1] 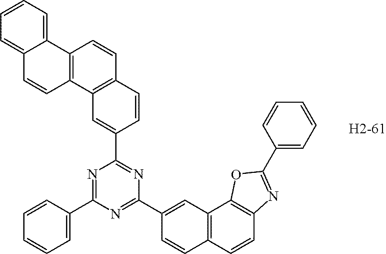 [SMILES: c1ccc(-c2nc(-c3ccc4ccc5c6ccccc6ccc5c4c3)nc(-c3ccc4ccc5nc(-c6ccccc6)oc5c4c3)n2)cc1] 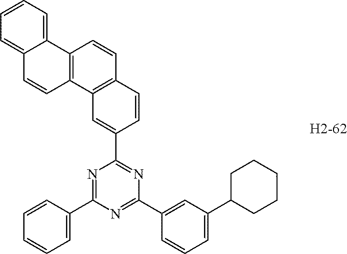 [SMILES: c1ccc(-c2nc(-c3cccc(C4CCCCC4)c3)nc(-c3ccc4ccc5c6ccccc6ccc5c4c3)n2)cc1] 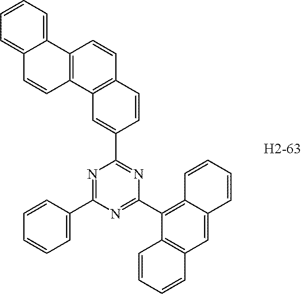 [SMILES: c1ccc(-c2nc(-c3ccc4ccc5c6ccccc6ccc5c4c3)nc(-c3c4ccccc4cc4ccccc34)n2)cc1] 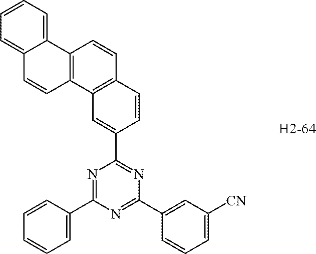 [SMILES: N#Cc1cccc(-c2nc(-c3ccccc3)nc(-c3ccc4ccc5c6ccccc6ccc5c4c3)n2)c1] 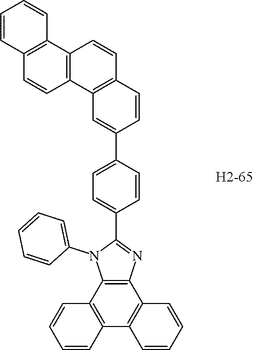 [SMILES: c1ccc(-n2c(-c3ccc(-c4ccc5ccc6c7ccccc7ccc6c5c4)cc3)nc3c4ccccc4c4ccccc4c32)cc1] 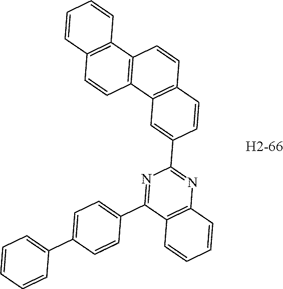 [SMILES: c1ccc(-c2ccc(-c3nc(-c4ccc5ccc6c7ccccc7ccc6c5c4)nc4ccccc34)cc2)cc1] 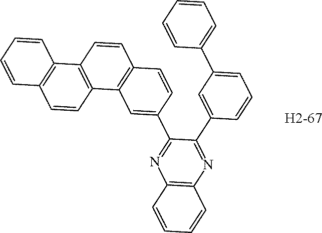 [SMILES: c1ccc(-c2cccc(-c3nc4ccccc4nc3-c3ccc4ccc5c6ccccc6ccc5c4c3)c2)cc1] 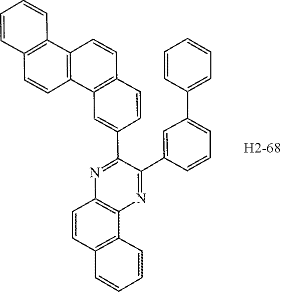 [SMILES: c1ccc(-c2cccc(-c3nc4c(ccc5ccccc54)nc3-c3ccc4ccc5c6ccccc6ccc5c4c3)c2)cc1] 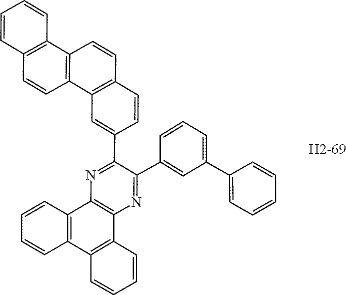 [SMILES: c1ccc(-c2cccc(-c3nc4c5ccccc5c5ccccc5c4nc3-c3ccc4ccc5c6ccccc6ccc5c4c3)c2)cc1] 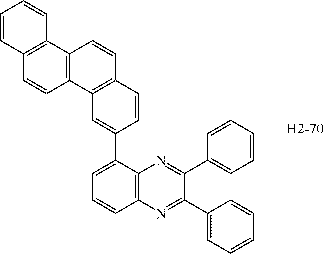 [SMILES: c1ccc(-c2nc3cccc(-c4ccc5ccc6c7ccccc7ccc6c5c4)c3nc2-c2ccccc2)cc1] 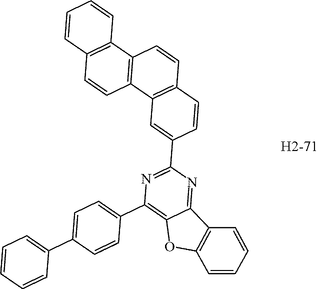 [SMILES: c1ccc(-c2ccc(-c3nc(-c4ccc5ccc6c7ccccc7ccc6c5c4)nc4c3oc3ccccc34)cc2)cc1] 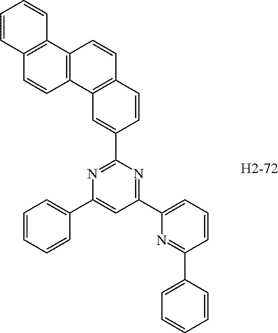 [SMILES: c1ccc(-c2cccc(-c3cc(-c4ccccc4)nc(-c4ccc5ccc6c7ccccc7ccc6c5c4)n3)n2)cc1] 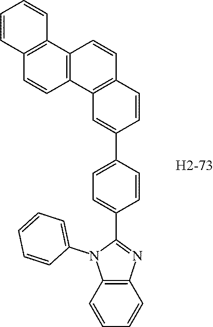 [SMILES: c1ccc(-n2c(-c3ccc(-c4ccc5ccc6c7ccccc7ccc6c5c4)cc3)nc3ccccc32)cc1] 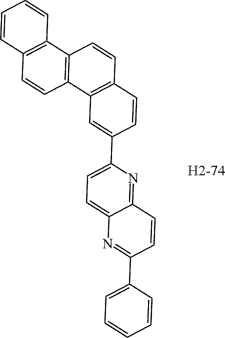 [SMILES: c1ccc(-c2ccc3nc(-c4ccc5ccc6c7ccccc7ccc6c5c4)ccc3n2)cc1] 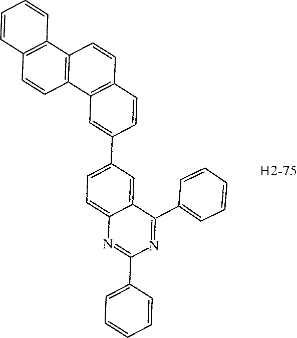 [SMILES: c1ccc(-c2nc(-c3ccccc3)c3cc(-c4ccc5ccc6c7ccccc7ccc6c5c4)ccc3n2)cc1] 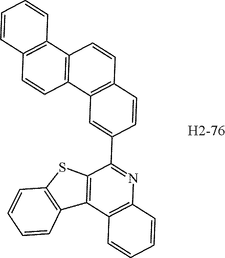 [SMILES: c1ccc2c(c1)ccc1c3cc(-c4nc5ccccc5c5c4sc4ccccc45)ccc3ccc21] 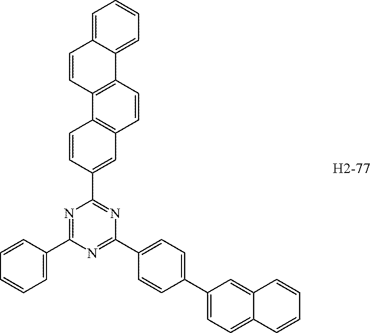 [SMILES: c1ccc(-c2nc(-c3ccc(-c4ccc5ccccc5c4)cc3)nc(-c3ccc4c(ccc5c6ccccc6ccc45)c3)n2)cc1] 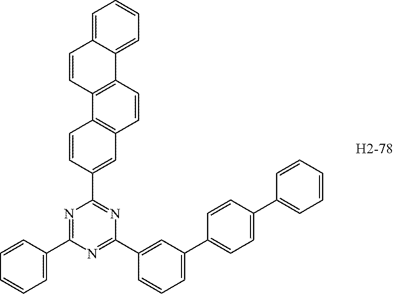 [SMILES: c1ccc(-c2ccc(-c3cccc(-c4nc(-c5ccccc5)nc(-c5ccc6c(ccc7c8ccccc8ccc67)c5)n4)c3)cc2)cc1] 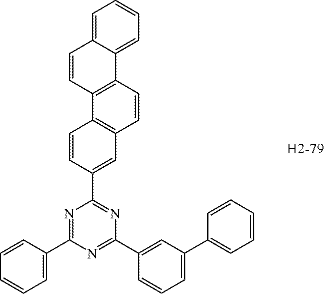 [SMILES: c1ccc(-c2cccc(-c3nc(-c4ccccc4)nc(-c4ccc5c(ccc6c7ccccc7ccc56)c4)n3)c2)cc1] 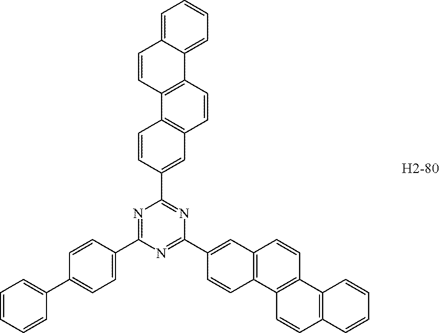 [SMILES: c1ccc(-c2ccc(-c3nc(-c4ccc5c(ccc6c7ccccc7ccc56)c4)nc(-c4ccc5c(ccc6c7ccccc7ccc56)c4)n3)cc2)cc1] 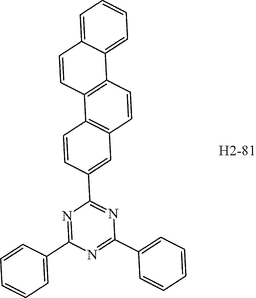 [SMILES: c1ccc(-c2nc(-c3ccccc3)nc(-c3ccc4c(ccc5c6ccccc6ccc45)c3)n2)cc1] 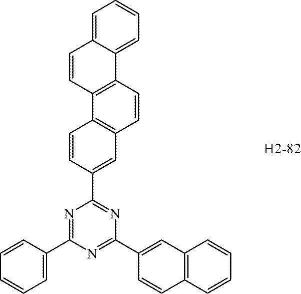 [SMILES: c1ccc(-c2nc(-c3ccc4ccccc4c3)nc(-c3ccc4c(ccc5c6ccccc6ccc45)c3)n2)cc1] 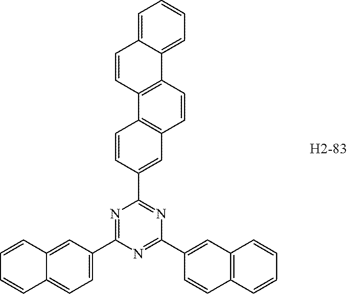 [SMILES: c1ccc2cc(-c3nc(-c4ccc5ccccc5c4)nc(-c4ccc5c(ccc6c7ccccc7ccc56)c4)n3)ccc2c1] 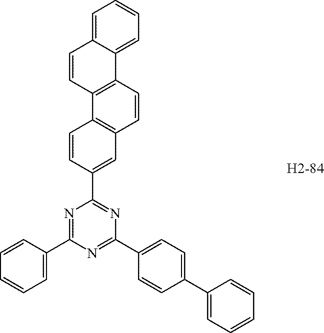 [SMILES: c1ccc(-c2ccc(-c3nc(-c4ccccc4)nc(-c4ccc5c(ccc6c7ccccc7ccc56)c4)n3)cc2)cc1] 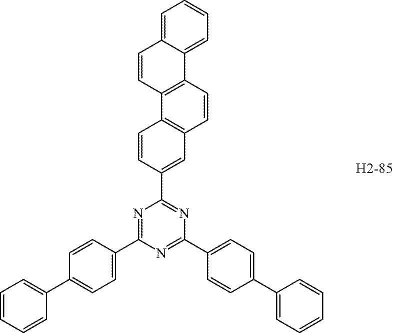 [SMILES: c1ccc(-c2ccc(-c3nc(-c4ccc(-c5ccccc5)cc4)nc(-c4ccc5c(ccc6c7ccccc7ccc56)c4)n3)cc2)cc1] 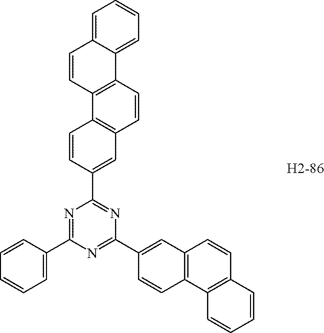 [SMILES: c1ccc(-c2nc(-c3ccc4c(ccc5ccccc54)c3)nc(-c3ccc4c(ccc5c6ccccc6ccc45)c3)n2)cc1] 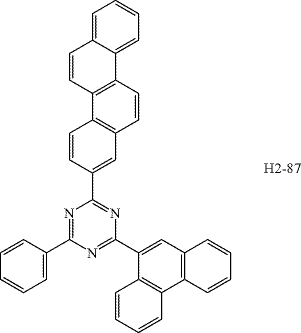 [SMILES: c1ccc(-c2nc(-c3ccc4c(ccc5c6ccccc6ccc45)c3)nc(-c3cc4ccccc4c4ccccc34)n2)cc1] 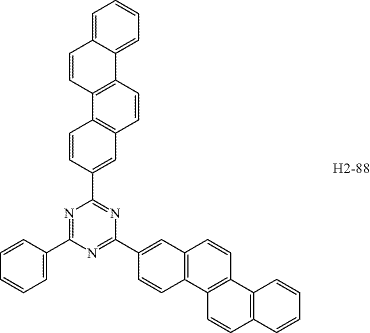 [SMILES: c1ccc(-c2nc(-c3ccc4c(ccc5c6ccccc6ccc45)c3)nc(-c3ccc4c(ccc5c6ccccc6ccc45)c3)n2)cc1] 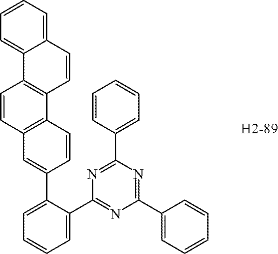 [SMILES: c1ccc(-c2nc(-c3ccccc3)nc(-c3ccccc3-c3ccc4c(ccc5c6ccccc6ccc45)c3)n2)cc1] 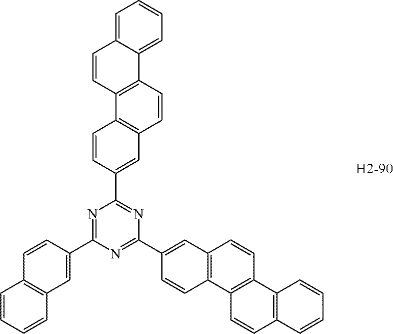 [SMILES: c1ccc2cc(-c3nc(-c4ccc5c(ccc6c7ccccc7ccc56)c4)nc(-c4ccc5c(ccc6c7ccccc7ccc56)c4)n3)ccc2c1] 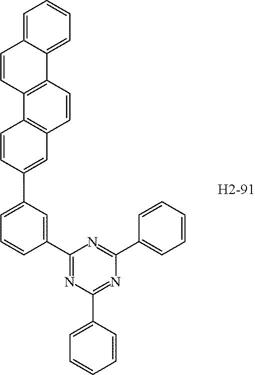 [SMILES: c1ccc(-c2nc(-c3ccccc3)nc(-c3cccc(-c4ccc5c(ccc6c7ccccc7ccc56)c4)c3)n2)cc1] 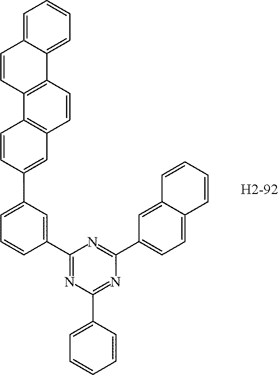 [SMILES: c1ccc(-c2nc(-c3cccc(-c4ccc5c(ccc6c7ccccc7ccc56)c4)c3)nc(-c3ccc4ccccc4c3)n2)cc1] 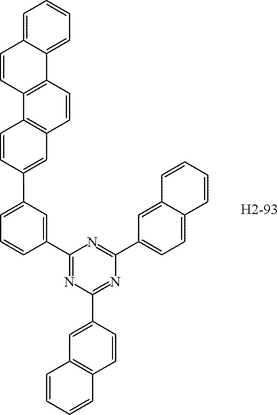 [SMILES: c1cc(-c2ccc3c(ccc4c5ccccc5ccc34)c2)cc(-c2nc(-c3ccc4ccccc4c3)nc(-c3ccc4ccccc4c3)n2)c1] 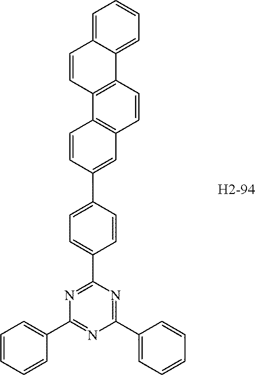 [SMILES: c1ccc(-c2nc(-c3ccccc3)nc(-c3ccc(-c4ccc5c(ccc6c7ccccc7ccc56)c4)cc3)n2)cc1] 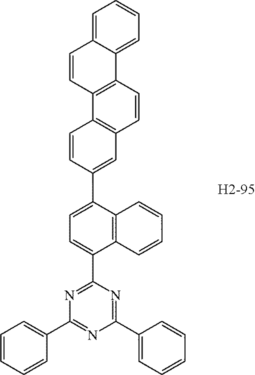 [SMILES: c1ccc(-c2nc(-c3ccccc3)nc(-c3ccc(-c4ccc5c(ccc6c7ccccc7ccc56)c4)c4ccccc34)n2)cc1] 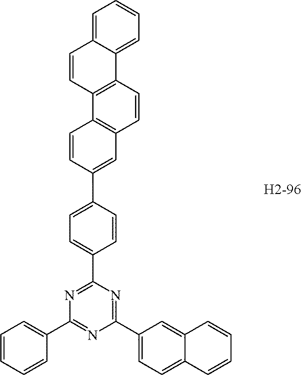 [SMILES: c1ccc(-c2nc(-c3ccc(-c4ccc5c(ccc6c7ccccc7ccc56)c4)cc3)nc(-c3ccc4ccccc4c3)n2)cc1] 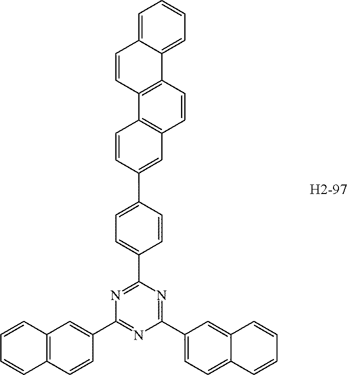 [SMILES: c1ccc2cc(-c3nc(-c4ccc(-c5ccc6c(ccc7c8ccccc8ccc67)c5)cc4)nc(-c4ccc5ccccc5c4)n3)ccc2c1] 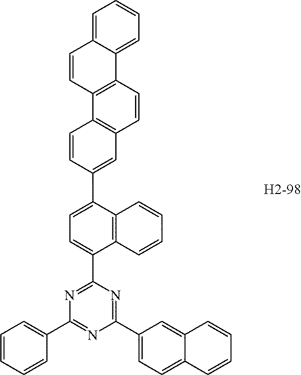 [SMILES: c1ccc(-c2nc(-c3ccc4ccccc4c3)nc(-c3ccc(-c4ccc5c(ccc6c7ccccc7ccc56)c4)c4ccccc34)n2)cc1] 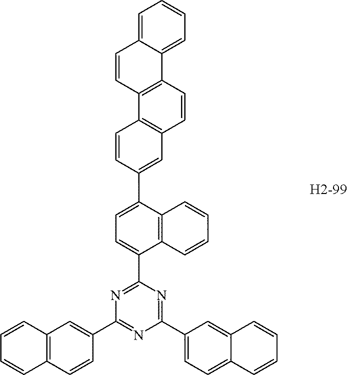 [SMILES: c1ccc2cc(-c3nc(-c4ccc5ccccc5c4)nc(-c4ccc(-c5ccc6c(ccc7c8ccccc8ccc67)c5)c5ccccc45)n3)ccc2c1] 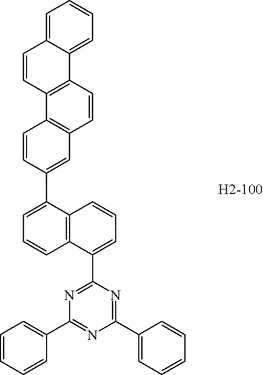 [SMILES: c1ccc(-c2nc(-c3ccccc3)nc(-c3cccc4c(-c5ccc6c(ccc7c8ccccc8ccc67)c5)cccc34)n2)cc1] 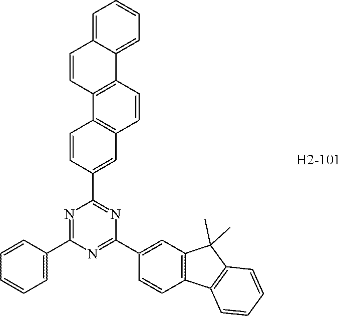 [SMILES: CC1(C)c2ccccc2-c2ccc(-c3nc(-c4ccccc4)nc(-c4ccc5c(ccc6c7ccccc7ccc56)c4)n3)cc21] 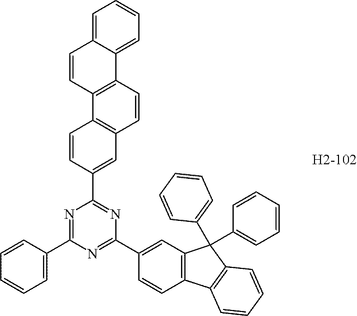 [SMILES: c1ccc(-c2nc(-c3ccc4c(c3)C(c3ccccc3)(c3ccccc3)c3ccccc3-4)nc(-c3ccc4c(ccc5c6ccccc6ccc45)c3)n2)cc1] 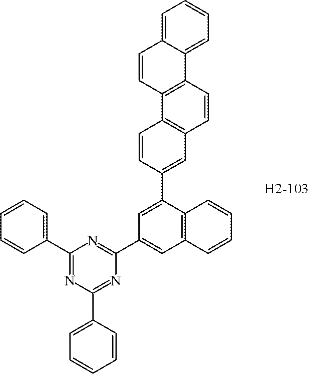 [SMILES: c1ccc(-c2nc(-c3ccccc3)nc(-c3cc(-c4ccc5c(ccc6c7ccccc7ccc56)c4)c4ccccc4c3)n2)cc1] 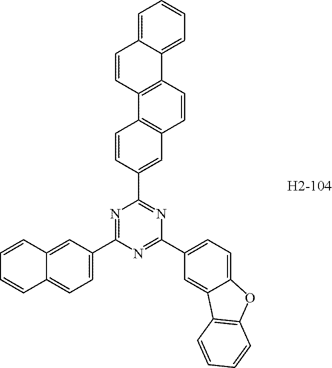 [SMILES: c1ccc2cc(-c3nc(-c4ccc5c(ccc6c7ccccc7ccc56)c4)nc(-c4ccc5oc6ccccc6c5c4)n3)ccc2c1] 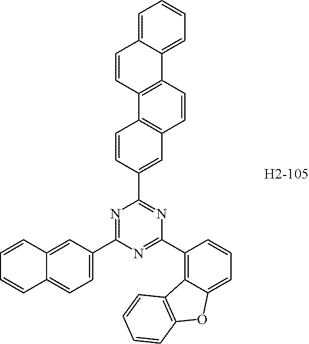 [SMILES: c1ccc2cc(-c3nc(-c4ccc5c(ccc6c7ccccc7ccc56)c4)nc(-c4cccc5oc6ccccc6c45)n3)ccc2c1] 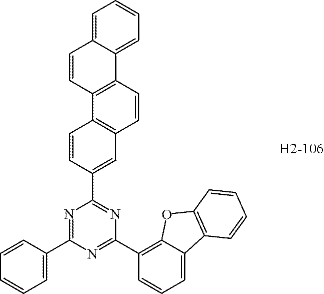 [SMILES: c1ccc(-c2nc(-c3ccc4c(ccc5c6ccccc6ccc45)c3)nc(-c3cccc4c3oc3ccccc34)n2)cc1] 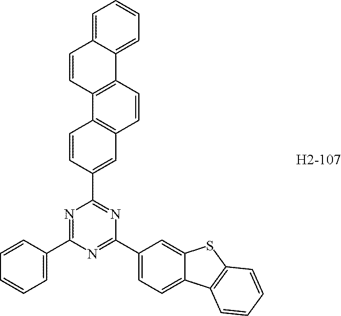 [SMILES: c1ccc(-c2nc(-c3ccc4c(ccc5c6ccccc6ccc45)c3)nc(-c3ccc4c(c3)sc3ccccc34)n2)cc1] 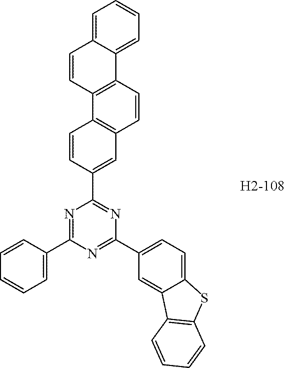 [SMILES: c1ccc(-c2nc(-c3ccc4c(ccc5c6ccccc6ccc45)c3)nc(-c3ccc4sc5ccccc5c4c3)n2)cc1] 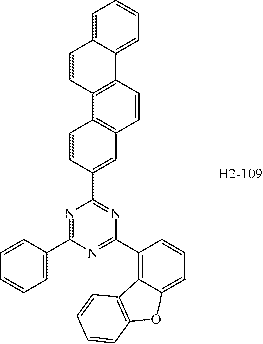 [SMILES: c1ccc(-c2nc(-c3ccc4c(ccc5c6ccccc6ccc45)c3)nc(-c3cccc4oc5ccccc5c34)n2)cc1] 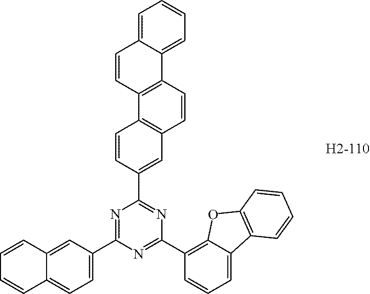 [SMILES: c1ccc2cc(-c3nc(-c4ccc5c(ccc6c7ccccc7ccc56)c4)nc(-c4cccc5c4oc4ccccc45)n3)ccc2c1] 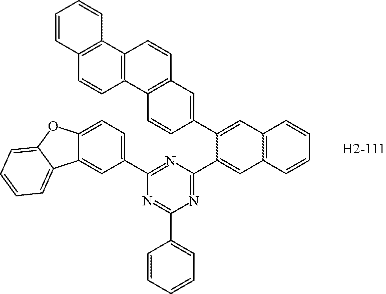 [SMILES: c1ccc(-c2nc(-c3ccc4oc5ccccc5c4c3)nc(-c3cc4ccccc4cc3-c3ccc4c(ccc5c6ccccc6ccc45)c3)n2)cc1] 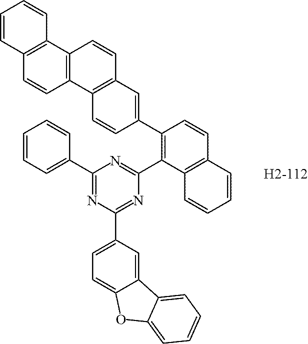 [SMILES: c1ccc(-c2nc(-c3ccc4oc5ccccc5c4c3)nc(-c3c(-c4ccc5c(ccc6c7ccccc7ccc56)c4)ccc4ccccc34)n2)cc1] 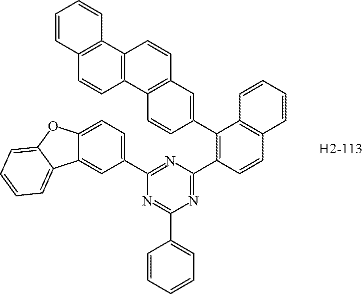 [SMILES: c1ccc(-c2nc(-c3ccc4oc5ccccc5c4c3)nc(-c3ccc4ccccc4c3-c3ccc4c(ccc5c6ccccc6ccc45)c3)n2)cc1] 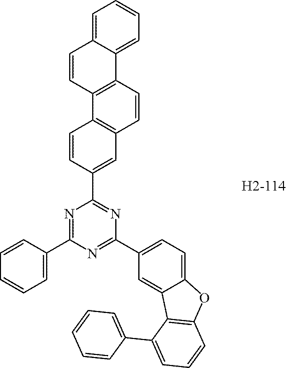 [SMILES: c1ccc(-c2nc(-c3ccc4c(ccc5c6ccccc6ccc45)c3)nc(-c3ccc4oc5cccc(-c6ccccc6)c5c4c3)n2)cc1] 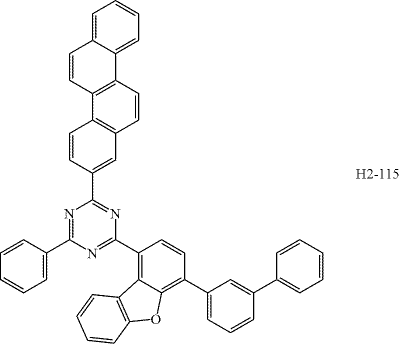 [SMILES: c1ccc(-c2cccc(-c3ccc(-c4nc(-c5ccccc5)nc(-c5ccc6c(ccc7c8ccccc8ccc67)c5)n4)c4c3oc3ccccc34)c2)cc1] 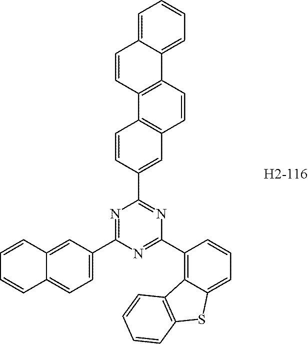 [SMILES: c1ccc2cc(-c3nc(-c4ccc5c(ccc6c7ccccc7ccc56)c4)nc(-c4cccc5sc6ccccc6c45)n3)ccc2c1] 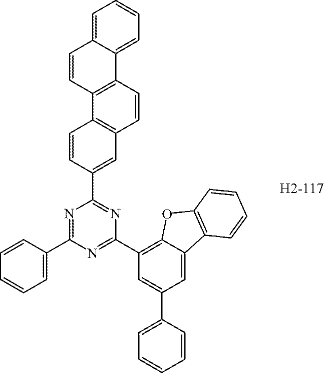 [SMILES: c1ccc(-c2cc(-c3nc(-c4ccccc4)nc(-c4ccc5c(ccc6c7ccccc7ccc56)c4)n3)c3oc4ccccc4c3c2)cc1] 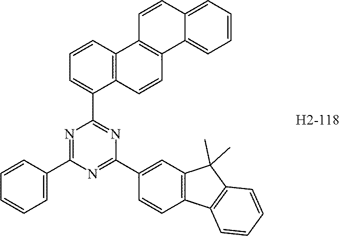 [SMILES: CC1(C)c2ccccc2-c2ccc(-c3nc(-c4ccccc4)nc(-c4cccc5c4ccc4c6ccccc6ccc54)n3)cc21] 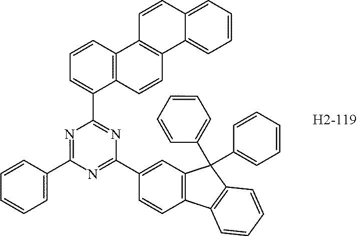 [SMILES: c1ccc(-c2nc(-c3ccc4c(c3)C(c3ccccc3)(c3ccccc3)c3ccccc3-4)nc(-c3cccc4c3ccc3c5ccccc5ccc43)n2)cc1] 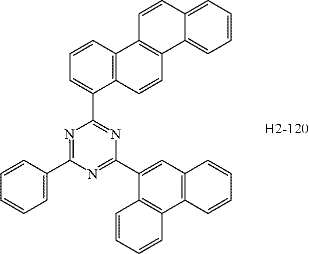 [SMILES: c1ccc(-c2nc(-c3cc4ccccc4c4ccccc34)nc(-c3cccc4c3ccc3c5ccccc5ccc43)n2)cc1] 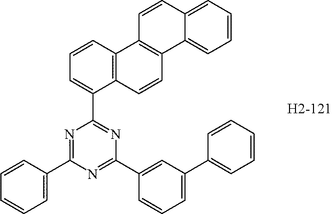 [SMILES: c1ccc(-c2cccc(-c3nc(-c4ccccc4)nc(-c4cccc5c4ccc4c6ccccc6ccc54)n3)c2)cc1] 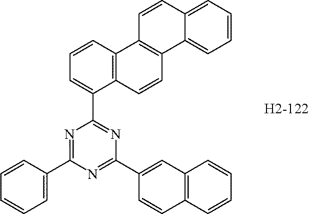 [SMILES: c1ccc(-c2nc(-c3ccc4ccccc4c3)nc(-c3cccc4c3ccc3c5ccccc5ccc43)n2)cc1] 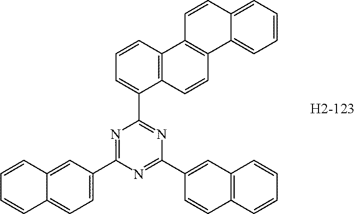 [SMILES: c1ccc2cc(-c3nc(-c4ccc5ccccc5c4)nc(-c4cccc5c4ccc4c6ccccc6ccc54)n3)ccc2c1] 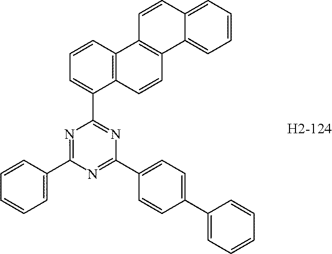 [SMILES: c1ccc(-c2ccc(-c3nc(-c4ccccc4)nc(-c4cccc5c4ccc4c6ccccc6ccc54)n3)cc2)cc1] 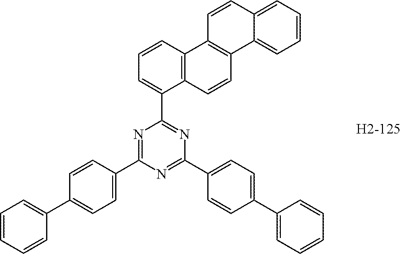 [SMILES: c1ccc(-c2ccc(-c3nc(-c4ccc(-c5ccccc5)cc4)nc(-c4cccc5c4ccc4c6ccccc6ccc54)n3)cc2)cc1] 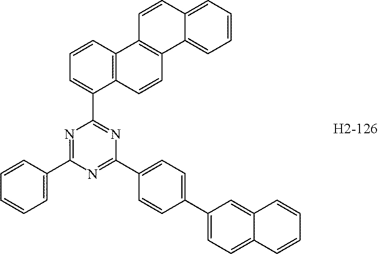 [SMILES: c1ccc(-c2nc(-c3ccc(-c4ccc5ccccc5c4)cc3)nc(-c3cccc4c3ccc3c5ccccc5ccc43)n2)cc1] 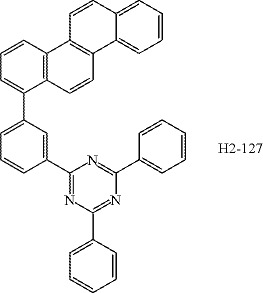 [SMILES: c1ccc(-c2nc(-c3ccccc3)nc(-c3cccc(-c4cccc5c4ccc4c6ccccc6ccc54)c3)n2)cc1] 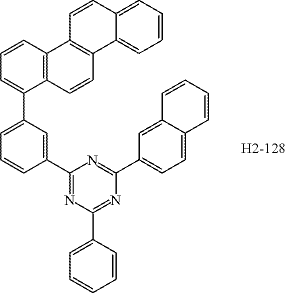 [SMILES: c1ccc(-c2nc(-c3cccc(-c4cccc5c4ccc4c6ccccc6ccc54)c3)nc(-c3ccc4ccccc4c3)n2)cc1] 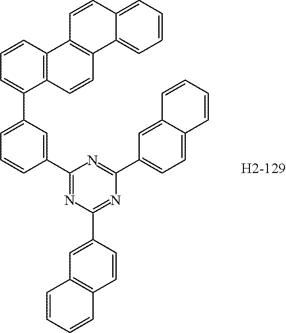 [SMILES: c1cc(-c2nc(-c3ccc4ccccc4c3)nc(-c3ccc4ccccc4c3)n2)cc(-c2cccc3c2ccc2c4ccccc4ccc32)c1] 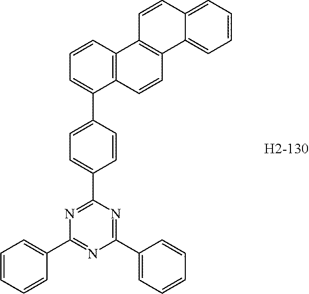 [SMILES: c1ccc(-c2nc(-c3ccccc3)nc(-c3ccc(-c4cccc5c4ccc4c6ccccc6ccc54)cc3)n2)cc1] 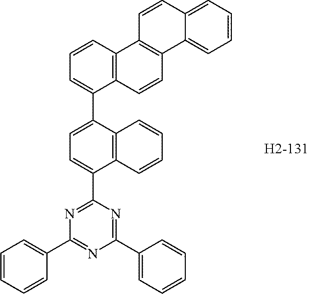 [SMILES: c1ccc(-c2nc(-c3ccccc3)nc(-c3ccc(-c4cccc5c4ccc4c6ccccc6ccc54)c4ccccc34)n2)cc1] 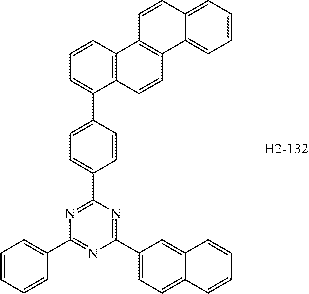 [SMILES: c1ccc(-c2nc(-c3ccc(-c4cccc5c4ccc4c6ccccc6ccc54)cc3)nc(-c3ccc4ccccc4c3)n2)cc1] 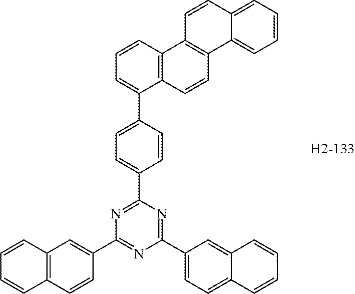 [SMILES: c1ccc2cc(-c3nc(-c4ccc(-c5cccc6c5ccc5c7ccccc7ccc65)cc4)nc(-c4ccc5ccccc5c4)n3)ccc2c1] 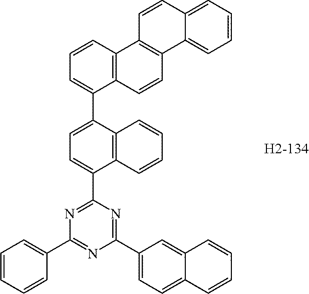 [SMILES: c1ccc(-c2nc(-c3ccc4ccccc4c3)nc(-c3ccc(-c4cccc5c4ccc4c6ccccc6ccc54)c4ccccc34)n2)cc1] 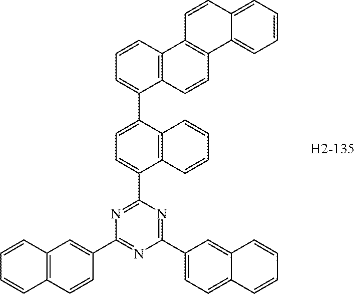 [SMILES: c1ccc2cc(-c3nc(-c4ccc5ccccc5c4)nc(-c4ccc(-c5cccc6c5ccc5c7ccccc7ccc65)c5ccccc45)n3)ccc2c1] 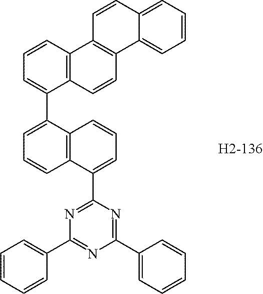 [SMILES: c1ccc(-c2nc(-c3ccccc3)nc(-c3cccc4c(-c5cccc6c5ccc5c7ccccc7ccc65)cccc34)n2)cc1] 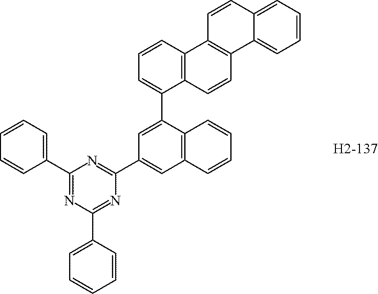 [SMILES: c1ccc(-c2nc(-c3ccccc3)nc(-c3cc(-c4cccc5c4ccc4c6ccccc6ccc54)c4ccccc4c3)n2)cc1] 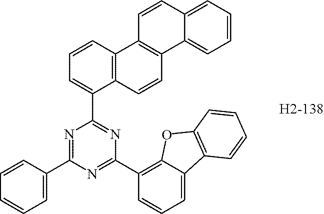 [SMILES: c1ccc(-c2nc(-c3cccc4c3ccc3c5ccccc5ccc43)nc(-c3cccc4c3oc3ccccc34)n2)cc1] 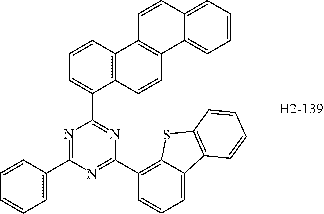 [SMILES: c1ccc(-c2nc(-c3cccc4c3ccc3c5ccccc5ccc43)nc(-c3cccc4c3sc3ccccc34)n2)cc1] 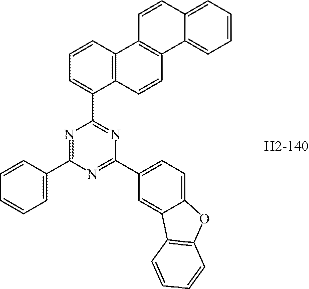 [SMILES: c1ccc(-c2nc(-c3ccc4oc5ccccc5c4c3)nc(-c3cccc4c3ccc3c5ccccc5ccc43)n2)cc1] 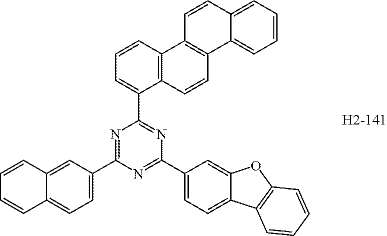 [SMILES: c1ccc2cc(-c3nc(-c4ccc5c(c4)oc4ccccc45)nc(-c4cccc5c4ccc4c6ccccc6ccc54)n3)ccc2c1] 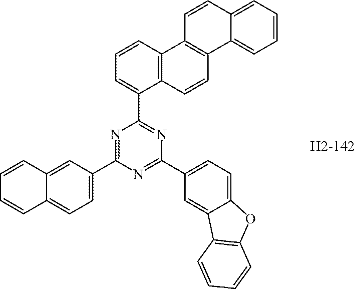 [SMILES: c1ccc2cc(-c3nc(-c4ccc5oc6ccccc6c5c4)nc(-c4cccc5c4ccc4c6ccccc6ccc54)n3)ccc2c1] 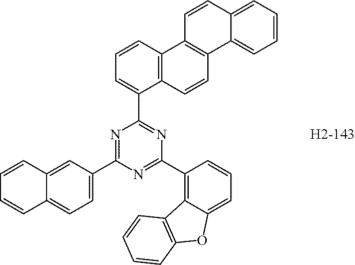 [SMILES: c1ccc2cc(-c3nc(-c4cccc5c4ccc4c6ccccc6ccc54)nc(-c4cccc5oc6ccccc6c45)n3)ccc2c1] 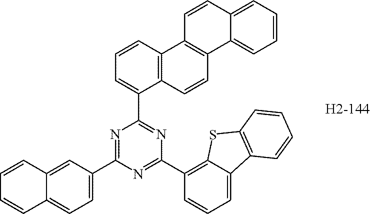 [SMILES: c1ccc2cc(-c3nc(-c4cccc5c4ccc4c6ccccc6ccc54)nc(-c4cccc5c4sc4ccccc45)n3)ccc2c1] 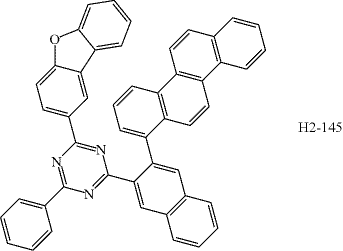 [SMILES: c1ccc(-c2nc(-c3ccc4oc5ccccc5c4c3)nc(-c3cc4ccccc4cc3-c3cccc4c3ccc3c5ccccc5ccc43)n2)cc1] 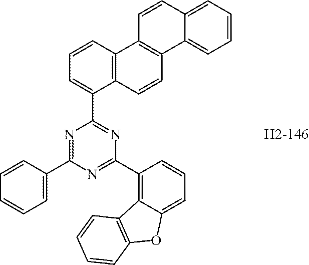 [SMILES: c1ccc(-c2nc(-c3cccc4c3ccc3c5ccccc5ccc43)nc(-c3cccc4oc5ccccc5c34)n2)cc1] 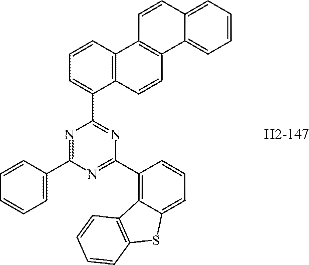 [SMILES: c1ccc(-c2nc(-c3cccc4c3ccc3c5ccccc5ccc43)nc(-c3cccc4sc5ccccc5c34)n2)cc1] 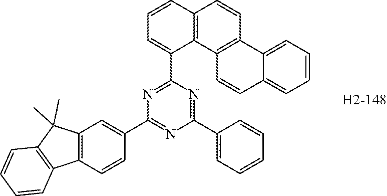 [SMILES: CC1(C)c2ccccc2-c2ccc(-c3nc(-c4ccccc4)nc(-c4cccc5ccc6c7ccccc7ccc6c45)n3)cc21] 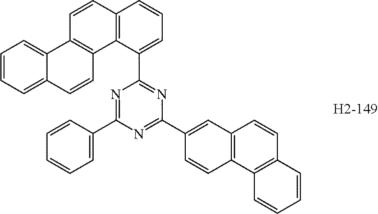 [SMILES: c1ccc(-c2nc(-c3ccc4c(ccc5ccccc54)c3)nc(-c3cccc4ccc5c6ccccc6ccc5c34)n2)cc1] 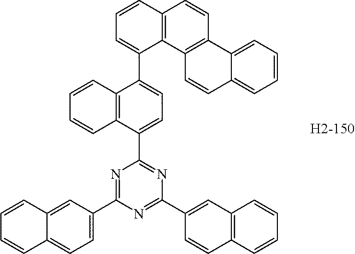 [SMILES: c1ccc2cc(-c3nc(-c4ccc5ccccc5c4)nc(-c4ccc(-c5cccc6ccc7c8ccccc8ccc7c56)c5ccccc45)n3)ccc2c1] 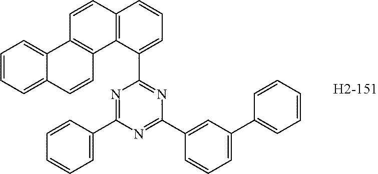 [SMILES: c1ccc(-c2cccc(-c3nc(-c4ccccc4)nc(-c4cccc5ccc6c7ccccc7ccc6c45)n3)c2)cc1] 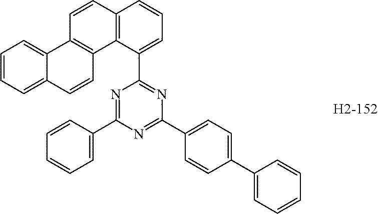 [SMILES: c1ccc(-c2ccc(-c3nc(-c4ccccc4)nc(-c4cccc5ccc6c7ccccc7ccc6c45)n3)cc2)cc1] 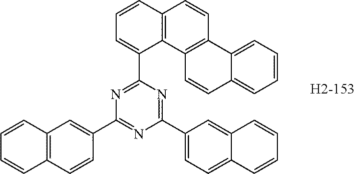 [SMILES: c1ccc2cc(-c3nc(-c4ccc5ccccc5c4)nc(-c4cccc5ccc6c7ccccc7ccc6c45)n3)ccc2c1] 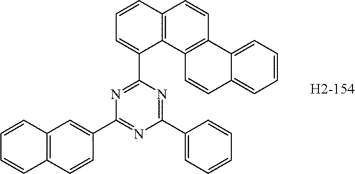 [SMILES: c1ccc(-c2nc(-c3ccc4ccccc4c3)nc(-c3cccc4ccc5c6ccccc6ccc5c34)n2)cc1] 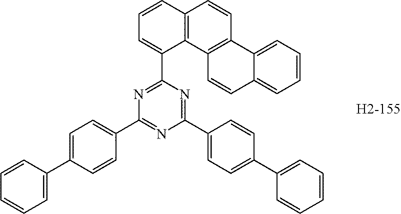 [SMILES: c1ccc(-c2ccc(-c3nc(-c4ccc(-c5ccccc5)cc4)nc(-c4cccc5ccc6c7ccccc7ccc6c45)n3)cc2)cc1] 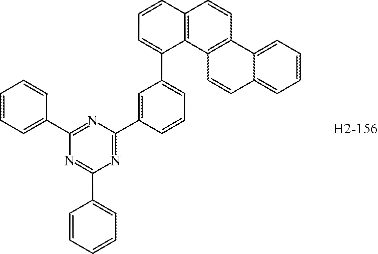 [SMILES: c1ccc(-c2nc(-c3ccccc3)nc(-c3cccc(-c4cccc5ccc6c7ccccc7ccc6c45)c3)n2)cc1] 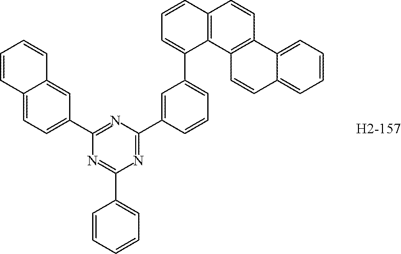 [SMILES: c1ccc(-c2nc(-c3cccc(-c4cccc5ccc6c7ccccc7ccc6c45)c3)nc(-c3ccc4ccccc4c3)n2)cc1] 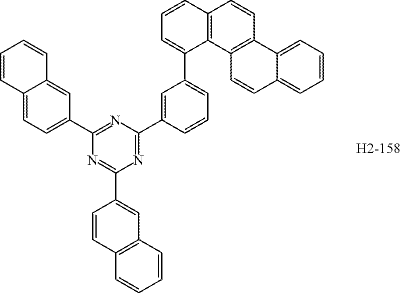 [SMILES: c1cc(-c2nc(-c3ccc4ccccc4c3)nc(-c3ccc4ccccc4c3)n2)cc(-c2cccc3ccc4c5ccccc5ccc4c23)c1] 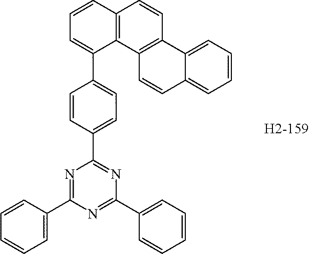 [SMILES: c1ccc(-c2nc(-c3ccccc3)nc(-c3ccc(-c4cccc5ccc6c7ccccc7ccc6c45)cc3)n2)cc1] 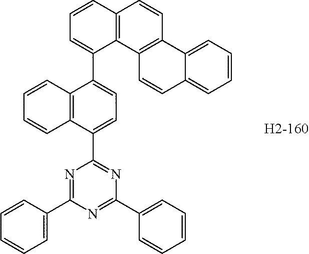 [SMILES: c1ccc(-c2nc(-c3ccccc3)nc(-c3ccc(-c4cccc5ccc6c7ccccc7ccc6c45)c4ccccc34)n2)cc1] 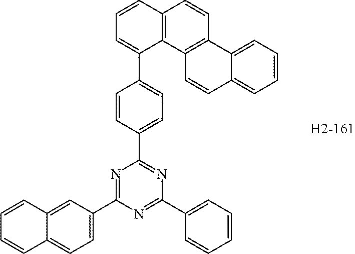 [SMILES: c1ccc(-c2nc(-c3ccc(-c4cccc5ccc6c7ccccc7ccc6c45)cc3)nc(-c3ccc4ccccc4c3)n2)cc1] 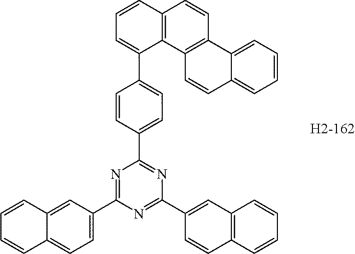 [SMILES: c1ccc2cc(-c3nc(-c4ccc(-c5cccc6ccc7c8ccccc8ccc7c56)cc4)nc(-c4ccc5ccccc5c4)n3)ccc2c1] 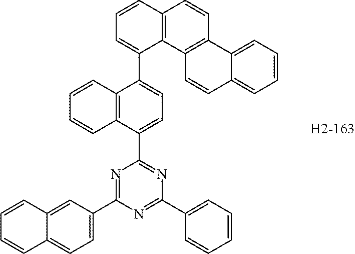 [SMILES: c1ccc(-c2nc(-c3ccc4ccccc4c3)nc(-c3ccc(-c4cccc5ccc6c7ccccc7ccc6c45)c4ccccc34)n2)cc1] 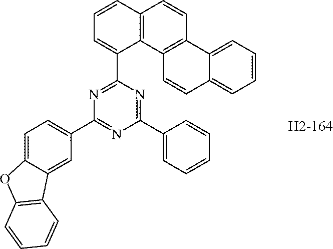 [SMILES: c1ccc(-c2nc(-c3ccc4oc5ccccc5c4c3)nc(-c3cccc4ccc5c6ccccc6ccc5c34)n2)cc1] 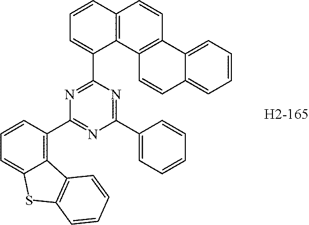 [SMILES: c1ccc(-c2nc(-c3cccc4ccc5c6ccccc6ccc5c34)nc(-c3cccc4sc5ccccc5c34)n2)cc1] 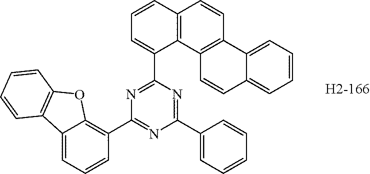 [SMILES: c1ccc(-c2nc(-c3cccc4c3oc3ccccc34)nc(-c3cccc4ccc5c6ccccc6ccc5c34)n2)cc1] 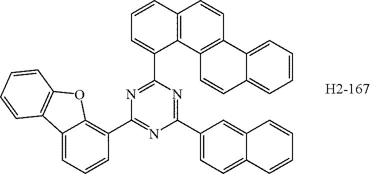 [SMILES: c1ccc2cc(-c3nc(-c4cccc5c4oc4ccccc45)nc(-c4cccc5ccc6c7ccccc7ccc6c45)n3)ccc2c1] 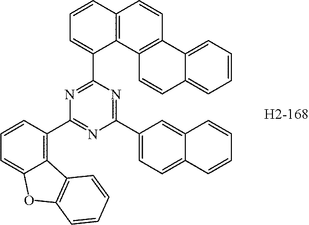 [SMILES: c1ccc2cc(-c3nc(-c4cccc5ccc6c7ccccc7ccc6c45)nc(-c4cccc5oc6ccccc6c45)n3)ccc2c1] 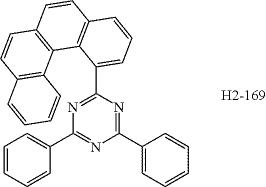 [SMILES: c1ccc(-c2nc(-c3ccccc3)nc(-c3cccc4ccc5ccc6ccccc6c5c34)n2)cc1] 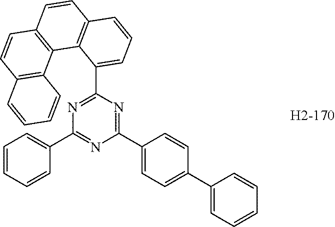 [SMILES: c1ccc(-c2ccc(-c3nc(-c4ccccc4)nc(-c4cccc5ccc6ccc7ccccc7c6c45)n3)cc2)cc1] 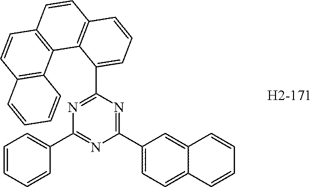 [SMILES: c1ccc(-c2nc(-c3ccc4ccccc4c3)nc(-c3cccc4ccc5ccc6ccccc6c5c34)n2)cc1] 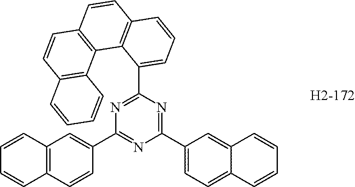 [SMILES: c1ccc2cc(-c3nc(-c4ccc5ccccc5c4)nc(-c4cccc5ccc6ccc7ccccc7c6c45)n3)ccc2c1] 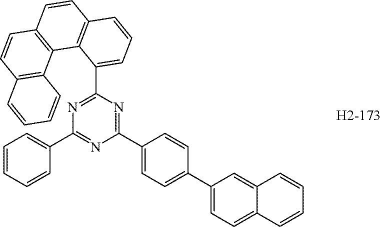 [SMILES: c1ccc(-c2nc(-c3ccc(-c4ccc5ccccc5c4)cc3)nc(-c3cccc4ccc5ccc6ccccc6c5c34)n2)cc1] 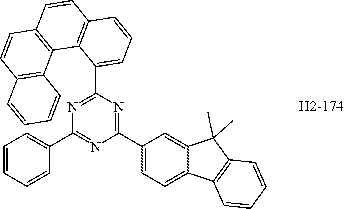 [SMILES: CC1(C)c2ccccc2-c2ccc(-c3nc(-c4ccccc4)nc(-c4cccc5ccc6ccc7ccccc7c6c45)n3)cc21] 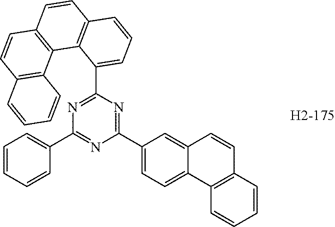 [SMILES: c1ccc(-c2nc(-c3ccc4c(ccc5ccccc54)c3)nc(-c3cccc4ccc5ccc6ccccc6c5c34)n2)cc1] 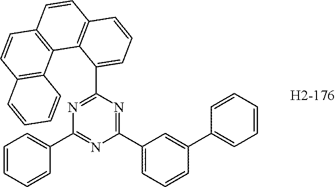 [SMILES: c1ccc(-c2cccc(-c3nc(-c4ccccc4)nc(-c4cccc5ccc6ccc7ccccc7c6c45)n3)c2)cc1] 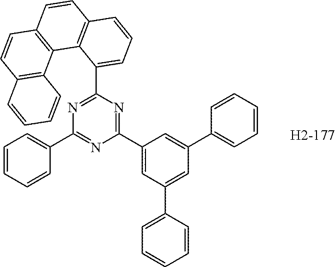 [SMILES: c1ccc(-c2cc(-c3ccccc3)cc(-c3nc(-c4ccccc4)nc(-c4cccc5ccc6ccc7ccccc7c6c45)n3)c2)cc1] 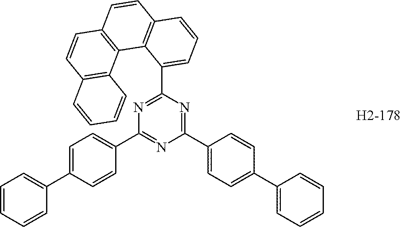 [SMILES: c1ccc(-c2ccc(-c3nc(-c4ccc(-c5ccccc5)cc4)nc(-c4cccc5ccc6ccc7ccccc7c6c45)n3)cc2)cc1] 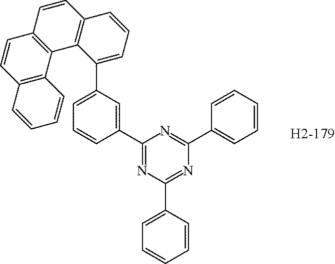 [SMILES: c1ccc(-c2nc(-c3ccccc3)nc(-c3cccc(-c4cccc5ccc6ccc7ccccc7c6c45)c3)n2)cc1] 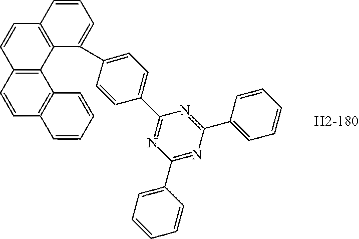 [SMILES: c1ccc(-c2nc(-c3ccccc3)nc(-c3ccc(-c4cccc5ccc6ccc7ccccc7c6c45)cc3)n2)cc1] 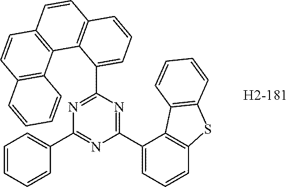 [SMILES: c1ccc(-c2nc(-c3cccc4sc5ccccc5c34)nc(-c3cccc4ccc5ccc6ccccc6c5c34)n2)cc1] 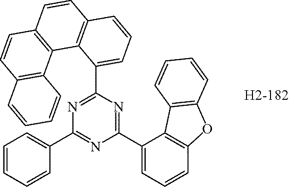 [SMILES: c1ccc(-c2nc(-c3cccc4oc5ccccc5c34)nc(-c3cccc4ccc5ccc6ccccc6c5c34)n2)cc1] 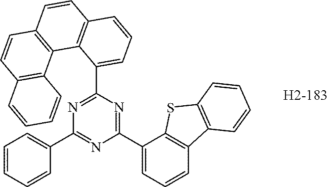 [SMILES: c1ccc(-c2nc(-c3cccc4c3sc3ccccc34)nc(-c3cccc4ccc5ccc6ccccc6c5c34)n2)cc1] 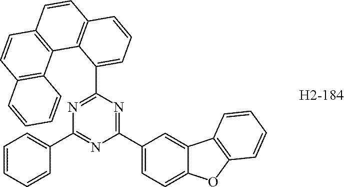 [SMILES: c1ccc(-c2nc(-c3ccc4oc5ccccc5c4c3)nc(-c3cccc4ccc5ccc6ccccc6c5c34)n2)cc1] 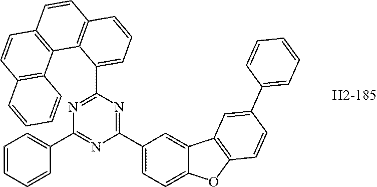 [SMILES: c1ccc(-c2ccc3oc4ccc(-c5nc(-c6ccccc6)nc(-c6cccc7ccc8ccc9ccccc9c8c67)n5)cc4c3c2)cc1] 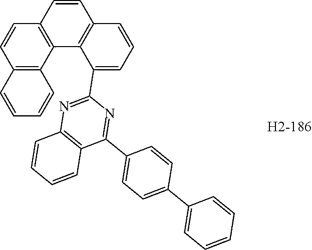 [SMILES: c1ccc(-c2ccc(-c3nc(-c4cccc5ccc6ccc7ccccc7c6c45)nc4ccccc34)cc2)cc1] 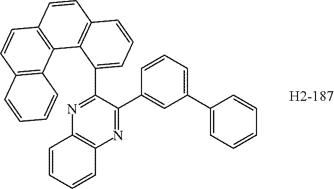 [SMILES: c1ccc(-c2cccc(-c3nc4ccccc4nc3-c3cccc4ccc5ccc6ccccc6c5c34)c2)cc1] 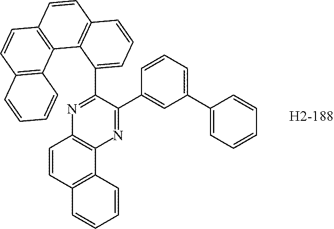 [SMILES: c1ccc(-c2cccc(-c3nc4c(ccc5ccccc54)nc3-c3cccc4ccc5ccc6ccccc6c5c34)c2)cc1] 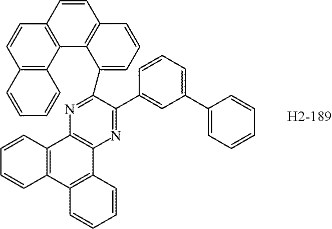 [SMILES: c1ccc(-c2cccc(-c3nc4c5ccccc5c5ccccc5c4nc3-c3cccc4ccc5ccc6ccccc6c5c34)c2)cc1] 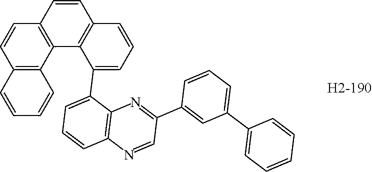 [SMILES: c1ccc(-c2cccc(-c3cnc4cccc(-c5cccc6ccc7ccc8ccccc8c7c56)c4n3)c2)cc1] 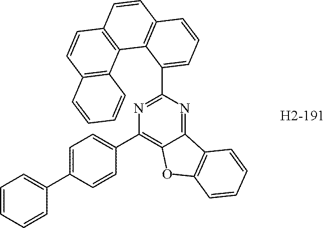 [SMILES: c1ccc(-c2ccc(-c3nc(-c4cccc5ccc6ccc7ccccc7c6c45)nc4c3oc3ccccc34)cc2)cc1] 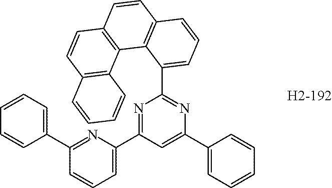 [SMILES: c1ccc(-c2cccc(-c3cc(-c4ccccc4)nc(-c4cccc5ccc6ccc7ccccc7c6c45)n3)n2)cc1] 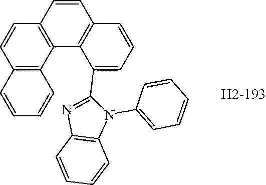 [SMILES: c1ccc(-n2c(-c3cccc4ccc5ccc6ccccc6c5c34)nc3ccccc32)cc1] 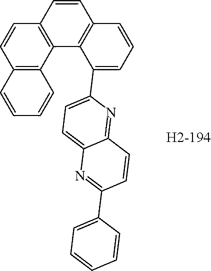 [SMILES: c1ccc(-c2ccc3nc(-c4cccc5ccc6ccc7ccccc7c6c45)ccc3n2)cc1] 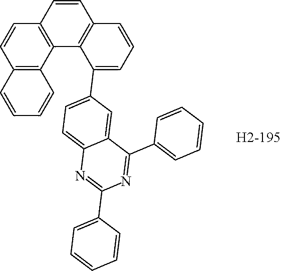 [SMILES: c1ccc(-c2nc(-c3ccccc3)c3cc(-c4cccc5ccc6ccc7ccccc7c6c45)ccc3n2)cc1] 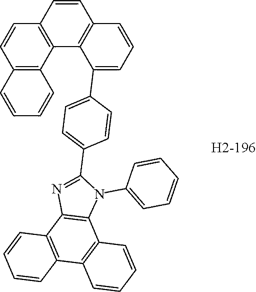 [SMILES: c1ccc(-n2c(-c3ccc(-c4cccc5ccc6ccc7ccccc7c6c45)cc3)nc3c4ccccc4c4ccccc4c32)cc1] 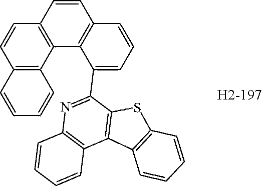 [SMILES: c1ccc2c(c1)ccc1ccc3cccc(-c4nc5ccccc5c5c4sc4ccccc45)c3c12] 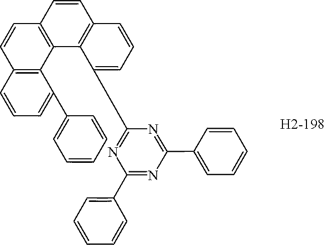 [SMILES: c1ccc(-c2nc(-c3ccccc3)nc(-c3cccc4ccc5ccc6cccc(-c7ccccc7)c6c5c34)n2)cc1] 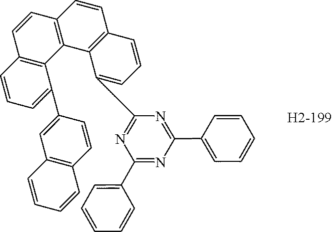 [SMILES: c1ccc(-c2nc(-c3ccccc3)nc(-c3cccc4ccc5ccc6cccc(-c7ccc8ccccc8c7)c6c5c34)n2)cc1] 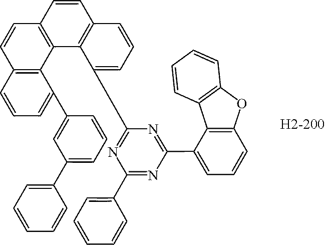 [SMILES: c1ccc(-c2cccc(-c3cccc4ccc5ccc6cccc(-c7nc(-c8ccccc8)nc(-c8cccc9oc%10ccccc%10c89)n7)c6c5c34)c2)cc1] 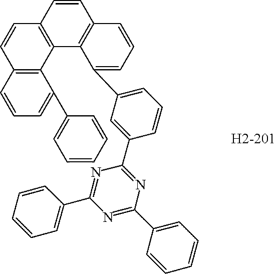 [SMILES: c1ccc(-c2nc(-c3ccccc3)nc(-c3cccc(-c4cccc5ccc6ccc7cccc(-c8ccccc8)c7c6c45)c3)n2)cc1] 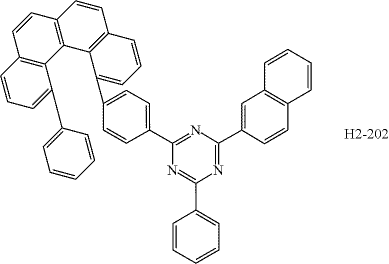 [SMILES: c1ccc(-c2nc(-c3ccc(-c4cccc5ccc6ccc7cccc(-c8ccccc8)c7c6c45)cc3)nc(-c3ccc4ccccc4c3)n2)cc1] 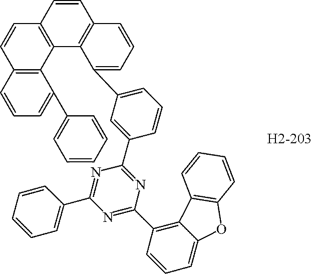 [SMILES: c1ccc(-c2nc(-c3cccc(-c4cccc5ccc6ccc7cccc(-c8ccccc8)c7c6c45)c3)nc(-c3cccc4oc5ccccc5c34)n2)cc1] 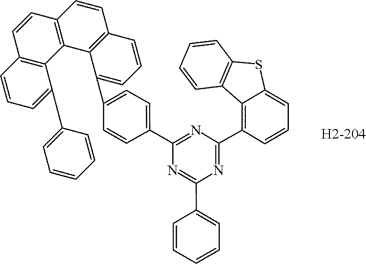 [SMILES: c1ccc(-c2nc(-c3ccc(-c4cccc5ccc6ccc7cccc(-c8ccccc8)c7c6c45)cc3)nc(-c3cccc4sc5ccccc5c34)n2)cc1] 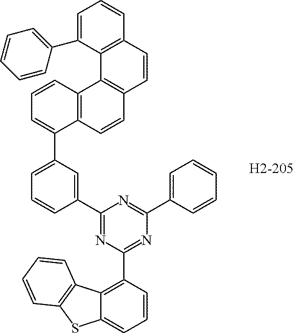 [SMILES: c1ccc(-c2nc(-c3cccc(-c4cccc5c4ccc4ccc6cccc(-c7ccccc7)c6c45)c3)nc(-c3cccc4sc5ccccc5c34)n2)cc1] 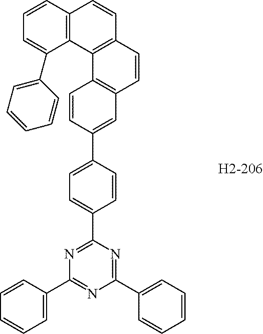 [SMILES: c1ccc(-c2nc(-c3ccccc3)nc(-c3ccc(-c4ccc5c(ccc6ccc7cccc(-c8ccccc8)c7c65)c4)cc3)n2)cc1] 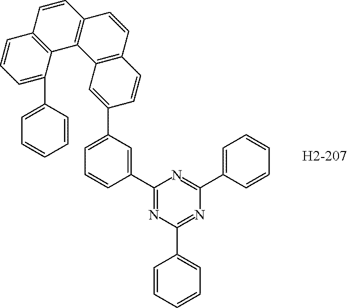 [SMILES: c1ccc(-c2nc(-c3ccccc3)nc(-c3cccc(-c4ccc5ccc6ccc7cccc(-c8ccccc8)c7c6c5c4)c3)n2)cc1] 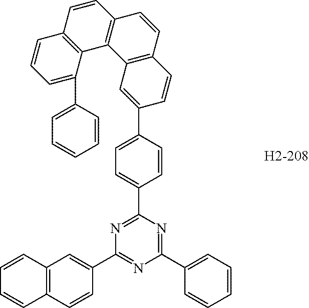 [SMILES: c1ccc(-c2nc(-c3ccc(-c4ccc5ccc6ccc7cccc(-c8ccccc8)c7c6c5c4)cc3)nc(-c3ccc4ccccc4c3)n2)cc1] 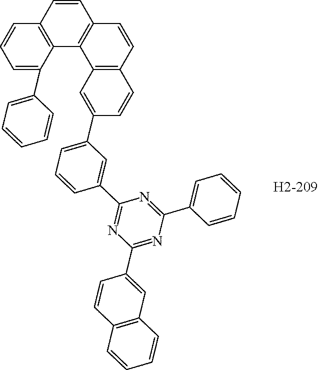 [SMILES: c1ccc(-c2nc(-c3cccc(-c4ccc5ccc6ccc7cccc(-c8ccccc8)c7c6c5c4)c3)nc(-c3ccc4ccccc4c3)n2)cc1] 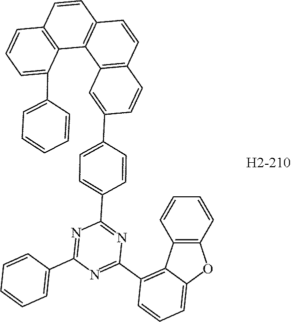 [SMILES: c1ccc(-c2nc(-c3ccc(-c4ccc5ccc6ccc7cccc(-c8ccccc8)c7c6c5c4)cc3)nc(-c3cccc4oc5ccccc5c34)n2)cc1] 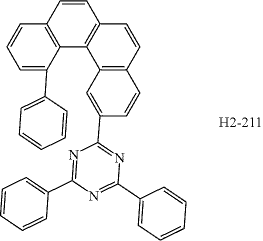 [SMILES: c1ccc(-c2nc(-c3ccccc3)nc(-c3ccc4ccc5ccc6cccc(-c7ccccc7)c6c5c4c3)n2)cc1] 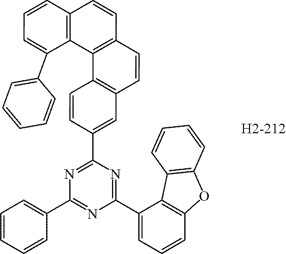 [SMILES: c1ccc(-c2nc(-c3ccc4c(ccc5ccc6cccc(-c7ccccc7)c6c54)c3)nc(-c3cccc4oc5ccccc5c34)n2)cc1] 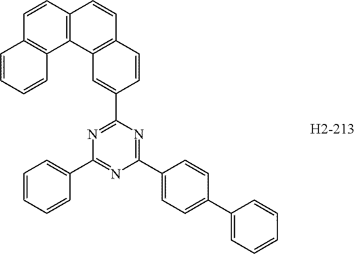 [SMILES: c1ccc(-c2ccc(-c3nc(-c4ccccc4)nc(-c4ccc5ccc6ccc7ccccc7c6c5c4)n3)cc2)cc1] 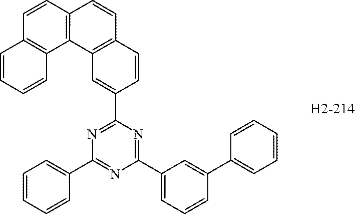 [SMILES: c1ccc(-c2cccc(-c3nc(-c4ccccc4)nc(-c4ccc5ccc6ccc7ccccc7c6c5c4)n3)c2)cc1] 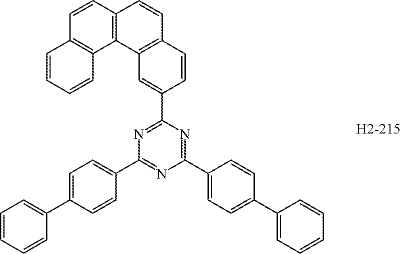 [SMILES: c1ccc(-c2ccc(-c3nc(-c4ccc(-c5ccccc5)cc4)nc(-c4ccc5ccc6ccc7ccccc7c6c5c4)n3)cc2)cc1] 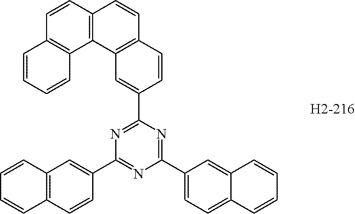 [SMILES: c1ccc2cc(-c3nc(-c4ccc5ccccc5c4)nc(-c4ccc5ccc6ccc7ccccc7c6c5c4)n3)ccc2c1] 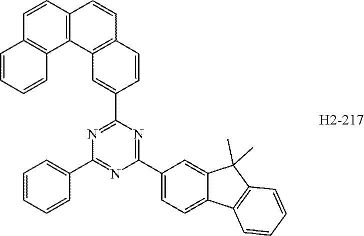 [SMILES: CC1(C)c2ccccc2-c2ccc(-c3nc(-c4ccccc4)nc(-c4ccc5ccc6ccc7ccccc7c6c5c4)n3)cc21] 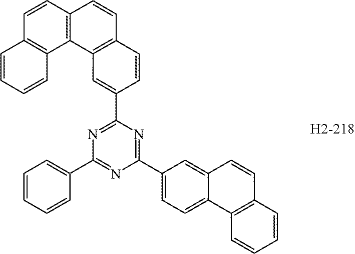 [SMILES: c1ccc(-c2nc(-c3ccc4c(ccc5ccccc54)c3)nc(-c3ccc4ccc5ccc6ccccc6c5c4c3)n2)cc1] 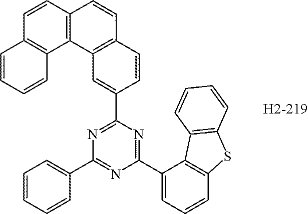 [SMILES: c1ccc(-c2nc(-c3ccc4ccc5ccc6ccccc6c5c4c3)nc(-c3cccc4sc5ccccc5c34)n2)cc1] 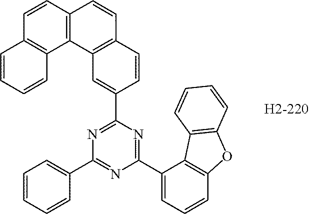 [SMILES: c1ccc(-c2nc(-c3ccc4ccc5ccc6ccccc6c5c4c3)nc(-c3cccc4oc5ccccc5c34)n2)cc1] 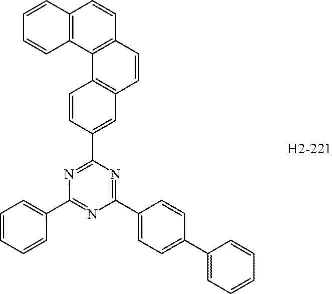 [SMILES: c1ccc(-c2ccc(-c3nc(-c4ccccc4)nc(-c4ccc5c(ccc6ccc7ccccc7c65)c4)n3)cc2)cc1] 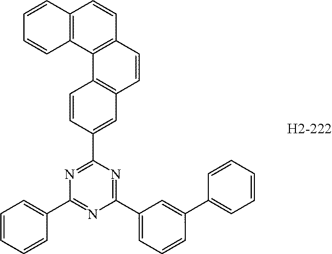 [SMILES: c1ccc(-c2cccc(-c3nc(-c4ccccc4)nc(-c4ccc5c(ccc6ccc7ccccc7c65)c4)n3)c2)cc1] 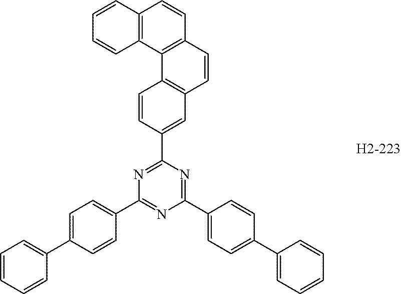 [SMILES: c1ccc(-c2ccc(-c3nc(-c4ccc(-c5ccccc5)cc4)nc(-c4ccc5c(ccc6ccc7ccccc7c65)c4)n3)cc2)cc1] 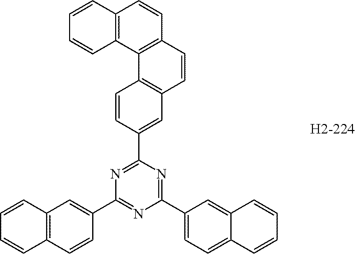 [SMILES: c1ccc2cc(-c3nc(-c4ccc5ccccc5c4)nc(-c4ccc5c(ccc6ccc7ccccc7c65)c4)n3)ccc2c1] 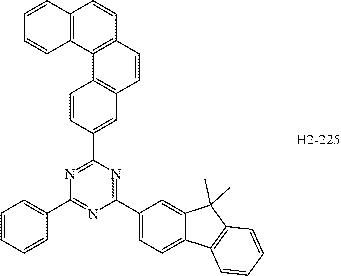 [SMILES: CC1(C)c2ccccc2-c2ccc(-c3nc(-c4ccccc4)nc(-c4ccc5c(ccc6ccc7ccccc7c65)c4)n3)cc21] 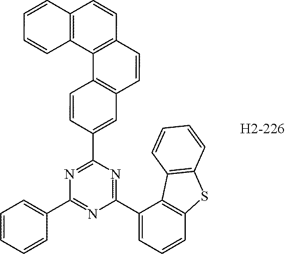 [SMILES: c1ccc(-c2nc(-c3ccc4c(ccc5ccc6ccccc6c54)c3)nc(-c3cccc4sc5ccccc5c34)n2)cc1] 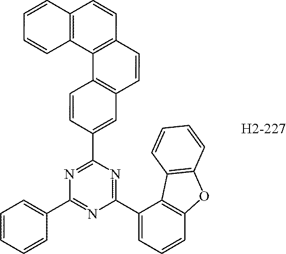 [SMILES: c1ccc(-c2nc(-c3ccc4c(ccc5ccc6ccccc6c54)c3)nc(-c3cccc4oc5ccccc5c34)n2)cc1] 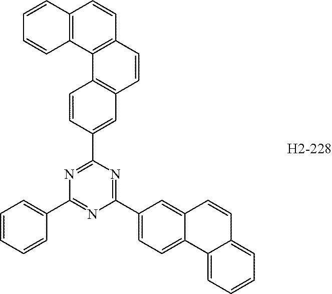 [SMILES: c1ccc(-c2nc(-c3ccc4c(ccc5ccccc54)c3)nc(-c3ccc4c(ccc5ccc6ccccc6c54)c3)n2)cc1] 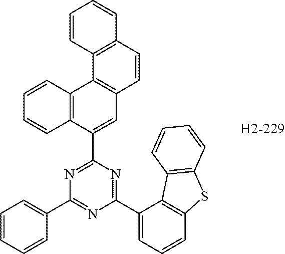 [SMILES: c1ccc(-c2nc(-c3cc4ccc5ccccc5c4c4ccccc34)nc(-c3cccc4sc5ccccc5c34)n2)cc1] 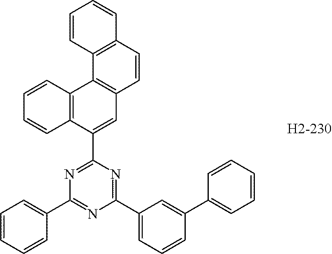 [SMILES: c1ccc(-c2cccc(-c3nc(-c4ccccc4)nc(-c4cc5ccc6ccccc6c5c5ccccc45)n3)c2)cc1] 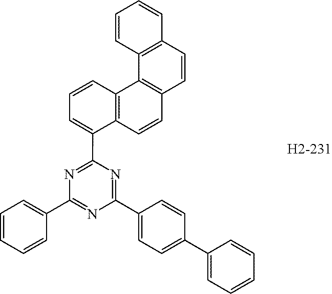 [SMILES: c1ccc(-c2ccc(-c3nc(-c4ccccc4)nc(-c4cccc5c4ccc4ccc6ccccc6c45)n3)cc2)cc1] 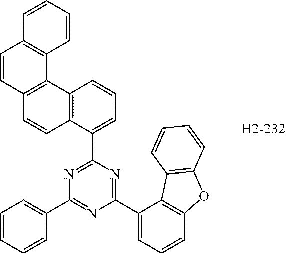 [SMILES: c1ccc(-c2nc(-c3cccc4c3ccc3ccc5ccccc5c34)nc(-c3cccc4oc5ccccc5c34)n2)cc1] 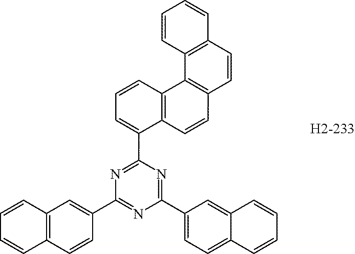 [SMILES: c1ccc2cc(-c3nc(-c4ccc5ccccc5c4)nc(-c4cccc5c4ccc4ccc6ccccc6c45)n3)ccc2c1] 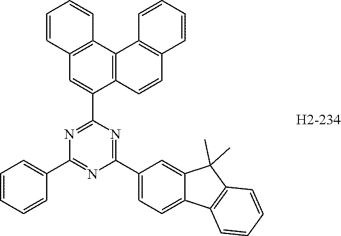 [SMILES: CC1(C)c2ccccc2-c2ccc(-c3nc(-c4ccccc4)nc(-c4cc5ccccc5c5c4ccc4ccccc45)n3)cc21] 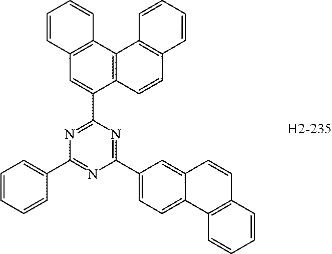 [SMILES: c1ccc(-c2nc(-c3ccc4c(ccc5ccccc54)c3)nc(-c3cc4ccccc4c4c3ccc3ccccc34)n2)cc1] 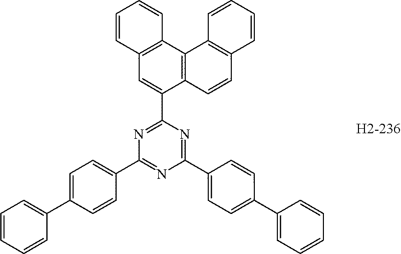 [SMILES: c1ccc(-c2ccc(-c3nc(-c4ccc(-c5ccccc5)cc4)nc(-c4cc5ccccc5c5c4ccc4ccccc45)n3)cc2)cc1] 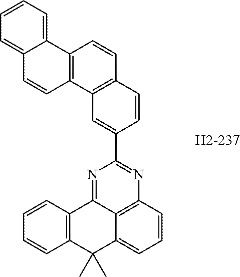 [SMILES: CC1(C)c2ccccc2-c2nc(-c3ccc4ccc5c6ccccc6ccc5c4c3)nc3cccc1c23] 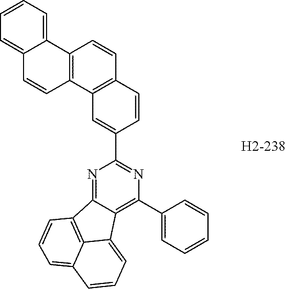 [SMILES: c1ccc(-c2nc(-c3ccc4ccc5c6ccccc6ccc5c4c3)nc3c2-c2cccc4cccc-3c24)cc1] 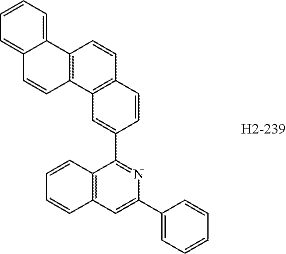 [SMILES: c1ccc(-c2cc3ccccc3c(-c3ccc4ccc5c6ccccc6ccc5c4c3)n2)cc1] 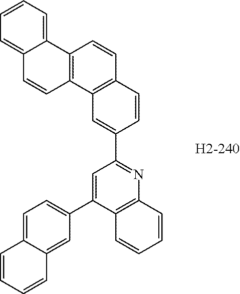 [SMILES: c1ccc2cc(-c3cc(-c4ccc5ccc6c7ccccc7ccc6c5c4)nc4ccccc34)ccc2c1] 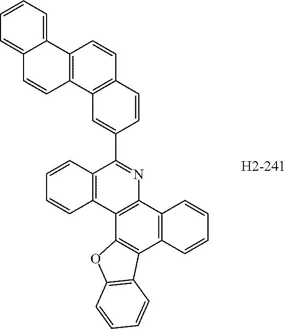 [SMILES: c1ccc2c(c1)ccc1c3cc(-c4nc5c6ccccc6c6c7ccccc7oc6c5c5ccccc45)ccc3ccc21] 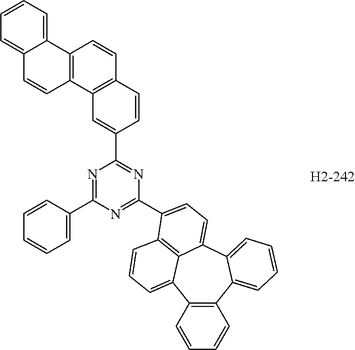 [SMILES: c1ccc(-c2nc(-c3ccc4ccc5c6ccccc6ccc5c4c3)nc(-c3ccc4c5c(cccc35)-c3ccccc3-c3ccccc3-4)n2)cc1] 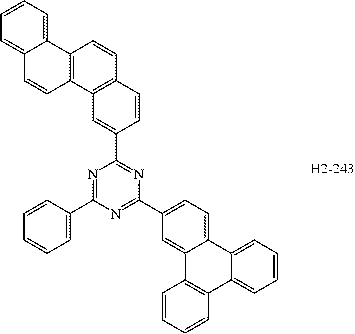 [SMILES: c1ccc(-c2nc(-c3ccc4ccc5c6ccccc6ccc5c4c3)nc(-c3ccc4c5ccccc5c5ccccc5c4c3)n2)cc1] 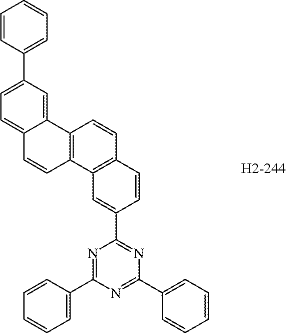 [SMILES: c1ccc(-c2ccc3ccc4c5cc(-c6nc(-c7ccccc7)nc(-c7ccccc7)n6)ccc5ccc4c3c2)cc1] 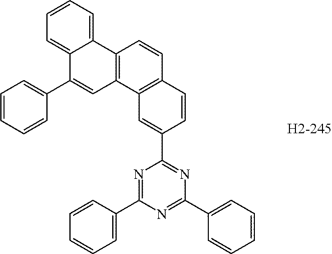 [SMILES: c1ccc(-c2nc(-c3ccccc3)nc(-c3ccc4ccc5c6ccccc6c(-c6ccccc6)cc5c4c3)n2)cc1] 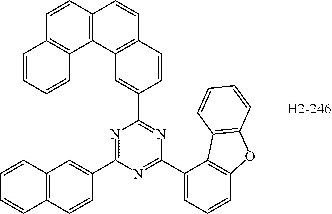 [SMILES: c1ccc2cc(-c3nc(-c4ccc5ccc6ccc7ccccc7c6c5c4)nc(-c4cccc5oc6ccccc6c45)n3)ccc2c1] 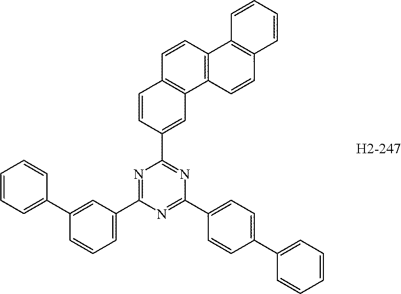 [SMILES: c1ccc(-c2ccc(-c3nc(-c4cccc(-c5ccccc5)c4)nc(-c4ccc5ccc6c7ccccc7ccc6c5c4)n3)cc2)cc1] 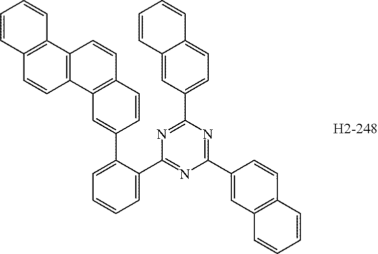 [SMILES: c1ccc(-c2nc(-c3ccc4ccccc4c3)nc(-c3ccc4ccccc4c3)n2)c(-c2ccc3ccc4c5ccccc5ccc4c3c2)c1] 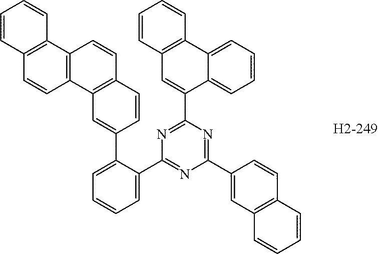 [SMILES: c1ccc(-c2nc(-c3ccc4ccccc4c3)nc(-c3cc4ccccc4c4ccccc34)n2)c(-c2ccc3ccc4c5ccccc5ccc4c3c2)c1] 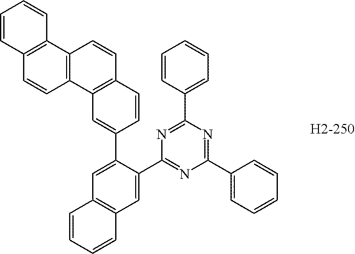 [SMILES: c1ccc(-c2nc(-c3ccccc3)nc(-c3cc4ccccc4cc3-c3ccc4ccc5c6ccccc6ccc5c4c3)n2)cc1] 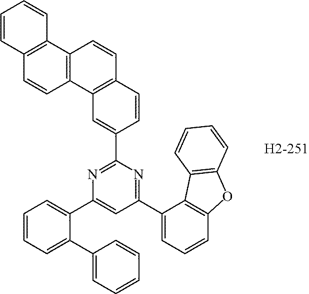 [SMILES: c1ccc(-c2ccccc2-c2cc(-c3cccc4oc5ccccc5c34)nc(-c3ccc4ccc5c6ccccc6ccc5c4c3)n2)cc1] 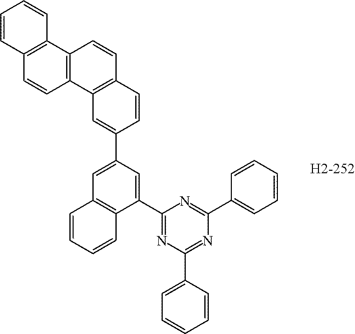 [SMILES: c1ccc(-c2nc(-c3ccccc3)nc(-c3cc(-c4ccc5ccc6c7ccccc7ccc6c5c4)cc4ccccc34)n2)cc1] 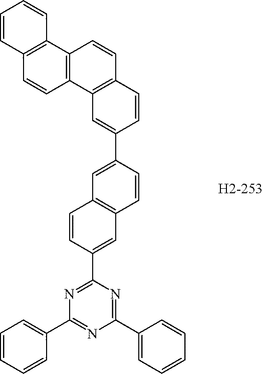 [SMILES: c1ccc(-c2nc(-c3ccccc3)nc(-c3ccc4cc(-c5ccc6ccc7c8ccccc8ccc7c6c5)ccc4c3)n2)cc1] 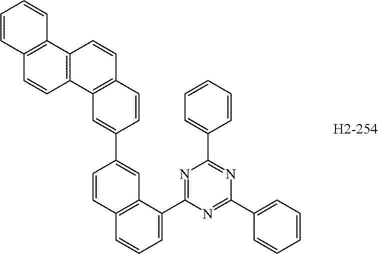 [SMILES: c1ccc(-c2nc(-c3ccccc3)nc(-c3cccc4ccc(-c5ccc6ccc7c8ccccc8ccc7c6c5)cc34)n2)cc1] 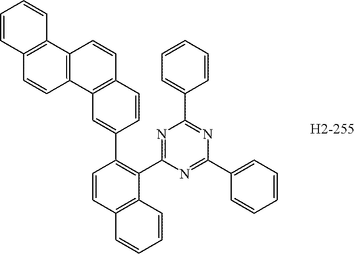 [SMILES: c1ccc(-c2nc(-c3ccccc3)nc(-c3c(-c4ccc5ccc6c7ccccc7ccc6c5c4)ccc4ccccc34)n2)cc1] 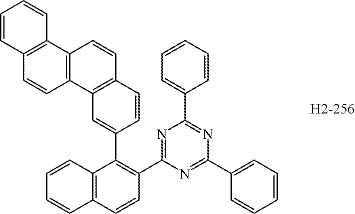 [SMILES: c1ccc(-c2nc(-c3ccccc3)nc(-c3ccc4ccccc4c3-c3ccc4ccc5c6ccccc6ccc5c4c3)n2)cc1] 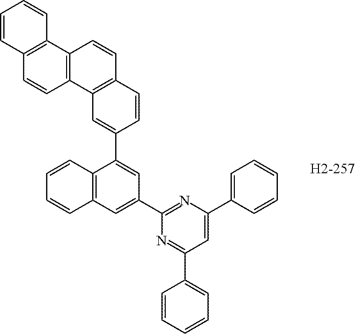 [SMILES: c1ccc(-c2cc(-c3ccccc3)nc(-c3cc(-c4ccc5ccc6c7ccccc7ccc6c5c4)c4ccccc4c3)n2)cc1] 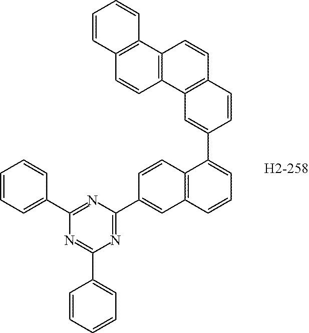 [SMILES: c1ccc(-c2nc(-c3ccccc3)nc(-c3ccc4c(-c5ccc6ccc7c8ccccc8ccc7c6c5)cccc4c3)n2)cc1] 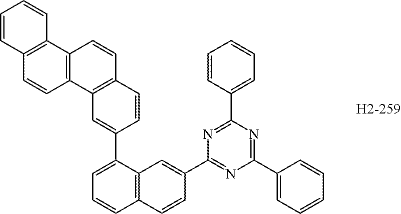 [SMILES: c1ccc(-c2nc(-c3ccccc3)nc(-c3ccc4cccc(-c5ccc6ccc7c8ccccc8ccc7c6c5)c4c3)n2)cc1] 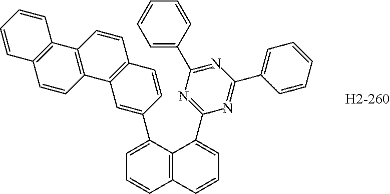 [SMILES: c1ccc(-c2nc(-c3ccccc3)nc(-c3cccc4cccc(-c5ccc6ccc7c8ccccc8ccc7c6c5)c34)n2)cc1] 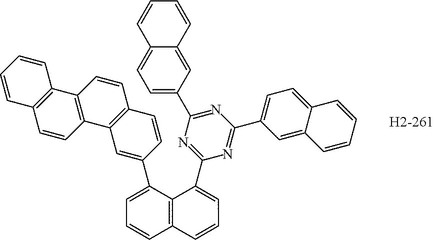 [SMILES: c1ccc2cc(-c3nc(-c4ccc5ccccc5c4)nc(-c4cccc5cccc(-c6ccc7ccc8c9ccccc9ccc8c7c6)c45)n3)ccc2c1] 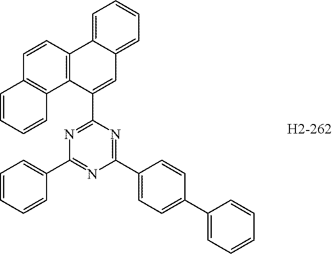 [SMILES: c1ccc(-c2ccc(-c3nc(-c4ccccc4)nc(-c4cc5ccccc5c5ccc6ccccc6c45)n3)cc2)cc1] 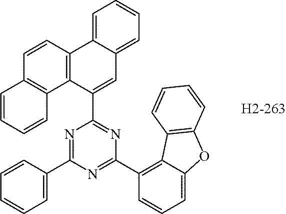 [SMILES: c1ccc(-c2nc(-c3cccc4oc5ccccc5c34)nc(-c3cc4ccccc4c4ccc5ccccc5c34)n2)cc1] 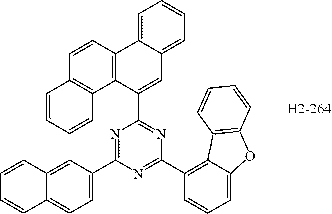 [SMILES: c1ccc2cc(-c3nc(-c4cccc5oc6ccccc6c45)nc(-c4cc5ccccc5c5ccc6ccccc6c45)n3)ccc2c1] 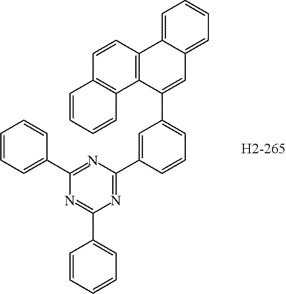 [SMILES: c1ccc(-c2nc(-c3ccccc3)nc(-c3cccc(-c4cc5ccccc5c5ccc6ccccc6c45)c3)n2)cc1] 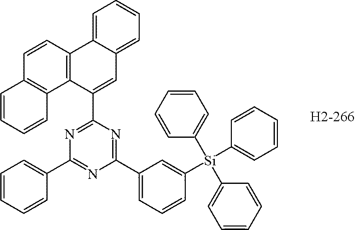 [SMILES: c1ccc(-c2nc(-c3cccc([Si](c4ccccc4)(c4ccccc4)c4ccccc4)c3)nc(-c3cc4ccccc4c4ccc5ccccc5c34)n2)cc1] 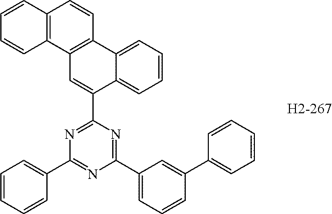 [SMILES: c1ccc(-c2cccc(-c3nc(-c4ccccc4)nc(-c4cc5c6ccccc6ccc5c5ccccc45)n3)c2)cc1] 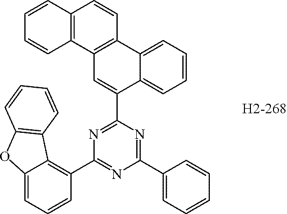 [SMILES: c1ccc(-c2nc(-c3cc4c5ccccc5ccc4c4ccccc34)nc(-c3cccc4oc5ccccc5c34)n2)cc1] 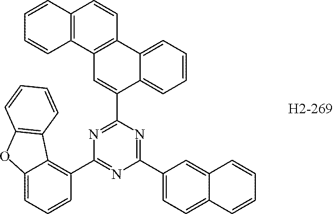 [SMILES: c1ccc2cc(-c3nc(-c4cc5c6ccccc6ccc5c5ccccc45)nc(-c4cccc5oc6ccccc6c45)n3)ccc2c1] 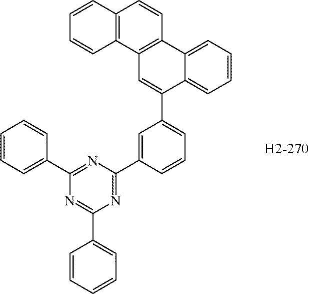 [SMILES: c1ccc(-c2nc(-c3ccccc3)nc(-c3cccc(-c4cc5c6ccccc6ccc5c5ccccc45)c3)n2)cc1] 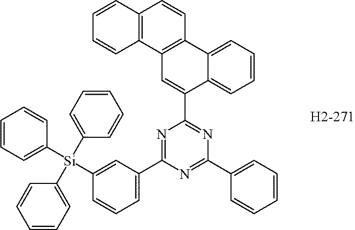 [SMILES: c1ccc(-c2nc(-c3cccc([Si](c4ccccc4)(c4ccccc4)c4ccccc4)c3)nc(-c3cc4c5ccccc5ccc4c4ccccc34)n2)cc1] 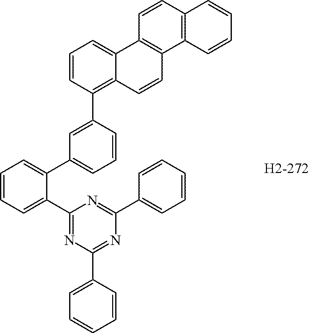 [SMILES: c1ccc(-c2nc(-c3ccccc3)nc(-c3ccccc3-c3cccc(-c4cccc5c4ccc4c6ccccc6ccc54)c3)n2)cc1] 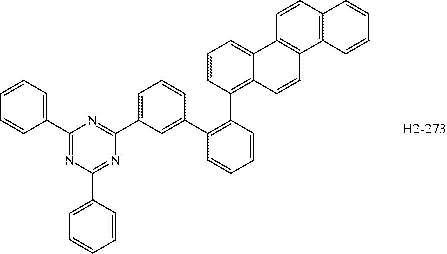 [SMILES: c1ccc(-c2nc(-c3ccccc3)nc(-c3cccc(-c4ccccc4-c4cccc5c4ccc4c6ccccc6ccc54)c3)n2)cc1] 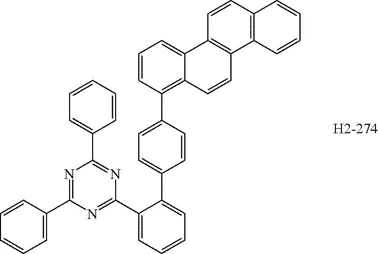 [SMILES: c1ccc(-c2nc(-c3ccccc3)nc(-c3ccccc3-c3ccc(-c4cccc5c4ccc4c6ccccc6ccc54)cc3)n2)cc1] 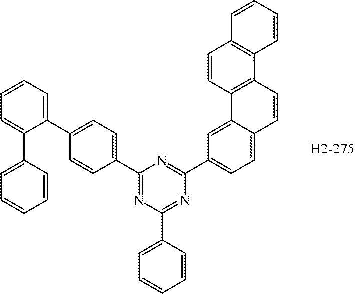 [SMILES: c1ccc(-c2nc(-c3ccc(-c4ccccc4-c4ccccc4)cc3)nc(-c3ccc4ccc5c6ccccc6ccc5c4c3)n2)cc1] 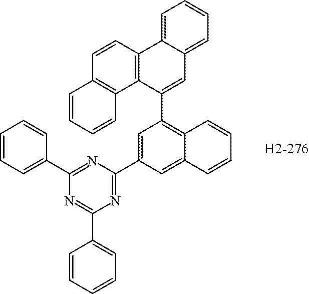 [SMILES: c1ccc(-c2nc(-c3ccccc3)nc(-c3cc(-c4cc5ccccc5c5ccc6ccccc6c45)c4ccccc4c3)n2)cc1] 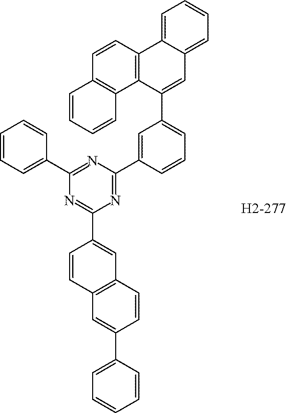 [SMILES: c1ccc(-c2ccc3cc(-c4nc(-c5ccccc5)nc(-c5cccc(-c6cc7ccccc7c7ccc8ccccc8c67)c5)n4)ccc3c2)cc1] 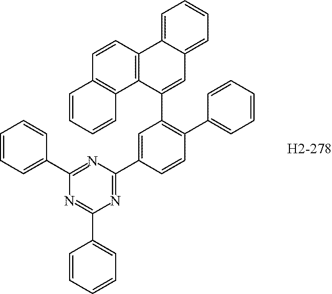 [SMILES: c1ccc(-c2nc(-c3ccccc3)nc(-c3ccc(-c4ccccc4)c(-c4cc5ccccc5c5ccc6ccccc6c45)c3)n2)cc1] 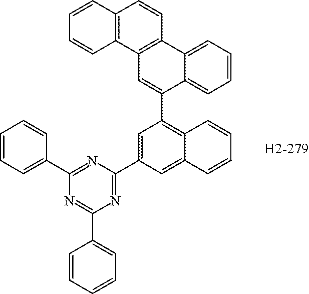 [SMILES: c1ccc(-c2nc(-c3ccccc3)nc(-c3cc(-c4cc5c6ccccc6ccc5c5ccccc45)c4ccccc4c3)n2)cc1] 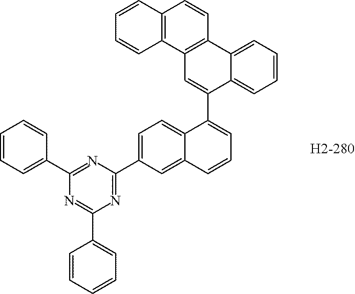 [SMILES: c1ccc(-c2nc(-c3ccccc3)nc(-c3ccc4c(-c5cc6c7ccccc7ccc6c6ccccc56)cccc4c3)n2)cc1] 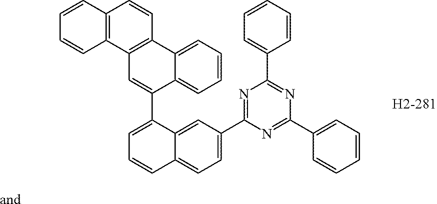
.

10. An organic electroluminescent device comprising an anode, a cathode, and at least one light-emitting layer between the anode and the cathode, wherein the at least one light-emitting layer comprises the plurality of host materials according to claim 1.

11. An organic electroluminescent material comprising an organic electroluminescent compound represented by the following formula 1: 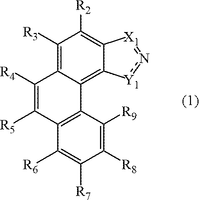
in formula 1,
X₁ and Y₁ each independently represent -O-, -S- or -CR₁=,
provided that one of X₁ and Y₁ represents -CR₁=, and the other of X₁ and Y₁ represents -O- or -S-;
R₁ represents a substituted or unsubstituted (C1-C30)alkyl, a substituted or unsubstituted (C6-C30)aryl, or a substituted or unsubstituted (3- to 30-membered)heteroaryl;
R₂ to R₉ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), a substituted or unsubstituted mono-or di- (C1-C30)alkylamino, a substituted or unsubstituted mono- or di- (C2-C30)alkenylamino, a substituted or unsubstituted (C1-C30)alkyl(C2-C30)alkenylamino, a substituted or unsubstituted mono- or di- (C6-C30)arylamino, a substituted or unsubstituted (C1-C30)alkyl(C6-C30)arylamino, a substituted or unsubstituted mono- or di- (3- to 30-membered)heteroarylamino, a substituted or unsubstituted (C1-C30)alkyl(3- to 30-membered)heteroarylamino, a substituted or unsubstituted (C2-C30)alkenyl(C6-C30) arylamino, a substituted or unsubstituted (C2-C30)alkenyl(3- to 30-membered)heteroarylamino, a substituted or unsubstituted (C6-C30)aryl(3- to 30-membered)heteroarylamino, or -L₁-N(Ar₁)(Ar₂); or may be linked to an adjacent substituent(s) to form a ring(s);
L₁ each independently represents a single bond, a substituted or unsubstituted (C6-C30)arylene, or a substituted or unsubstituted (3- to 30-membered)heteroarylene; and
Ar₁ and Ar₂ each independently represent a substituted or unsubstituted (C6-C30) aryl, or a substituted or unsubstituted (3- to 30-membered)heteroaryl.

12. The organic electroluminescent material according to claim 11, wherein the compound represented by formula 1 is at least one selected from the following compounds: 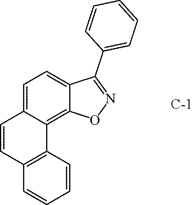 [SMILES: c1ccc(-c2noc3c2ccc2ccc4ccccc4c23)cc1] 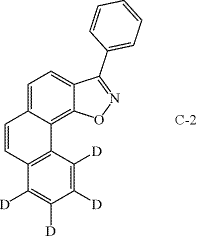 [SMILES: [2H]c1c([2H])c([2H])c2c(ccc3ccc4c(-c5ccccc5)noc4c32)c1[2H]] 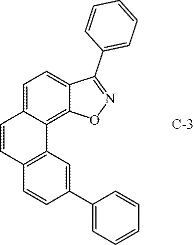 [SMILES: c1ccc(-c2ccc3ccc4ccc5c(-c6ccccc6)noc5c4c3c2)cc1] 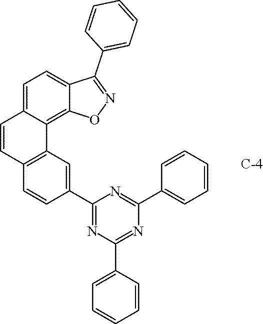 [SMILES: c1ccc(-c2nc(-c3ccccc3)nc(-c3ccc4ccc5ccc6c(-c7ccccc7)noc6c5c4c3)n2)cc1] 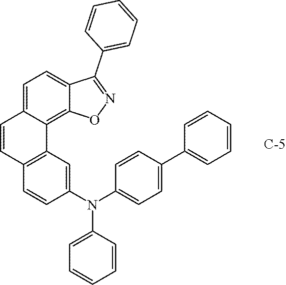 [SMILES: c1ccc(-c2ccc(N(c3ccccc3)c3ccc4ccc5ccc6c(-c7ccccc7)noc6c5c4c3)cc2)cc1] 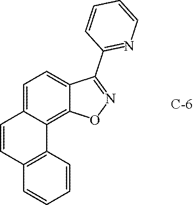 [SMILES: c1ccc(-c2noc3c2ccc2ccc4ccccc4c23)nc1] 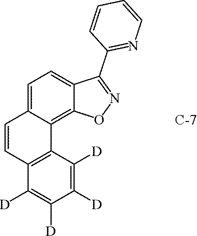 [SMILES: [2H]c1c([2H])c([2H])c2c(ccc3ccc4c(-c5ccccn5)noc4c32)c1[2H]] 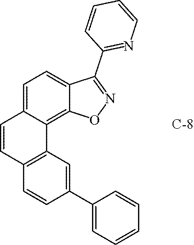 [SMILES: c1ccc(-c2ccc3ccc4ccc5c(-c6ccccn6)noc5c4c3c2)cc1] 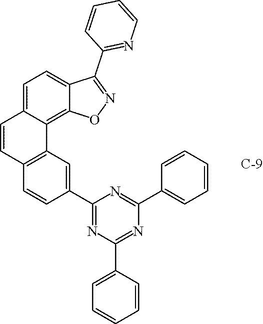 [SMILES: c1ccc(-c2nc(-c3ccccc3)nc(-c3ccc4ccc5ccc6c(-c7ccccn7)noc6c5c4c3)n2)cc1] 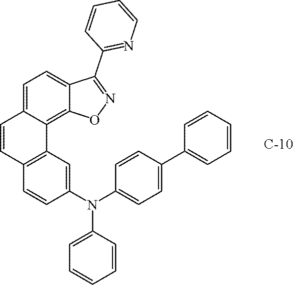 [SMILES: c1ccc(-c2ccc(N(c3ccccc3)c3ccc4ccc5ccc6c(-c7ccccn7)noc6c5c4c3)cc2)cc1] 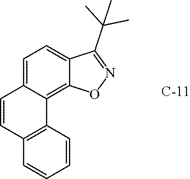 [SMILES: CC(C)(C)c1noc2c1ccc1ccc3ccccc3c12] 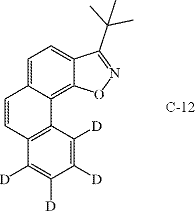 [SMILES: [2H]c1c([2H])c([2H])c2c(ccc3ccc4c(C(C)(C)C)noc4c32)c1[2H]] 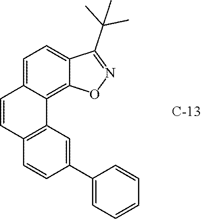 [SMILES: CC(C)(C)c1noc2c1ccc1ccc3ccc(-c4ccccc4)cc3c12] 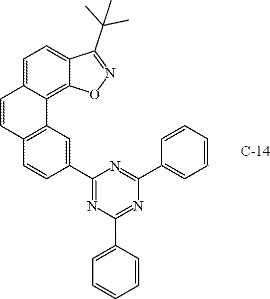 [SMILES: CC(C)(C)c1noc2c1ccc1ccc3ccc(-c4nc(-c5ccccc5)nc(-c5ccccc5)n4)cc3c12] 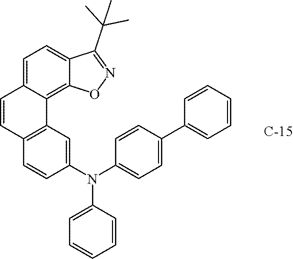 [SMILES: CC(C)(C)c1noc2c1ccc1ccc3ccc(N(c4ccccc4)c4ccc(-c5ccccc5)cc4)cc3c12] 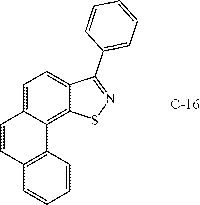 [SMILES: c1ccc(-c2nsc3c2ccc2ccc4ccccc4c23)cc1] 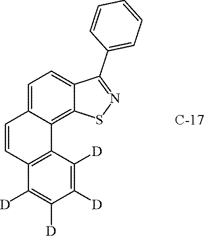 [SMILES: [2H]c1c([2H])c([2H])c2c(ccc3ccc4c(-c5ccccc5)nsc4c32)c1[2H]] 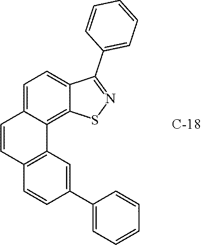 [SMILES: c1ccc(-c2ccc3ccc4ccc5c(-c6ccccc6)nsc5c4c3c2)cc1] 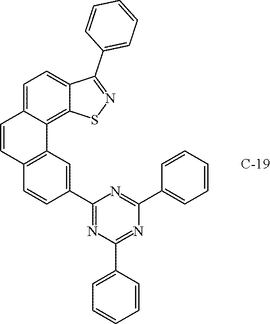 [SMILES: c1ccc(-c2nc(-c3ccccc3)nc(-c3ccc4ccc5ccc6c(-c7ccccc7)nsc6c5c4c3)n2)cc1] 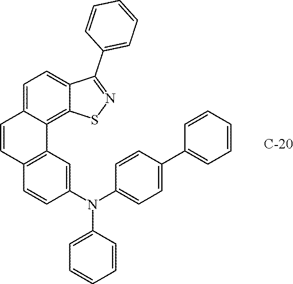 [SMILES: c1ccc(-c2ccc(N(c3ccccc3)c3ccc4ccc5ccc6c(-c7ccccc7)nsc6c5c4c3)cc2)cc1] 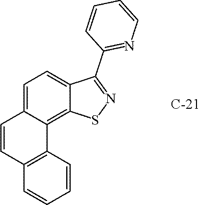 [SMILES: c1ccc(-c2nsc3c2ccc2ccc4ccccc4c23)nc1] 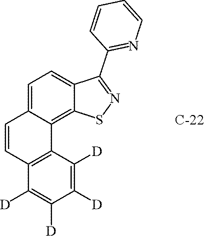 [SMILES: [2H]c1c([2H])c([2H])c2c(ccc3ccc4c(-c5ccccn5)nsc4c32)c1[2H]] 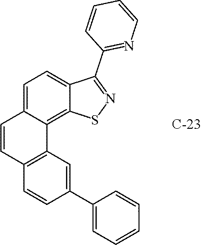 [SMILES: c1ccc(-c2ccc3ccc4ccc5c(-c6ccccn6)nsc5c4c3c2)cc1] 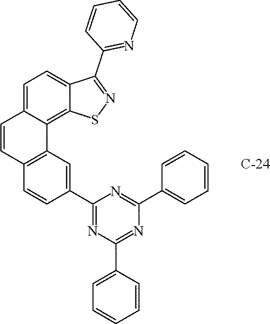 [SMILES: c1ccc(-c2nc(-c3ccccc3)nc(-c3ccc4ccc5ccc6c(-c7ccccn7)nsc6c5c4c3)n2)cc1] 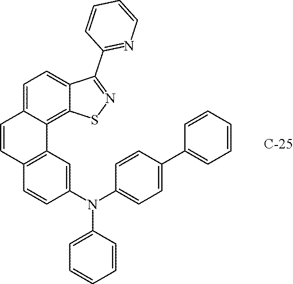 [SMILES: c1ccc(-c2ccc(N(c3ccccc3)c3ccc4ccc5ccc6c(-c7ccccn7)nsc6c5c4c3)cc2)cc1] 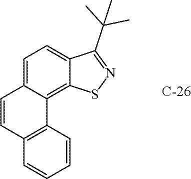 [SMILES: CC(C)(C)c1nsc2c1ccc1ccc3ccccc3c12] 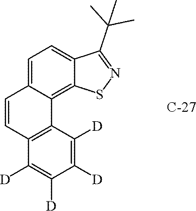 [SMILES: [2H]c1c([2H])c([2H])c2c(ccc3ccc4c(C(C)(C)C)nsc4c32)c1[2H]] 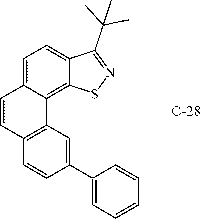 [SMILES: CC(C)(C)c1nsc2c1ccc1ccc3ccc(-c4ccccc4)cc3c12] 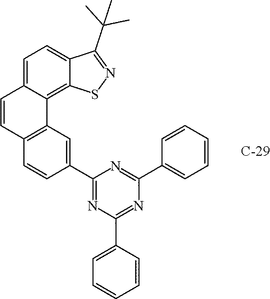 [SMILES: CC(C)(C)c1nsc2c1ccc1ccc3ccc(-c4nc(-c5ccccc5)nc(-c5ccccc5)n4)cc3c12] 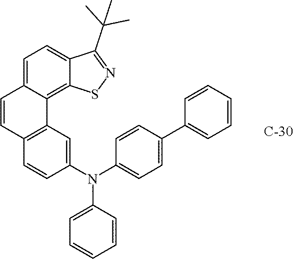 [SMILES: CC(C)(C)c1nsc2c1ccc1ccc3ccc(N(c4ccccc4)c4ccc(-c5ccccc5)cc4)cc3c12] 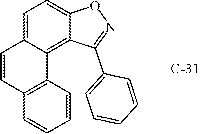 [SMILES: c1ccc(-c2noc3ccc4ccc5ccccc5c4c23)cc1] 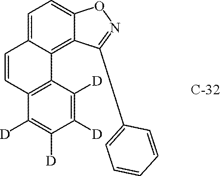 [SMILES: [2H]c1c([2H])c([2H])c2c(ccc3ccc4onc(-c5ccccc5)c4c32)c1[2H]] 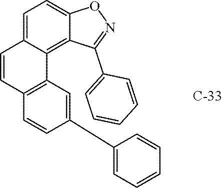 [SMILES: c1ccc(-c2ccc3ccc4ccc5onc(-c6ccccc6)c5c4c3c2)cc1] 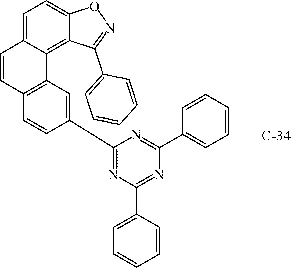 [SMILES: c1ccc(-c2nc(-c3ccccc3)nc(-c3ccc4ccc5ccc6onc(-c7ccccc7)c6c5c4c3)n2)cc1] 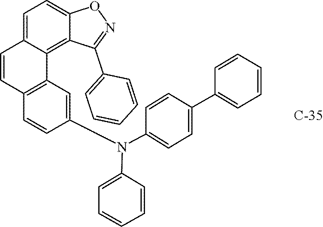 [SMILES: c1ccc(-c2ccc(N(c3ccccc3)c3ccc4ccc5ccc6onc(-c7ccccc7)c6c5c4c3)cc2)cc1] 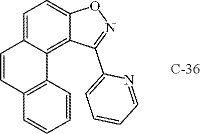 [SMILES: c1ccc(-c2noc3ccc4ccc5ccccc5c4c23)nc1] 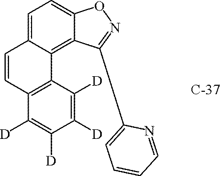 [SMILES: [2H]c1c([2H])c([2H])c2c(ccc3ccc4onc(-c5ccccn5)c4c32)c1[2H]] 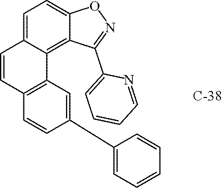 [SMILES: c1ccc(-c2ccc3ccc4ccc5onc(-c6ccccn6)c5c4c3c2)cc1] 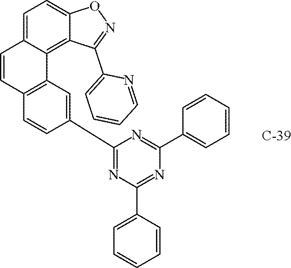 [SMILES: c1ccc(-c2nc(-c3ccccc3)nc(-c3ccc4ccc5ccc6onc(-c7ccccn7)c6c5c4c3)n2)cc1] 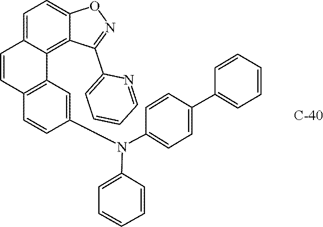 [SMILES: c1ccc(-c2ccc(N(c3ccccc3)c3ccc4ccc5ccc6onc(-c7ccccn7)c6c5c4c3)cc2)cc1] 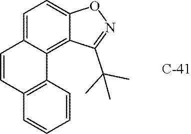 [SMILES: CC(C)(C)c1noc2ccc3ccc4ccccc4c3c12] 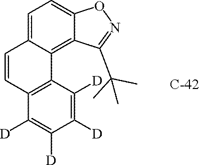 [SMILES: [2H]c1c([2H])c([2H])c2c(ccc3ccc4onc(C(C)(C)C)c4c32)c1[2H]] 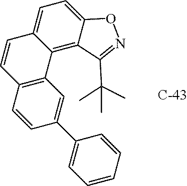 [SMILES: CC(C)(C)c1noc2ccc3ccc4ccc(-c5ccccc5)cc4c3c12] 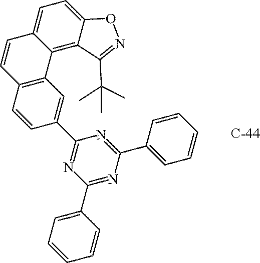 [SMILES: CC(C)(C)c1noc2ccc3ccc4ccc(-c5nc(-c6ccccc6)nc(-c6ccccc6)n5)cc4c3c12] 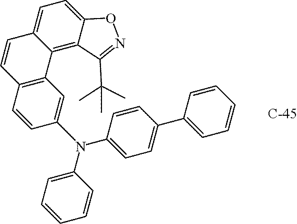 [SMILES: CC(C)(C)c1noc2ccc3ccc4ccc(N(c5ccccc5)c5ccc(-c6ccccc6)cc5)cc4c3c12] 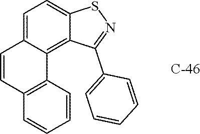 [SMILES: c1ccc(-c2nsc3ccc4ccc5ccccc5c4c23)cc1] 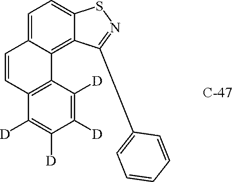 [SMILES: [2H]c1c([2H])c([2H])c2c(ccc3ccc4snc(-c5ccccc5)c4c32)c1[2H]] 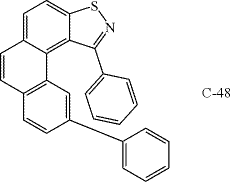 [SMILES: c1ccc(-c2ccc3ccc4ccc5snc(-c6ccccc6)c5c4c3c2)cc1] 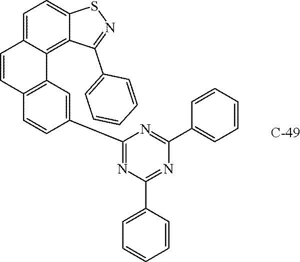 [SMILES: c1ccc(-c2nc(-c3ccccc3)nc(-c3ccc4ccc5ccc6snc(-c7ccccc7)c6c5c4c3)n2)cc1] 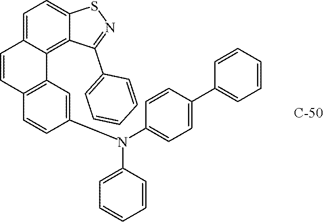 [SMILES: c1ccc(-c2ccc(N(c3ccccc3)c3ccc4ccc5ccc6snc(-c7ccccc7)c6c5c4c3)cc2)cc1] 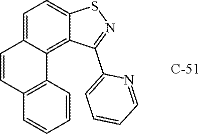 [SMILES: c1ccc(-c2nsc3ccc4ccc5ccccc5c4c23)nc1] 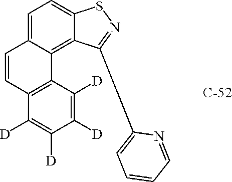 [SMILES: [2H]c1c([2H])c([2H])c2c(ccc3ccc4snc(-c5ccccn5)c4c32)c1[2H]] 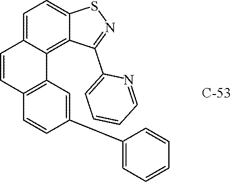 [SMILES: c1ccc(-c2ccc3ccc4ccc5snc(-c6ccccn6)c5c4c3c2)cc1] 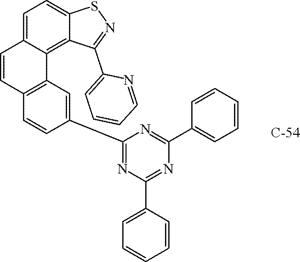 [SMILES: c1ccc(-c2nc(-c3ccccc3)nc(-c3ccc4ccc5ccc6snc(-c7ccccn7)c6c5c4c3)n2)cc1] 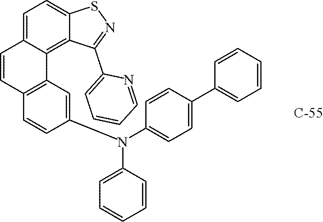 [SMILES: c1ccc(-c2ccc(N(c3ccccc3)c3ccc4ccc5ccc6snc(-c7ccccn7)c6c5c4c3)cc2)cc1] 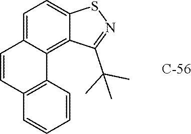 [SMILES: CC(C)(C)c1nsc2ccc3ccc4ccccc4c3c12] 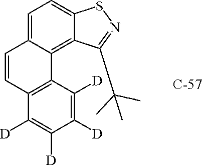 [SMILES: [2H]c1c([2H])c([2H])c2c(ccc3ccc4snc(C(C)(C)C)c4c32)c1[2H]] 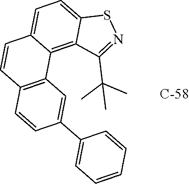 [SMILES: CC(C)(C)c1nsc2ccc3ccc4ccc(-c5ccccc5)cc4c3c12] 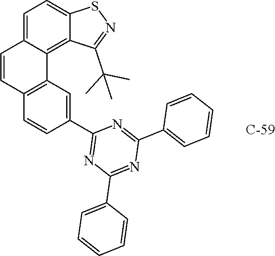 [SMILES: CC(C)(C)c1nsc2ccc3ccc4ccc(-c5nc(-c6ccccc6)nc(-c6ccccc6)n5)cc4c3c12] 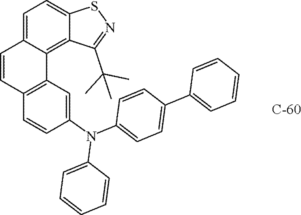 [SMILES: CC(C)(C)c1nsc2ccc3ccc4ccc(N(c5ccccc5)c5ccc(-c6ccccc6)cc5)cc4c3c12] 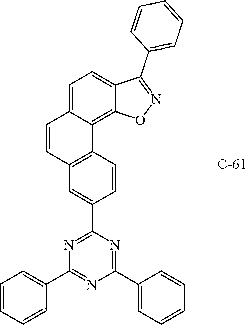 [SMILES: c1ccc(-c2nc(-c3ccccc3)nc(-c3ccc4c(ccc5ccc6c(-c7ccccc7)noc6c54)c3)n2)cc1] 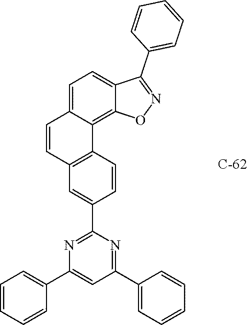 [SMILES: c1ccc(-c2cc(-c3ccccc3)nc(-c3ccc4c(ccc5ccc6c(-c7ccccc7)noc6c54)c3)n2)cc1] 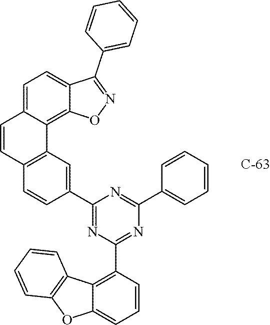 [SMILES: c1ccc(-c2nc(-c3ccc4ccc5ccc6c(-c7ccccc7)noc6c5c4c3)nc(-c3cccc4oc5ccccc5c34)n2)cc1] 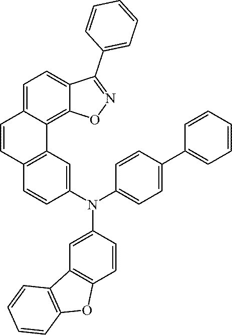 [SMILES: c1ccc(-c2ccc(N(c3ccc4oc5ccccc5c4c3)c3ccc4ccc5ccc6c(-c7ccccc7)noc6c5c4c3)cc2)cc1] 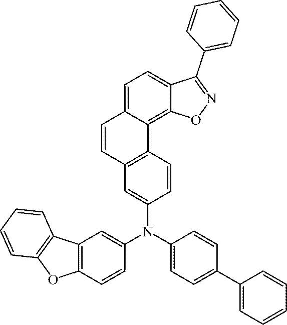 [SMILES: c1ccc(-c2ccc(N(c3ccc4c(ccc5ccc6c(-c7ccccc7)noc6c54)c3)c3ccc4oc5ccccc5c4c3)cc2)cc1] 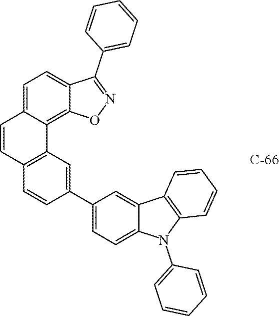 [SMILES: c1ccc(-c2noc3c2ccc2ccc4ccc(-c5ccc6c(c5)c5ccccc5n6-c5ccccc5)cc4c23)cc1] 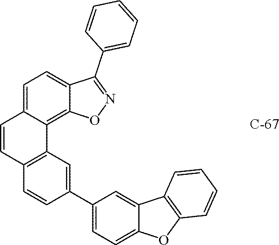 [SMILES: c1ccc(-c2noc3c2ccc2ccc4ccc(-c5ccc6oc7ccccc7c6c5)cc4c23)cc1] 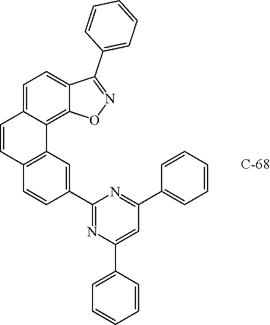 [SMILES: c1ccc(-c2cc(-c3ccccc3)nc(-c3ccc4ccc5ccc6c(-c7ccccc7)noc6c5c4c3)n2)cc1] 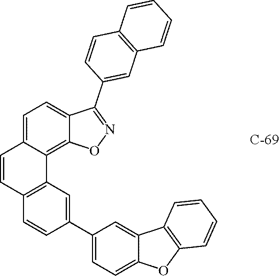 [SMILES: c1ccc2cc(-c3noc4c3ccc3ccc5ccc(-c6ccc7oc8ccccc8c7c6)cc5c34)ccc2c1] 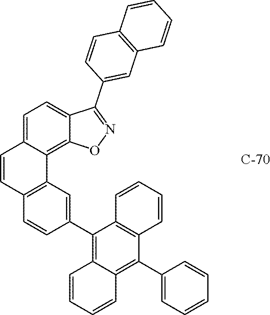 [SMILES: c1ccc(-c2c3ccccc3c(-c3ccc4ccc5ccc6c(-c7ccc8ccccc8c7)noc6c5c4c3)c3ccccc23)cc1] 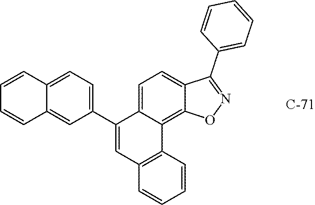 [SMILES: c1ccc(-c2noc3c2ccc2c(-c4ccc5ccccc5c4)cc4ccccc4c23)cc1] 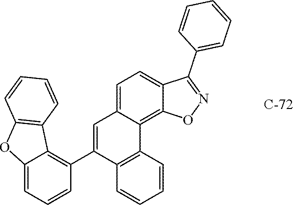 [SMILES: c1ccc(-c2noc3c2ccc2cc(-c4cccc5oc6ccccc6c45)c4ccccc4c23)cc1] 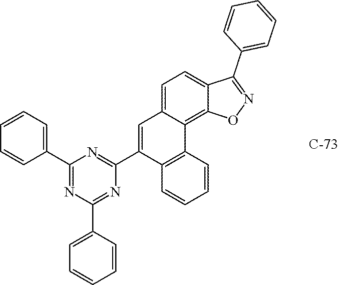 [SMILES: c1ccc(-c2nc(-c3ccccc3)nc(-c3cc4ccc5c(-c6ccccc6)noc5c4c4ccccc34)n2)cc1] 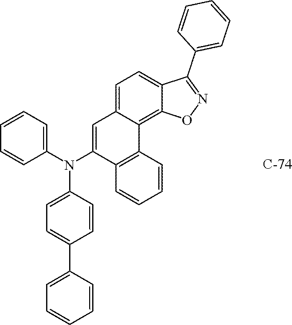 [SMILES: c1ccc(-c2ccc(N(c3ccccc3)c3cc4ccc5c(-c6ccccc6)noc5c4c4ccccc34)cc2)cc1]
and 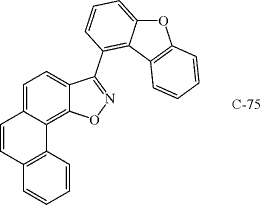 [SMILES: c1ccc2c(c1)ccc1ccc3c(-c4cccc5oc6ccccc6c45)noc3c12]
.

13. An organic electroluminescent device comprising an organic electroluminescent material according to claim 11.
